(19) United States
(12) Patent Application Publication
(10) Pub. No.: US 2019/0309273 A1
(43) Pub. Date: Oct. 10, 2019

(54) Variants of Phosphotriesterase for the Hydrolysis and Detoxification of Nerve Agents

(71) Applicant: The Texas A&M University System, College Station, TX (US)

(72) Inventors: Frank M. Raushel, College Station, TX (US); Andrew N. Bigley, North Zulch, TX (US)

(73) Assignee: The Texas A&M University System, College Station, TX (US)

(21) Appl. No.: 16/423,105

(22) Filed: May 27, 2019

Related U.S. Application Data
(63) Continuation-in-part of application No. 15/320,490, filed on Dec. 20, 2016, now Pat. No. 10,301,608.
(60) Provisional application No. 62/015,156, filed on Jun. 20, 2014.

(51) Int. Cl. C12N 9/16; A62D 3/02

(52) U.S. Cl. CPC C12N 9/16; A61K 38/465; C12Y 301/08001; A62D 3/02

(57) ABSTRACT

Variants of phosphotriesterase have been created that exhibit enhanced hydrolysis of V-type and G-type nerve agents over wild-type phosphotriesterase. V- and G-type nerve agents have an S_{P} and R_{P} enantiomer. The S_{P} enantiomers are more toxic. V-type nerve agents are among the most toxic substances known. Variants of phosphotriesterase can prefer to hydrolyze one enantiomer of VX over the other enantiomer.

### CROSS-REFERENCE TO RELATED APPLICATIONS

This application is a continuation-in-part of U.S. patent application Ser. No. 15/320,490, filed on Dec. 20, 2016, to be issued as U.S. Pat. No. 10,301,608 on May 28, 2019; which is the national stage of International Application PCT/US2015/036745, filed Jun. 19, 2015; which claims the benefit of U.S. Provisional Application No. 62/015,156, filed Jun. 20, 2014; all of which are incorporated by reference in their entirety.

### GOVERNMENT LICENSE RIGHTS

This invention was made with government support under GM068550 awarded by National Institutes of Health and under HDTRA1-14-1-0004 and CB3742 awarded by the Defense Threat Reduction Agency. The government has certain rights in the invention.

### FIELD

The disclosure relates generally to chemical warfare. More specifically, the disclosure relates to detoxification of organophosphate nerve agents.

### BACKGROUND

The G-type (sarin, cyclosarin, and soman) and V-type (VX and VR) organophosphonates are among the most toxic compounds known. The toxicity of these compounds is due to their ability to inactivate acetylcholine esterase, an enzyme required for proper nerve function.(1) Acetylcholine esterase breaks down the neurotransmitter acetylcholine into acetic acid and choline. Acetylcholine conducts a nerve impulse between the nerve and the muscle, stimulating the muscle. The organophosphonate binds to the hydroxyl group on a serine at a binding site on acetylcholine esterase, preventing acetylcholine from binding at that site. If acetylcholine esterase is inhibited by an organophosphonate, acetylcholine builds up at the synapses and neuromuscular junctions and the receptor is desensitized resulting in paralysis with an estimated lethal dermal exposure of about 6 milligrams of VX for an average human.

Contact with VX is about 200-fold more toxic than soman (GD) and 300-fold more toxic than sarin (GB).(2) The extreme potential for acute toxicity with VX is due, in part, to the low volatility of this compound, which allows it to persist indefinitely on common surfaces.(3) Methods currently utilized for the destruction of organophosphate nerve agents include high temperature incineration and treatment with strong base or concentrated bleach.(4,5) Medical treatment of VX toxicity is currently limited to the injection of atropine, which reduces neurological symptoms, and oximes, which can help to reactivate the inactivated acetylcholine esterase.(2) Butyrylcholine esterase, which is closely related to acetylcholine esterase, has proven effective in animal models as a stoichiometric scavenger of VX.(6) However, the large amount of enzyme required for treatment with a stoichiometric scavenger, and the limited supply of this protein, have prevented butyrylcholine esterase from being an effective antidote for medical use.(7,8)

Enzymatic hydrolysis of nerve agents provides numerous advantages over harsh physical or chemical methods of decontamination and could provide a catalytic antidote for medical use. Enzymes such as organophosphorus acid anhydrolase (OPAA), diisopropyl-fluorophosphatase (DFPase), and human paraoxonase (PON1) can hydrolytically neutralize the various G-type agents, (9,10,11,12) but except for PON1, they have no activity against the V-type agents.(11)

The enzyme phosphotriesterase (PTE) is capable of hydrolyzing a wide variety of organophosphonates including both the G-type and V-type nerve agents.(13,14) A substrate for PTE, the insecticide paraoxon (FIG. 3F) has an enzymatic efficiency that approaches the limits of diffusion (k_{cat}/Kₘ˜10⁸ M⁻¹ s⁻¹).(15)

The high toxicity and environmental persistence of VX makes the development of novel decontamination methods particularly important. PTE is capable of hydrolyzing VX. The enzymatic efficiency of PTE for VX is more than 5-orders of magnitude lower than with paraoxon. For the hydrolysis of the G-type agents by PTE, the values of k_{cat}/Kₘ are between 10⁴ and 10⁵ M⁻¹ s⁻¹.(13)

The G- and V-type nerve agents all contain a chiral phosphorus center where the S_{P}-enantiomers are significantly more toxic than the corresponding R_{P}-enantiomers.(16,17) In general, wild-type PTE preferentially hydrolyzes the R_{P}-enantiomers of these compounds. The overall selectivity depends on the relative size of the substituents attached to the phosphorus center, with larger differences in size resulting in greater stereoselectivity.(18)

Chiral chromophoric analogues of the G-type agents have been utilized to guide the evolution of PTE for the identification of variants that prefer the more toxic S_{P}-enantiomers of sarin, cyclosarin, and soman.(13,16,18) The catalytic activity of PTE for the more toxic S_{P}-enantiomer of cyclosarin (GF) has been increased by more than 4-orders of magnitude.(13) The catalytic efficiencies for the hydrolysis of the more toxic S_{P}-enantiomers by the enhanced variants of PTE for the hydrolysis of GB, GD, and GF approach 10⁶ M⁻¹ s⁻¹.(13)

Unfortunately, the activity of PTE against the V-type agents is about 3-orders of magnitude lower than that with the G-type agents (k_{cat}/Kₘ<10³M⁻¹s⁻¹). (14, 19). The net rate of VX hydrolysis by PTE is thought to be limited more by the chemistry of the leaving group than by the stereochemistry of the phosphorus center.(13,14,20) The X-ray crystal structure of PTE shows that this enzyme folds as a distorted (β/α)₈-barrel and that the bulk of the active site is formed from the 8 loops that connect the core β-strands to the subsequent α-helices.(21)

The twelve residues which make up the substrate binding site of PTE can be subdivided into three pockets that accommodate the small, large and leaving-group moieties of the substrate. (21)

The residues in the active site have been shown to be largely responsible for the observed substrate specificity.(22) Loop-7 is the largest of the loops that contribute to the substrate binding site, and is known to tolerate substantial sequence variation.(18,21,23) Previous attempts to evolve PTE for the hydrolysis of VX have utilized the insecticide demeton-S with modest success.(24,25)

The bacterial enzyme phosphotriesterase (PTE) from Pseudomonas diminuta has been the subject of extensive interrogation due to its ability to hydrolyze a wide array of neurotoxic organophosphate compounds.(57-60) The best substrates for PTE are organophosphate insecticides, such as paraoxon (FIG. 23A), where the catalytic efficiency can approach the limits of diffusion.(61) With other insecticides, such as chlorpyrifos, methyl parathion, and malathion, the activity of the wild-type enzyme is much lower, but evolution of the enzyme for enhanced hydrolysis of these compounds has proven highly successful, enabling PTE to be used for commercial bioremediation.(62-64) A more challenging, but equally successful effort, was made to evolve PTE for the decontamination of the G-type nerve agents, sarin and soman.(58, 65) Unlike most insecticides, the G-type nerve agents are chiral, and the utilization of substrate analogs was required to evolve variants of PTE with the appropriate stereochemical preferences.(58, 66) The PTE variant H257Y/L303T (YT) with a catalytic efficiency of 2×10⁶M⁻¹ s⁻¹ for the hydrolysis of sarin (GB) has proven highly effective for the detoxification of G-type nerve agents.(58) The YT variant has now been successfully adapted for in vivo prophylactic protection against G-agent intoxication, and shown to prevent toxicity from multiple exposures of GB for up to 7 days in animal models.(67)

The V-type agents are among the most toxic compounds ever synthesized and the toxicity of the S_{P}-enantiomers is at least 2-orders of magnitude greater than that of the R_{P}-enantiomers.(58, 66, 68) The catalytic efficiency of wild-type PTE (k_{cat}/Kₘ˜10²M⁻¹ s⁻¹ for R_{P}-VX and R_{P}-VR) falls approximately 3-orders of magnitude below that seen with the G-agents and the stereochemical preference is for the less toxic enantiomer.(57) In the challenge of modifying PTE for V-agent hydrolysis, the chemical step of the reaction must be optimized for P—S bond cleavage and the unresolved synergy amongst residues in the active site is likely to be a major hurdle. This situation is further complicated by the need to use substrate analogs for screening purposes and the vast number of variants that must be screened.

Enzyme evolution efforts have been conducted by four research groups using differing mutational strategies including rational design,(69) site-saturation mutagenesis(63,70), loop-targeted error prone PCR,(70) and computational design.(71) Excluding mutations introduced specifically to enhance protein stability, these efforts have identified modifications at 19 different amino acid positions with two to six possible amino acid residues at each site. In each case, multiple rounds of evolution were initiated. The residue positions have been targeted in differing order and often multiple mutations have been introduced at each step of the evolution process. While these efforts have yielded much success, the catalytic activity of the evolved PTE variants against VX and VR remains substantially lower than that achieved for hydrolysis of the G-agents.(57,58, 72, 73) Currently, no efforts have been made to dissect the contributions of individual residue changes on the collective properties of the evolved variants of PTE.

The crystal structure and active site residues of PTE are shown in FIG. 21.(75) In efforts to evolve PTE for V-agent hydrolysis, the most commonly targeted active site residues include Ile106, Phe132, His254, His257, Ser308 and a residue from an adjacent loop, Ile274.(63, 69, 70, 71) Enzymatic evolution experiments are often presented as straightforward, showing a linear increase in activity from a starting variant to a more “evolved” variant with enhanced catalytic activity or stability.(62, 70, 76-81) However, the actual complexity of the problem is relatively immense. As demonstrated by the sequence similarity networks (SSN) presented in FIG. 22, the complexity of the evolution process increases exponentially as more sites are included. Constructing a library that is limited to only amino acids previously identified in positive variants and only at the six sites known to be most important for V-agent hydrolysis results in 28 variants that differ from the wild-type enzyme by only a single amino acid. Increasing the deviation from one amino acid to two amino acids results in 322 possible variants with 1,992 ways to interconvert them. This complexity increases to 28,800 variants and 403,200 relationships for the full 6-site library. In the challenge of modifying PTE for V-agent hydrolysis, the chemical step of the reaction must be optimized for P—S bond cleavage and the unresolved synergy amongst residues in the active site is likely to be a major hurdle. This situation is further complicated by the need to use substrate analogs for screening purposes and the vast number of variants that must be screened.

Given this complexity, it is not surprising that the evolution of PTE for V-agent hydrolysis has been difficult and that no systematic method for enzymatic evolution has been developed. Recent efforts in the field of enzyme evolution have taken two approaches. In the first, large libraries of variants are constructed with the assumption that synergy is unimportant, and consequently the complexity of the library is linear rather than exponential. Only a limited number of variants are screened (˜1% coverage) with the assumption that the best variant can be achieved by recombining any positive mutations identified.(71, 76, 77) In the second approach, “small smart” libraries are constructed and screened to >90% coverage of the library. In this approach any synergistic effects between included mutations can be identified, but the sampling of evolutionary space is extremely limited by the small library size.(79, 82) Multiple rounds of evolution are required for both approaches, but this amplifies the problems of epistatic and synergistic effects. In the case of epistatic effects, the “fixing” of a mutation in an early round of evolution can severely diminish the potential for improvement in later rounds.(83-85) The presence of synergistic effects cannot be predicted a priori and if these sites are targeted in different rounds of evolution, then the most promising variants are likely to be missed.

A major goal in the field of enzyme evolution is the development of a methodology that allows the quantitative measurement of the effects of individual mutations and allows the prediction of beneficial variants. In addition, this methodology has to be able to deal with the presence of synergistic effects and large library sizes. This task is made more difficult because substrate analogs are often needed for screening. As the catalytic activity of an enzyme increases, the specificity also increases, and enzyme evolution can be severely hampered by selectivity toward the analog at the cost of activity toward the desired substrate.(85, 86) Neither of the current approaches in enzyme evolution offers the ability to predict activity for substrates, which were not used in the initial screening.

It would be advantageous to have enzymes that could optimize the hydrolysis of organophosphate nerve agents, including a new analogue and mutation strategies to optimize PTE for the hydrolysis of G-agents and V-agents such as VX and VR.

### SUMMARY

An embodiment of the disclosure is a synthetic amino acid sequence comprising mutations at one or more of positions 108, 132, 254, 257, 274, and 308 of VRN A80V/K185R/I274N) (SEQ ID NO: 26) and functioning by hydrolyzing an organophosphate nerve agent. In an embodiment, the sequence is that of variant BHR-73-MNW, BHR-74, BHR-23, BHR-53, BHR-73, BHR-45, BHR-52, or BHR-75. An embodiment includes a synthetic DNA sequence encoding the synthetic amino acid sequence. An embodiment includes a synthetic cDNA sequence comprising the coding sequence of the synthetic DNA sequence. An embodiment includes a plasmid comprising the synthetic DNA sequence.

An embodiment of the disclosure is a method of hydrolysis of an organophosphate nerve agent comprising contacting an organophosphate nerve agent with a synthetic DNA sequence encoding the synthetic amino acid sequence. In an embodiment, the organophosphate is selected from the group consisting of paraoxon, SP-VX, SP-VR, DEVX, DMVX, RP-OMVR, malathion, and ethoprophos.

An embodiment of the disclosure is a system for detoxifying an organophosphate nerve agent comprising contacting the synthetic amino acid sequence with an organophosphate nerve agent.

An embodiment is a kit for detoxifying an organophosphate nerve agent comprising the synthetic amino acid sequence.

An embodiment is a method of producing variants of phosphotriesterase, wherein the variants are capable of detoxifying an organophosphate nerve agent, comprising the steps of: obtaining a PTE gene; inserting the PTE gene into a vector; preparing a series of sequential mutational libraries wherein the PTE gene encodes a synthetic amino acid sequence; expressing the variant as a protein; screening the variant for catalytic activity against one selected from the group consisting of paraoxon, SP-VX, SP-VR, DEVX, DMVX, RP-OMVR, malathion, and ethoprophos to determine the hydrolytic activity; and selecting the variant for use in hydrolysis of an organophosphate nerve agent based upon its hydrolytic activity. In an embodiment, the variant synthetic amino acid sequence is at least 80% homogenous to the synthetic amino acid sequence In embodiment, the organophosphate is VX. In an embodiment, the hydrolysis is selective for the SP-enantiomer of VX. In an embodiment, the organophosphate is VR. In an embodiment, the hydrolysis is selective for the S_{P}-enantiomer of VR.

An embodiment of the disclosure is a synthetic amino acid sequence comprising the synthetic amino acid sequence of VQFL (SEQ ID NO: 2), capable of hydrolyzing organophosphates. In an embodiment, a synthetic DNA sequence encodes the synthetic amino acid sequence of SEQ ID NO: 2. In another embodiment, a synthetic cDNA sequence comprises the coding sequence of the synthetic DNA. In another embodiment, a plasmid comprises the synthetic DNA sequence.

An embodiment of the disclosure is a method of hydrolysis of an organophosphate nerve agent comprising contacting an organophosphate nerve agent with the synthetic amino acid sequence SEQ ID NO: 2; and hydrolyzing the organophosphate nerve agent. In an embodiment, the organophosphate is VX. In another embodiment, the organophosphate is VR. In yet another embodiment, the organophosphate is selected from the group consisting of GB, GD, and GF.

An embodiment of the disclosure is a system for detoxifying an organophosphate nerve agent comprising contacting the synthetic amino acid sequence of SEQ ID NO: 2 with an organophosphate nerve agent.

An embodiment of the disclosure is a kit for detoxifying an organophosphate nerve agent comprising the synthetic amino acid sequence of SEQ ID NO: 2.

An embodiment of the disclosure is the synthetic amino acid sequence of claim 1, further comprising mutations I106C (CVQFL (SEQ ID NO: 3)). In an embodiment, a synthetic DNA sequence encodes the synthetic amino acid sequence of SEQ ID NO: 3. In an embodiment, a synthetic cDNA sequence comprises the coding sequence of the synthetic DNA sequence. In another embodiment, a plasmid comprises the synthetic DNA sequence.

An embodiment of the disclosure is a method of hydrolysis of an organophosphate nerve agent comprising contacting an organophosphate nerve agent with the synthetic amino acid sequence of SEQ ID NO: 3; and hydrolyzing the organophosphate nerve agent. In an embodiment, the organophosphate is VX. In another embodiment, the organophosphate is VR. In yet another embodiment, the organophosphate is selected from the group consisting of GB, GD, and GF.

An embodiment of the disclosure is a system for detoxifying an organophosphate nerve agent comprising contacting the synthetic amino acid sequence of SEQ ID NO: 3 with an organophosphate nerve agent.

An embodiment of the disclosure is a kit for detoxifying an organophosphate nerve agent comprising the synthetic amino acid sequence of SEQ ID NO: 3.

An embodiment of the disclosure is the synthetic amino acid sequence of claim 1, further comprising mutations A80V, K185R, and I274N (VRN-VQFL (SEQ ID NO: 4)). In an embodiment, the synthetic DNA sequence encodes the synthetic amino acid sequence of SEQ ID NO: 4. In another embodiment, the synthetic cDNA sequence comprises the coding sequence of the synthetic DNA sequence. In yet another embodiment, a plasmid comprises the synthetic DNA sequence.

An embodiment of the disclosure is a method of hydrolysis of an organophosphate nerve agent comprising contacting an organophosphate nerve agent with the synthetic amino acid sequence of SEQ ID NO: 4; and hydrolyzing the organophosphate nerve agent. In an embodiment, the organophosphate is VX. In an embodiment, the organophosphate is VR. In an embodiment, the organophosphate is selected from the group consisting of GB, GD, and GF.

An embodiment of the disclosure is a system for detoxifying an organophosphate nerve agent comprising contacting the synthetic amino acid sequence of SEQ ID NO: 4 with an organophosphate nerve agent.

An embodiment of the kit for detoxifying an organophosphate nerve agent comprising the synthetic amino acid sequence of SEQ ID NO: 4.

An embodiment of the disclosure is the synthetic amino acid sequence comprising the synthetic amino acid sequence of L7ep-3a (SEQ ID NO: 5). In an embodiment, a synthetic DNA sequence encodes the synthetic amino acid sequence of SEQ ID NO: 5. In an embodiment, a synthetic cDNA sequence comprises the coding sequence of the synthetic DNA sequence. In yet another embodiment, a plasmid comprises the synthetic DNA sequence.

An embodiment of the disclosure is a method of hydrolysis of an organophosphate nerve agent comprising contacting an organophosphate nerve agent with the synthetic amino acid sequence of SEQ ID NO: 5; and hydrolyzing the organophosphate nerve agent. In an embodiment, the organophosphate is VX. In an embodiment, the organophosphate is VR. In another embodiment, the organophosphate is selected from the group consisting of GB, GD, and GF.

An embodiment of the disclosure is a system for detoxifying an organophosphate nerve agent comprising contacting the synthetic amino acid sequence of SEQ ID NO: 5 with an organophosphate nerve agent.

An embodiment of the disclosure is a kit for detoxifying an organophosphate nerve agent comprising the synthetic amino acid sequence of SEQ ID NO: 5.

An embodiment of the disclosure is a synthetic amino acid sequence comprising the synthetic amino acid sequence of L7ep-3a I106G (SEQ ID NO: 6), capable of hydrolyzing organophosphates. In an embodiment, a synthetic DNA sequence encodes the synthetic amino acid sequence of SEQ ID NO: 6. In another embodiment, a synthetic cDNA sequence comprising the coding sequence of the synthetic DNA sequence. In yet another embodiment, a plasmid comprising the synthetic DNA sequence.

An embodiment of the disclosure is a method of hydrolysis of an organophosphate nerve agent comprising contacting an organophosphate nerve agent with the synthetic amino acid sequence of SEQ ID NO: 6; and hydrolyzing the organophosphate nerve agent. In an embodiment, wherein the organophosphate is VX. In another embodiment, the organophosphate is VR. In yet another embodiment, the organophosphate is selected from the group consisting of GB, GD, and GF.

An embodiment of the disclosure is a system for detoxifying an organophosphate nerve agent comprising contacting the synthetic amino acid sequence of SEQ ID NO: 6 with an organophosphate nerve agent.

An embodiment of the disclosure is a kit for detoxifying an organophosphate nerve agent comprising the synthetic amino acid sequence of SEQ ID NO: 6.

An embodiment of the disclosure is a method of producing variants of phosphotriesterase, wherein the variants are capable of detoxifying an organophosphate nerve agent, comprising the steps of: obtaining a PTE gene; inserting the PTE gene into a vector; preparing a series of sequential mutational libraries wherein the PTE gene encodes a synthetic amino acid sequence of SEQ ID NO: 6; expressing the variant as a protein; screening the variant for catalytic activity against one selected from the group consisting of DEVX, DMVX, DEVR, and OMVR to determine the hydrolytic activity; and selecting the variant for use in hydrolysis of an organophosphate nerve agent based upon its hydrolytic activity. In an embodiment, the variant synthetic amino acid sequence is at least 80% homogenous to the synthetic amino acid sequence of SEQ ID NO: 6.

An embodiment of the disclosure is a method of producing variants of phosphotriesterase, wherein the variants are capable of detoxifying an organophosphate nerve agent, comprising the steps of: obtaining a PTE gene; inserting the PTE gene into a vector; preparing a series of sequential mutational libraries wherein the PTE gene encodes a synthetic amino acid sequence comprising the mutations I106C, F132V, H254Q, H257Y, A270V, L272M, I274N, and S308L (SEQ ID NO: 5); expressing the variant as a protein; screening the variant for catalytic activity against one selected from the group consisting of DEVX, DMVX, DEVR, and OMVR to determine the hydrolytic activity; and selecting the variant for use in hydrolysis of an organophosphate nerve agent based upon its hydrolytic activity. In an embodiment, the variant comprising the mutations I106C, F132V, H254Q, H257Y, A270V, L272M, I274N, and S308L synthetic amino acid sequence is at least 80% homogenous to the synthetic amino acid sequence of SEQ ID NO: 5)

The foregoing has outlined rather broadly the features of the present disclosure in order that the detailed description that follows can be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 depicts the hydrolysis of VX by phosphotriesterase to a nontoxic form;

FIG. 2A depicts the structure of (S_{P})-tabun (GA);

FIG. 2B depicts the structure of (S_{P})-sarin (GB);

FIG. 2C depicts the structure of (S_{P}S_{C})-soman (GD);

FIG. 2D depicts the structure of (S_{P}R_{C})-soman (GD);

FIG. 2E depicts the structure of paraoxon;

FIG. 2F depicts the structure of (S_{P})-cyclosarin (GF);

FIG. 2G depicts the structure of (S_{P})-VX;

FIG. 2H depicts the structure of (S_{P})-VR;

FIG. 3A depicts the structure of VX;

FIG. 3B depicts the structure of VR;

FIG. 3C depicts the structure of Demeton-S;

FIG. 3D depicts the structure of Demeton-S methyl;

FIG. 3E depicts the structure of diisopropyl amiton (DEVX);

FIG. 3F depicts the structure of paraoxon;

FIG. 3G depicts the structure of R_{P}-1;

FIG. 3H depicts the structure of S_{P}-1;

FIG. 4A depicts the structure of S_{P}-1 (Compound 1);

FIG. 4B depicts the structure of S_{P}-2 (Compound 2);

FIG. 4C depicts the structure of S_{P}-3 (Compound 3);

FIG. 4D depicts the structure of S_{P}S_{C}-4 (Compound 4);

FIG. 4E depicts the structure of S_{P}R_{C}-4 (Compound 4);

FIG. 4F depicts the structure of S_{P}-5 (Compound 5);

FIG. 4G depicts the structure of R_{P}-1 (Compound 1);

FIG. 4H depicts the structure of R_{P}-2 (Compound 2);

FIG. 4I depicts the structure of R_{P}-3 (Compound 3);

FIG. 4J depicts the structure of R_{P}R_{C}-4 (Compound 4);

FIG. 4K depicts the structure of R_{P}S_{C}-4 (Compound 4);

FIG. 4L depicts the structure of R_{P}-5 (Compound 5);

FIG. 5A depicts representative time courses for the complete hydrolysis of 160 μM racemic VX by the QF variant of PTE;

FIG. 5B depicts representative time courses for the complete hydrolysis of 160 μM racemic VX by the WT variant of PTE;

FIG. 5C depicts representative time courses for the complete hydrolysis of 160 μM racemic VX by the VRN-VQFL variant of PTE;

FIG. 5D depicts representative time courses for the complete hydrolysis of 160 μM racemic VX by the L7ep-3a variant of PTE;

FIG. 5E depicts representative time courses for the complete hydrolysis of 160 μM racemic VX by the L7ep-2b variant of PTE;

FIG. 5F depicts representative time courses for the complete hydrolysis of 160 μM racemic VX by the QF+L7ep-2b variant of PTE;

FIG. 6 depicts enhancement in the catalytic properties for the hydrolysis of VX and DEVX by variants of PTE;

FIG. 7 depicts representative Michaelis-Menton plots for the hydrolysis of DEVX by wild-type and evolved variants of PTE;

FIG. 8 depicts the hydrolysis of racemic VX by the VRN-VQFL variant observed by quantitative ³¹P{¹H} NMR spectroscopy;

FIG. 9 depicts construction of the multisite partially randomized PTE library;

FIG. 10 depicts the screening of the M317X mutant library against S_{P}-5 using GWT-d1 as the parental template.

FIG. 11A depicts screening of the six-site randomized library using GWT-f1 as the parental template with S_{P}-5;

FIG. 11B depicts screening of the error-prone PCR library using GWT-f4 as the parental template with S_{P}-5;

FIG. 12 depicts an outline of the parental lineage for the construction of mutants of PTE that are enhanced for the hydrolysis of S_{P}-4 and S_{P}-5;

FIG. 13A depicts bar graphs illustrating enhanced values for k_{cat}/Kₘ (M⁻¹s⁻¹) for the S_{P}-enantiomer of compound 1 (FIG. 4A);

FIG. 13B depicts bar graphs illustrating enhanced values for k_{cat}/Kₘ (M⁻¹s⁻¹) for the S_{P}-enantiomer of compound 2 (FIG. 4B);

FIG. 13C depicts bar graphs illustrating enhanced values for k_{cat}/Kₘ (M⁻¹s⁻¹) for the S_{P}-enantiomer of compound 3 (FIG. 4C);

FIG. 13D depicts bar graphs illustrating enhanced values for k_{cat}/Kₘ (M⁻¹s⁻¹) for the S_{P}-enantiomer of compound S_{P}R_{C}-4 (compound 4) (FIG. 4E);

FIG. 13E depicts bar graphs illustrating enhanced values for k_{cat}/Kₘ (M⁻¹s⁻¹) for the S_{P}-enantiomer of compound S_{P}S_{C}-4 (compound 4) (FIG. 4D);

FIG. 13F depicts bar graphs illustrating enhanced values for k_{cat}/Kₘ (M⁻¹s⁻¹) for the S_{P}-enantiomer of compound 5 (FIG. 4F); and

FIG. 14 depicts the amino acid sequence of organophosphate-degrading protein (opd) from Brevundimonas diminuta (Pseudomonas diminuta) (SEQ ID NO: 1).

FIG. 15A depicts the structure of paraoxon.

FIG. 15B depicts the structure of Sₚ-APVR.

FIG. 15C depicts the structure of Rₚ-APVR.

FIG. 15D depicts the structure of Sₚ-VX.

FIG. 15E depicts the structure of DEVX.

FIG. 15F depicts the structure of DMVX.

FIG. 15G depicts the structure of Sₚ-VR.

FIG. 15H depicts the structure of DEVR.

FIG. 15I depicts the structure of Rₚ-OMVR.

FIG. 16A depicts a structural alignment between wild-type PTE (white) and L7ep-3a mutant (blue).

FIG. 16B depicts an expanded view of Loop-7 and -8.

FIG. 17 depicts the substrate binding pockets of wild-type (white) and L7ep-3a (grey).

FIG. 18A depicts the metal center of wild-type PTE variants.

FIG. 18B depicts the metal center of QF variants.

FIG. 18C depicts the metal center of L7ep-3a variants.

FIG. 19A depicts S_{P}-VX docked in the active site of L7ep-3a.

FIG. 19B depicts S_{P}-VR docked into the active site of L7ep-3a I106G.

FIG. 20 depicts an equation of the interaction between S_{P}-VR and L7ep-3a PTE.

FIG. 21 depicts the active site of PTE with the bound substrate analog diethyl 4-methylbenzylphosphonate. Zinc ions are shown as grey spheres. Substrate binding residues, as well as the interface residue Ile274, are shown as sticks. Residues modified in this study are shown in red. Figure prepared from PDB id: 1DPM using UCSF-Chimera18.

FIG. 22A depicts a sequence similarity network of a limited six-site library of PTE. Each square represents a unique variant in the library and a line represents a relationship of only one amino acid difference. Wild-type PTE (red) and 28 variants, which differ by only one amino acid (blue). Numbers indicate the amino acid position where the variants differ from wild-type PTE.

FIG. 22B depicts a sequence similarity network of a limited six-site library of PTE. Each square represents a unique variant in the library and a line represents a relationship of only one amino acid difference. Variants from panel 22A (blue) and in yellow are 322 variants, which differ from wild-type PTE by two amino acids.

FIG. 22C depicts a sequence similarity network of a limited six-site library of PTE. Each square represents a unique variant in the library and a line represents a relationship of only one amino acid difference. Variants from panel 22B and 1948 variants (peach) which differ from wild-type by three amino acids.

FIG. 22D depicts a sequence similarity network of a limited six-site library of PTE. Each square represents a unique variant in the library and a line represents a relationship of only one amino acid difference. Variants from panel 22C and 6541 variants (green) which differ from wild-type PTE by four amino acids.

FIG. 22E depicts a sequence similarity network of a limited six-site library of PTE. Each square represents a unique variant in the library and a line represents a relationship of only one amino acid difference. Variants from panel 22D and 11560 variants (purple) which differ from wild-type PTE by 5 amino acids.

FIG. 22F depicts a sequence similarity network of a limited six-site library of PTE. Each square represents a unique variant in the library and a line represents a relationship of only one amino acid difference. Complete six-site library including the 8400 (red) variants, which differ from wild-type PTE by six amino acids.

FIG. 23A depicts the chemical structure of paraoxon.

FIG. 23B depicts the chemical structure of S_{P}-VX.

FIG. 23C depicts the chemical structure of S_{P}-VR.

FIG. 23D depicts the chemical structure of DEVX.

FIG. 23E depicts the chemical structure of DMVX.

FIG. 23F depicts the chemical structures of R_{P}-OMVR.

FIG. 23G depicts the chemical structures of malathion.

FIG. 23H depicts the chemical structures of ethoprophos.

FIG. 24 depicts a sequence similarity network of variants identified in PTE library. Lines represent a difference of one amino acid. Nodes are colored by activity: >1.5×10³ M⁻¹ s⁻¹ for hydrolysis of R_{P}-OMVR is shown in peach. Top 10% for hydrolysis of racemic OMVR is shown in red. Top 10% for hydrolysis of DEVX is shown in green. Top 10% for hydrolysis of DMVX is shown in orange. Top 10% for hydrolysis of malathion is shown in black. Top 10% for hydrolysis of ethoprophos is shown in dark blue. Nodes with a border have high activity for more than one substrate. Variants with >9×10⁴ M⁻¹ s⁻¹ for hydrolysis of S_{P}-VX are shown at 1.5× size. Variants with >4.5×10³ M⁻¹ s⁻¹ for hydrolysis of S_{P}-VR are shown at 2× size.

FIG. 25 depicts heat maps of the 2 position amino acid combinations found in the library. First position listed is shown on vertical axis, second position listed is shown as vertical axis. Single letter amino acid codes are highlighted as red if rarely found in the library and green if over-represented. Black combinations are absent. Peach combinations are present at <½ expected number. Yellow shows combinations within 2-fold of expected values, and combinations shown in green are present at more than 2-fold expected number.

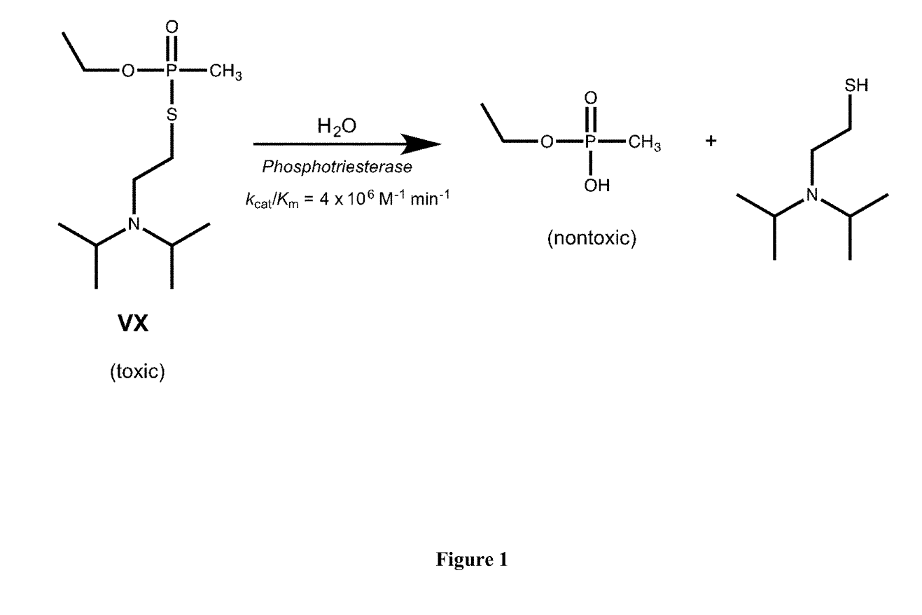

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure can be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3rd Edition.

As used herein the term, “wild-type” means and refers to the non-mutated version of a gene as it appears in nature.

As used herein the term, “enantiomer” means and refers to either of a pair of chemical compounds that have molecular structures that are nonsuperimposable mirror images.

As used herein the term, “G-agent” or “G-type” means and refers to nerve agents of the G (German) series. The series includes but is not limited to GA (tabun), GB (sarin), GF (cyclosarin) and GD (soman).

As used herein the term, “V-type” means and refers to nerve agents of the V series. The series includes but is not limited to VX, VE, V-gas, VG, VR, and VM.

### TABLES

Table 1. Mutations present in additional variants identified

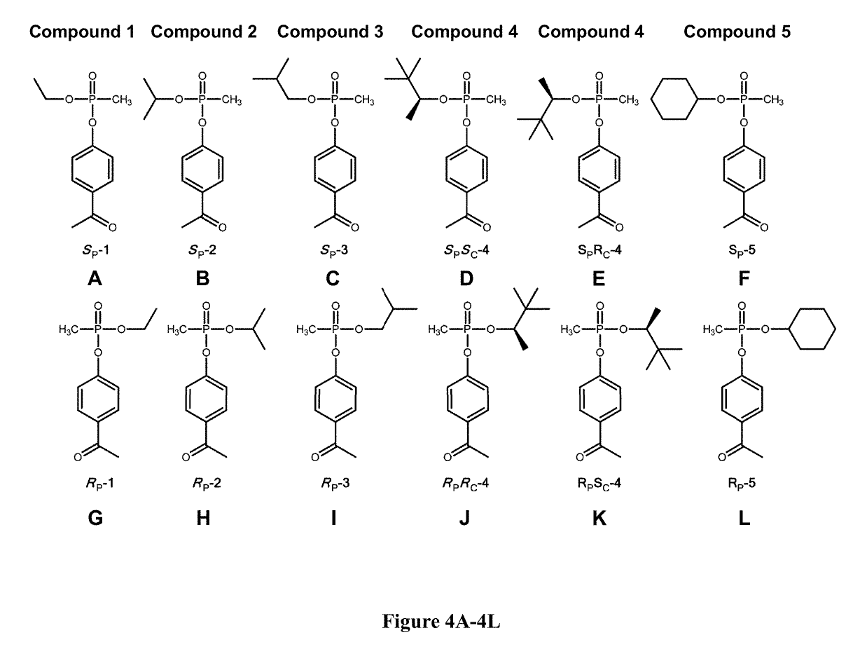

Table 2. Activity of PTE Variants against VX Analogues DEVX and Compound 1 (R_{P}-1 and SP-1) (see FIGS. 4A and 4G for structures)

Table 3. Activity of additional PTE Variants with DEVX

Table 4. Kinetic Parameters for PTE Variants with Paraoxon and Demeton-S

Table 5. Activity of PTE Variants with Racemic VX

Table 6. Identification of Mutants

Table 7. Activity of Wild-Type and Mutant Enzymes with Racemic G-Agents

Table 8. Kinetic Constants for Hydrolysis of GB, GD, and GF

Table 9. Values of kcat (s-1) for Wild-Type PTE and its Mutants

Table 10. Values of kcat/Km (M-1s-1) for Wild-Type PTE and its Mutants

Table 11. Kinetic constants for PTE variants with APVR

Table 12. Kinetic parameters for PTE variants with paraoxon and DEVX

Table 13. Kinetic constants with the racemic nerve agents VX and VR

Table 14. X-ray crystallography data for L7ep-3a and L7-ep-3a I106G

Table 15. Kinetic constants for PTE variants with V-agent analogs

Table 16. Amino acid positions targeted and allowed residues in library construction

Table 17. Kinetic constants for PTE variants with achiral substrates

Table 18. Kinetic constants for purified PTE variants with paraoxon

Table 19. Kinetic constants for PTE variants with chiral substrates

Table 20. Kinetic constants for purified PTE variants with OMVR.

Table 21. Kinetic constants for purified PTE variants with DEVX.

Table 22. Kinetic constants for purified PTE variants with DMVX.

Table 23. Kinetic constants for purified PTE variants with malathion

Table 24. Kinetic constants for purified PTE variants with paraoxon

Table 25. Activity coefficients for mutations from the wild-type residue at each position

Table 26. Kinetic constants for I106G variants with OMVR

Table 27. Kinetic constants for purified PTE variants with VX.

Table 28. Kinetic constants for PTE variants with DEVX.

Table 29. Kinetic constants for PTE variants with DMVX

Table 30. Kinetic constants for PTE variants with malathion

Table 31. Kinetic constants for PTE variants with paraoxon

Table 32. Kinetic constants for PTE variants with OMVR

Table 33. Kinetic constants for PTE variants with VX

Table 34. Kinetic constants for PTE variants with VR

Table 35. Distributions of amino acids found at each position in library

Table 36. Mutations found in substrate specific variants

The V-type organophosphorus nerve agents are among the most hazardous compounds known. Previous efforts to evolve the bacterial enzyme phosphotriesterase (PTE) for the hydrolytic decontamination of VX resulted in the variant L7ep-3a, which has a k_{cat} value more than 2-orders of magnitude higher than wild-type PTE. Because of the relatively small size of the O-ethyl, methylphosphonate center in VX, stereoselectivity is not a major concern. However, the Russian V-agent, VR, contains a larger O-isobutyl, methylphosphonate center making stereoselectivity a significant issue since the SP-enantiomer is more toxic than the R_{P}-enantiomer. The three-dimensional structure of the L7ep-3a variant was determined to a resolution of 2.01 Å (PDB id: 4ZST). The active site of the L7ep-3a mutant has revealed a network of hydrogen bonding interactions between Asp-301, Tyr-257, Gln-254 and the hydroxide that bridges the two metal ions. A series of new analogs that mimic VX and VR has helped to identify critical structural features for the development of new enzyme variants that are further optimized for the catalytic detoxification of VR and VX. The best of these mutants has been shown to hydrolyze the more toxic SP-enantiomer of VR more than 600-fold faster than the wild-type phosphotriesterase. The organophosphorus nerve agents are among the most toxic compounds known. Compounds such as sarin, soman and VX are all chiral methyl phosphonates where the toxicity of the SP-enantiomer is much greater than for the R_{P}-enantiomer.(33) Recent events have dramatically demonstrated the continuing importance of developing rapid and environmentally compatible methods for the decontamination of these compounds.(34) This situation is particularly true for the V-type nerve agents, where the lethal dose is approximately 6 mg/person, and these compounds have been shown to persist for long periods of time.(35, 36) Significant advances have been made in developing enzymatic methods of decontamination for the G-type and VX nerve agents using the bacterial enzyme phosphotriesterase (PTE).(37, 38) While wild-type PTE has reasonable activity against the G-type nerve agents (kcat/Km˜105 M-1 s-1), this enzyme preferentially hydrolyzes the less toxic R_{P}-enantiomers.(39) Directed evolution of PTE to specifically target the G-type nerve agents has led to the identification of the variant H257Y/L303T (YT), which has proven highly efficient at the hydrolysis of the more toxic SP-enantiomer of sarin (GB), soman (GD), and cyclosarin (GF) with values of kcat/Km that exceed 106 M-1 s-1.(38)

Wild-type PTE exhibits little stereoselectivity against the relatively small phosphonate center of VX, but the elevated pKa of the thiol-leaving group provides a significant challenge for enzyme-catalyzed hydrolysis (kcat/Km˜102 M-1 s-1).(37) Mutation of residues contained within the active site of PTE resulted in the isolation of the variant H245Q/H257F (QF), which exhibited a 100-fold improvement for the hydrolysis of VX, relative to the wild-type enzyme (see Table 1 for identity of variants).(37) Additional active site variations resulted in the identification of the mutant CVQFL (QF+I106C/F132V/S308L) with a similar catalytic efficiency for the hydrolysis of VX, but a three-fold improvement in kcat. The best variant identified to date for the hydrolysis of VX is VRN-VQFL (QF+F132V/S308L+A80V/K185R/I274N). This mutant combines expression-enhancing mutations (A80V/K185R/I274N) with additional changes in the active site to achieve a kcat/Km of 7×104 M-1 s-1 for the hydrolysis of the SP-enantiomer of VX. (37, 40, 41) Expansion of the mutation strategy to targeted error-prone PCR, led to the identification of the variant L7ep3a (CVQFL+H257Y/A270V/L272M/I274N), which has a kcat value enhanced 152-fold relative to wild-type PTE.(37) How these combined mutations, some of which do not fall in the active site, are able to bring about such a dramatic improvement in catalytic ability is not clear.

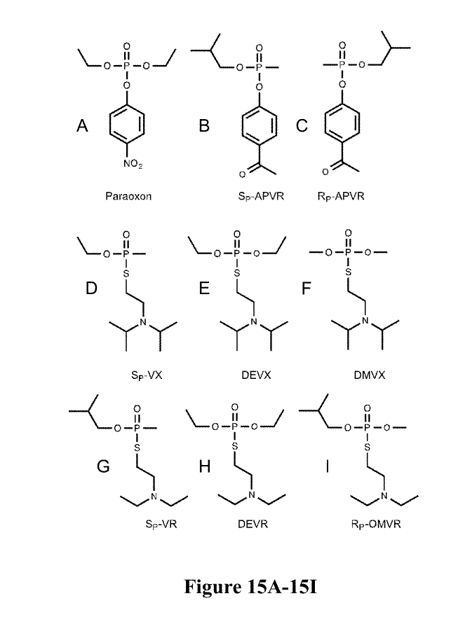

In addition to VX, the V-agents include the Russian (VR) and Chinese versions. Exemplified by VR, these additional V-agents contain a smaller thiol leaving group, and a larger ester group attached to the phosphorus center (FIGS. 15A-15I). Wild-type PTE has enzymatic activity for the hydrolysis of racemic VR similar to VX, but the larger isobutyl group attached to the phosphorus center results in a 25-fold preference for the less toxic R_{P}-enantiomer.(39, 42) The catalytic activity of wild-type PTE for the hydrolysis of the more toxic SP-enantiomer of VR (k_{cat}/Kₘ=4.3 M⁻¹ s⁻¹) is significantly lower than for the hydrolysis of VX. PTE variants which contain many of the same mutations as the VX-enhanced variants have been reported to have substantially improved catalytic activity against S_{P}-VR.(43) Currently, there is a lack of three-dimensional structural data that can be used to explain how the existing set of mutants are able to enhance the rate of hydrolysis of the phosphorothiolate bond in VX.

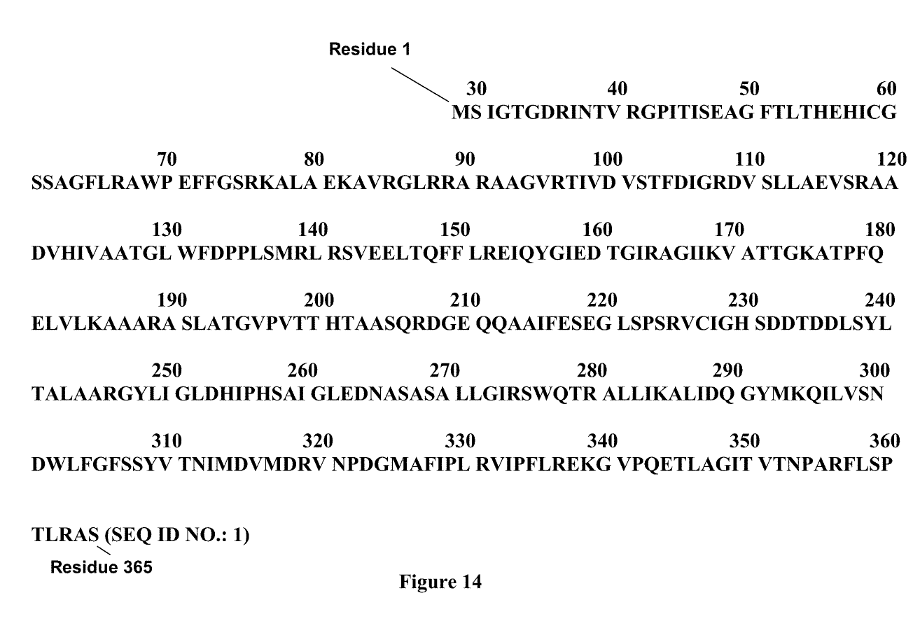

PTE variants can be used to decontaminate areas, equipment, and personnel after they come in contact with V-type or G-type nerve agents. This is especially useful for military or homeland security applications. The decontamination occurs without exposing the area, equipment, or personnel to harsh chemicals. Mutations in the sequence of PTE have been made to increase the ability of PTE to hydrolyze, and thus decontaminate, an area, equipment, or personnel. PTE was subjected to directed evolution for the improvement of catalytic activity against selected compounds through the manipulation of active site residues. A series of sequential two-site mutational libraries encompassing twelve active site residues of PTE was created. The libraries were screened for catalytic activity against a new VX analogue (DEVX), which contains the same thiolate leaving group of VX coupled to a di-ethoxy phosphate core rather than the ethoxy, methylphosphonate core of VX. The catalytic activity with DEVX was enhanced 26-fold relative to wild-type PTE. Further improvements were facilitated by targeted error-prone PCR mutagenesis of Loop-7 and additional PTE variants were identified with up to a 78-fold increase in the rate of DEVX hydrolysis. The best mutant hydrolyzed the racemic nerve agent VX with a value of kcat/Km of 7×104 M-1 s-1; a 230-fold improvement relative to the wild-type PTE. The sequence of wild-type PTE (organophosphate-degrading protein (opd)), without the leader peptide residues (1-29), is found in FIG. 14. The highest turnover number achieved by the mutants tested was 137 s-1; an enhancement of 152-fold relative to wild-type PTE. The stereoselectivity for the hydrolysis of the two enantiomers of VX was relatively low.

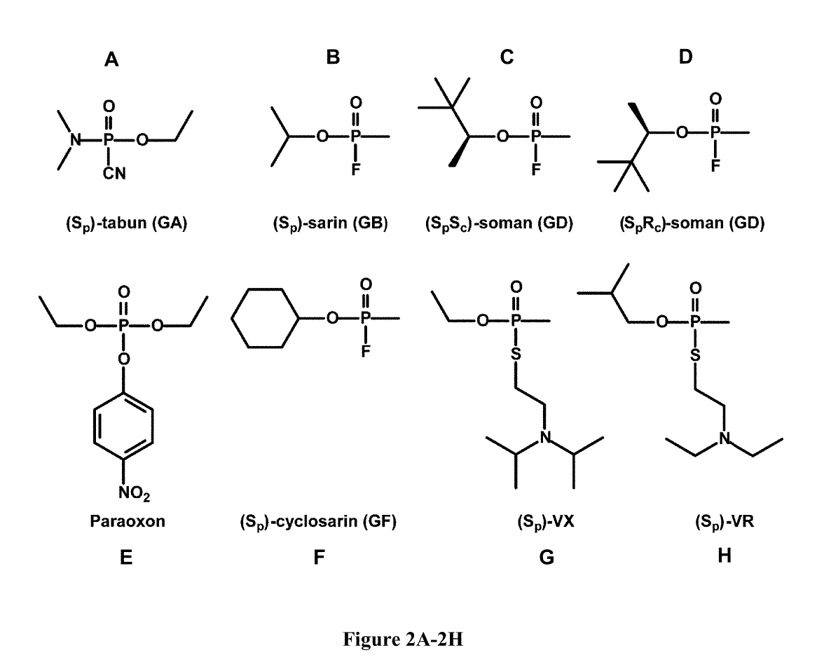

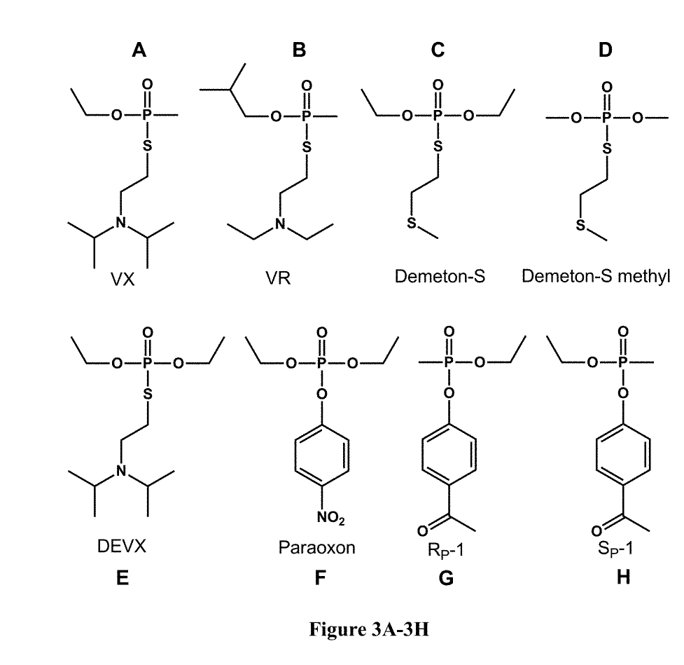

The P—S bond in VX is chemically more stable than the P—F bond found in the G-agents.(20) (FIGS. 2G, 2A-2D) Demeton-S contains the requisite P—S bond but does not contain the tertiary amine of VX, which is likely to be protonated at the relevant pH values. (FIGS. 3C, 3A) DEVX, containing the authentic leaving group of VX, yields results more directly applicable to the hydrolysis of VX itself (FIGS. 3E, 3A)

The QF variant of PTE shows improved hydrolysis against the SP-enantiomer of a chiral VX analogue.(18) Table 1 lists the amino acid changes present in the variants. This mutant was found to be significantly better for the hydrolysis of the P—S bond in DEVX and demeton-S than wild-type PTE. The synergistic mutations in this variant suggested that further improvements in catalytic activity could be facilitated by simultaneously mutating pairs of residues in the active site. The initial mutant libraries targeted pairs of residues in the active site that modulated the size and shape of the three substrate binding pockets. Sequential optimization of the active site residues resulted in an 18-fold improvement in catalytic activity against DEVX. Combining the best variant (VQFL) with expression enhancing mutations resulted in the variant VRN-VQFL. The VRN-VQFL variant exhibited a 26-fold improvement for the hydrolysis of DEVX.

TABLE 1
Mutations present in additional variants identified.

| Variant | Mutations present |
|---|---|
| WT | Wild type |
| ARN | A80V/K185R/I274N |
| QF | H254Q/H257F |
| QF.1 | H254Q/H257F/F306W/Y309H |
| LQF | F132L/H2540/H257F |
| VQF | F132V/H254Q/H257F |
| QF.a | W131H/F132L/H254Q/H257F |
| QF.b | W131H/F132I/H254Q/H257F |
| LQF.1 | F132L/H254Q/H257L |
| LQF.2 | F132L/H254R/H257A |
| LQF.3 | F132L/H254R/H257L |
| LQF.4 | F132L/H254R/H257Y |
| LQF.a | F132L/H254Q/H257F/L271V |
| LQF.b | F132L/H254Q/H257F/L271M |
| LQF.c | F132L/H254Q/H257F/L271A |
| LQFL | F132L/H254Q/H257F/S308L |
| LQF.d | F132L/H254Q/H257F/L271R/S308N |
| VQFL | F132V/H254Q/H257F/S308L |
| CVQFL | I106C/F132V/H254Q/H257F/S308L |
| VQFL.1 | I106G/F132V/H254Q/H257F/S308L |
| VQFL.2 | I106S/F132V/H254Q/H257F/S308L |
| VQFL.3 | I106A/F132V/H254Q/H257F/L303T/S308L |
| VRN-VQFL | A80V/F132V/K185R/H254Q/H257F/I274N/S308L |
| VRNGS-VQFL | A80V/F132V/K185R/D208G/H254Q/H257F/I274N/S308L/R319S |
| L7ep-1 | F132V/H254S/H257W/A266T/L271P/S308L |
| L7ep-2 | I106C/F132V/H254R/H257F/N265D/A270D/L272M/S276T/S308L |
| L7ep-3 | I106C/F132V/H254Q/H257Y/A270V/L272M/S308L |
| L7ep-4 | I106C/F132V/H254Q/H257F/I260N/D264N/I274N/S308L |
| L7ep-5 | I106C/F132V/H254Q/H257Y/E264G/S308L |
| L7ep-6 | I106C/F132V/H254Q/H257F/A266E/S269T/S308L |
| L7ep-7 | I106C/F132V/H2540/H257F/I260V/S269T/S308L |
| L7ep-8 | I106C/F132V/H254Q/H257Y/I260V/S308L |
| L7ep-9 | I106C/F132V/H254Q/H257F/S269T/I274T/S308L |
| L7ep-10 | I106C/F132V/H254Q/H257Y/E263K/S308L |
| L7ep-11 | I106C/F132V/H254Q/H257Y/A266R/S308L |
| L7ep-12 | I106C/F132V/H254Q/H257F/S269T/I274S/S308L |
| L7ep-2a | I106C/F132V/H254R/H257F/N265D/A270D/L272M/I274T/S276T/S308L |
| L7ep-2b | I106C/F132V/H254R/H257F/N265D/A270D/I274N/S276T/S308L |
| L7ep-2c | I106C/F132V/H254Q/H257F/N265D/A270D/L272M/S276T/S308L |
| L7ep-2d | I106C/F132V/H254R/H257F/N265D/A270D/L272M/I274S/S276T/S308L |
| L7ep-2e | I106C/F132V/H254R/H257F/N265D/A270D/1274P/S276T/S308L |
| L7ep-2f | I106C/F132V/H254R/H257F/N265D/A270D/I274S/S276T/S308L |
| L7ep-2g | I106C/F132V/H254R/H257F/N265D/A270D/I274Q/S276T/S308L |
| L7ep-2h | I106C/F132V/H254R/H257F/N265D/A270D/L272M/S276H/S308L |
| L7ep-2i | I106C/F132V/H254R/H257F/N265D/A270D/L272M/S276S/S308L |
| L7ep-2j | I106C/F132V/H254R/H257F/N265D/A270D/L272M/S276P/S308L |
| L7ep-3a | I106C/F132V/H254Q/H257Y/A270V/L272M/I274N/S308L |
| L7ep-3b | I106C/F132V/H254Q/H257Y/A270D/L272M/S308L |
| L7ep-3c | I106C/F132V/H254Q/H257Y/N265D/L272M/S308L |
| L7ep-3d | I106C/F132V/H254Q/H257Y/A270V/L272M/I274T/S308L |

Additional strategies were used to further enhance the activity of PTE against the phosphorothiolate bond. Error-prone PCR is a useful technique for enzyme evolution, but the mutation frequencies are typically restricted to 1-3 base pair changes per gene because of the significant chance of introducing deleterious mutations. Substantial improvements in enzyme activity can require numerous amino acid changes, which are not typically achievable by error-prone PCR. Targeting error-prone PCR to only Loop-7 (residues 253-276) resulted in a mutation library with an average of 6 mutations per gene but still retained >20% active colonies. The hydrolysis of DEVX for one of the variants (L7ep-3) was improved to 36-fold over wild-type PTE due to 3 additional amino acid changes. The best variant identified (L7ep-2) was improved 63-fold for the hydrolysis of DEVX by 5 additional amino acid changes. Further optimization of L7ep-2 and L7ep-3 resulted in additional mutations that improved the activity to 78-fold (L7ep-2a) and 71-fold (L7ep-3a) over wild-type PTE, and achieved turnover numbers for the hydrolysis of the phosphorothiolate bond in excess of 100 s⁻¹.

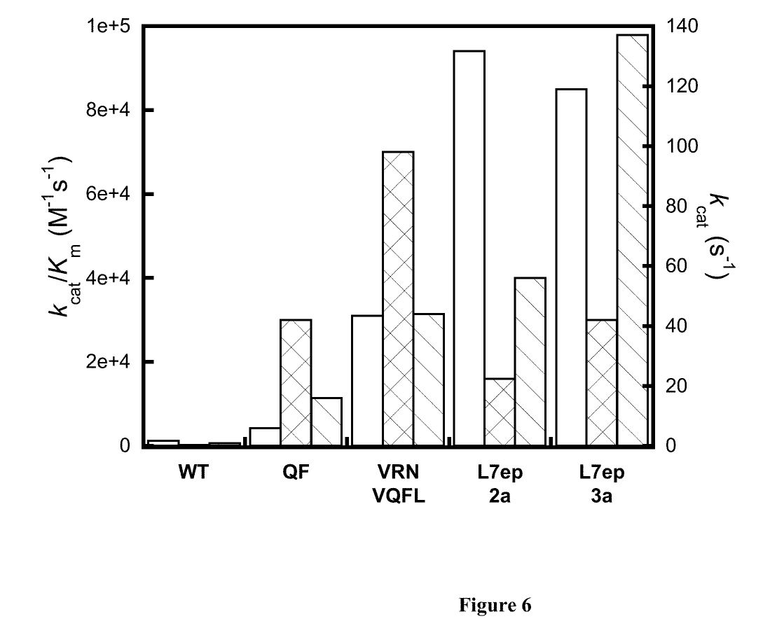

Hydrolysis of VX. A full kinetic characterization of wild-type and improved variants of PTE using racemic VX was conducted. Wild-type PTE exhibited low activity against VX, but there was a dramatic improvement with the QF mutant. The mutations in the large group pocket resulted in substantial improvements to kcat. The best variant identified (VRN-VQFL) against VX combines active site mutations in all three pockets and has a kcat/Km value that is increased 235-fold over wild-type PTE (FIG. 6). FIG. 6 depicts enhancement in the catalytic properties for the hydrolysis of VX and DEVX by variants of PTE. The values of kcat/Km for evolved variants of PTE are presented for DEVX (open bars) and VX (cross-hatched bars). The kcat values for the hydrolysis of VX are shown as right-hatched bars. (FIG. 6).

The Loop-7 optimized variants show good activity against VX, but did not demonstrate improved activity relative to the VRN-VQFL variant. The changes to Loop-7 resulted in substantial improvements in kcat but little change in the catalytic efficiency.

The L7ep-3a variant has a kcat of 137 s-1 for the hydrolysis of VX. This value is the highest ever reported for the enzymatic hydrolysis of VX.(11,14,24,25) Single concentration experiments with the H254R/H257L mutant of PTE showed an improvement of 10-fold against racemic VX.(24) Another variant exhibited a 26-fold improvement over wild-type PTE at 0.5 mM VX.(25) By contrast, the VRN-VQFL and L7ep-3 variants are improved by more than 200-fold in the value of kcat/Km. Human PON1 (paraoxonase) has been evolved in the laboratory for the hydrolysis of VX, but the reported value of kcat/Km for the best variant is 2.5×103 M-1 s-1, whereas the best PTE variant (VRN-VQFL) identified in this investigation has a value of kcat/Km of 7×104 M-1s-1.(11)

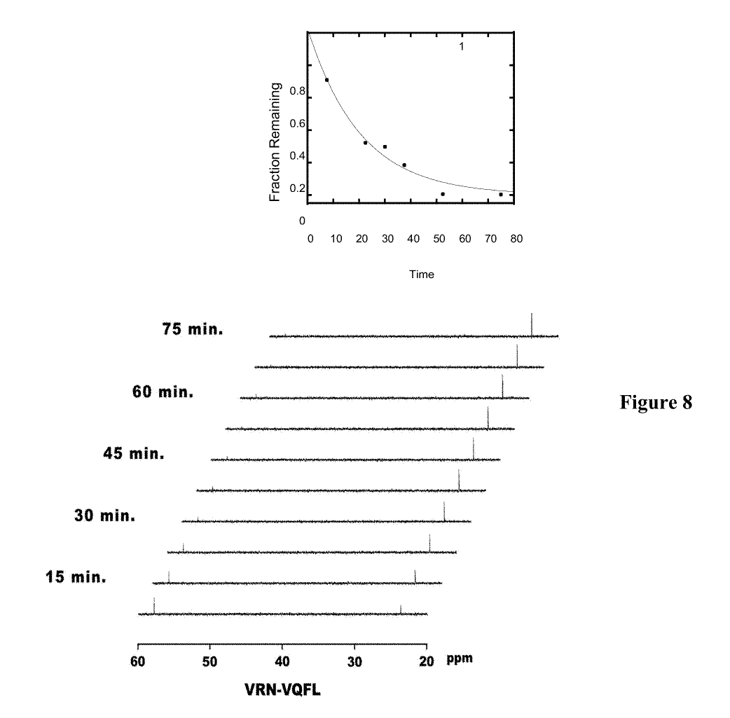

Stereochemical Preferences of Active Site Mutants. The toxicity of the organophosphate nerve agents depends on the stereochemistry of the phosphorus center.(17) With VX, it is estimated that the SP-enantiomer is about 100-fold more toxic than the R_{P}-enantiomer. The QF mutant prefers to hydrolyze the more toxic SP-enantiomer of VX by a factor of 12, relative to the R_{P}-enantiomer, whereas the L7ep-2b mutant prefers to hydrolyze the R_{P}-enantiomer by a factor of 12. The stereochemical preferences for the hydrolysis of VX are fully consistent with the stereoselective properties of these two mutants for the hydrolysis of SP-1 and RP-1, suggesting that the variants VRN-VQFL, L7ep-3, and L7ep-3a also prefer the SP-enantiomer of VX. While the modest selectivity prevented definitive assignment of the preferred enantiomer, complete neutralization of VX by the VRN-VQFL mutant via the hydrolysis of both enantiomers was demonstrated by 31P-NMR spectroscopy (FIG. 8).

The reconstruction of PTE for the hydrolysis of VX has resulted in dramatic improvements in the values of kcat and kcat/Km, relative to the wild type enzyme. It is proposed that the increase in the catalytic constants has been achieved by an increase in the rate constant for cleavage of the P—S bond (k3) rather than changes in the formation of the ternary complex (k1, k2) or the rate constant for product release (k5) as illustrated in a minimal kinetic mechanism (Scheme). The thiol leaving group of VX has a higher pKa than the fluoride leaving group of the G-agents, and the p-nitrophenol group of paraoxon. It has been demonstrated with the wild-type PTE that the chemical step (k3) is rate limiting for substrates with leaving groups having pKa values higher than 8.(20) Disruption of the hydrogen bonded network from D301-H254-D233 reduced the rate of hydrolysis of substrates with leaving groups having low pKa values but increased the rate of hydrolysis of substrates with leaving groups of higher pKa values.(30) Introduction of a glutamine at residue position 254 (as in the initial QF mutant), which apparently cannot support the transport of a proton away from the active site, may now facilitate the protonation of the thiol group by Asp-301 as the phosphorothiolate bond is cleaved.

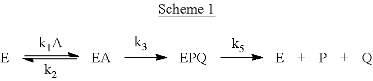

The turnover numbers for some slow substrates of PTE are thought to be reflective of the ability of the enzyme to align the substrate with the nucleophilic hydroxyl group attached to the binuclear metal center.(13) There is a strong likelihood that for some of the variants, subtle changes in the conformation of the active site will facilitate a better alignment between the substrate and attacking hydroxide, thereby achieving higher enzymatic rates of hydrolysis. In particular, the Loop-7 variants have been modified at residues that are somewhat distant from the active site, but are expected to bring about changes in the positioning of the Loop-7 α-helix.(13,28) This alignment effect would, of course, differ between the di-ethoxy phosphorus center of DEVX and the methylphosphonate core of VX (FIGS. 3E, 3A), which may explain the differences in the k_{cat} values for DEVX and VX with the variants L7ep-2a and L7ep-3a. (Table 2) These changes have resulted in variants with high enzymatic efficiency and exceptional kinetic constant for the hydrolysis of VX.

TABLE 2
Activity of PTE Variants against VX analogues DEVX and
compound 1 (see FIGS. 3, 4A and 4G for structures)*

| | DEVX | | | R_{P}-1 | | | S_{P}-1 | | |
|---|---|---|---|---|---|---|---|---|---|
| Variant | k_{cat} | Kₘ | k_{cat}/Kₘ | k_{cat} | Kₘ | k_{cat}/Kₘ | k_{cat} | Kₘ | k_{cat}/Kₘ |
| WT | 1.1 | 0.87 | 1.2 × 10³ | 100 | 3700 | 2.7 × 10⁴ | 92 | 320 | 2.9 × 10⁵ |
| QF | 6.1 | 1.4 | 4.2 × 10³ | 120 | 230 | 5.3 × 10⁵ | 34 | 18 | 1.8 × 10⁶ |
| LQF | 15 | 1.7 | 9.0 × 10³ | 112 | 910 | 1.2 × 10⁵ | 27 | 13 | 2.1 × 10⁶ |
| VQF | 18 | 1.0 | 1.9 × 10⁴ | 82 | 340 | 2.4 × 10⁵ | 25 | 11 | 2.2 × 10⁶ |
| LQFL | 10 | 0.76 | 1.4 × 10⁴ | 76 | 160 | 4.7 × 10⁵ | 45 | 7.4 | 6.1 × 10⁶ |
| VQFL | 14 | 0.65 | 2.2 × 10⁴ | 69 | 129 | 5.3 × 10⁵ | 32 | 7.4 | 4.3 × 10⁶ |
| CVQFL | 16 | 0.76 | 2.1 × 10⁴ | 54 | 170 | 3.2 × 10⁵ | 39 | 24 | 1.6 × 10⁶ |
| VRN-VQFL | 22 | 0.73 | 3.1 × 10⁴ | 124 | 160 | 7.8 × 10⁵ | 65 | 26 | 2.5 × 10⁶ |
| VRNGS-VQFL | 11 | 0.99 | 1.1 × 10⁴ | 204 | 350 | 5.8 × 10⁵ | 93 | 22 | 4.3 × 10⁶ |
| L7ep-1 | 16 | 0.60 | 3.2 × 10⁴ | 590 | 8600 | 6.8 × 10⁵ | 670 | 2800 | 2.3 × 10⁵ |
| L7ep-2 | 48 | 0.63 | 7.6 × 10⁴ | 240 | 730 | 3.3 × 10⁵ | 90 | 400 | 2.3 × 10⁵ |
| L7ep-3 | 29 | 0.69 | 4.3 × 10⁴ | 143 | 560 | 2.5 × 10⁵ | 50 | 22 | 2.3 × 10⁶ |
| L7ep-2a | 135 | 1.4 | 9.4 × 10⁴ | 235 | 950 | 2.5 × 10⁵ | 180 | 1090 | 1.7 × 10⁵ |
| L7ep-2b | 76 | 1.0 | 7.4 × 10⁴ | 136 | 610 | 2.2 × 10⁵ | 95 | 1090 | 8.6 × 10⁴ |
| L7ep-3a | 51 | 0.6 | 8.5 × 10⁴ | 290 | 1500 | 1.9 × 10⁵ | 90 | 36 | 2.5 × 10⁶ |
| L7ep-3b | 64 | 1.0 | 6.2 × 10⁴ | 202 | 1800 | 1.1 × 10⁵ | 48 | 34 | 1.4 × 10⁶ |

*Standard errors from fits of the data to eq 1 are less than 20% of the stated values.

The structure of the L7ep3a variant was determined. The structure aided in understanding the chemical mechanism for the enhancement of phosphorothiolate bond cleavage. With this new structural information, a series of PTE variants was created to incorporate changes in the active site of PTE that would more easily accommodate the O-isobutyl group of VR. To facilitate the further development of PTE for the hydrolysis of various V-agents, a new series of analogs were designed and synthesized. Enhanced variants of PTE have been identified that have more than a 600-fold improvement in the catalytic activity for the hydrolysis of the toxic SP-enantiomer of VR.

The bacterial enzyme phosphotriesterase (PTE) is noted for its ability to hydrolyze many organophosphate compounds including insecticides and chemical warfare agents. PTE has been the subject of multiple enzyme evolution attempts, which have been highly successful against specific insecticides and the G-type nerve agents. Similar attempts targeting the V-type nerve agents have failed to reach the same degree of success. Enzyme evolution is an inherently complex problem, which is complicated by synergistic effects, the need to use analogs in high throughput screening, and a lack of quantitative data to direct future efforts. Previous evolution experiments with PTE have assumed an absence of synergy and minimally screened large libraries, which provides no quantitative information on the effects of individual mutations. Here a systemic approach has been applied to a 28,800 member six-site PTE library. The library is screened against multiple V-agent analogs, and a combination of sequence and quantitative activity analysis is used to extract data on the effects of individual mutations. It is demonstrated that synergistic relationships dominate the evolutionary landscape of PTE, and that analog activity profiles can be used to identify variants with high activity for substrates. Using these approaches, multiple variants improved more than 1500-fold in k_{cat}/Kₘ for the hydrolysis of VX were identified, including one variant that is 9200-fold improved relative to wild-type PTE and is specific for the S_{P}-enantiomer of VX. Multiple variants highly active for S_{P}-VR were identified, the best of which is 13,400-fold improved in k_{cat}/Kₘ relative to wild-type PTE.

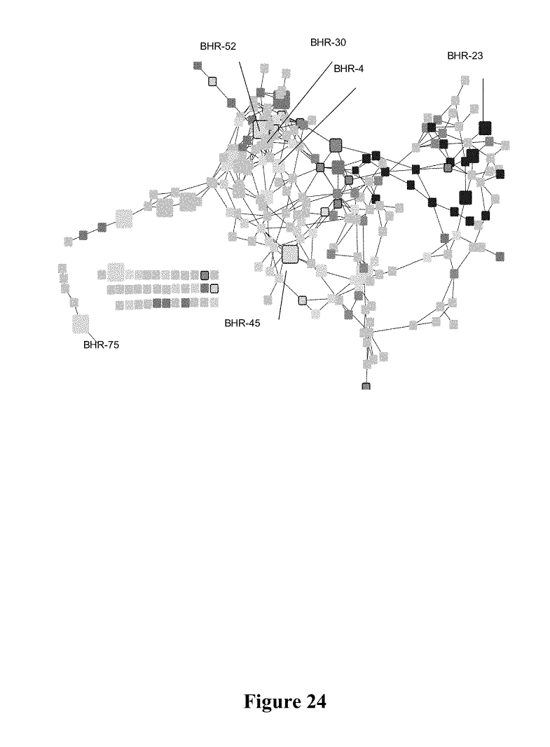

The challenge of modifying PTE for V-agent hydrolysis is that the chemical step of the reaction must be optimized for P—S bond cleavage and the unresolved synergy amongst residues in the active site is likely to be a major hurdle. This situation is further complicated by the need to use substrate analogs for screening purposes and the vast number of variants that must be screened. Here a methodology and formalism have been developed and applied to a PTE library that recombines the active site mutations observed in known variants with enhanced V-agent hydrolysis. Complete screening of the resultant 28,800-member library against five phosphorothiolate substrates was carried out. Variants with high activity and widely differing specificities have been found, and sequence signatures for unique activities identified. Single-site mutants were not independently constructed but using a combination of sequence and quantitative activity analysis of selected variants has allowed effects of single amino acid substitutions to be determined. The quantitative activity analysis has enabled the successful prediction of additional variants, which show strong activity against the highly toxic S_{P}-enantiomers of VX and VR. An analog activity profile (AAP) methodology is developed to successfully overcome the challenges associated with synergy/epistatic effects, and effectively allows analogs to identify enhanced variants for compounds that were not directly used in the screening process. The AAP methodology is demonstrated to correctly identify enhanced variants using (S_{P})-VX (FIG. 23B) as a target including a variant that is 9,200-fold improved over the wild-type enzyme. Library variants have been combined with other mutations that appear in other V-agent enhanced variants to yield a family of variants that are highly effective for the hydrolysis of (S_{P})-VR (FIG. 23C), with the best variant exhibiting a 13,500-fold improvement over wild-type PTE.

The development of the current approach takes into account the true complexity of enzyme evolution problems. The construction and complete screening of a V-agent directed PTE library against multiple substrates has yielded an enormous amount of data, which can be used to direct the search for valuable variants. Quantitative sequence analysis was possible with this approach despite 69% of the total variants screened not showing measurable activity. The combination of sequence analysis and activity coefficients allows for a quantitative analysis of the effects of individual mutations. Generating this information from the complete library allows the prediction of variants that are of high activity, while avoiding the pitfalls of epistatic and synergistic effects, which have plagued other efforts. The challenge of needing to use analogs in screening has been overcome by the development of the analog activity profile approach to screening. Each of the 33,300 colonies screened generated a unique analog activity profile. The subset of variants that have been purified serves as a reference set that can be characterized with any substrate of interest. The set of analog activity profiles of improved variants are then used to query the total library for variants of interest. It has been shown that with as little as five variants serving as the basis of the profile, more active variants can quickly be identified. There is no practical reason that once such a library has been established and screened with relevant substrates that the same library cannot be searched for additional activities. Using this approach, nine variants improved in k_{cat}/Kₘ by more than 1,000-fold for VX hydrolysis have been identified, while 14 variants have been identified that have improved by more than 1000-fold for S_{P}-VR. Inclusion of additional mutations, while they may stabilize the protein, offer at best minimal additional activity, but the best variant for hydrolysis of S_{P}-VR, BHR-73-MNW, exhibits a k_{cat}/Kₘ for S_{P}-VR of 5.8×10⁴ M⁻¹s⁻¹, which is 13,400-fold improved over the wild-type PTE.

### EXAMPLES

### Example 1

Most chemicals can be obtained from Sigma Chemical Company. The pfuTurbo DNA polymerase can be obtained from Agilent Technologies and the various restriction enzymes can be acquired from New England Biolabs. The two enantiomers of compound 1 (FIGS. 4A and 4G) can be synthesized.(18) VX and VR samples can be Chemical Agent Standard Analytical Reference Material (CASARM).

### Example 2

Active Site Library Construction. The nucleotides in the gene for PTE were modified to replace the nucleotides encoding the leader peptide (amino acid residues 1-29) with nucleotides encoding a methionine. (FIG. 14) Nucleotides encoding amino acid residues 29-365 of PTE were inserted into a pET 20b+ vector between the NdeI and EcoRI restriction sites as previously described.(18) The amino acid residue numbering of the sequence including the leader sequence has been retained. The 306X/309X, 131X/132X and 254X/257X double substitution libraries were constructed by site-directed mutagenesis using single sets of primers containing NNS (N=any base, S=G or C) codons at the positions of interest. The 271X/308X library was constructed by sequential reactions. The 106X/303X and 60X/317X libraries were constructed using a PCR overlap extension technique.(26) Plasmids from at least 10 colonies of each library were sequenced to ensure the randomization at the positions of interest. The identities of specific mutations for the variants are given in Table 1. The name of the phosphotriesterase gene is opd, the GenBank Accession Number is AER10490.1, and the Protein Model Portal Accession Number is G8DNV8.

### Example 3

Construction of Targeted Error-Prone Library. To construct the Loop-7 error-prone library, a set of 30-bp primers corresponding to the DNA sequences upstream and downstream of Loop-7 (amino acid residues 253-276) were used to amplify the PTE gene in three fragments. The amino acid residue numbering of the sequence including the leader sequence has been retained. The DNA coding region of Loop-7 was amplified in an error-prone PCR reaction while the two remaining fragments were amplified using standard PCR techniques. The final gene was constructed using PCR overlap-extension, resulting in a gene library with errors only in the coding region for residues 253-276.

### Example 4

Optimization of Error-Prone Variants. The five residue positions (254, 265, 270, 272, and 276) identified in the best Loop-7 error prone variant and residues 257 and 274 were further optimized by construction of two two-site (254X/257X and 272X/274X) and three single-site libraries (265X, 270X, 276X). The amino acid residue numbering of the sequence including the leader sequence has been retained. Libraries were constructed via QuikChange mutagenesis using degenerate primers to allow all 20 amino acids at the positions of interest. Approximately 200 colonies from each single-site library were screened, and approximately 1200 colonies from each two-site library were screened. The two enhanced variants identified in the Loop-7 error prone library were used as the template for a second round of targeted error-prone PCR of Loop-7.

### Example 5

Library Screening. Plasmid libraries were transformed into BL21 (DE3) E. coli competent cells and grown on LB plates. For all library transformations, the amount of DNA was kept low (<10 ng) to avoid the potential complication of double transformants.(27) Single colonies were used to inoculate 0.75 mL cultures of Super Broth (32 g tryptone, 20 g yeast extract, 5 g NaCl, and 0.4 g NaOH in 1 L H₂O) supplemented with 0.5 mM CoCl₂ in a 96-well block format. Cultures were grown at 37° C. for 8 hours. The temperature was reduced to 30° C. and protein expression induced by addition of 1 mM IPTG. Following 16 hours of additional growth, the bacteria were harvested by diluting a portion of the culture in a 1:1 ratio with 50 mM HEPES pH 8.0, 100 μM CoCl₂, 10% BugBuster® 10×(EMD Chemicals). Cultures were tested for activity against DEVX using a standard 250 μL assay that consisted of 50 mM HEPES, pH 8.0, 100 μM CoCl₂, 0.3 mM 5,5′-dithiobis(2-nitro-benzoic acid)(DTNB) and 0.2-0.5 mM DEVX. The reactions were initiated by the addition of 10 μL of cell lysate. Reactions proceeded at room temperature until color was clearly visible (1-4 hours). Product formation was determined by the change in absorbance at 412 nm using a plate reader. The variant used as the starting template for each library was included as a control on each plate. To account for differential culture growth, the final change in absorbance was normalized using the OD₆₀₀ for each culture compared to the average OD₆₀₀ of controls. The colonies giving the best results were re-grown as 5 mL overnight cultures and the plasmids harvested and sequenced to identify the variants.

### Example 6

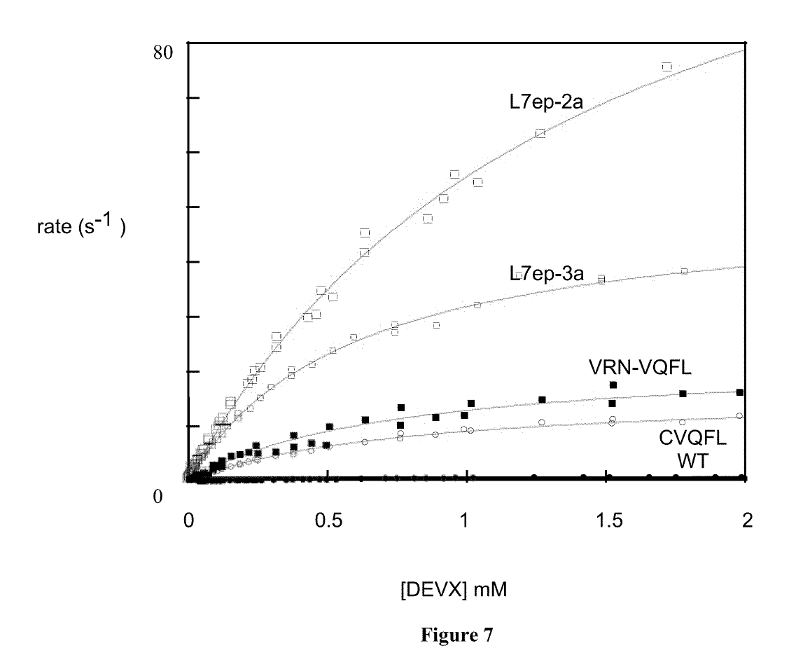

Kinetic Measurements. All assays with DEVX, paraoxon, S_{P}-1, R_{P}-1, malathion, and demeton-S were 250 μL in total volume and followed for 15 minutes in a 96-well plate reader at 30° C. Assays with VX were conducted in a volume of 500 μL in 1 mL cuvettes. DEVX, demeton-S, malathion, and VX assays monitored the release of the product thiol at 412 nm (Δε₄₁₂=14,150 M⁻¹ cm⁻¹) by the inclusion of DTNB in the reaction mixture (50 mM HEPES, pH 8.0, 100 μM CoCl₂, and 0.3 mM DTNB). Assays with paraoxon and compound 1 (FIGS. 4A and 4G) were conducted in 50 mM CHES, pH 9.0, and 100 μM CoCl₂. Assays of compound 1 (FIGS. 4A and 4G) contained 10% methanol. Paraoxon hydrolysis was followed by the release of p-nitrophenol at 400 nm (Δε₄₀₀=17,000 M⁻¹ s⁻¹) and the hydrolysis of compound 1 (FIGS. 4A and 4G) was followed at 294 nm (Δε₂₉₄=7,710 M⁻¹ cm⁻¹). Reactions were initiated by the addition of enzyme. The data were fit to equation 1 to obtain values of Kₘ, k_{cat}, and k_{cat}/Kₘ. A representative data set is provided in FIG. 7. FIG. 7 depicts representative Michaelis-Menton plots for the hydrolysis of DEVX by wild-type and evolved variants of PTE. Reaction conditions were 50 mM Hepes (pH 8), 100 μM CoCl₂, 0.3 mM DTNB in a total volume of 250 μL at 30° C. (FIG. 7). Reactions were initiated by addition of appropriately diluted enzyme. Enzyme concentrations in the reactions were; wild-type=54 nM, CVQFL=5.0 nM, VRN-VQFL=6.29 nM, L7ep-3a=1.88 nM, and L7ep-2a 2.26 nM. The solid line represents the fit of the data to equation 1.

v/Eₜ=k_{cat}(A)/(Kₘ+A) (Equation 1)

### Example 7

Stereoselective Hydrolysis of Racemic VX and VR. Low initial concentrations (19 to 160 μM) of racemic VX and VR were hydrolyzed by variants of PTE in a solution containing 0.1 mM CoCl₂, 0.3 mM DTNB, and 50 mM Hepes, pH 8.0. The reactions were followed to completion and the fraction of VX and VR hydrolyzed plotted as a function of time. The time courses were fit to equations 2 and 3 where F is the fraction of substrate hydrolyzed, a and b are the magnitudes of the exponential phases, t is time, and k₁ and k2 are the rate constants for each phase.

F=a(1−e^{-k}^{₁}^{t}) (Equation 2)

F=a(1−e^{-k}^{₁}^{t})+b(1−e^{-k}^{₂}^{t}) (Equation 3)

To identify which one of the two enantiomers of VX or VR was preferentially hydrolyzed by the PTE variants, one gram of racemic VX was hydrolyzed in a 400 mL reaction mixture containing 50 mM bis-tris-propane (pH 8.0), 100 μM CoCl₂, and 36 nM of the QF mutant (H254Q/H257F) at 33° C. The reaction was monitored by determining the concentration of the thiol product with DTNB. When the reaction was approximately 50% complete, the remaining VX was extracted with 200 mL of ethyl acetate. The volume of the extract was reduced to approximately 2 mL by rotary evaporation at 41° C. The unreacted VX was analyzed with a polarimeter and observed to rotate plane polarized light in a positive direction (+0.055° to +0.075°) which corresponds to an enantiomeric preference for hydrolysis of the S_{P}-enantiomer of VX by the QF mutant.(17)

### Example 8

Construction and Screening of Active Site Libraries. The variant QF (H254Q/H257F) was previously identified as being improved against the chiral centers in VX and VR.(18) Testing the catalytic activity of this mutant with the VX analogue, DEVX, revealed that this variant has an enhanced activity for the hydrolysis of the phosphorothiolate bond, relative to wild-type PTE. The amino acid residue numbering of the sequence including the leader sequence has been retained. The variant QF then served as the starting point for the construction of the F306X/Y309X and W131X/F132X double-substitution protein libraries. Screening 920 colonies from the F306X/Y309X library with DEVX failed to identify any variant that was improved relative to the QF parent. From the W13X/F132X library, a total of 1100 colonies were screened with DEVX and the two best mutants were identified as LQF (QF+F132L) and VQF (QF+F132V). The LQF variant served as the starting template for the 254X/257X library. Approximately 1650 colonies were screened from this library with DEVX, but none proved to be better for the hydrolysis of DEVX.

The 271X/308X library was created using sequential QuikChange procedures; first at position 271 then at position 308 using the LQF template. Approximately 2200 colonies from this library were screened and the best variant was LQFL (LQF+S308L). Incorporation of the new mutation (S308L) into the previously identified VQF variant further enhanced the catalytic activity. The variant VQFL (VQF+S308L) was utilized as the parent for the 106X/303X library. Approximately 1100 colonies were screened with DEVX and the best variant identified was CVQFL (VQFL+I106C). The variant CVQFL was carried forward in the construction of the 60X/317X library. Nearly 1500 colonies from this library were screened with DEVX, but improved variants were not detected.

A number of mutations are known to improve protein expression levels for PTE, including A80V, K185R, and I274N.(28,29) These mutations do not typically result in significant changes in the kinetic constants for a given substrate, but they dramatically improve the amount of enzyme produced per liter of cell culture. Adding these expression-enhancing mutations to VQFL resulted in an additional variant, VRN-VQFL (A80V/K185R/I274N+VQFL) with a 26-fold improvement in the value of k_{cat}/Kₘ, relative to the wild-type PTE. The inclusion of two additional expression-enhancing mutations (D208G/R319S) resulted in a decrease in catalytic activity.(29) Kinetic constants for the PTE variants with DEVX as the target substrate are presented in Table 2. Kinetic constants for additional variants are provided in Table 3.

TABLE 3
Activity of additional PTE Variants with DEVX.

| Variant | k_{cat}(s⁻¹) | Kₘ(mM) | k_{cat}/Kₘ(M⁻¹s⁻¹) | |
|---|---|---|---|---|
| QF.1 | 0.67 ± 0.01 | 2.2 ± 0.1 | (3.05 ± 0.01) | × 10² |
| QF.a | 1.23 ± 0.02 | 2.3 ± 0.1 | (5.35 ± 0.02) | × 10² |
| QF.b | 0.7 ± 0.2 | 1.9 ± 0.1 | (3.9 ± 0.1) | × 10² |
| LQF.1 | 10.1 ± 0.2 | 2.32 ± 0.08 | (4.4 ± 0.2) | × 10² |
| LQF.2 | 4.2 ± 0.2 | 4.1 ± 0.3 | (1.02 ± 0.08) | × 10³ |
| LQF.3 | 8.4 ± 0.3 | 3.0 ± 0.2 | (2.8 ± 0.2) | × 10² |
| LQF.4 | 21.7 ± 0.4 | 2.91 ± 0.09 | (7.5 ± 0.2) | × 10² |
| LQF.a | 9.4 ± 0.2 | 1.62 ± 0.07 | (5.8 ± 0.3) | × 10² |
| LQF.b | 14.5 ± 0.4 | 2.8 ± 0.1 | (5.2 ± 0.3) | × 10² |
| LQF.c | 25.2 ± 0.8 | 2.8 ± 0.2 | (9.2 ± 0.6) | × 10² |
| LQF.d | 4.1 ± 0.3 | 13 ± 1 | (3.1 ± 0.4)10x² | |
| VQFL.1 | 24.9 ± 0.5 | 2.0 ± 0.1 | (1.23 ± 0.05) | × 10⁴ |
| VQFL.2 | 20.4 ± 0.6 | 2.6 ± 0.1 | (7.9 ± 0.5) | × 10² |
| VQFL.3 | 0.93 ± 0.03 | 2.3 ± 0.1 | (4.1 ± 0.3) | × 10² |
| L7ep-4 | 18.8 ± 0.3 | 0.89 ± 0.03 | (2.11 ± 0.08) | × 10⁴ |
| L7er-5 | 13.4 ± 0.1 | 1.06 ± 0.02 | (1.26 ± 0.03) | × 10⁴ |
| L7ep-6 | 17.2 ± 0.2 | 0.63 ± 0.02 | (2.73 ± 0.08) | × 10⁴ |
| L7ep-7 | 18.4 ± 0.2 | 0.66 ± 0.02 | (2.81 ± 0.09) | × 10⁴ |
| L7ep-8 | 16.2 ± 0.3 | 1.11 ± 0.04 | (1.45 ± 0.05) | × 10⁴ |
| L7ep-9 | 16.3 ± 0.2 | 0.54 ± 0.02 | (3.0 ± 0.1) | × 10⁴ |
| L7ep-10 | 9.6 ± 0.1 | 1.23 ± 0.03 | (7.8 ± 0.2) | × 10² |
| L7ep-11 | 17.1 ± 0.2 | 1.21 ± 0.03 | (1.42 ± 0.04) | × 10⁴ |
| L7ep-12 | 5.74 ± 0.08 | 0.50 ± 0.02 | (7.8 ± 0.2) | × 10² |
| L72p-2c | 16.1 ± 0.6 | 1.6 ± 0.1 | (9.8 ± 0.8) | × 10² |
| L7ep-2d | 94 ± 3 | 1.7 ± 01 | (5.4 ± 0.3) | × 10⁴ |
| L7ep-2e | 44 ± 2 | 1.24 ± 0.09 | (3.6 ± 0.3) | × 10⁴ |
| L7ep-2f | 80 ± 2 | 1.58 ± 0.08 | (5.0 ± 0.3) | × 10⁴ |
| L7ep-2g | 80 ± 2 | 1.58 ± 0.08 | (5.3 ± 0.4) | × 10⁴ |
| L7ep-2h | 82 ± 2 | 0.94 ± 0.05 | (8.7 ± 0.3) | × 10⁴ |
| L7ep-2i | 44 ± 1 | 0.88 ± 0.04 | (5.0 ± 0.3) | × 10⁴ |
| L7ep-2j | 31 ± 1 | 0.8 ± 0.05 | (4.0 ± 0.3) | × 10⁴ |
| L7ep-3c | 35.2 ± 0.5 | 0.79 ± 0.02 | (4.5 ± 0.1) | × 10⁴ |
| L73p-3d | 25.2 ± 0.5 | 0.58 ± 0.03 | (4.4 ± 0.2) | × 10⁴ |

### Example 9

Construction of Targeted Error-Prone Library. The CVQFL variant was used as the parent for the construction of an error-prone library with an average of six mutations per gene, targeted exclusively to Loop-7 of PTE (residues 253-276). The amino acid residue numbering of the sequence including the leader sequence has been retained. Approximately 4000 colonies from this library were screened with DEVX and a total of 12 variants were identified as being more active than the parent, CVQFL. The values of k_{cat}/Kₘ for the best variants, L7ep-1, L7ep-2 and L7ep-3, were improved 27-, 63-, and 36-fold, respectively, for the hydrolysis of DEVX.

The variant L7ep-2 (CVQFL+H254R/N265D/A270D/L272M/S276T) has 5 amino acid changes to the sequence of Loop-7, relative to the parent. These five sites, and residue positions 257 and 274, were subjected to further optimization. Two, two-site libraries (R254X/F257X, and M272X/I274X) and three, single-site libraries (D265X, D270X, and T276X) were constructed to ensure that the optimum amino acid residue is represented at each position. Screening the libraries with DEVX revealed no improvements at residue positions 254, 257, 265, or 270, but numerous improved combinations were identified in the 272X/274X library. One of these variants, L7ep-2a (L7ep-2+I274T), has a k_{cat} of 135 s⁻¹ and a k_{cat}/Kₘ 78-fold improved over wild type enzyme. To further optimize the L7ep-3 variant (CVQFL+H257Y/A270V/L272M), Loop-7 was subjected to a second round of targeted error-prone PCR. Screening with DEVX identified two variants, L7ep-3a (L7ep-3+I274N) and L7ep-3b (L7ep-3+A270D) that were substantially improved over the parent enzyme. Similar experiments were attempted using L7ep-2 as the starting template, but no improved variants were identified. The kinetic constants for the hydrolysis of DEVX by these mutants are presented in Table 2 and Table 3.

### Example 10

Stereoselectivity of PTE Variants for Chiral VX Analogues. In this investigation there was no attempt to include a stereochemical preference in the screening of mutant libraries. Wild-type PTE is known to have a slight preference for the S_{P}-enantiomer of the VX chiral center.(18) To determine that there were no perturbations in stereoselectivity, the variants with improved catalytic activity against DEVX were analyzed using the chromophoric analogues R_{P}-1 and S_{P}-1, and the results are presented in Table 2. With the exception of L7ep-2, L7ep-2a, and L7ep-2b, the evolved variants have values of k_{cat}/Kₘ that are greater for the S_{P}-enantiomer than for the R_{P}-enantiomer.

### Example 11

Enzymatic Specificity. To assess changes in substrate specificity, the enzyme variants were tested with paraoxon and demeton-S as alternative substrates. The results are provided in Table 4. The variants from the active site evolution experiments maintain a high enzymatic efficiency for paraoxon, although it is reduced nearly an order of magnitude from wild-type enzyme. The catalytic activity using demeton-S did not show any significant improvement for most of the variants tested. The exceptions are L7ep-2, L7ep-2a, and L7ep-2b, which have increased k_{cat} and decreased Kₘ values for demeton-S.

TABLE 4
Kinetic parameters for PTE variants with paraoxon and demeton-S^{a}.

| | Paroxon | | | Demeton-S | | |
|---|---|---|---|---|---|---|
| | k_{cat} | Kₘ | k_{cat}/Kₘ | k_{cat} | Kₘ | k_{cat}/Kₘ |
| Variant | (s⁻¹) | (μM) | (M⁻¹s⁻¹) | (s⁻¹) | (mM) | (M⁻¹s⁻¹) |
| WT | 6700 | 100 | 6.7 × 10⁷ | 1.4 | 1.2 | 1.1 × 10³ |
| QF | 41 | 5.3 | 7.7 × 10⁶ | 6.3 | 2.6 | 2.7 × 10³ |
| LQF | 72 | 11.1 | 6.5 × 10⁶ | 4.4 | 3.3 | 1.3 × 10³ |
| VQF | 108 | 10.5 | 1.0 × 10⁷ | 10 | 6.1 | 1.7 × 10³ |
| LQFL | 90 | 11 | 8.2 × 10⁶ | 5.2 | 3.1 | 1.7 × 10³ |
| VQFL | 66 | 5.4 | 1.2 × 10⁷ | 4.2 | 3.9 | 1.1 × 10³ |
| CVQFL | 38 | 5.6 | 6.8 × 10⁶ | 6.1 | 2.5 | 2.4 × 10³ |
| VRN-VQFL | 116 | 8 | 1.5 × 10⁷ | 7.1 | 2.8 | 2.5 × 10³ |
| VRNGS-VQFL | 227 | 8.5 | 2.7 × 10⁷ | 6.8 | 3.1 | 2.2 × 10³ |
| L7ep-1 | 1590 | 116 | 1.4 × 10⁷ | 0.12 | 1.0 | 1.2 × 10² |
| L7ep-2 | 245 | 93 | 2.6 × 10⁶ | 73 | 0.84 | 8.7 × 10⁴ |
| L7ep-3 | 146 | 11.2 | 1.3 × 10⁷ | 3.4 | 2.4 | 1.4 × 10³ |
| L7ep-2a | 243 | 46 | 5.3 × 10⁶ | 32 | 0.58 | 5.4 × 10⁴ |
| L7ep-2b | 280 | 13.5 | 2.1 × 10⁷ | 53 | 0.57 | 9.2 × 10⁴ |
| L7ep-3a | 85 | 7.2 | 1.2 × 10⁷ | 1.8 | 2.5 | 7.4 × 10² |

*Standard errors from fits of the data fit to equation 1 are less than 10% of the stated values

### Example 12

Hydrolysis of Racemic VX. Wild-type PTE and selected variants were characterized using racemic VX as a substrate and the results are presented in Table 5. Wild-type PTE has a low k_{cat} (0.9 s⁻¹) and relatively high Kₘ, resulting in a diminished k_{cat}/Kₘ for the hydrolysis of VX. The variant QF dramatically improves both k_{cat} and Kₘ values resulting in a 100-fold increase in k_{cat}/Kₘ. The VRN-VQFL variant had the highest value of k_{cat}/Kₘ that was increased more than 230-fold over wild-type enzyme. VRN-VQFL includes the following mutations A80V, K185R, I274N, F132V, H254Q, H257F, and S308L. The L7ep-3a had the highest k_{cat} and was increased more than 150-fold, relative to wild-type PTE. The QF mutant was shown to preferentially hydrolyze the S_{P}-enantiomer of VX by polarimetry.

TABLE 5
Activity of PTE variants with racemic VX^{a}.

| | | | | | Stereo- |
|---|---|---|---|---|---|
| | | | | | chemical |
| Variant | k_{cat} (s⁻¹) | Kₘ (mM) | k_{cat}/Kₘ (M⁻¹s⁻¹) | | Preference^{b} |
| WT | 0.9 ± 0.1 | 2.9 ± 0.9 | 3 ± 1 | × 10² | ND^{c} |
| QF | 16 ± 1 | 0.5 ± 0.1 | 3.0 ± 0.6 | × 10⁴ | 12:1 (S_{P}) |
| CVQFL | 45 ± 6 | 2.1 ± 0.7 | 2.2 ± 0.8 | × 10⁴ | 1:1 |
| VRN-VQFL | 44 ± 1 | 0.59 ± 0.09 | 7 ± 1 | × 10⁴ | 3:1 |
| L7ep-1 | 11 ± 1 | 0.8 ± 0.2 | 1.4 ± 0.3 | × 10⁴ | ND |
| L7ep-2 | 25 ± 2 | 1.3 ± 0.3 | 2.2 ± 0.3 | × 10⁴ | 5:1 |
| L7ep-3 | 31 ± 2 | 0.5 ± 0.2 | 6 ± 2 | × 10⁴ | 4:1 |
| L7ep-2a | 56 ± 4 | 3.4 ± 0.7 | 1.6 ± 0.4 | × 10⁴ | 4:1 |
| L7ep-2b | 56 ± 14 | 8 ± 3 | 7 ± 4 | × 10³ | 12:1 (R_{P}) |
| L7ep-3a | 137 ± 22 | 5 ± 2 | 3 ± 1 | × 10⁴ | 4:1 |

^{a}Standard errors from fits of the data to equation 1.
^{b}Identity of preferred enantiomer was not determined for variants with less than a 10-fold preference.
^{c}ND = not determined.

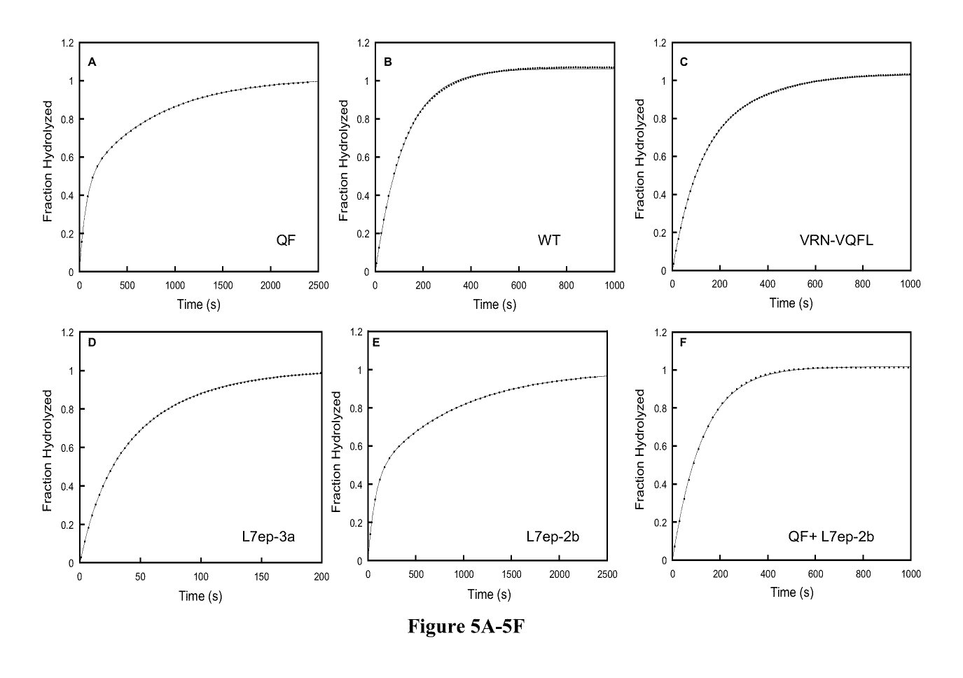

Stereoselective hydrolysis of racemic VX by the PTE mutants was evaluated by analyzing the time courses for the complete hydrolysis of VX at concentrations below the Michaelis constant. The presence of stereoselectivity in these time courses is manifested as the appearance of two exponential phases as observed with the QF variant where the ratio of rate constants is 12:1 (FIG. 5A).

FIG. 5. Representative time courses for the complete hydrolysis of racemic VX by selected PTE variants. (A) QF; (B) WT; (C) VRN-VQFL; (D) L7ep-3a (E) L7ep-2b; and (F) L7ep-2b and QF. The time courses for panels B and F were fit to equation 2, while the data for panels A, C, D, and E were fit to equation 3.

The CVQFL variant exhibited no selectivity (FIG. 5B), whereas the variants VRN-VQFL (3:1) and L7ep-3a (4:1) displayed relatively low selectivity (FIGS. 2C and 2D). For the L7ep-2b mutant, the observed stereoselectivity was 12:1 (FIG. 5E). The enantiomeric specificity of the L7ep-2b variant was determined by its ability to complement the hydrolysis of the slower R_{P}-enantiomer of VX after the addition of the variant QF. Plots of the fractional hydrolysis of VX as a function of time give two well-defined phases for each of these two variants (FIG. 5A and FIG. 5E). Mixing the two variants together resulted in a single well-defined monophasic curve demonstrating that the two variants prefer the opposite enantiomers (FIG. 5F). Therefore, the L7ep-2b variant preferentially hydrolyzes the Rₚ-enantiomer of VX. The ratios of rate constants for the L7ep-2, L7ep-3, and L7ep-2a mutants were 5:1, 4:1, and 4:1, respectively (Table 5). In the absence of enantiomerically pure VX, the modest selectivities for these variants prevented the definitive assignment of the preferred enantiomer.

### Example 13

Construction of Active Site libraries by Overlap Extension. Mutagenic primers contained an NNS codon at the position of interest and extended 15 bp to either side of this codon. The PTE gene was amplified in three segments using standard PCR techniques (10 ng template and 125 ng each primer in a 50 μL reaction using pfuTurbo polymerase). The first segment extended from the 5′ end of the gene to 15 bp beyond the first mutagenic position. The second segment extended from 15 bp upstream of the first mutagenic position to 15 bp downstream of the second mutagenic position. The third segment extended from 15 bp upstream of the second mutagenic site to the 3′ end of the gene. The 5′ and 3′ primers included NdeI and EcoRI restriction sites respectively. A second PCR reaction was performed using the generated segments as the template DNA. The three fragments were combined in equimolar ratio (500 ng total) and amplified for 30 cycles with pfuTurbo using the primers for the 5′ and 3′ ends of the PTE gene. The overlaps between the fragments allowed for the formation of a single product corresponding to the size of the complete PTE gene. The product and vector were then digested with NdeI and EcoRI, gel purified and ligated together.

### Example 14

Construction of Error Prone Library. Primer pairs used to amplify Loop-7 corresponding to the DNA sequence for residues 242-252 and 277-287. The reaction contained 20 ng template (CVQFL variant), 1 μM forward and reverse primers, 0.35 mM dATP, 0.4 mM dCTP, 0.2 mM dGTP, 1.35 mM dTTP, 1 mM MgCl₂, 1× GoGreen Taq Buffer (Promega, Madison Wis.) 1.5 mM MnCl₂ and 1 μL Go Taq in 50 μL reaction. Thermocycler program was 2 min initial denaturation at 95° C., followed by 30 cycles of 95° C. for 45 s, 60° C. for 1 min, 72° C. for 3 min, and a final elongation at 72° C. for 10 min. The remaining portions of the PTE gene were amplified using standard PCR techniques with the reverse primers for the Loop-7 fragment and the 5′ and 3′ end primer, resulting in three overlapping fragments. The final gene product was constructed by the overlap extension technique as described above. The mutated gene was digested with NdeI and EcoRI and ligated into pET 20 b (40 μL reaction containing 60 ng vector DNA, 3× molecular excess of PTE gene product, 4 μL T4 DNA ligase buffer and 2 μL T4 DNA ligase (NEB). Sequencing confirmed an average of 6 base pair changes per gene in loop-7. The identities of mutants from this library are given in Table 3.

### Example 15

Enzyme Expression and Purification. BL21 (DE3) cells containing plasmid with wild-type or variant PTE were grown for ˜8 hours in 5 mL LB broth. 1 L cultures of Terrific Broth (12 g Tryptone, 24 g yeast extract, 4 mL glycerol, 2.3 g KH2PO4, 12.5 g K₂HPO₄ in 1 L H₂O) supplemented with 1.0 mM CoCl₂ were inoculated with 1 mL of the growing culture. Cells were grown overnight at 30° C. with shaking. Protein expression was induced by addition of 1.0 mM IPTG and expression proceeded for an additional 24 hours. Cells were harvested by centrifugation at 11,000 g for 10 minutes. Cell pellets were stored at −80° C. prior to use. Cells from 1 L of culture were resuspended in 100 mL purification buffer (50 mM HEPES (pH 8.5), 100 μM CoCl₂). Cellular lysis was achieved by sonication on ice for a total of 20 minutes using a medium power setting. Cell debris was removed by centrifugation at 18,500 g for 10 minutes. Protamine sulfate (0.45 g in 20 mL purification buffer) was added dropwise and incubated for 20 minutes to remove nucleic acids. Precipitated materials were removed by centrifugation at 18,500 g for 10 minutes. Supernatant was brought to 60% saturation with ammonium sulfate and stirred in the cold for 30 minutes to precipitate PTE. Protein was removed from the supernatant by centrifugation at 18,500 g for 20 minutes. The supernatant was decanted and the pellet re-dissolved in 5 mL purification buffer. Up to 5 mL of the protein solution was loaded on a Superdex 200 (16/60) preparatory size exclusion column on a GE Health Care (Piscataway, N.J.) AKTA FLPC system. Peak fractions were collected and assayed for activity against paraoxon. Fractions with the most activity were further purified using a gravity-fed DEAE column pre-equilibrated in purification buffer.

### Example 16

Synthesis of DEVX. DEVX was made by the reaction of diethylchlorophosphate with N,N-diisopropylaminoethanthiol. 1.5 grams of N,N-diisopropylaminoethanthiol was added to 100 mL diethyl ether and cooled in a dry ice acetone bath and purged with N2 gas. To this mixture, 7.5 ml of a 2.5 M solution of butyryl lithium in hexane was added. 1.5 g of diethylchlorophosphate was mixed with 30 mL diethyl ether in a separate flask purged with N₂ and cooled in a dry ice acetone bath. The cooled diethylchlorophosphate solution was then added to the thiol solution and the reaction stirred at room temperature for 3 hr. The reaction was then brought to 400 mL with ethyl ether and extracted with water to remove side products. Product was then extracted into the aqueous phase with 0.5 M HCl and ethyl acetate. The aqueous phase was neutralized with sodium bicarbonate and extracted with chloroform. The organic phase was dried over MgSO₄ filtered and evaporated yielding the desired product as a pure oil.

¹H NMR (300 MHz, CDCl₃): 4.05-4.174 (4H, m, OCH₂CH₃), 3.20-2.50 (6H, m, SCH₂CH₂N(CH)₂), 1.41-1.36 (6H, t, J=6.9 Hz, OCH₂CH₃), 1.05-1.03 (12H, d, J=4.8 Hz, CH(CH₃)₂

³¹P NMR (121.4 MHz CDCl₃): 29.77 ppm.

### Example 17

Purification of racemic VX. VX samples were Chemical Agent Standard Analytical Reference Material (CASARM) and were of the highest purity available, typically 99.9+/−5.4 weight % by oxidation-reduction titration, traceable to National Institute of Standards and Technology through 0.1 N iodine solution SRM 136e. However, as received, the VX gave high background readings at 412 nm at the concentrations required for kinetic analysis and therefore required further purification as follows: 80.1 μL neat VX was added to 120 μL isopropyl alcohol (to aid in dissolution), then added to 800 μL of 3 mM DTNB in 50 mM HEPES, pH 8.0. To this solution was added approximately 1 gram of Dowex® 1×4 chloride form beads (Sigma-Aldrich) and agitated gently for several minutes until the beads turned red. The VX was subsequently decanted and added to more beads until essentially all the yellow color was removed to the beads and the VX solution was almost colorless. A standard curve was then generated using 6, 30, 60 and 96 μM dilutions of VX, reacted to completion enzymatically. VX concentration in the bead-treated solution was determined by linear regression analysis using the standard curve from the direct dilutions of VX.

### Example 18

NMR Data Acquisition: All spectra were recorded on non-spinning samples at 25±2° C. with a Varian Unity INOVA 600 spectrometer (600 MHz ¹H operating frequency) fitted with a triple resonance, z-gradient probe. Routine ¹H free induction decay (FID) data sets of 16,384 complex points were collected as summations of eight or 16 acquisitions recorded with 10 ppm spectral windows, 90° pulse widths of 12 μsec, and 2 sec relaxation delays before archiving to computer disk. FID data sets were apodized with a line broadening factor of 0.3 Hz before Fourier transformation into spectra, manual phase correction into pure absorption mode, and chemical shift referencing to external tetramethylsilane.

³¹P FID data sets of 65,536 complex points were collected as summations of 32 acquisitions using 100 ppm spectral windows and 90° pulse widths of 30 μsec. All ³¹P data acquisitions incorporated inverse-gated 1H decoupling (decoupling only during FID acquisition) with a low power composite pulse sequence to increase signal-to-noise ratios without signal enhancements from ¹H-³¹P nuclear Overhauser effects.(31) Spin-lattice relaxation times (T1) for the VX ³¹P signal and that for the O-ethyl methylphosphonate (EMP) hydrolysis product were measured with the inversion recovery pulse sequence [180°-τ-90°-acquisition] incorporating nine randomized τ delays. For quantitative ³¹P spectra, data sets were collected with relaxation delays >5T₁ for all ³¹P signals in the spectra (˜12 sec) to allow complete signal relaxation, and the spectrometer carrier frequency was centered between the VX substrate signal (ca. 57 ppm) and that of the O-ethyl,methylphosphonate (EMP) hydrolysis product (ca. 23 ppm) to minimize off-resonance effects. The ³¹P{¹H} (¹H decoupled, ³¹P observe) data sets were apodized with a 5 Hz line broadening factor before Fourier transformation into spectra and manual phase correction into pure absorption mode. ³¹P chemical shift values in spectra were referenced to external 85% phosphoric acid at −0.73 ppm.(32)

### Example 19

NMR Observation of Enzymatic Hydrolysis of VX: The enzymatic hydrolysis of VX in the presence of PTE enzymes was observed by using NMR spectroscopy to follow VX disappearance, or the appearance of its EMP hydrolysis product, over time. Enzymatic reactions were initiated by adding 0.1-25.0 μL of a single enzyme solution to a 1 mL aliquot of a racemic VX solution and briefly mixing before transferring to a NMR sample tube. This was immediately placed into the NMR spectrometer, and quantitative ³¹P{′H} FID data sets were acquired at 7.5 min time intervals over 20-75 min. Enzymatic hydrolysis rates were calculated directly from the integral values of the quantitative ³¹P{¹H} signals, and included subtraction of the measured spontaneous rate (˜55 μmole hr⁻¹) determined in separate experiments. The VX signal intensity decreases throughout the entire time course of the experiment until 75.0 min., where ≥99% of the intensity has disappeared (FIG. 8). EMP signal intensity increases over this same time frame, and at 75 min., it is the only signal observed in the spectrum.

Oligonucleotide pairs that contained the mutated codons at the specified sites were used as primers to amplify the genes for the wild-type enzyme and the following mutant enzymes: QF, YT, and GWT. The identities of the mutants are listed in Table 6. The mutations were added to each template sequentially to make the following mutant proteins: RN, QFRN, YTRN, GWT-d1, and GWT-d2.

TABLE 6
Identification of Mutants

| Abbreviation | Mutations |
|---|---|
| RN | K185R/1274N |
| QF | H254Q/H257F |
| GWT | H254G/H257W/L303T |
| QF-RN | H254Q/H257F/K185R/I274N |
| YT-RN | H257Y/L303T/K185R/I274N |
| GWT-d1 | H254G/H257W/L303T/K185R/I274N |
| GWT-d2 | H254G/H257W/L303T/K185R/I274N/A80V |
| GWT-d3 | H254G/H257W/L303T/K185R/I274N/A80V/S61T |
| GWT-f1 | H254G/H257W/L303T/M317L/K185R/I274N |
| GWT-f2 | H254G/H257W/L303T/M317L |
| GWT-f3 | H254G/H257W/L303T/M317L/I106C/F132I/L271I/ |
| | K185R/I274N |
| GWT-f4 | H254G/H257W/L303T/M317L/I106C/F1321/L271I/ |
| | K185R/I274N/A80V |
| GWT-f5 | H254G/H257W/L303T/M317L/I106C/F132I/L271I/ |
| | K185R/I274N/A80V/R67H |

### Example 20

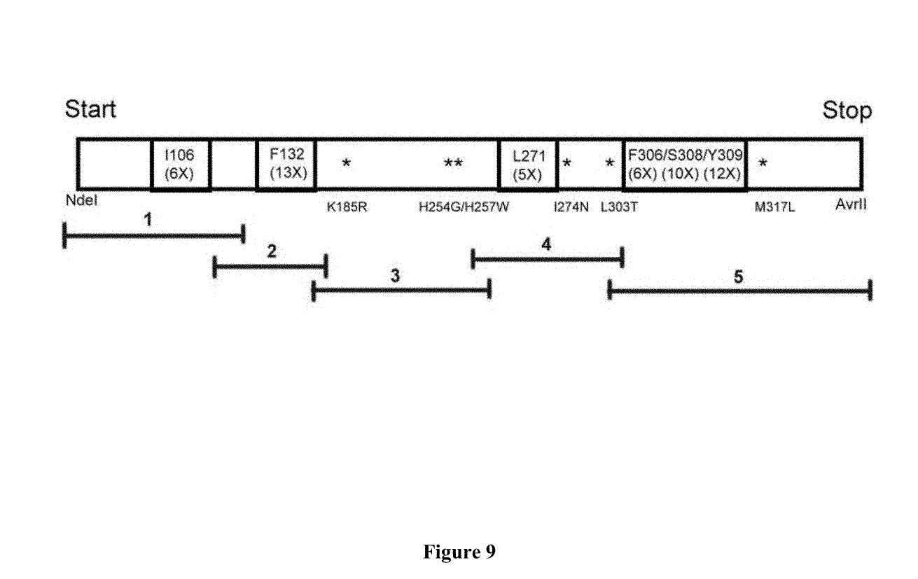

The multi site partially randomized PTE library was constructed by combining five separate segments of the gene for PTE as illustrated in FIG. 9 using primerless PCR for 15 cycles and then amplified by PCR for 55 cycles using primers specific for the 5′ and 3′ termini. The potential size of this multisite library is 1.9×10⁵ variants. The numbers below the residue identifier indicate the number of amino acids that were allowed during the construction of the library. The amplified PTE library was digested with NdeI and Avr II restriction enzymes and ligated into the GpdQ-pETDuet plasmid using T4 DNA ligase. The ligation mixture was purified using the QIAquick Kit (Qiagen) and then transformed into freshly made E. coli Top 10 competent cells (Life Technologies). The transformants were incubated at 37° C. for 1 hour and then plated on Luria-Bertani ampicillin agarose. Approximately 5.7×10⁵ colony forming units were collected and grown in LB medium for 6 hours at 37° C. The plasmids from the PTE library were extracted using the Promega Wizard Plus Miniprep Kit.

### Example 21

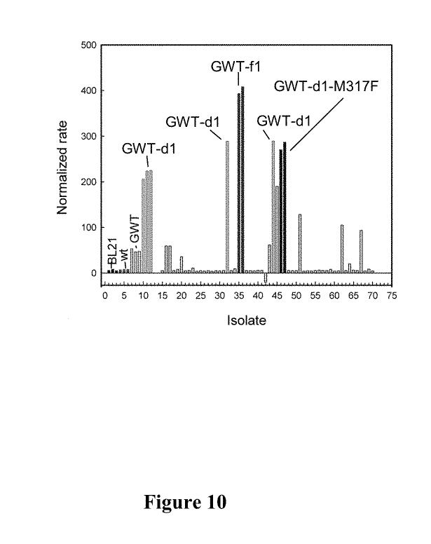

Mutant libraries were constructed using GWT-d1 as the starting template to identify more active PTE variants for the hydrolysis of S_{P}-5. Nine amino acid residues in the substrate binding pocket were considered as potential “hot spots” for the construction of these PTE libraries. The single substitution library M317X was constructed first, followed by four double substitution libraries (W131X/F132X, F306X/Y309X, S308X/Y309X, and I106X/Y308X). Approximately 60 colonies from the M317X library and around 550 colonies from each of the double-substitution libraries were picked and subsequently screened with S_{P}-5. The variants of GWT with catalytic activities higher than background from the first round of screening were isolated and then rescreened with the same substrate. (Table 6). No improvement in the hydrolysis of S_{P}-5 (FIG. 4F) was found in the double-substitution libraries, W131X/F132X, F306X/Y309X, S308X/Y309X, and I106X/Y308X. FIG. 10 illustrates the screening of the M317X single-substitution library. The bars represent the relative catalytic activities of the GWT-d1, GWT-f1, and GWT-d1-M317F mutants as labeled. Those mutants represented by the unlabeled bars were not characterized or sequenced. The best mutant identified in this screen contained a leucine substitution for Met-317 and is denoted GWT-f1. FIG. 10 depicts the screening of the M317X mutant library against S_{P}-5 using GWT-d1 as the parental template. The bars represent the relative catalytic activities of the GWT-d1, GWT-d1-M317F mutants, respectively.

### Example 22

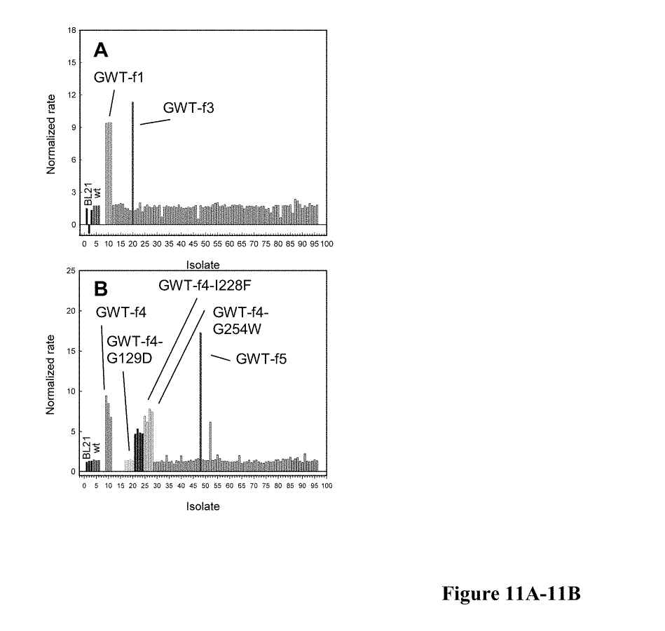

The GWT-f1 mutant was partially randomized at six sites simultaneously. The total library contained 1.9×10⁵ potential variants. Eight colonies from this library were selected to verify that the targeted sites were randomized. The PTE/GpdQ-pETDuet plasmid library was transformed into E. coli BL21(DE3) cells. Approximately 5.8×10⁵ CFU were plated on phosphate-free minimal medium with 1 mM Sₚ-5 as the sole phosphorus source. The colonies that contained beneficial mutations for the hydrolysis of S_{P}-5 were identified as being larger in size than a background colony of the parent GWT-f1 mutant. Approximately 30 of these colonies were selected for growth in 96-well blocks and subsequently assayed for catalytic activity with S_{P}-5. The screening of the partially randomized multisite library with S_{P}-5 is shown in FIG. 11A. The first nine samples include the empty vector control, wild-type PTE, and the GWT-f1 parent. A single variant was found to have more activity than the GWT-f1 parent. This mutant (GWT-f3) contained three additional changes in the amino acid sequence: I106C, F132I, and L271I. The A80V mutation was added to the GWT-f3 mutant to create the GWT-f4 variant.

In FIG. 11A, screening of the six-site randomized library using GWT-f1 as the parental template with S_{P}-5. The bars represent the relative catalytic activities of the GWT-f1 and GWT-f3 mutants as labeled.

The GWT-f4 mutant served as the template for error-prone PCR (epPCR). Random mutagenesis of the GWT-f4 gene was conducted using the Mutazyme II DNA polymerase. Ten colonies from this library were selected to establish an average mutation rate of ˜1.5 mutations/1000 bp. The epPCR generated PTE/GpdQ-pETDuet library was transformed into E. coli BL21(DE3). Approximately 6×10⁵ CFU were plated on phosphate-free minimal medium plates with 1 mM S_{P}-5 as the sole phosphorus source. Colonies larger in size than the parental strain (GWT-f4) were assayed with S_{P}-5, and the results are shown in FIG. 11B. The first 20 samples include the empty vector, wild-type PTE, GWT-f3, GWT-f3-G129D, GWT-f3-I288F, and GWT-f3-H254W. The new variant, GWT-f5, contained a single mutation, relative to GWT-f4, at Arg-67 with a change to histidine.

In FIG. 11B, screening of the error-prone PCR library using GWT-f4 as the parental template with S_{P}-5. The bars represent the relative catalytic activities of the GWT-f4, GWT-f4-G129D, GWT-f4-I228F, GWT-f4-G254W, and GWT-f5 mutants as labeled.

### Example 23

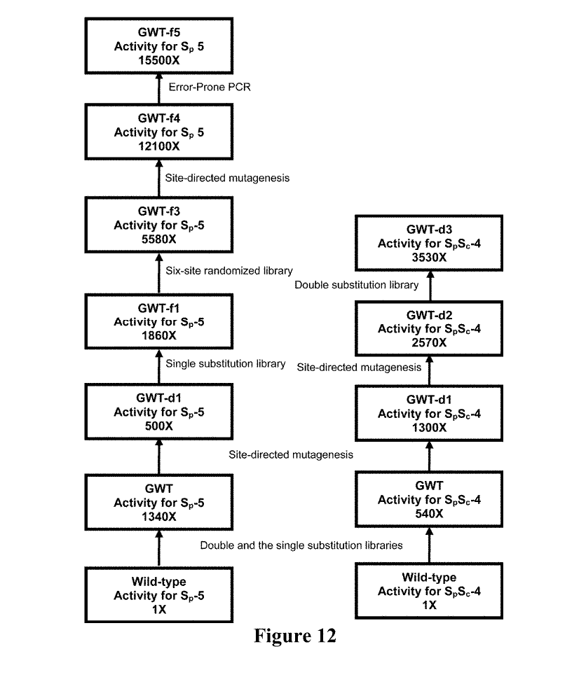

The outline for the discovery of PTE variants with enhanced activity for the hydrolysis of the S_{P}S_{C}-4 and SP-5 (FIGS. 4D and 4F) by directed evolution is depicted in FIG. 12.

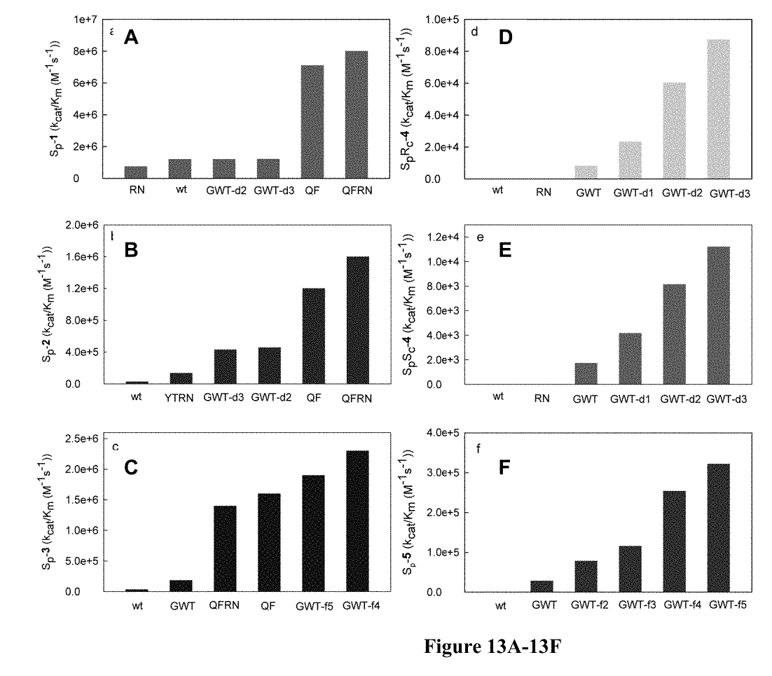

The structural modifications to GWT within the substrate binding pocket, surface and dimer interface have substantially enhanced substrate binding and catalytic turnover. The changes in the k_{cat}/Kₘ values of the S_{P}-enantiomers of the organophosphonates are depicted in FIG. 13A-13F.

Kinetic constants for sarin (GB), soman (GD), and cyclosarin (GF) were determined by monitoring the release of free fluoride at 25° C. in 50 mM bis-tris-propane buffer (pH 7.2) using a fluoride electrode. Stereospecificity for hydrolysis of nerve agents was obtained by following the complete hydrolysis of 0.5 mM racemic mixtures of GB or GF. Reactions were conducted in 50 mM bis-tris-propane buffer (pH 7.2) and followed by the release of fluoride.

### Example 24

Screening and Kinetic Analysis of Wild-Type and Mutant Enzymes on GB, GD, and GF. For the purposes of initial screening, specific activities were determined with 3 mM substrate (Table 7). The YT mutant was very active with all three substrates, and the YT-RN mutant had higher activity than the wild type with GD. Therefore, kinetic constants were determined for these enzyme-substrate combinations (Table 8). The stereospecificity of variants was determined by following the fluoride released during the complete hydrolysis of GB or GF using the 0.5 mM racemic substrate. Substantial differences in the rates for the individual enantiomers result in biphasic curves. The specificity of GWT toward GF is known from polarimetry experiments,(9) while the stereopreference of the remaining variants was determined by the ability of the variant to complement the slow phase in the GWT-catalyzed reaction (Table 7). YT has the same stereopreference as GWT for GF and has previously been shown to have the same preference as GWT for GD.(5) The stereopreference for the variants toward GB was determined by the ability to complement the slow phase in the YT-catalyzed reaction (Table 7). Adding mutations to the variants YT and YT-RN will likely provide variants that have improved activity with G-agents over wild-type PTE. Mutations at various locations within the sequence of the YT mutant will be added and their activity measured to identify variants that exhibit high activity with G-agents. Such variants would be useful in the decontamination of people, items, and locations contaminated with one or more G-agents. In an embodiment, further improvements in catalytic activity could be gained by simultaneously mutating pairs of residues in the active site. In an embodiment, error-prone PCR could be utilized to obtain mutations within PTE. In an embodiment, mutations within the active site of PTE could provide increased catalytic activity toward a G-agent. In an embodiment, methods disclosed regarding mutating PTE and determining activity with V-agents can also be utilized with mutating PTE and determining activity with G-agents.

TABLE 7
Activity of Wild-Type and Mutant Enzymes with
Racemic G-Agents*

| | | | | GB** | GF** |
|---|---|---|---|---|---|
| | | | | preferred | preferred |
| Enzyme | GB | GD | GF | enantiomer | enantiomer |
| WT | 303 | 14 | 363 | NA*** | R_{P} |
| YT | 843 | 212 | 240 | S_{P} | S_{P} |
| YT-RN | 263 | 115 | 116 | S_{P} | S_{P} |
| QF-RN | 32 | 1.0 | 41 | NA*** | NA*** |
| GWT | 20 | 2.0 | 44 | S_{P} | S_{P} |
| GWT-d1 | 57 | 2.0 | 7 | S_{P} | S_{P} |
| GWT-d2 | 52 | 1.0 | 211 | S_{P} | S_{P} |
| GWT-d3 | 48 | 8 | 35 | S_{P} | S_{P} |
| GWT-f3 | 142 | 10 | 94 | S_{P} | S_{P} |
| GWT-f5 | 240 | 19 | 59 | S_{P} | S_{P} |

*In micromoles per minute per milligram of protein.
**Determined with 0.5 mM racemic substrate.
***No significant stereopreference under these conditions.

TABLE 8
Kinetic Constants for Hydrolysis of GB, GD, and GF*

| | | | | k_{cat}/Kₘ | |
|---|---|---|---|---|---|
| Enzyme | substrate | k_{cat} (s⁻¹) | Kₘ (μM) | (M⁻¹ s⁻¹) | |
| WT | GB | 430 ± 50 | 1800 ± 400 | 2.4 (0.6) | × 10⁵ |
| WT | GD | 12 ± 1 | 800 ± 200 | 1.5 (0.4) | × 10⁴ |
| WT | GF | 210 ± 30 | 900 ± 300 | 2.3 (0.8) | × 10⁵ |
| YT | GB | 520 ± 30 | 260 ± 50 | 2.0 (0.4) | × 10⁶ |
| YT | GD | 240 ± 20 | 460 ± 90 | 5 (1) | × 10⁵ |
| YT | GF | 130 ± 10 | 170 ± 50 | 8 (2) | × 10⁵ |
| YTRN | GD | 100 ± 10 | 300 ± 100 | 4 (2) | × 10⁵ |

*Racemic mixtures of GB, GD, and GF were used for these measurements.

The kinetic parameters of the purified wild-type PTE and its mutants with the entire set of chiral organophosphonate compounds shown in FIGS. 4A-4L are provided in Tables 7, 9, and 10.

TABLE 9
Values of k_{cat} (s⁻¹) for Wild-Type PTE and Its Mutants*

| | | | | | QF | YT | | GWT- |
|---|---|---|---|---|---|---|---|---|
| | WT | QF | YT | RN | RN | RN | WT | d1 |
| R_{P}-1 | 1.5e2 | 1.7e2 | 7.3e0 | 9.0e1 | 2.0e2 | 1.2e1 | 1.4e1 | 1.4e1 |
| S_{P}-1 | 6.7e2 | 3.2e1 | 4.1e2 | 8.2e2 | 4.5e1 | 1.0e3 | 1.9e2 | 2.9e2 |
| R_{P}-2 | 1.0e2 | 4.8e1 | 1.8e1 | 6.6e1 | 6.6e1 | 2.0e1 | ND | 2.1e1 |
| S_{P}-2 | 4.0e1 | 7.2e0 | 3.7e2 | 2.0e1 | 1.1e1 | 7.0e2 | 9.2e1 | 8.6e1 |
| R_{P}-3 | 9.3e1 | 7.0e1 | 5.1e1 | 4.8e1 | 1.3e2 | 4.3e1 | 2.0e1 | 8.0e0 |
| S_{P}-3 | 2.2e1 | 6.3e0 | 1.0e2 | 1.6e1 | 1.3e1 | 7.7e2 | 5.0e1 | 5.5e1 |
| R_{P}R_{C}-4 | 3.4e0 | 5.5e-1 | 4.1e-1 | 4.5e0 | 1.1e0 | 5.8e-1 | 2.0e0 | 2.4e0 |
| R_{P}S_{C}-4 | 4.5e-1 | 1.7e-1 | ND | 4.2e-1 | 3.3e-1 | 1.9e0 | 2.1e-1 | 1.4e0 |
| S_{P}R_{C}-4 | 7.7e-1 | 6.3e-1 | 6.3e0 | 1.3e0 | 1.2e0 | 4.3e0 | 1.2e1 | 1.4e1 |
| S_{P}S_{P}-4 | 1.6e-2 | 3.3e-1 | 2.1e0 | ND | 5.e-1 | 3.2e0 | 2.9e0 | 6.5e0 |
| R_{P}-5 | ND | 3.8e1 | 5.9e0 | 2.5e1 | 2.2e1 | 1.7e1 | 8.1e-1 | ND |
| S_{P}-5 | ND | ND | 5.1e0 | 1.3e-1 | 4.1e-1 | 7.2e0 | 1.9e1 | 3.1e0 |

| | GWT- | GWT- | GWT- | GWT- | GWT- | GWT- | GWT- |
|---|---|---|---|---|---|---|---|
| | d2 | d3 | f1 | f2 | f3 | f4 | f5 |
| R_{P}-1 | 1.3e1 | 9.6e0 | 1.3e1 | 2.2e2 | 7.9e1 | ND | ND |
| S_{P}-1 | 5.3e2 | 4.0e2 | 3.6e2 | 4.4e2 | 6.6e2 | 1.1e3 | 7.2e2 |
| R_{P}-2 | 2.2e1 | 5.9e1 | 9.8e0 | 3.3e1 | ND | ND | ND |
| S_{P}-2 | 1.3e2 | 2.0e2 | 3.0e2 | 2.3e2 | 6.2e2 | 1.1e3 | 5.9e2 |
| R_{P}-3 | 1.3e1 | 2.2e1 | 2.9e1 | 3.4e1 | 1.3e1 | 1.5e1 | ND |
| S_{P}-3 | 5.8e1 | 6.0e1 | 8.0e1 | 8.8e1 | 1.4e2 | 2.5e2 | 1.8e2 |
| R_{P}R_{C}-4 | 4.3e0 | ND | 4.0e0 | ND | ND | ND | 2.9e0 |
| R_{P}S_{C}-4 | ND | ND | 8.9e-1 | 1.9e0 | ND | ND | 1.9e0 |
| S_{P}R_{C}-4 | 5.0e1 | 6.4e1 | 3.1e1 | 3.1e1 | 1.6e1 | 4.7e1 | 1.7e1 |
| S_{P}S_{P}-4 | 1.2e1 | 2.4e1 | 5.7e1 | 8.1e0 | 4.2e0 | 5.6e0 | 6.1e0 |
| R_{P}-5 | 9.7e-1 | ND | ND | ND | 2.2e0 | ND | 3.3e0 |
| S_{P}-5 | 4.7e1 | 4.4e1 | 2.6e1 | 3.1e1 | 4.4e1 | 1.2e2 | 1.2e2 |

*The standard errors, from fits of the data to v/Eₜ = (k_{cat}[A]/(Kₘ + [A]), are ess than 20% of the stated values. ND, not determined.

TABLE 10
Values of k_{cat}/Kₘ (M⁻¹ s⁻¹) for Wild-Type PTE and Its Mutants*

| | | | | | QF | YT | | GWT- | GWT- | GWT- | GWT- | GWT- | GWT- | GWT- | GWT- |
|---|---|---|---|---|---|---|---|---|---|---|---|---|---|---|---|
| | WT | QF | YT | RN | RN | RN | WT | d1 | d2 | d3 | f1 | f2 | f3 | f4 | f5 |
| R_{P}-1 | 4.9e5 | 1.5e6 | 2.4e3 | 1.7e5 | 3.0e6 | 4.3e3 | 1.5e3 | 2.5e3 | 6.1e3 | 9.1e3 | 1.0e5 | 5.2e4 | 6.1e3 | 7.2e3 | 8.5e3 |
| S_{P}-1 | 1.2e6 | 7.1e6 | 1.6e5 | 7.4e5 | 8.0e6 | 1.7e5 | 2.2e5 | 1.9e5 | 1.2e6 | 1.2e6 | 7.2e5 | 6.2e5 | 1.3e5 | 2.3e5 | 2.9e5 |
| R_{P}-2 | 5.8e5 | 8.2e5 | 2.5e3 | 2.8e5 | 1.6e6 | 3.4e3 | 1.8e3 | 3.5e3 | 6.0e3 | 8.1e3 | 1.4e4 | 4.7e3 | 3.4e3 | 4.2e3 | 6.1e3 |
| S_{P}-2 | 2.7e4 | 1.2e6 | 1.1e5 | 2.6e4 | 1.6e6 | 1.4e5 | 5.9e4 | 8.6e4 | 4.6e5 | 4.3e5 | 1.4e5 | 1.9e5 | 1.2e5 | 2.4e5 | 4.2e5 |
| R_{P}-3 | 8.5e5 | 3.1e5 | 1.3e4 | 4.0e5 | 1.3e6 | 1.3e4 | 1.5e3 | 3.0e3 | 5.4e3 | 7.6e3 | 3.3e3 | 3.8e3 | 8.2e2 | 2.6e3 | 2.2e3 |
| S_{P}-3 | 3.4e4 | 1.6e6 | 7.3e4 | 4.8e4 | 1.4e6 | 3.9e5 | 1.8e5 | 5.0e5 | 1.1e6 | 1.2e6 | 6.7e5 | 1.0e6 | 1.1e6 | 2.3e6 | 1.9e6 |
| R_{P}R_{C}-4 | 1.3e3 | 1.9e2 | 5.8e1 | 3.0e3 | 2.8e2 | 3.0e2 | 2.2e2 | 6.8e2 | 6.4e2 | 5.6e2 | 4.2e2 | 4.1e2 | 6.3e2 | 7.9e2 | 8.5e2 |
| R_{P}S_{C}-4 | 2.0e2 | 5.5e1 | 1.6e1 | 4.6e2 | 7.4e1 | 1.9e2 | 1.3e2 | 1.2e2 | 1.5e2 | 1.4e2 | 2.1e2 | 1.4e2 | 1.4e2 | 2.0e2 | 1.8e2 |
| S_{P}R_{C}-4 | 1.1e2 | 1.6e3 | 1.8e3 | 2.3e2 | 1.8e3 | 1.2e3 | 8.1e3 | 2.3e4 | 6.0e4 | 8.7e4 | 1.3e4 | 1.4e4 | 3.2e3 | 5.0e3 | 3.8e3 |
| S_{P}S_{P}-4 | 3.2e0 | 6.2e1 | 2.5e2 | 1.5e1 | 9.6e1 | 4.8e2 | 1.7e3 | 4.2e3 | 8.1e3 | 1.1e4 | 2.6e3 | 2.5e3 | 1.5e3 | 1.5e3 | 1.2e3 |
| R_{P}-5 | 1.6e4 | 5.2e3 | 1.9e3 | 1.7e4 | 9.0e3 | 3.5e3 | 2.5e2 | 3.0e2 | 5.4e2 | 5.5e2 | 6.0e2 | 8.6e2 | 4.5e2 | 5.1e2 | 7.7e2 |
| S_{P}-5 | 2.1e1 | 3.3e2 | 5.8e3 | 2.8e1 | 3.6e2 | 1.4e4 | 2.8e4 | 1.0e4 | 5.2e4 | 1.5e5 | 3.9e4 | 7.7e4 | 1.2e5 | 2.5e5 | 3.2e5 |

*The standard errors, from fits of the data to eq 1, are less than 20% of the stated values.

### Example 25

Materials. Growth media and antibiotics were procured from Research Products Incorporated. DNA polymerase was obtained from Agilent. Other supplies for the molecular biology experiments were acquired from New England Biolabs. DEVX and N,N-diisopropylaminoethanethiol were synthesized as previously reported.(37, 44) The individual enantiomers of p-acetophenyl VR (APVR) were synthesized as previously reported.(39) Samples of VX and VR were Chemical Agent Standard Analytical Reference Material (CASARM) of the highest purity available, and were further purified as described previously.(37) Unless otherwise noted, all other chemicals were purchased from Sigma Aldrich. The organophosphorus nerve agents used in this investigation are highly toxic and should be used with the proper safety precautions. PTE: phosphotriesterase; DEVX: O,O-diethyl-VX; DMVX: O,O-dimethyl-VX; DEVR: O,O-diethyl-VR; and OMVR: O-methyl-VR.

### Example 26

Synthesis of Dimethyl VX. Dimethyl VX (DMVX) was made by the reaction of dimethyl chlorophosphate with N,N-diisopropyl aminoethanethiol. N,N-diisopropylaminoethanethiol (1.5 grams; 9.3 mmol) was added to 100 mL of diethyl ether and allowed to cool in a dry ice/acetone bath before being purged with N2. To this mixture was added 7.5 mL (2.0 equivalents) of a 2.5 M solution of butyl lithium in hexanes and the reaction allowed to come to room temperature before re-cooling in a dry ice/acetone bath. Dimethyl chlorophosphate (2.0 g; 1.5 equivalents) was mixed with 30 mL of diethyl ether in a separate flask and cooled. The dimethyl chlorophosphate solution was then added to the thiol solution and the reaction stirred at room temperature for 3 hours. The reaction was brought to 400 mL with diethyl ether and extracted with water. The product was extracted into the aqueous phase with 0.5 M HCl. The aqueous phase was neutralized with sodium bicarbonate and extracted with dichloromethane. The organic phase was dried over Na₂SO₄, filtered, and evaporated to dryness. The product was further purified by silica gel chromatography. The product was dissolved in dichloromethane and eluted from the column using using a 0-5% step gradient of methanol in dichloromethane. Fractions containing the desired product were combined and the solvent evaporated to provide the product as an oil. Overall isolated yield was 8%. ¹H NMR (300 MHz, CDCl₃): 3.84-3.77 (6H, d, J=12.6 Hz, OCH₃), 3.08-2.62 (6H, m, SCH₂CH₂N(CH)₂), 1.05-0.98 (12H, d, J=6.9 Hz, CH(CH₃)₂. ³¹P NMR (121.4 MHz, CDCl₃): 32.74 ppm.

### Example 27

Synthesis of Diethyl VR. Diethyl VR (DEVR) was made by the reaction of diethyl chlorophosphate with N,N-diethylaminoethanethiol. N,N-diethylaminoethanethiol was prepared from the hydrochloride salt by dissolving the compound in a saturated NaHCO₃ solution and extraction with diethyl ether. The organic phase was dried over Na₂SO₄ and evaporated in vacuo at room temperature. The remaining oil was distilled (50° C.) under high vacuum and recovered as a pure liquid in a dry ice cooled trap. N,N-diethylaminoethanethiol (1.1 grams; 8.35 mmol) was added to 100 mL of diethyl ether and cooled in a dry ice/acetone bath before being purged with N2. To this mixture was added 10 mL (3.0 equivalents) of a 2.5 M solution of butyl lithium in hexanes and the reaction allowed to come to room temperature before re-cooling in a dry ice/acetone bath. Diethyl chlorophosphate (2.9 g; 2 equivalents) was mixed with 30 mL of diethyl ether in a separate flask, purged with N2, and then cooled in a dry ice/acetone bath. The cooled diethyl chlorophosphate solution was added to the thiol solution and the reaction stirred at room temperature for 3 hours. The reaction was brought to 400 mL with diethyl ether and extracted with water. The product was extracted into the aqueous phase with 0.5 M HCl. The aqueous phase was neutralized with sodium bicarbonate and extracted with dichloromethane. The organic phase was dried over Na₂SO₄, filtered, and then evaporated, yielding the product as an oil. Further purification was conducted using silica gel chromatography as described above. Overall yield of the isolated product was 7%. ¹H NMR (300 MHz, CDCl₃): 4.27-4.10 (4H, m, OCH₂CH₃), 2.99-2.52 (8H, m, SCH₂CH₂N(CH₂)₂), 1.43-1.34 (6H, t, J=7.5 Hz, OCH₂CH₃), 1.11-1.01 (6H, t, J=7.2 Hz, CH₂CH₃). ³1P NMR (121.4 MHz, CDCl₃): 28.50 ppm.

### Example 28

Synthesis of O-methyl VR. O-methyl VR (OMVR) was synthesized by the reaction of methyl isobutyl chlorophosphate with N,N-diethylaminoethanethiol. N,N-diethylaminoethanethiol was prepared from the hydrochloride salt as described above. Methyl isobutyl chlorophosphate was prepared by dissolving 750 μL (8.4 mmol) of isobutanol in 50 mL of diethyl ether. The atmosphere was purged with N2 and then the mixture was chilled in a dry ice/acetone bath. A total of 3.3 mL (1.0 equivalent) of 2.5 M butyl lithium in hexanes and 1.5 g (1.0 equivalents) of methyl dichlorophosphate was added, and the reaction stirred for three hours at room temperature.

In a separate flask, 1.2 g (1.0 equivalents) of N,N-diethylaminoethanethiol was dissolved in 50 mL of diethyl ether and chilled in a dry ice/acetone bath. A total of 5 mL (1.5 equivalents) of 2.5 M butyl lithium was added and the reaction warmed to room temperature. The methyl isobutyl chlorophosphate and the thiol solutions were chilled in a dry ice/acetone bath and combined. The reaction was allowed to proceed at room temperature for 3 hours. The reaction was then brought to 400 mL with diethyl ether and washed with water. The product was extracted with 0.5 M HCl, neutralized with sodium bicarbonate, and then extracted with dichloromethane. The organic phase was dried over Na2SO4 and evaporated to yield the product as an oil. Overall yield of final product was 15%. ¹H NMR (300 MHz, CDCl₃): 3.92-3.72 (5H, m, OCH₂CH(CH₃)₂, OCH₃), 2.95-2.45 (8H, m, SCH₂CH₂N(CH₂)₂), 2.04-1.88 (CH, OCH₂CH(CH₃)₂), 1.06-0.97 (6H, t, J=7.0 Hz, NCH₂CH₃), 0.97-0.90 (6H, d, J=6.8 Hz, OCH₂CH(CH₃)₂. ³¹P NMR (121.4 MHz CDCl₃): 30.30 ppm.

### Example 29

Mutagenesis, Expression and Enzyme Purification. The gene for PTE was cloned into the expression vector pET 20b between the NdeI and EcoRI restriction sites as previously described.(39) The new variants of PTE were generated by introducing the mutations I106C, 1106G, and L308S into the appropriate templates by site directed mutagenesis using the Quick Change (Agilent) protocol. DNA sequencing at the Gene Technologies Laboratory at Texas A&M University verified the specific mutations. The proteins were expressed and purified as previously described. (37) Briefly, the variants were freshly transformed into E. coli BL21 (DE3) cells by electroporation, and single colonies used to inoculate 5.0 mL cultures of LB medium. After 8 hours of growth at 37° C., 1.0 mL of this culture was used to inoculate 1 L cultures of Terrific Broth supplemented with 1.0 mM CoCl₂. The bacterial cultures were grown at 30° C. IPTG was added to a final concentration of 1.0 mM after 24 hours and growth continued for 40 hours. The cells were harvested by centrifugation and stored at −80° C. prior to purification. Cells were resuspended in 100 mL of purification buffer (50 mM HEPES, pH 8.5, with 100 μM CoCl₂) and then lysed by sonication. The cell debris was cleared by centrifugation, nucleic acids were precipitated by protamine sulfate (0.45 g in 20 mL purification buffer per liter of culture), and then removed by centrifugation. The PTE mutants were precipitated with ammonium sulfate (60% saturation) and recovered by centrifugation. The pellet was resuspended in ˜5 mL of purification buffer, filtered (0.45 μm) and loaded onto a GE Superdex 200 (16/60) preparatory size exclusion column using a BioRad NCG FPLC system. Fractions with catalytic activity for the hydrolysis of paraoxon were pooled and then eluted from a 3.0 g (dry weight) DEAE Sephadex A25 resin that was pre-equilibrated in purification buffer. Protein purity was verified by SDS-PAGE.

Generation and Characterization of New PTE Variants. The PTE variants QF, CVQFL, VRN-VQFL and L7ep-3a were previously shown as having substantially improved activity for the hydrolysis of VX.(37) The stereoselectivity of these mutants for the chiral center contained in VR was determined using the isolated enantiomers of APVR (Table 11). With the exception of QF, the VX-optimized variants of PTE prefer to hydrolyze the R_{P}-enantiomer of the chiral center for VR. The crystal structure of L7ep-3a suggests that the mutations I106C and S308L affect the size of the small-group pocket. In order for the S_{P}-enantiomer of VR to productively bind in the active site, the larger isobutyl group must be positioned in the small-group pocket of PTE. In an effort to maximize the hydrolysis of S_{P}-VR, a series of variants was created to change the substitutions made to residues 106 and 308 in the variants previously optimized for the hydrolysis of VX.

TABLE 11
Kinetic constants for PTE variants with APVR¹.

| | R_{P}-APVR | | | S_{P}-APVR | | | | |
|---|---|---|---|---|---|---|---|---|
| | k_{cat} | Kₘ | k_{cat}/Kₘ | k_{cat} | Kₘ | k_{cat}/Kₘ | | |
| Enzyme | (s⁻¹) | (mM) | (M⁻¹s⁻¹) | (s⁻¹) | (mM) | (M⁻¹s⁻¹) | Ratio² | |
| Wild-type | 84 | 1.7 | 4.9 × 10⁴ | 25 | 4.5 | 6 × 10³ | 8:1 | R |
| QF | 57 | 0.36 | 1.6 × 10⁵ | 8.7 | 0.030 | 2.9 × 10⁵ | 1.8:1 | S |
| CVQFL | 46 | 0.18 | 2.6 × 10⁵ | 21 | 0.19 | 1.1 × 10⁵ | 2.5:1 | R |
| CVQFL C106I* | 50 | 0.15 | 3.3 × 10⁵ | 14 | 1.5 | 9.5 × 10³ | 35:1 | R |
| CVQFL-I106G | 174 | 1.4 | 1.2 × 10⁵ | 36 | 0.20 | 1.8 × 10⁵ | 1.5:1 | S |
| CVQFL-L308S* | 122 | 2.0 | 6.0 × 10⁴ | 8.1 | 0.19 | 4.2 × 10⁴ | 1.4:1 | R |
| CVQFL-I106G/L308S | 100 | 4 | 2.4 × 10⁴ | 40 | 0.24 | 1.6 × 10⁵ | 6.7:1 | S |
| VRN-VQFL | 55 | 0.16 | 3.4 × 10⁵ | 29 | 1.7 | 1.7 × 10⁴ | 20:1 | R |
| VRN-VQFL-I106C | 72 | 0.23 | 3.2 × 10⁵ | 33 | 0.23 | 1.4 × 10⁵ | 2.3:1 | R |
| VRN-VQFL-I106G | 56 | 0.41 | 1.4 × 10⁵ | 159 | 0.18 | 9.0 × 10⁵ | 6.6:1 | S |
| VRN-VQFL-L308S | 17 | 0.52 | 3.2 × 10⁴ | 5.0 | 0.13 | 3.9 × 10⁴ | 1.2:1 | S |
| VRN-VQF-I106C/L308S | 160 | 1.7 | 1.0 × 10⁵ | 51 | 1.5 | 3.4 × 10⁴ | 3:1 | R |
| VRN-VQFL-I106G/L308S | 45 | 2.1 | 2.2 × 10⁴ | 53 | 0.18 | 3.0 × 10⁵ | 14:1 | S |
| L7ep-3a | 101 | 0.62 | 1.6 × 10⁵ | 56 | 0.5 | 1.1 × 10⁵ | 1.5:1 | R |
| L7ep-3aI106G | 58 | 0.71 | 8.2 × 10⁴ | 166 | 0.31 | 5.3 × 10⁵ | 6.5:1 | S |

*The mutations C106I and L308S are revertants back to the wild-type amino acid sequence. ¹Errors from curve fitting are less than 10% with the exception of CVQFL-I106G/L308S, which has an error of 20% due to the high Kₘ value. ²The ratio is k_{cat}/Kₘ values for fast enantiomer and slow enantiomer, with the preferred enantiomer identified.

### Example 30

Enzymatic Activity. Catalytic activity with paraoxon was followed by monitoring the release of p-nitrophenol at 400 nm (ΔE400=17,000 M⁻¹ cm⁻¹) in 250 μL reaction volumes containing 50 mM CHES, pH 9.0, 100 μM CoCl2, and 0-1.0 mM paraoxon. Activity with APVR utilized the same reaction conditions as paraoxon with the release of the leaving group monitored at 294 nm (ΔE294=7,710 M⁻¹ cm⁻¹). Activity with DEVX, DMVX, DEVR, and OMVR was measured in 250 μL reactions containing 50 mM HEPES, pH 8.0, 100 μM CoCl₂, 0.3 mM DTNB and 0-1.0 mM substrate. The release of the thiol leaving group was followed by inclusion of DTNB in the assay mixture (ΔE412=14,150 M⁻¹ s⁻¹). All assays were initiated by the addition of the appropriately diluted enzyme and monitored in a 96-well format using a Molecular Devices SpectraMax 364 Plus plate reader. Reactions were monitored for 15 minutes at 30° C. and the linear portion of the time course was used to calculate the initial rate. Kinetic constants were determined by fitting the data to the Michaelis-Menten equation.(45) When saturation could not be observed, the data was fit to a linear equation and the slope taken as k_{cat}/Kₘ.

The catalytic activities of these variants were first characterized against the insecticide paraoxon and the V-agent analog DEVX (Table 12), and then the stereochemical preferences were determined using APVR (Table 11).

TABLE 12
Kinetic parameters for PTE variants with paraoxon and DEVX¹.

| | Paraoxon | | | DEVX | | |
|---|---|---|---|---|---|---|
| | k_{cat} | Kₘ | k_{cat}/Kₘ | k_{cat} | Kₘ | k_{cat}/Kₘ |
| Enzyme | (s⁻¹) | (uM) | (M⁻¹s⁻¹) | (s⁻¹) | (mM) | (M⁻¹s⁻¹) |
| Wild-type | 2230 | 81 | 2.8 × 10⁷ | 1.1 | 0.87 | 1.2 × 10³ |
| QF | 41 | 5.3 | 7.7 × 10⁶ | 6.1 | 1.4 | 4.2 × 10³ |
| CVQFL | 38 | 5.6 | 6.8 × 10⁶ | 16 | 0.76 | 2.1 × 10⁴ |
| CVQFL-C106I* | 66 | 5.4 | 1.2 × 10⁷ | 14 | 0.65 | 2.2 × 10⁴ |
| CVQFL-L308S* | 33 | 6.7 | 4.8 × 10⁶ | 13 | 3.2 | 4.1 × 10³ |
| CVQF- | 48 | 25 | 1.9 × 10⁶ | nd | nd | 3.1 × 10² |
| I106G/L308S | | | | | | |
| CVQFL- | 108 | 11 | 1.0 × 10⁷ | 19 | 1.0 | 1.9 × 10⁴ |
| C106I/L308S | | | | | | |
| CVQFL-I106G | 456 | 22 | 2.0 × 10⁷ | 31 | 2.8 | 1.1 × 10⁴ |
| VRN-VQFL | 116 | 8 | 1.5 × 10⁷ | 22 | 0.73 | 3.1 × 10⁴ |
| VRN-VQFL- | 103 | 5.3 | 1.9 × 10⁷ | 23 | 0.80 | 2.8 × 10⁴ |
| I106C | | | | | | |
| VRN-VQFL- | 446 | 21 | 2.1 × 10⁷ | nd | nd | 5.0 × 10³ |
| I106G | | | | | | |
| VRN-VQFL- | 15 | 1.7 | 8.8 × 10⁶ | 5.1 | 0.35 | 1.5 × 10⁴ |
| L308S | | | | | | |
| VRN-VQFL- | 35 | 3.6 | 9.7 × 10⁶ | 8.6 | 1.6 | 5.4 × 10³ |
| I106C/L308S | | | | | | |
| VRN-VQFL- | 58 | 9.2 | 6.2 × 10⁶ | nd | nd | 4.1 × 10² |
| I106G/L308S | | | | | | |
| L7ep-3a | 142 | 7.2 | 1.2 × 10e⁷ | 5 | 0.60 | 8.5 × 10⁴ |
| L7ep-3a I106G | 545 | 33 | 1.67 × 10⁷ | nd | nd | 7.7 × 10³ |

*The mutations C106I and L308S are revertants to the wild-type amino acid sequence.
¹Errors from curve fitting were less than 10%.

Inclusion of the I106C mutation substantially reduced the preference for the hydrolysis of the R_{P}-enantiomer, while it tended to have relatively little effect on the hydrolysis of DEVX. For example, the CVQFL variant has a 2.5-fold preference for the R_{P}-enantiomer of the VR chiral center, but CVQFL-C106I has a 35-fold preference for the R_{P}-enantiomer. Despite this large difference in stereoselectivity, both of these variants hydrolyze DEVX with a k_{cat}/Kₘ of ˜2×10⁴ M⁻¹ s⁻¹. The removal of the S308L mutation from the VX-optimized variants also results in a substantial diminished preference toward the R_{P}-enantiomer, but in most cases it also resulted in diminished activity against DEVX. VRN-VQFL prefers the R_{P}-enantiomer of APVR by 20-fold. The variant VRN-VQFL L308S has a preference of 1.2-fold, but the activity against DEVX was also diminished 2-fold. When glycine was substituted at position 106, the stereochemical preference shifted to the S_{P}-enantiomer of APVR. L7ep-3a prefers the R_{P}-enantiomer by 1.5-fold, but L7ep-3a I106G prefers the S_{P}-enantiomer by 6.5-fold. Unfortunately, the I106G mutation reduced the activity against DEVX by more than an order of magnitude in these variants.

### Example 31

Stereoselelctive Hydrolysis of Racemic VX and VR. Low initial concentrations (19 to 160 μM) of racemic VX and VR were hydrolyzed by variants of PTE in a solution containing 0.1 mM CoCl₂, 0.3 mM DTNB, and 50 mM Bis-Tris-propane, pH 8.0. The reactions were followed to completion and the fraction hydrolyzed plotted as a function of time. The time courses were fit to equations 1 and 2 where F is the fraction hydrolyzed, a and b are the magnitudes of the exponential phases, t is time, and k₁ and k₂ are the rate constants for each phase.(39)

F=a(1−e^{-k}^{₁}^{t}) (1)

F=a(1−e^{-k}^{₁}^{t})+b(1−e^{-k}^{₁}^{t}) (2)

Stereochemical preferences were determined using the previously described complementation method.(37) Briefly, variants with large stereoselective preferences were placed in a reaction with mutants of PTE of known stereoselective preferences under conditions where each variant alone would exhibit a similar rate for the first exponential phase. If the variants prefer the same enantiomer, the resulting time course will exhibit two distinct phases. If the variants have the opposite enantiomers as the preferential substrate, the time courses will exhibit a single exponential phase.

Catalytic Activity with VX and VR. The most promising new variants were tested with racemic VX and VR. The assays were conducted for the complete hydrolysis of a single low concentration of these agents to enable the observation of exponential time courses, corresponding to the hydrolysis of each enantiomer contained within the racemic mixture. The kinetic constants are presented in Table 13. For enzyme variants with large stereochemical preferences, the identity of the preferred enantiomer was determined by the ability of the variant to complement the slow phase of a variant of known preference. Wild-type PTE is known to prefer to hydrolyze the R_{P}-enantiomer of the VR chiral center, while QF is known to prefer the S_{P}-enantiomer of VX.(37, 39) None of the variants tested, with the exception of QF, exhibited large stereochemical preferences for hydrolysis of the two enantiomers of VX. Removal of the S308L mutation (VRN-VQFL L308S) resulted in a 2-fold reduction in catalytic activity for the faster enantiomer of VX and a 5-fold reduction in the rate of hydrolysis for the slower enantiomer. Introduction of the I106G mutation (VRN-VQFL-I106G) led to the complete hydrolysis of racemic VX without detectable selectivity, but at a rate 6-fold slower than VRN-VQFL had for the slower enantiomer.

TABLE 13
Kinetic constants with the racemic nerve agents VX and VR¹.

| | VX | | | VR | | |
|---|---|---|---|---|---|---|
| | k_{cat}/Kₘ₁ | k_{cat}/Kₘ₂ | | k_{cat}/Kₘ₁ | k_{cat}/Kₘ₂ | |
| Enzyme | (M⁻¹s⁻¹) | (M⁻¹s⁻¹) | Ratio | (M⁻¹s⁻¹) | (M⁻¹s⁻¹) | Ratio² |
| Wild-type | 8.4 × 10¹ | nd | | 1.1 × 10² | 4.3 × 10⁰ | 25:1 R |
| QF | 1.7 × 10⁴ | 1.5 × 10³ | 12:1 S | 4.8 × 10² | 7.9 × 10¹ | 6:1 |
| CVQFL | 1.0 × 10⁵ | | 1:1 | 5.5 × 10³ | 8.9 × 10² | 6:1 |
| VRN-VQFL | 1.1 × 10⁵ | 4.3 × 10⁴ | 4:1 | 2.4 × 10³ | nd | >30:1 R |
| VRN-VQFL-I106G | 6.6 × 10³ | | 1:1 | 2.0 × 10³ | 4.1 × 10² | 5:1 |
| VRN-VQFL-L308S* | 6.2 × 10⁴ | 8.9 × 10³ | 7:1 | 2.7 × 10² | | 1:1 |
| VRN-VQFL- | | | | | | |
| I106G/L308S | 4.9 × 10³ | 1.7 × 10³ | 3:1 | 2.1 × 10³ | 6.7 × 10¹ | 31:1 S |
| L7ep-3a | 8.3 × 10⁵ | 2.2 × 10⁵ | 4:1 | 2.2 × 10³ | 2.1 × 10² | 10:1 |
| L7ep-3a I106G | 2.0 × 10⁴ | 6.2 × 10³ | 3:1 | 2.6 × 10³ | 6.9 × 10² | 3.8:1 |

*The mutation L308S is a revertant to the wild type amino acid sequence.
¹Errors from curve fitting are less than 5%.
²The ratio is k_{cat}/Kₘ values for fast enantiomer and slow enantiomer. If the preferred enantiomer is not listed it has not been determined.

With racemic VR, the VRN-VQFL variant exhibited a 20-fold enhancement in the rate of hydrolysis compared to the wild-type enzyme, but this mutant was found to only hydrolyze the relatively nontoxic R_{P}-enantiomer of VR. Removal of the S308L mutation (VRN-VQFL L308S) enabled the hydrolysis of both enantiomers of VR, with complete loss of stereoselectivity. This represents a 64-fold improvement toward the hydrolysis of the toxic S_{P}-enantiomer compared to wild-type PTE. The introduction of the I106G mutation in the VRN-VQFL-I106G/L308S variant resulted in a strong preference for the hydrolysis of the S_{P}-enantiomer relative to the R_{P}-enantiomer. VRN-VQFL-I106G, which has both the I106G and S308L mutations, has much less stereochemical preference, but the kinetic data for the hydrolysis of the two enantiomers of APVR indicate that the stereochemical preference is for the SP-enantiomer. The best variant identified for the hydrolysis of VR was L7ep-3a I106G, which has a k_{cat}/Kₘ of 2.6×103 M⁻¹ s⁻¹ for the faster enantiomer. While the stereoselectivity was not sufficient to determine the stereochemical preference with VR directly, the kinetic data with the two enantiomers of APVR has identified the preferred enantiomer as the highly toxic S_{P}-enantiomer. The L7ep-3a I106G mutant thus has a 620-fold enhanced rate of hydrolysis of S_{P}-VR relative to wild-type PTE.

### Example 32

X-ray Crystallography. The PTE mutants L7ep-3a and L7ep-3a I106G were crystallized at 18° C. using the vapor diffusion method. In the crystallization experiments, 1.0 μL of protein (10 mg/mL with 1.0 mM CoCl2) was mixed with 1.0 μL of the precipitant solution (100 mM sodium cacodylate pH 5.5-7.0, 0.2 M magnesium acetate, 15-30% PEG 8000), and then equilibrated against 500 μL of the same precipitant solution using Intelliplates. Protein crystals appeared within a week and grew to maximum dimensions (200 μm×15 μm×15 μm) after 21 days. Prior to data collection, the crystals were soaked for 30 seconds in a cryoprotectant solution of the mother liquor containing 30% ethylene glycol and then frozen in liquid nitrogen. Diffraction data were collected locally at 120 K on an R-AXIS IV detector with Cu Kα X-rays produced from a rotating anode generator. X-ray data reduction and scaling were performed with HKL2000.(46) Structures of the PTE mutants L7ep-3a and L7ep-3aG were determined by molecular replacement using the coordinates of wild type PTE (PDB id: 1DPM) as the search model. The structures were built using COOT and refined with simulated annealing, B-factor randomization, and coordinate shaking using PHENIX.(47, 48) Later stages of refinement were also done in PHENIX using individual coordinate, anisotropic B-factor, and occupancy optimization. The PTE mutant structures were refined with R_{work}/R_{free} values of 13.5-21.5% with excellent geometry (Table 14).

TABLE 14
X-ray crystallography data for L7ep-3a and L7ep-3a I106G.

| Variant | L7ep-3a | L7ep-3a I106G |
|---|---|---|
| Resolution, Å (Highest | 50.00-2.01 | 50.00-2.01 |
| resolution shell) | (2.04-2.01) | (2.04-2.01) |
| Space group | P2₁ | P2₁ |
| Cell dimensions | | |
| a | 45.53 | 45.85 |
| b | 80.64 | 80.63 |
| c | 78.73 | 78.84 |
| γ | 106.60 | 106.94 |
| R_{sym} | 0.087 | 0.057 |
| I/σI | 13.2 (3.0) | 18.9 (2.8) |
| Completeness, % | 98.3 (96.1) | 95.8 (91.1) |
| (Highest resolution shell) | | |
| Redundancy | 3.5 (3.2) | 3.7 (3.2) |
| (Highest resolution shell) | | |

Refinement

| | | |
|---|---|---|
| Resolution, Å | 29.61-2.01 | 29.57-2.01 |
| No. of reflections | 35,603 | 34,657 |
| R_{work}/R_{free} | 0.1574/0.2145 | 0.1348/0.1838 |
| No. of nonhydrogen atoms | | |
| Total | 5420 | 5395 |
| Water | 383 | 384 |
| B-factors | | |
| Protein | 27.56 | 28.08 |
| Co²⁺ | 30.72 | 30.60 |
| Root mean square deviations | | |
| Bond lengths, Å | 0.006 | 0.007 |
| Bond angles, ° | 1.09 | 1.10 |
| Ramachandran | 97.4 | 97.1 |
| Favored, % | | |
| Allowed, % | 2.6 | 2.9 |
| Outliers, % | 0 | 0 |

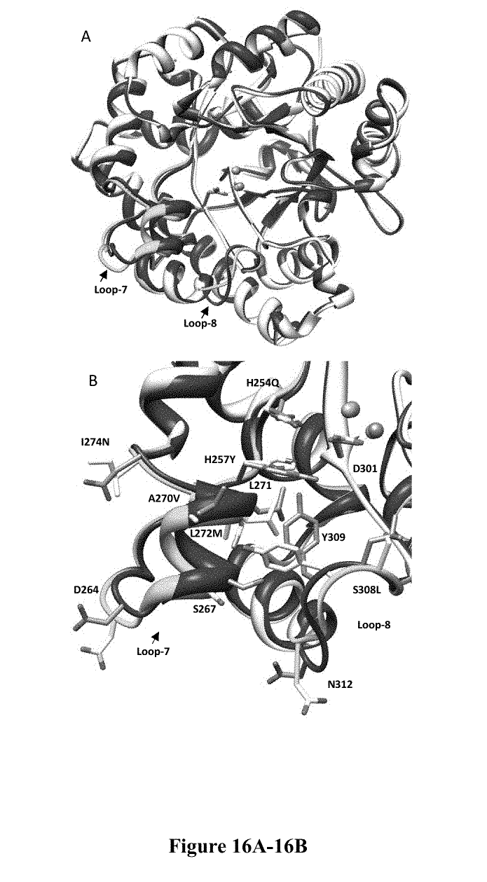

Three-Dimensional Structure of the PTE Mutant L7ep-3a. The PTE variant L7ep-3a has the highest reported k_{cat} for the hydrolysis of the nerve agent VX.(37) In an effort to elucidate the mechanism by which the activity of this mutant is enhanced, the enzyme was crystallized and the structure determined to a resolution of 2.01 Å by X-ray diffraction methods (PDB id: 4ZST). FIG. 16 depicts (A) Structural alignment between wild-type PTE (light) and L7ep-3a mutant (dark). Selective active site residues are labeled and shown. Loop-7 and -8 are shown. (B) Expanded view of Loop-7 and -8. Mutated residues and residues with significant structural perturbations are shown as sticks. The wild type structure is taken from PDB id: 1DPM and the L7ep-3a structure is taken from PDB id: 4ZST. The overall structure of L7ep-3a is very similar to wild-type PTE (FIG. 16A). The core of the (β/α)8-barrel matches very well between the two structures with a Cα RMSD of 0.64 Å. The only significant change in the backbone structure is apparent in the conformations of Loop-7 and Loop-8. In this variant, Loop-7, including the Loop-7 α-helix, is pulled toward the active site (FIG. 16B). A portion of Loop-8 is similarly pulled toward the active site. Loop-8 also participates in the dimer interface, but the cross-interface interactions are all retained in the L7ep-3a mutant.

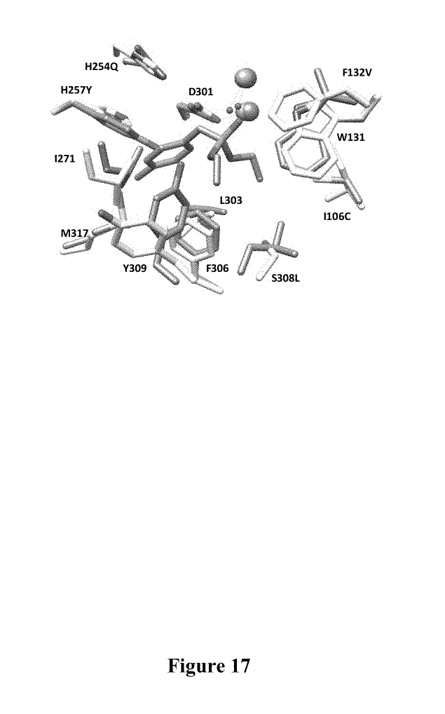

The binding site for the substrate in wild-type PTE is divided into the large-group pocket (His-254, His-257, Leu-271, and Met-317), the leaving-group pocket (Trp-131, Phe-132, Phe-306, and Tyr-309), and the small-group pocket (Gly-60, Ile-106, Lue-303 and Ser-308).(50) The mutations H257Y and S308L, coupled with the shifting of the Loop-7 α-helix, have induced significant changes in the substrate binding pockets in the active site of the L7ep-3a mutant. The side-chain of Tyr-309 is repositioned so that the phenolic group now extends into the active site rather than towards Loop-7, as previously observed in the structure of wild-type PTE (FIGS. 16B and 17).

FIG. 17 depicts the substrate binding pockets of wild-type (white) and L7ep-3a (grey). The large-group pocket residues are His-254, His-257, Ile-271, and Met-317. The small-group pocket residues are Gly-60, Ile-106, Leu-303, and Ser-308. The leaving group pocket resides are Trp-131, Phe-132, Phe-306 and Tyr-309. The Wild-type structure (PDB id: 1DPM) is shown with the inhibitor, diethyl 4-methylbenzylphosphonate, bound in the active site.

The reorientation of Tyr-309, along with the substitution of a tyrosine for His-257 and the repositioning of Leu-271 into the active site, has dramatically compressed the size of the large-group and leaving-group pockets. The leaving-group pocket is also constricted by the presence of the S308L mutation, which adds bulk to both the leaving-group and small-group pockets. However, the apparent contraction of the leaving-group pocket is partially relieved by the F132V mutation. Similarly, the I106C mutation opens additional space to the small-group pocket.

Three-Dimensional Structure of L7ep-3a I106G. In an effort to understand the physical basis for the observed improvement in the catalytic activity of L7ep-3a I106G, the three-dimensional structure was solved by X-ray crystallography (PDB id: 4ZSU). The core structure is very similar to wild-type PTE (Cα RMSD=0.66 Å) and the loop structure matches that observed with L7ep-3a.

### Example 33

Computational Docking of High Energy Intermediates. The pentavalent high-energy reaction intermediate states for the hydrolysis of VX and VR were computationally docked into the three-dimensional structures of wild-type PTE (PDB id: 1DPM), H254Q/H257F (PDB id: 2OQL), L7ep-3a (PDB id: 4ZST), and L7ep-3aG (PDB id: 4ZSU). The high energy intermediate states for the hydrolysis of the R_{P}- and S_{P}-enantiomers were manually generated as trigonal bipyrimidal structures with the attacking hydroxyl group protonated and the original phosphoryl oxygen substituent carrying a full negative charge using ChemBio Ultra 14.0. Computational docking was done using the program AutoDock Vina.(49) The appropriateness of the docked poses was evaluated by the value of the distance of the attacking hydroxyl group from the α- and β-metal ions, the distance of the phosphoryl oxygen from the β-metal and the orientation of the side ester substituents into the large and small group pockets contained in the active site of PTE.

### Example 34

Evaluation of New V-agent Analogs. The majority of research targeting the catalytic hydrolysis of organophosphorus nerve agents must be done using analogs for both regulatory and safety reasons. Intrinsic to the use of substrate analogs is the imperfect representation of catalytic activity with the authentic nerve agent. To address which structural factors of the VR and VX analogs are most significant, the compounds DMVX, DEVR, and OMVR were synthesized and analyzed as substrates for the optimized mutants of PTE and compared with the ability of these mutants to hydrolyze the most toxic forms of VR and VX. The catalytic activity using these compounds was determined with a series of variants for which the hydrolysis of authentic nerve agents was available (Table 15). Wild-type PTE has a much lower catalytic activity with DMVX (k_{cat}/Kₘ=1.9×101 M⁻¹ s⁻¹) than was observed with DEVX (k_{cat}/Kₘ 1.2×103 M⁻¹ s⁻¹). The best variant with DMVX was L7ep-3a, which has a k_{cat}=28 s⁻¹ and a k_{cat}/Kₘ=1.3×104 M⁻¹ s⁻¹. These values are 1,300- and 680-fold better, respectively, than wild-type PTE. Wild-type PTE exhibited better catalytic activity with DEVR (k_{cat}/Kₘ=5.6×102 M⁻¹ s⁻¹). L7ep-3a also had the best catalytic activity with this analog (k_{cat}/Kₘ=1.5×104 M⁻¹ s⁻¹). With the exception of wild-type PTE (k_{cat}/Kₘ=6.8×101 M⁻¹s⁻¹), racemic OMVR gave the least activity for most of the variants tested. The combination of poor solubility of this compound and high Kₘ values limited analysis to the determination of k_{cat}/Kₘ for most variants. The best mutant for the hydrolysis of OMVR was L7ep-3a with a k_{cat}/Kₘ=5.8×102 M⁻¹ s⁻¹. However, given the switch in stereochemical preference, it is highly likely that L7ep-3a I106G has the best catalytic activity with the R_{P}-enantiomer of OMVR (which corresponds to the same relative stereochemistry as the S_{P}-enantiomer of VR.

TABLE 15
Kinetic constants for PTE variants with V-agent analogs¹.

| | DMVX | | | DEVR | | | OMVR² | | |
|---|---|---|---|---|---|---|---|---|---|
| | k_{cat} | Kₘ | k_{cat}/Kₘ | k_{cat} | Kₘ | k_{cat}/Kₘ | k_{cat} | Kₘ | k_{cat}/Kₘ |
| Enzyme | (s⁻¹) | (mM) | (M⁻¹s⁻¹) | (s⁻¹) | (mM) | (M⁻¹s⁻¹) | (s⁻¹) | (mM) | (M⁻¹s⁻¹) |
| Wild-type | 0.021 | 1.1 | 1.9 × 10¹ | 0.201 | 0.38 | 5.6 × 10² | nd | nd | 6.8 × 10¹ |
| QF | 0.21 | 0.80 | 2.6 × 10² | 0.82 | 0.37 | 2.2 × 10³ | 0.15 | 1.7 | 7.1 × 10¹ |
| CVQFL | 17 | 3.7 | 4.4 × 10³ | 1.49 | 0.19 | 7.8 × 10³ | nd | nd | 2.1 × 10² |
| VRN-VQFL | 5.1 | 0.91 | 5.5 × 10³ | 3.1 | 0.36 | 8.7 × 10³ | nd | nd | 5.1 × 10² |
| VRN-VQF-L308S* | 3.0 | 1.8 | 1.6 × 10³ | 1.94 | 0.21 | 9.2 × 10³ | nd | nd | 1.4 × 10² |
| VRN-VQFL-I106G | 0.68 | 1.6 | 4.4 × 10² | 0.47 | 0.8 | 5.9 × 10² | nd | nd | 3.3 × 10² |
| VRN-VQFL-I106G/ | 0.3 | 1.15 | 2.6 × 10² | 0.115 | 0.97 | 1.2 × 10² | nd | nd | 1.9 × 10² |
| L308S | | | | | | | | | |
| L7ep-3a | 28 | 2.1 | 1.3 × 10⁴ | 6.8 | 0.44 | 1.5 × 10⁴ | 1.0 | 1.5 | 5.8 × 10² |
| L7ep-3a I106G | nd | nd | 1.4 × 10³ | 4 | 6 | 6.7 × 10² | nd | nd | 2.7 × 10² |

*L308S mutation is a revertant to the wild-type amino acid sequence.
¹Errors from curve fitting were less than 10% except for k_{cat} and Kₘ for L7ep3aG with DEVR.
²k_{cat}/Kₘ was determined from linear fit at low concentration of racemic substrate.

Comparison of Substrate Analogs. The VX analog DEVX was successfully used to identify PTE variants for the hydrolysis of VX, but it over-estimated the activity of the wild-type enzyme and failed to detect a 100-fold increase in the catalytic activity with the QF variant.(37) DEVX was initially synthesized to mimic the O-ethyl substituent in VX, but the bulk of the diethyl phosphorus center is larger than the volume of the methylphosphonate moiety of VX. DMVX also contains an achiral phosphorus center, but the volume of the dimethyl center is more representative of authentic VX. The catalytic activity of the PTE variants with DMVX was surprisingly low, but this analog captures the much lower catalytic activity of wild-type PTE for the hydrolysis of VX and the substantial increase in activity with the QF variant. The catalytic properties for the hydrolysis of DMVX are also able to predict the high k_{cat} for the hydrolysis of VX by the L7ep-3a variant. While DEVX was intended to ensure accommodation of the larger O-ethyl substituent, the data for DMVX suggests that for mimicking VX, the overall size of the phosphorus center is more important. While the asymmetry of the phosphonate center is obviously a major contributor to the catalytic activity using VX as a substrate, the dimethyl center provided a reasonable prediction of the catalytic activity.

The compound DEVR was synthesized to test the significance of the diethylamino vs. diisopropylamino groups contained within the VR and VX leaving groups, respectively. The smaller leaving group of DEVR resulted in 2-fold less activity compared to DEVX for wild-type PTE. Similar differences were obtained for most of the other variants. L7ep-3a I106G was the only variant where the catalytic activity with DEVR was less than 10% of the catalytic activity with DEVX. The relative activity between variants was similar with either leaving group but the reduced activity, especially manifested in the value of k_{cat}, suggests that the interactions of the enzyme with the leaving group maybe partially responsible for aligning the phosphorus center for nucleophilic attack. The smaller leaving group probably contributes to the lower enzymatic activity observed with VR, but comparison to the catalytic activity with DEVR also highlights the dominance of the phosphorus center. The introduction of I106G in the L7ep-3a variant resulted in a 23-fold loss of activity with DEVR, but the activity with authentic VR was improved.

In an attempt to better reflect the asymmetric phosphorus center of VR, the analog OMVR was synthesized. The PTE variants exhibited the least activity with the analog OMVR, which employs a slightly larger but asymmetric phosphorus center and the authentic leaving group of VR. Kinetic constants with racemic OMVR are about an order of magnitude smaller than is obtained with authentic VR, but the relative activity between variants is much more consistent than is seen with the other analogs. VRN-VQFL and L7ep-3a both have substantially better activity than wild-type PTE or QF for both OMVR and authentic VR. Introduction of I106G or removal of S308L in the VRN-VQFL variant resulted in somewhat diminished rates for both authentic VR and OMVR.

### Example 35

Active Site Hydrogen Bonding Network. The kcat values for wild-type PTE with phosphorothiolate substrates are approximately 104-fold lower than that with its best substrates.(37, 51) In the hydrolysis of substrates such as paraoxon there is no need to protonate the leaving group.(52)

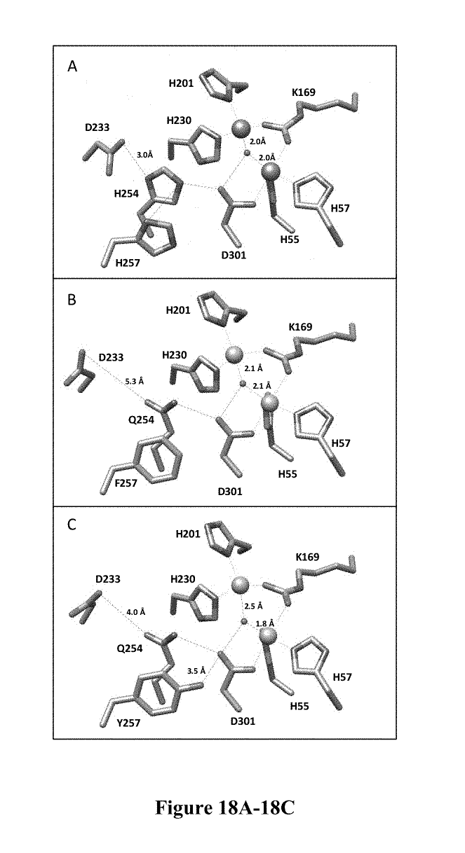

FIG. 18 depicts the metal center of wild-type PTE (A), QF (B), and L7ep-3a (C) variants. The residues binding to the α-metal (His-55, His-57, and Asp-301), the β-metal (His-201 and His-230), and the bridging carboxylated Lys-169 are shown. The proton shuttle residues His-257, His-254, and Asp-233 are also shown. The wild-type structure is obtained from PDB id: 1DPM and the QF structure is obtained from PDB id: 2OQL.

In the proposed reaction mechanism for wild-type PTE, the proton from the attacking nucleophilic water is passed to Asp-301, and, in turn, to His-254 (FIG. 18A).(53) The proton is then transferred to Asp-233 and on to bulk solvent. The variant QF has been postulated to have significantly improved catalytic activity with VX in part because of the disruption of this proton shuttle. In the crystal structure of the mutant QF, His-254 of the wild-type enzyme is a glutamine, which cannot participate in proton shuttling, and Asp-233 is moved out of hydrogen bonding distance (FIG. 18B). It is thought that the “trapping” of the proton in the active site is useful for the hydrolysis of slow substrates like VX, where protonation of the leaving group will contribute to improved k_{cat} values. The crystal structure of L7ep-3a, which has a kcat value for the hydrolysis of VX about an order of magnitude higher than QF, shows a remarkable rearrangement of the hydrogen bonding pattern in the active site. In L7ep-3a, Gln-254 hydrogen bonds to Asp-301, and Asp-233 has moved back into hydrogen bonding distance to Gln-254. Tyr-257 is also hydrogen bonded to Asp-301. The dramatic alteration of the hydrogen bonding network of Asp-301 results in the displacement of the nucleophile water toward the α-metal. This asymmetrical binding to the binuclear metal center is likely to result in the bridging hydroxyl being a stronger nucleophile. In the L7ep-3a I106G mutant, a similar hydrogen bonding pattern is observed, but Asp-233 is moved out of hydrogen bonding distance and the displacement of the bridging hydroxyl is not nearly as pronounced. The asymmetric positioning of the bridging hydroxyl has previously been observed in the crystal structure of dihydroorotase in the presence of bound dihydroorotate.(54)

### Example 36

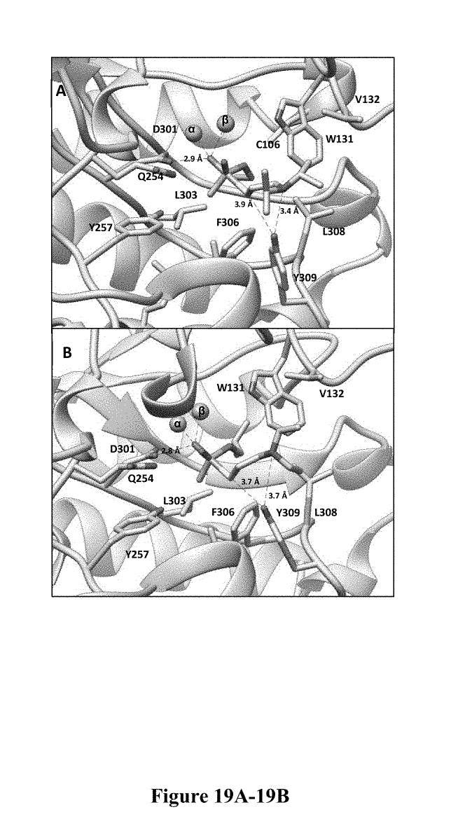

Docking of Substrates in the Active Site. In an effort to gain insight into the altered activity of L7ep-3a and L7ep-3a I106G, the substrates VX and VR were docked into the active site using the program AutoDock Vina. Computational docking was conducted using the trigonal bipyrimidal intermediates formed during the hydrolysis of both enantiomers of these compounds.(55) Productive poses were assessed by the placement of the attacking hydroxyl group between the two metal ions and the orientation of the phosphoryl oxygen toward the β-metal.(56) For the wild-type and QF variants both enantiomers of VX and VR could be reasonably docked into the active site (data not shown). However, for L7ep-3a and L7ep-3a I106G, only the S_{P}-enantiomer of VR could be reasonable positioned in the active site. For both of these variants, the constriction of the active site and the repositioning of Tyr-309 appear to play important roles in the activity against the V-agents (FIG. 19).

FIG. 19 depicts (A) S_{P}-VX docked in the active site of L7ep-3a. (B) S_{P}-VR docked into the active site of L7ep-3a I106G. The substrate binding residues are shown. The distances from Tyr-309 and Asp-301 are shown in units of Ångstoms.

The mutation F132V provides the extra room to accommodate the isopropyl amino group of VX. This effect is not as obvious with the smaller substituent contained within the leaving group in VR. The changes in the active site also enable the sulfur and the nitrogen of the leaving group to potentially hydrogen bond with the side chain phenol of Tyr-309, suggesting that this interaction may be partially responsible for the dramatically improved activity. The additional space in the small-group pocket due to the I106G mutation accommodates the isobutyl group in S_{P}-VR, while the combination of the H257Y mutation and repositioning of Tyr-309 make binding of the R_{P}-enantiomer more difficult.

The determination of the three-dimensional structure of the L7ep-3a mutant has led to a greater understanding of the underlying mechanisms by which the hydrolysis of V-agents is enhanced.

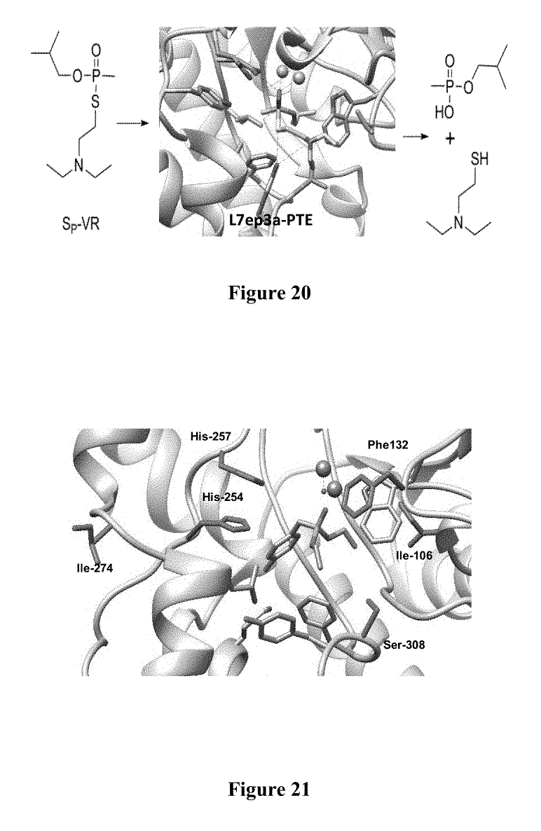

FIG. 20 depicts an equation showing the interaction between S_{P}-VR and L7ep3a-PTE resulting in hydrolysis of S_{P}-VR.

The determination of this structure has directly led to the rational construction of the new L7ep-3a I106G mutant, which is enhanced 620-fold for the hydrolysis of the toxic S_{P}-enantiomer of VR, relative to the wild-type enzyme. Previous work with the insecticide demeton-S demonstrated the importance of the leaving group on the catalytic activity of PTE, and our results with the new analogs of VR and VX has further demonstrated that even small changes in the leaving group can have dramatic effect on the activity of the enzyme.(37) The crystal structures of L7ep-3a and L7ep-3a I106G have provided a physical basis for these observations. The computational docking results have suggested how the remodeled active site is able to exploit hydrogen bonding interactions with Tyr-309. The disruption of the proton shuttle, along with new hydrogen bonds to Asp-301, are apparently able to enhance the attack of the bridging hydroxide on phosphorothiolate substrates. The initial data with the new racemic analog OMVR suggests that the combined asymmetric phosphorus center and the authentic leaving group of VR will allow for much more accurate predictions of the activity against VR and enable the more rapid development of new variants that are more fully optimized for the hydrolysis of VR.

### Example 37

Isolation of the R_{P}-enantiomer of OMVR. The R_{P}-enantiomer of OMVR was isolated via chiral resolution of racemic OMVR with the A80V/F132C/K185R/H254R/1274Q/S308L variant of PTE (BHR-19). About 10 mg of racemic OMVR was dissolved in 1.0 mL methanol and then diluted to 20 mL in 5.0 mM HEPES, pH 8.0. Approximately 0.6 equivalents of DTNB were added. The PTE variant was added and the reaction followed until 50% complete. Dowex-1 (chloride) was added to the reaction mixture and stirred until all color was absorbed. The protein and Dowex-1 were removed by ultrafiltration using a Viva Spin 20 10 MWCO ultra centrifugation device (GE Healthcare). The resulting solution was verified to be the isolated R_{P}-enantiomer and free from DTNB by assay with the L7ep3a variant of PTE.⁷⁰

All variants of PTE were expressed and purified as previously described.(70) In general, chemicals were obtained from Sigma Aldrich. Restriction enzymes were from New England Biolabs. Pfu Turbo DNA polymerase was from Agilent Technologies. DNA oligonucleotides were from IDT. Diethyl-VX (DEVX, FIG. 23D), dimethyl-VX (DMVX, FIG. 23E), and O-methyl-VR (OMVR, FIG. 23F) were synthesized as previously reported. (57, 70) Malathion (FIG. 23G), ethoprophos (FIG. 23H) and paraoxon (FIG. 23A) were purchased from Sigma Aldrich. Samples of VX and VR were Chemical Agent Standard Analytical Reference Material of the highest purity available, and were further purified as described previously.(70) All DNA sequences were verified by sequencing at Eton Biosciences. Bugbuster (1× Bugbuster, 50 mM HEPES/KOH, pH 8.5, 100 μM CoCl₂) was prepared by mixing 10× Bugbuster Protein Extraction Reagent (EMD Biosciences) with stock buffer and CoCl₂. The structures of the compounds used as substrates are illustrated in FIG. 23A-23H.

### Example 38

Creation of Enzyme Variant Library. The variant enzyme library was created using the highly expressed mutant A80V/K185R/I274N (VRN) as the starting template.(70) The library was created using sets of partially degenerate primers for each of the six residue positions to be mutated (Table 16). Initially, the 30 unique His254 and His257 (254/257) variants were created and verified using a Quick Change protocol (Agilent Technologies). To facilitate more efficient screening, individual variants from the possible 254/257 combinations were pooled into three separate libraries. The first library contained 11 variants (254/257=Arg/His, Asn/Cys, Asn/His, Gln/His, Gly/His, Gly/Leu, His/Cys, His/His, Ser/His, Ser/Phe, or Ser/Trp), the second contained 10 variants (254/257=Arg/Cys, Arg/Leu, Arg/Phe, Arg/Trp, His/Phe, His/Leu, His/Trp, Gln/Phe or Ser/Leu), and the final pool contained 9 variants (254/257=Asn/Leu, Asn/Phe, Asn/Trp, Gln/Cys, Gln/Leu, Gln/Trp, Gly/Cys, Gly/Phe, Gly/Trp, or Ser/Cys).

TABLE 16
Amino acid positions targeted and allowed residues in library construction.

| | | Amino | |
|---|---|---|---|
| Position | Codon* | Acids | Sequence of forward primer used |
| 106 | GBS | A G V | GTTTCCACCTTCGACGBSGGCCGTGACGTTTCC (SEQ ID |
| | | | NO: 10) |
| | TSC | S C | GTTTCCACCTTCGACTSCGGCCGTGACGTTTCC (SEQ ID |
| | | | NO: 11) |
| | ATC | I | GTTTCCACCTTCGACATCGGCCGTGACGTTTCC (SEQ ID |
| | | | NO: 12) |
| 132 | GHW | A D E V | GCTACCGGTCTGTGGGHWGACCCGCCGCTGAGC (SEQ |
| | | | ID NO: 13) |
| | TKK | C F L W | GCTACCGGTCTGTGGTKKGACCCGCCGCTGAGC (SEQ |
| | | | ID NO: 14) |
| 254 | CRM | H Q R | CTGATCGGTCTGGACCRMATCCCGCACTCCGCTATC |
| | | | (SEQ ID NO: 15) |
| | ARC | N S | CTGATCGGTCTGGACARCATCCCGCACTCCGCTATC |
| | | | (SEQ ID NO: 16) |
| | GGC | G | CTGATCGGTCTGGACGGCATCCCGCACTCCGCTATC |
| | | | (SEQ ID NO: 17) |
| 254/257 | CRM/TKK | H Q R / C F | GATCGGTCTGGACCRMATCCCGTKKTCCGCTATCGGTC |
| | | L W | (SEQ ID NO: 18) |
| | ARC/TKK | N S / C F L | GATCGGTCTGGACARCATCCCGTKKTCCGCTATCGGTC |
| | | W | (SEQ ID NO: 19) |
| | GGC/TKK | G / C F L W | GATCGGTCTGGACGGCATCCCGTKKTCCGCTATCGGTC |
| | | | (SEQ ID NO: 20) |
| 274 | ANC | I N S T | CCGCTCTGCTGGGTANCCGTTCCTGGCAGACCC (SEQ ID |
| | | | NO: 21) |
| | CAG | Q | CCGCTCTGCTGGGTCAGCGTTCCTGGCAGACCC (SEQ ID |
| | | | NO: 22) |
| 308 | RTT | V I | CTGTTCGGTTTCTCGRTTTACGTTACCAACATC (SEQ ID |
| | | | NO: 23) |
| | AGC | S | CTGTTCGGTTTCTCGAGCTACGTTACCAACATC (SEQ ID |
| | | | NO: 24) |
| | CTG | L | CTGTTCGGTTTCTCGCTGTACGTTACCAACATC (SEQ ID |
| | | | NO: 25) |

*Codes for nucleotide mixes for degenerate primers: B = (G + C + T), S = (G + C), H = (A + T + C), W = (A + T), N = (A + G + C + T), R = (A + G), M = (A + C), K = (G + T).

The final library was constructed using a sequential primer overlap extension technique.(89) The gene was amplified in three fragments using a 5′-primer containing an NdeI cut-site, the mutagenic primers listed in Table 16, and a 3′-primer containing an EcoRI cut-site. The generated fragments spanned the sequences from the 5′-end of the gene to that corresponding to amino acid residue position 106, residue position 106 to residue position 274, and from residue position 274 to the 3′-end of the gene. A total of 150 ng of these fragments were combined in a 1:2:1 ratio and used as the template in an overlap extension PCR with the 5′ and 3′ primers to achieve a full-size gene product. The extended PCR product was then used as a template for a second round of overlap extension PCR to add the additional mutations. The fragments in the second round were from the 5′-end to amino acid position 132, from amino acid position 132 to amino acid position 308, and from amino acid position 308 to the 3′ end of the gene. Efforts to generate the final gene product via overlap extension PCR proved unsuccessful. As an alternative, the NheI cut-site, which occurs at the codons for amino acids 204 and 205, was utilized. The 5′-end of the gene, extending from the 5′-end to the amino acid residue position 308, was generated separately from the 3′-end (amino acid residue position 132 to the 3′-end) via PCR overlap extension. The final products were digested with either NheI and NdeI (5′) or EcoRI (3′). The gel purified products were ligated into pET-20b, which was digested with NdeI and EcoRI and similarly purified. Sequencing of randomly selected colonies demonstrated that greater than 90% of the isolated plasmids contained a correct gene construct.

### Example 39

Growth of Colonies and Preparation of Lysates. The ligated library plasmid was transformed into BL21 (DE3) Escherichia coli cells and plated to obtain approximately 100-300 colonies on a plate. Single colonies were picked and grown in 750 μL of Super Broth at 30° C. prior to addition of IPTG to a final concentration of 1.0 mM and cultures allowed to grow for an additional 4 h. As controls, the parent variant (VRN) and a variant with high activity against DEVX (L7ep3a) were grown within each block.(70) Frozen permanents of each colony were made by mixing 100 μL of the cell culture with 100 μL of freeze-down solution (60% glycerol, 100 mM MgSO₄, 20 mM Tris, pH 5.6) and stored at −80° C. Cell density was measured by making a 1:10 dilution of each culture in 50 mM HEPES (pH 8.5) and the OD₆₀₀ was recorded. Cell lysates were prepared by mixing 100 μL of each culture with 100 μL of 1× Bugbuster. For rescreening of selected variants, cells were harvested prior to lysis. In these cases, 100 μL of cells were placed in a V-bottom 96-well block and centrifuged for 10 min in a spin-bucket rotor at 1375 g. The medium was decanted and cells stored at −80° C. prior to use. Lysate was prepared by addition of 100 μL of 1× Bugbuster and pipetted to mix. Lysate was diluted 1:2 with PTE purification buffer (50 mM HEPES (pH 8.5), 100 μM CoCl₂) prior to use.

### Example 40

Screening Library for Catalytic Activity. The prepared lysates were screened for activity against 100 μM DEVX (FIG. 23D), 100 μM DMVX (FIG. 23E), 200 μM OMVR (FIG. 23F), 200 μM malathion (FIG. 23G), and 200 μM ethoprophos (FIG. 2311). Screening assays were 250 μL total volume containing 50 mM HEPES (pH 8.0), 100 μM CoCl₂, and 0.3 mM DTNB. Substrates were pre-dissolved in a small volume of MeOH prior to adding to the assay mixture. Reactions were initiated by the addition of 25 μL of cell lysate. Reactions were followed at 412 nm for up to 4 h.

### Example 41

Quantitative Activity Analysis. The library screen relies on the activity of crude lysates containing variants against a panel of substrates, while the substrates and assay conditions are kept constant, the amount of enzyme added in each case is ultimately unknown. For the purposes of analysis, the initial absorbance reading and readings following given periods of time are recorded, and then to accommodate the potential differences in culture growth the readings from a given plate are normalized by the A₆₀₀ reading of each respective well according to eq. 1, where Aᵢⱼ is the activity of colony i with substrate j, (ΔA₄₁₂)ᵢⱼ is the change in absorbance at 412 nm over time for colony i with substrate j, ODᵢ is the optical density at 600 nm for a colony i, and OD_{C} is the average optical density for the four control variants on the plate. Any wells lacking substantial growth are eliminated from further analysis and the time varied between 2-4 h for any given plate.

$${A}_{ij} = \frac{{( Δ {A}_{412} )}_{ij}}{{OD}_{i} / {OD}_{C}} \qquad ( eq . 1 )$$

While eq. 1 corrects for differential growth on a given plate, in reality, each plate will have somewhat different readings due to discrepancies in the timing of the growth and assay readings, which differ from day to day. For comparative purposes, the values from differing plates need to be scaled to each other. Therefore, the normalized values for each well are divided by the average value obtained for the L7ep3a control variant with DEVX (FIG. 23D). Eq. 2 yields the scaled value for each colony for each substrate, where Aₛᵢⱼ is the scaled activity of colony i with substrate j, and A_{c} is the average normalized activity of the L7ep3a control with DEVX for the culture plate as defined according to eq. 1.

$${A}_{sij} = \frac{{A}_{ij}}{{A}_{c}} \qquad ( eq . 2 )$$

### Example 42

Analysis of Identified Variants. For many variants, the activity data were collected redundantly due to some variants being identified multiple times in a given screen, and some replicates were obtained from the direct rescreening of colonies. Where multiple replicates were identified, the average value of the variant was calculated according to eq. 3, where Aᵥⱼ is the average activity of all colonies of variant v with substrate j, and n is the total number of replicates observed for the variant. To assess the spread of the individual measurements, the fold activity, Fᵢⱼ, is calculated according to eq. 4. To avoid artifacts due to the division of very small numbers, any colonies with normalized and scaled activity less than 0.05 were eliminated from consideration.

$$\begin{matrix}{A}_{vj} = \frac{{\sum }_{i = 1}^{n} {A}_{sij}}{n} & ( eq . 3 ) \\ {F}_{ij} = \frac{{A}_{sij}}{A vj} & ( eq . 4 )\end{matrix}$$

Eq. 3 provides a quantification of the activity of each variant with each substrate, but provides no information on how that activity compares to any other. The relative activity of each variant with each substrate was calculated according to eq. 5, where Aᵥₜⱼ is the activity of variant v with substrate j divided by average total activity of variants with all substrates (m), and n is the total number of sequenced variants. To avoid generating errors in calculations, Aᵥⱼ was arbitrarily set to 0.01 when a variant did not have activity with the substrate.

$${A}_{( vt ) j} = \frac{{A}_{vj}}{{\sum }_{v = 1}^{n} {\sum }_{j = 1}^{m} ( \frac{{A}_{vj}}{n} )} \qquad ( eq . 5 )$$

### Example 43

Determination of Effects for Single Mutations. Single site mutants were not constructed in this study, and therefore, to determine the effects of single mutations, pairs of sequenced variants, which differ only at a single amino acid position, were identified. The activity coefficient between variants C₍ₖₗ₎ where k and l represent the variants in question were calculated according to eq. 6, where Aᵥₜ_{ₖ}ⱼ is the activity of the first variant (vₖ) and Aᵥₜ_{ₗ}ⱼ is the activity of the second variant (vₗ) with substrate j.

$${C}_{( kl ) j} = \frac{{A}_{{vt}_{k} j}}{{A}_{{vt}_{l} j}} \qquad ( eq . 6 )$$

For single site mutants, the activity coefficient C₍ₖₗ₎ₚⱼ is used to represent the effect of a switch from amino acid k to amino acid l at position p. Given the large number of possible mutations and limited number of sequence pairs identified, not all substitutions were observed. In cases where a particular substitution was not observed, the coefficient for that mutation was calculated by multiplication or division of the observed substitutions involving the amino acids in question, according to eq. 7.

C₍ₖₗ₎ₚⱼ=C₍ₖₘ₎ₚⱼ/C₍ₗₘ₎ₚⱼ OR C₍ₖₗ₎ₚⱼ=C₍ₖₘ₎ₚⱼ*C₍ₘₗ₎ₚⱼ (eq. 7)

The activity coefficients have been calculated for all possible single-site mutations with respect to the wild-type amino acid according to eq. 8, where Aᵥₜ_{_{w}}ₚⱼ is the activity of the first variant containing the wild-type reside at position p, and Aᵥₜ_{ₖ}ₙⱼ is the activity of the second variant (vₖ), which contains residue k at position p with substrate j.

$${C}_{( wk ) pj} = \frac{{A}_{{vt}_{w} pj}}{{A}_{{vt}_{k} pj}} \qquad ( eq . 8 )$$

To evaluate the ability of a mutation to increase the overall activity of the enzyme, the overall coefficient was calculated according to eq. 9, where C_{(wk)pj} is the coefficient for substrate j for switching the amino acid at position p from the wild type to amino acid k. Due to insufficient activity with the majority of variants, the data for ethoprophos (8) were excluded from the calculations of C_{(wk)p}.

$${C}_{( wk ) p} = exp ( \frac{{\sum }_{j = 1}^{m} ( ln {C}_{( wk ) pj} )}{m} ) \qquad ( eq . 9 )$$

Theoretically, in the absence of any synergistic effects, the activity of any mutation could be predicted by dividing the activity of the known variant by the coefficient for the desired substitution, and multiple mutations would be predicted by the product of the series of coefficients, where Aᵥₜ_{ₖ}ⱼ is the activity of the initial variant with substrate j, C₍ₖₗ₎ₚⱼ is the activity coefficient for substrate j for switching amino acid k for l at position n, m is the total number of positions to be changed, and Aᵥ_{ₗ}ₜⱼ is the activity of the final variant.

$${A}_{{vt}_{l} j} = \frac{{A}_{{vt}_{k} j}}{{\prod }_{n = 1}^{m} {C}_{( kl ) pj}} \qquad ( eq . 10 )$$

### Example 44

Large Scale Growth and Purification of Variants. For variants of interest, 1-L cultures were grown in Terrific Broth supplemented with 1.0 mM CoCl₂ at 30° C., as previously described.⁷⁰ Purifications were carried out in 50 mM HEPES (pH 8.5) supplemented with 100 μM CoCl₂ according to published protocols. For variants showing poor expression, purified protein was concentrated to >2 mg/mL using a Viva Spin 20 Ultrafiltration device (GE Healthcare) and all purified variants were flash frozen in liquid nitrogen and stored at −80° C. prior to use.

### Example 45

Kinetic Characterization of Purified Variants. All purified variants were characterized with the substrates paraoxon (FIG. 23A), racemic VX (FIG. 23B), racemic VR (FIG. 23C), DEVX (FIG. 23D), DMVX (FIG. 23E), racemic OMVR (FIG. 23F) and malathion (FIG. 23G). For DEVX, DMVX, and malathion steady state kinetic parameters were determined in 250 μL total volume reactions, containing 50 mM HEPES/KOH (pH 8.0), 0.3 mM DTNB and 100 μM CoCl₂ at 30° C. Substrate concentrations varied from 1.0 mM to 8 μM. Reactions were initiated by addition of appropriately diluted enzyme and followed by the release of the thiol leaving group with DTNB at 412 nm (E₄₁₂=14,150 M⁻¹ cm⁻¹) using a Molecular Devices Spectramax 384 Plus 96-well plate reader. Reactions were typically followed for 15 min and fit to the Michaelis-Menten equation. Reactions with paraoxon (FIG. 23A) were conducted in CHES/KOH (pH 9.0) with 100 μM CoCl₂ at 30° C., by following the release of p-nitrophenol (E₄₀₀=17,000 M⁻¹ cm⁻¹). Reaction rates with racemic OMVR (FIG. 23F) were measured by total hydrolysis assays using ˜60 μM racemic substrate as previously described.(57) Assays were conducted in a total volume of 1.0 mL in 50 mM HEPES/KOH (pH 8.0) and 0.3 mM DTNB. Assays were followed at 412 nm for 30 min with readings every 5 s. Data were converted to fraction hydrolyzed as a function of time and fit to either a single (eq. 11) or double exponential (eq. 12) decay as previously described.(65) To determine the stereochemical preferences of the enzyme variants, reactions were allowed to proceed to approximately 50% completion prior to the addition of sufficient quantities of a PTE variant of known stereoselectivity to quickly hydrolyze one enantiomer. The preference was determined by the differential amount of each enantiomer remaining at the 50% point of the reaction.

### Example 46

Stereoselective Hydrolysis of Racemic VX and VR. Low initial concentrations (19 to 160 μM) of racemic VX and VR were hydrolyzed by variants of PTE in a solution containing 0.1 mM CoCl₂, 0.3 mM DTNB, and 50 mM Bis-Tris-propane, pH 8.0. The reactions were followed to completion and the fraction hydrolyzed plotted as a function of time. The time courses were fit to eqs. 11 and 12 where F is the fraction hydrolyzed, a and b are the magnitudes of the exponential phases, t is time, and k1 and k2 are the rate constants for each phase.(65)

F=a(1−e^{-k}^{₁}^{t}) (eq. 11)

F=a(1−e^{-k}^{₁}^{t})+b(1−e^{-k}^{₂}^{t}) (eq. 12)

Stereochemical preferences for VX and VR were determined by running reactions with racemic VX or VR with variants of interest until the reaction was ˜50% complete. Residual substrate was then extracted and analyzed by chiral LC-MS as previously described.(90)

### Example 47

Sequence Analysis and Construction of Sequence Similarity Networks. The library variants are described by a six-letter code consisting of the amino acid present at each of the six sites subject to mutation in order from the N-terminus to the C-terminus. Because of the large number of variants, the total list of variants possible was generated by an iterative logic function using Excel. Each variant in the library has 28 other variants, which differ by only one amino acid. The total list of all pairs of variants, which differ by only one amino acid was then scored and sorted to enable the elimination of all duplicates. The final list of variants and their single amino acid partners were then loaded into the program Cytoscape to form a network map.(91) Network maps were displayed with a yfile Organic Layout. Similar operations were conducted to generate a list of sequenced variants along with the relationship to all other variants.

### Example 48

Determination of Cysteine Reactivity with DTNB and Effects on Catalysis. In order to determine if the added cysteine residues in the new variants of PTE were reactive towards DTNB, the wild-type like variant (VRN), BHR-73, one variant with an added cysteine at position-106 (BHR-70), and two variants with cysteine at position-132 (BHR-69 and BHR-74) were analyzed. The enzymes were diluted to 1 mg/mL (˜28 μM) into an assay containing 0.3 mM DTNB and 50 mM HEPES, pH 8.0. The reactions of the free thiol groups were monitored at 412 nm (E₄₁₂=14150 M⁻¹ cm⁻¹). To determine what effect the reaction with DTNB has on the catalytic activity of the variants, the activity of BHR-69 and BHR-74 were measured with DEVX and OMVR in the absence of DTNB by a discontinuous total hydrolysis assay. Initially, reaction conditions were determined that resulted in the total hydrolysis of either 40 μM DEVX or 70 μM OMVR in ˜30 min in reactions of 1.0 mL in 50 mM HEPES, pH 8.0, using 0.3 mM DTNB. Discontinuous reactions were then conducted in a total volume of 30 mL with 50 mM HEPES, pH 8.0. Reactions with DEVX were initiated by the addition of either 147 nM BHR-74 or 67 nM BHR-69. The reactions with OMVR were initiated by the addition of either 294 nM BHR-74 or 6.7 μM BHR-69. The reactions were monitored by removing 500 μL aliquots at 30 s intervals and then mixed with an equal volume of 0.6 mM DTNB. The absorbance was measured at 412 nm and the data were fit to eq. 11.

### Example 49

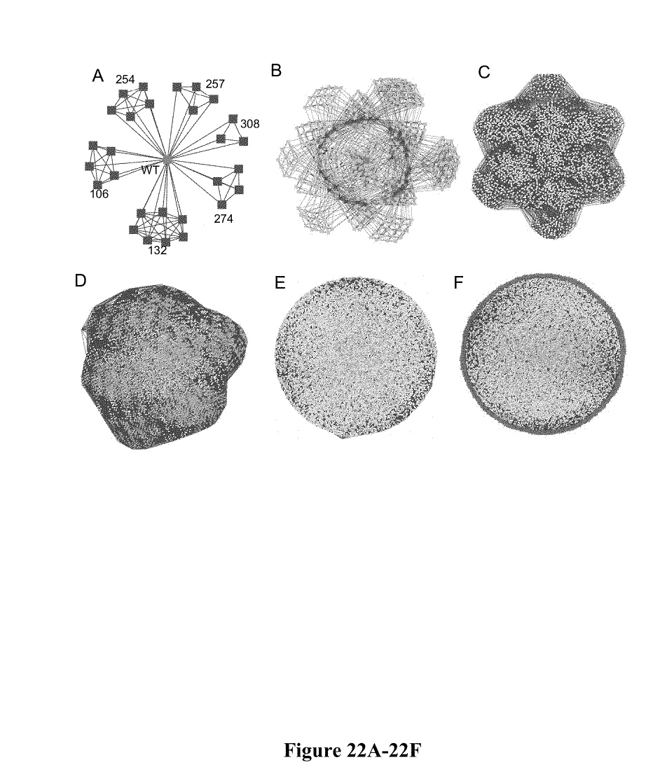

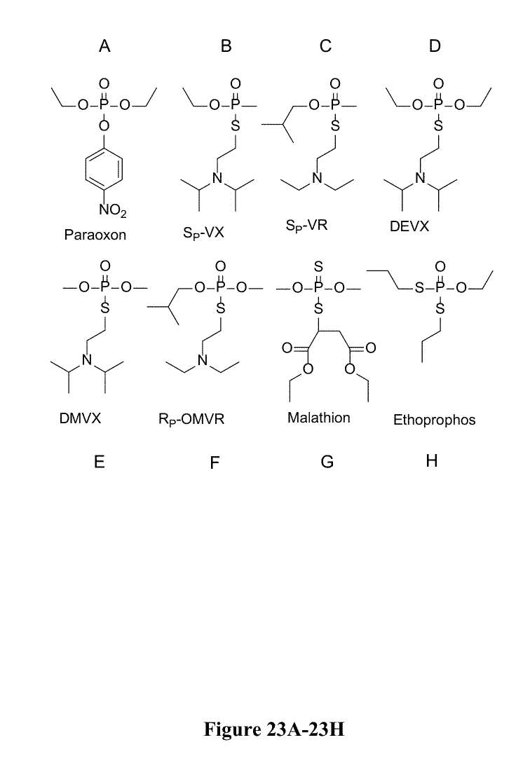

Library Design and Construction. Previous studies have attempted to enhance PTE for the hydrolysis of V-type nerve agents, but to date there has been no comprehensive effort to determine the contribution of individual mutations or the best combination thereof. Here a limited variant library was constructed targeting five active site residues implicated in V-agent hydrolysis and residue 274, which, while not in the active site, is known to affect activity (FIG. 21).(70) The active site residues targeted are 106, 132, 254, 257, and 308. The amino acids at each position were selected based on their reported appearance in variants that improve V-agent hydrolysis (Table 16).(57, 63, 70-73) The library consists of 28,800 variants with a total of 403,200 unique single amino acid substitution relationships (FIG. 22A-22F). Construction consisted of three sequential steps to introduce pairs of mutants to the parent. In the first step, the 30 unique 254/257 variants were isolated and pooled as the template for subsequent steps. To facilitate efficient screening, the library was divided into three parts based on the 254/257 combinations allowed. The complete library was successfully constructed via two additional overlap extension PCR steps, which introduced the remaining mutations as pairs. Sequencing of randomly selected colonies revealed that ˜90% of colonies contained a correct construction.

### Example 50

Screening the Library. While the ultimate target of this project was the V-type nerve agents, especially the S_{P}-enantiomer of VR (FIG. 23C), the use of analogs was required for the screening protocols, due to the high toxicity and security concerns associated with authentic agents. As such, a panel of related organic phosphorothiolate compounds was selected to serve as analogs for the V-agents. The analogs selected were DEVX (FIG. 23D), which has the same leaving group and an ethyl substituent as VX (FIG. 23B); DMVX (FIG. 23E), which contains the authentic leaving group of VX (FIG. 23B), but attached to the smaller dimethyl phosphorus core; OMVR (FIG. 23F), which has the authentic leaving group and an O-isobutyl group as VR (FIG. 23C) as well as a chiral phosphorus center; and the two common phosphorothiolate insecticides, malathion (FIG. 23G) and ethoprophos (FIG. 23H).

A total of 33,300 variants (1.2-fold coverage) were screened against the complete panel of substrate analogs, which corresponds to an expectation of approximately 65% of the total possible variants being screened. To maximize the data available during the screening process, colonies were selected for sequencing and purification from the initial two segments of the library by qualitative analysis of the raw screening data, primarily focusing on broad catalytic activity. For the first two rounds of screening, colonies selected for sequencing were retransformed and rescreened against the screening analogs. Because of high stereoselectivity, the rescreen for the final section of the library was only conducted for variants active with OMVR (FIG. 23F) and the rescreen was limited to the isolated R_{P}-enantiomer of OMVR (FIG. 23D).

Given the small number of variants typically reported in enzyme evolutions studies, there was a surprisingly large number of highly active variants identified in the screen. DEVX (FIG. 23D) gave the highest activity with 295 variants having higher activity than the positive control in the screen. The control variants used in screening did not demonstrate significant activity against OMVR (FIG. 23F) or malathion (FIG. 23G), however 68 variants were identified with activity for OMVR higher than the controls with DEVX, and 138 variants demonstrated activity with malathion, which outperformed the controls with DEVX. Much less activity was observed with DMVX (FIG. 23E) and ethoprophos (FIG. 23H). Nonetheless, 499 variants had measurable activity with DMVX and 361 variants demonstrated activity with ethoprophos (FIG. 23H).

### Example 51

Reproducibility of Screen Activity. The raw absorbance readings from the screening protocol were normalized for culture growth and scaled to allow comparison between different plates according to eqs. 1 and 2, respectively. To determine the level of significance that can be attached to a given result, cases where multiple colonies containing the same variant were identified. In addition, the rescreening of the colonies of interest also provided replicates for selected colonies. In total, 175 variants were found to have been screened more than once. The range of replicates varied from 2-7 per variant. The average activity for a given variant with a given substrate and the relative activity from each colony of that variant were calculated according to eqs. 3 and 4, respectively. This analysis found that for DEVX, 87% of the replicates fall within 1.5-fold of the variant average and 95% fall within 2-fold. Similar values were found with OMVR (95% within 2-fold) and DMVX (93% within 2-fold). The variability with malathion (FIG. 23G) was found to be much greater with only 70% of variants falling within 2-fold. There were insufficient replicated variants with ethoprophos to determine the spread for this substrate. Consequently, a 2-fold difference was considered significant for further analysis.

### Example 52

Selection, Purification, and Characterization of Library Variants. As indicated above, to maximize the amount of information available during the screening process, variants were selected for purification based on the raw screen data during the course of screening. From the initial segment of the library, 33 variants were selected for purification. In general, variants were selected for broad specificity and high activity. However, two variants were selected for nearly exclusive malathion activity (BHR-9, BHR-33), one was selected based on ethoprophos activity (BHR-32), one for DEVX exclusive activity (BHR-26), and two that were active for only the V-agent analogs (BHR-8, BHR-20). Steady state kinetic analyses were conducted with DEVX (FIG. 23D), DMVX (FIG. 23E), malathion (FIG. 23G), and paraoxon (FIG. 23A). Data are presented in Tables 22-25. Against DEVX (FIG. 23D) multiple variants were identified, which were more than 100-fold improved with the best variant being BHR-19 with k_{cat}/Kₘ of 2×10⁵ M⁻¹ s⁻¹, which is 167-fold improved over wild-type PTE and has a Kₘ value of 320 μM (Table 17). Many variants were substantially improved in k_{cat} with values up to ˜100 s⁻¹. In general, the variants selected for broad specificity performed better than those selected for specific substrates. As seen previously, the activity against DMVX (FIG. 23E) falls below that of other substrates,¹ but the best variant was BHR-4 with a k_{cat}/Kₘ of 9.1×10³ M⁻¹ s⁻¹, which is 479-fold improved over wild-type PTE (Table 17). The k_{cat} values with DMVX (FIG. 23E) were approximately 10-fold below that seen with DEVX (FIG. 23D) with a maximal value of 10.5 s⁻¹ observed for BHR-19. The two variants specifically selected for malathion activity showed more than 100-fold improvement. The best variant was BHR-33 with a k_{cat} of 7.0 s⁻¹ and a k_{cat}/Kₘ of 1.5×10⁴ M⁻¹ s⁻¹, which is 500-fold improved over wild-type PTE (Table 17). Other variants showed little activity for malathion. The highest activity of any compound tested was observed with paraoxon with all variants having enzymatic efficiency greater than 10⁶ M⁻¹ s⁻¹ (Table 25).

TABLE 17
Kinetic constants for PTE variants with achiral substrates.

| | | DEVX | | | | DMVX | | | | Malathion | | | |
|---|---|---|---|---|---|---|---|---|---|---|---|---|---|
| | | | | | Fold | | | | Fold | | | | Fold |
| | | k_{cat} | Kₘ | k_{cat}/Kₘ | im- | k_{cat} | Kₘ | k_{cat}/Kₘ | im- | k_{cat} | Kₘ | k_{cat}/Kₘ | im- |
| Variant | Sequence^{a} | (s⁻¹) | (mM) | (M⁻¹s⁻¹) | proved | (s⁻¹) | (mM) | (M⁻¹s⁻¹) | proved | (s⁻¹) | (mM) | (M⁻¹s⁻¹) | proved |
| WT | ifhhis | 1.1 | 0.87 | 1.2 × 10³ | — | 0.021 | 1.1 | 1.9 × 10¹ | — | 0.047 | 1.0 | 3.0 × 10¹ | — |
| BHR-4 | iCQhiL | 78 | 0.60 | 1.3 × 10³ | 108 | 3.9 | 0.43 | 9.1 × 10³ | 479 | nd | nd | 5.5 × 10¹ | 2 |
| BHR-7 | iCRhTL | 45 | 0.29 | 1.6 × 10³ | 133 | 9.1 | 1.3 | 7.0 × 10³ | 368 | nd | nd | 1.1 × 10³ | 37 |
| BHR-19 | iCRhQL | 65 | 0.32 | 2.0 × 10³ | 167 | 10.5 | 1.3 | 8.1 × 10³ | 426 | 3.1 | 1.9 | 1.6 × 10³ | 53 |
| BHR-23 | iVRhSL | 19 | 0.48 | 4.0 × 10⁴ | 33 | 2.9 | 2.5 | 1.2 × 10³ | 63 | 1.7 | 1.7 | 1.0 × 10³ | 33 |
| BHR-33 | VfRhTL | 4.8 | 1.6 | 3.1 × 10³ | 3 | 0.12 | 1.0 | 1.2 × 10² | 6 | 7 | 0.5 | 1.5 × 10⁴ | 500 |
| BHR-39 | ACQFQL | 75 | 0.35 | 2.2 × 10³ | 183 | 5.9 | 1.0 | 5.9 × 10³ | 311 | 0.16 | 0.7 | 2.1 × 10² | 7 |
| BHR-40 | ACRFQL | 99 | 0.23 | 4.2 × 10³ | 350 | 6.3 | 0.99 | 6.3 × 10³ | 332 | 1.2 | 0.16 | 8.0 × 10³ | 267 |
| BHR-41 | ACRFSL | 105 | 0.22 | 4.8 × 10³ | 400 | 4.7 | 0.74 | 6.3 × 10³ | 332 | 1.69 | 0.25 | 6.8 × 10³ | 227 |
| BHR-42 | AERLTs | 40 | 3.0 | 1.3 × 10⁴ | 11 | nd | nd | 4.8 × 10¹ | 3 | 0.0034 | 0.18 | 1.9 × 10² | 6 |
| BHR-44 | ALQFTs | 8 | 2.2 | 2.8 × 10³ | 2 | nd | nd | 1.9 × 10² | 10 | 0.0043 | 0.25 | 1.7 × 10¹ | 1 |
| BHR-45 | ACQFNL | 52 | 0.25 | 2.1 × 10³ | 178 | 6.2 | 0.71 | 8.7 × 10³ | 458 | 0.086 | 0.3 | 2.9 × 10² | 10 |
| BHR-53 | ACRLNL | 69 | 0.25 | 2.7 × 10³ | 225 | 2.3 | 2.0 | 1.1 × 10³ | 58 | 1.39 | 0.32 | 4.3 × 10³ | 143 |
| BHR-54 | ACQLTs | 0.21 | 0.42 | 4.9 × 10² | 0 | 0.21 | 0.44 | 4.7 × 10² | 25 | 0.006 | 0.11 | 3.1 × 10¹ | 1 |
| BHR-67 | iCQFNs | 29 | 0.28 | 1.0 × 10³ | 86 | 3.0 | 0.52 | 5.7 × 10³ | 300 | 0.0033 | 0.06 | 6.0 × 10¹ | 2 |
| BHR-69 | iCQFis | 11 | 0.27 | 4.0 × 10⁴ | 33 | 0.85 | 0.32 | 2.7 × 10³ | 142 | 0.0014 | 0.04 | 3.5 × 10¹ | 1 |
| BHR-70 | CVRLSL | 54 | 1.1 | 4.9 × 10⁴ | 41 | 0.4 | 3.0 | 1.5 × 10² | 8 | 3.3 | 0.2 | 1.7 × 10⁴ | 567 |
| BHR-72 | ACGhNs | 8 | 1.2 | 6.8 × 10³ | 6 | 0.1 | 0.3 | 3.3 × 10² | 17 | 0.0043 | 0.013 | 3.3 × 10² | 11 |
| BHR-73 | AEGhNs | 2.9 | 2.5 | 1.2 × 10³ | 1 | nd | nd | 7.2 × 10¹ | 4 | 0.0008 | 0.016 | 5.0 × 10¹ | 2 |
| BHR-74 | ACQFNs | 20 | 0.9 | 2.2 × 10⁴ | 18 | 1.0 | 0.71 | 1.4 × 10³ | 74 | 0.0039 | 0.001 | 4.0 × 10³ | 133 |
| BHR-75 | AEQFQs | 16 | 4.0 | 3.6 × 10³ | 3 | nd | nd | 3.9 × 10² | 20 | 0.0007 | 0.11 | 6.0 × 10⁰ | 0 |
| BHR-73- | AEGhNs- | 20 | 20 | 1.2 × 10³ | 1 | 1.2 | 7.0 | 1.6 × 10² | 8 | 0.0011 | 0.06 | 1.8 × 10¹ | 0.6 |
| W | W | | | | | | | | | | | | |
| BHR-73- | AEGhNs- | 7 | 4.0 | 1.5 × 10³ | 1 | 0.23 | 1.8 | 1.3 × 10² | 7 | nd | nd | 1.1 × 10⁰ | 0.04 |
| NW | NW | | | | | | | | | | | | |
| BHR-73- | AEGhNs- | 10 | 4.6 | 2.0 × 10³ | 2 | 0.44 | 2.9 | 1.4 × 10² | 7 | 0.0009 | 0.4 | 2.3 × 10⁰ | 0.1 |
| MNW | MNW | | | | | | | | | | | | |
| BHR-74- | ACQFNs- | 14 | 0.31 | 4.5 × 10⁴ | 38 | 2.3 | 0.5 | 4.6 × 10³ | 242 | nd | nd | 1.4 × 10¹ | 0.5 |
| NW | NW | | | | | | | | | | | | |

^{a}Sequence given as single letter amino acid code for residues present at position 106/132/254/257/274/308.
For letters following the numerical designation
W = Y309W,
N = T173N,
M = C59M.
nd = not determined.
Errors associated with kinetic measurements are reported in supplementary tables.

TABLE 18
Kinetic constants for purified PTE variants with paraoxon.

| | | k_{cat} | Kₘ | k_{cat}/Kₘ | | | k_{cat} | Kₘ | k_{cat}/Kₘ |
|---|---|---|---|---|---|---|---|---|---|
| Number | Sequence^{a} | (s⁻¹) | (μM) | (M⁻¹s⁻¹) | Number | Sequence^{a} | (s⁻¹) | (mM) | (M⁻¹s⁻¹) |
| BHR-1 | iCRhSs | 410 ± 7 | 41 ± 4 | 1.0 ± 0.1 × 10⁷ | BHR-39 | ACQFQL | 419 ± 5 | 0.029 ± 0.001 | 1.45 ± 0.07 × 10⁷ |
| BHR-2 | iCRhTs | 415 ± 7 | 30 ± 3 | 1.4 ± 0.2 × 10⁷ | BHR-40 | ACRFQL | 890 ± 20 | 0.020 ± 0.001 | 4.4 ± 0.3 × 10⁷ |
| BHR-3 | SCRhNL | 770 ± 10 | 46 ± 4 | 1.7 ± 0.1 × 10⁷ | BHR-41 | ACRFSL | 800 ± 10 | 0.021 ± 0.001 | 3.8 ± 0.2 × 10⁷ |
| BHR-4 | iCQhiL | 5300 ± 200 | 190 ± 20 | 2.8 ± 0.3 × 10⁷ | BHR-42 | AERLTs | 2090 ± 20 | 0.194 ± 0.005 | 1.08 ± 0.03 × 10⁷ |
| BHR-5 | iCQhSL | 6000 ± 200 | 310 ± 30 | 1.9 ± 0.2 × 10⁷ | BHR-43 | AVQhNs | 9860 ± 80 | 0.352 ± 0.006 | 2.80 ± 00.6 × 10⁷ |
| BHR-6 | sCQhNs | 17000 ± 800 | 720 ± 70 | 2.4 ± 0.2 × 10⁷ | BHR-44 | ALQFTs | 249 ± 2 | 0.018 ± 0.001 | 1.40 ± 0.04 × 10⁷ |
| BHR-7 | iCRhTL | 230 ± 6 | 26 ± 3 | 9.0 ± 0.1 × 10⁶ | BHR-45 | ACQFNL | 318 ± 7 | 0.022 ± 0.002 | 1.4 ± 0.1 × 10⁷ |
| BHR-8 | VCQhNL | 7500 ± 200 | 390 ± 30 | 1.9 ± 0.2 × 10⁷ | BHR-46 | SERLTL | 2290 ± 20 | 0.227 ± 0.005 | 1.01 ± 0.03 × 10⁷ |
| BHR-9 | CfRhQl | 790 ± 20 | 24 ± 3 | 3.3 ± 0.3 × 10⁷ | BHR-47 | GEQLQs | 1260 ± 20 | 0.044 ± 0.002 | 2.9 ± 0.1 × 10⁷ |
| BHR-10 | iChhNs | 6500 ± 200 | 290 ± 20 | 2.2 ± 0.2 × 10⁷ | BHR-48 | LWQLTS | 706 ± 5 | 0.019 ± 0.001 | 3.9 ± 0.1 × 10⁷ |
| BHR-11 | CAQhQL | 690 ± 10 | 36 ± 3 | 1.9 ± 0.2 × 10⁷ | BHR-49 | GCQhiL | 1770 ± 50 | 0.061 ± 0.005 | 2.9 ± 0.2 × 10⁷ |
| BHR-12 | ACQhTs | 400 ± 6 | 30 ± 3 | 1.3 ± 0.1 × 10⁷ | BHR-50 | iVQFNI | 94 ± 1 | 0.0087 ± 0.0003 | 1.09 ± 0.04 × 10⁷ |
| BHR-13 | iCGhNs | 5100 ± 200 | 670 ± 60 | 7.6 ± 0.7 × 10⁷ | BHR-51 | iCRLNI | 384 ± 6 | 0.081 ± 0.004 | 4.8 ± 0.2 × 10⁶ |
| BHR-14 | GCQhNL | 1750 ± 30 | 61 ± 6 | 2.9 ± 0.3 × 10⁷ | BHR-52 | ACRhTs | 1100 ± 30 | 0.038 ± 0.004 | 2.9 ± 0.3 × 10⁷ |
| BHR-15 | AARhQL | 1400 ± 300 | 56 ± 6 | 2.5 ± 0.3 × 10⁷ | BHR-53 | ACRLNL | 3600 ± 300 | 0.13 ± 0.02 | 2.9 ± 0.5 × 10⁷ |
| BHR-16 | VCRhNL | 375 ± 6 | 26 ± 3 | 1.4 ± 0.1 × 10⁷ | BHR-54 | ACQLTs | 305 ± 6 | 0.032 ± 0.002 | 9.53 ± 0.06 × 10⁶ |
| BHR-17 | ACRhiL | 1070 ± 20 | 68 ± 7 | 1.6 ± 0.2 × 10⁷ | BHR-55 | iLGFQs | 352 ± 7 | 0.065 ± 0.005 | 5.4 ± 0.4 × 10⁶ |
| BHR-18 | CLRhQL | 553 ± 8 | 19 ± 2 | 2.9 ± 0.3 × 10⁷ | BHR-56 | iLGFSs | 370 ± 9 | 0.049 ± 0.004 | 7.6 ± 0.6 × 10⁶ |
| BHR-19 | iCRhQl | 392 ± 6 | 24 ± 2 | 1.6 ± 0.1 × 10⁷ | BHR-57 | ACQHSs | 15100 ± 400 | 0.64 ± 0.3 | 2.4 ± 0.1 × 10⁷ |
| BHR-20 | iCRhQs | 480 ± 8 | 41 ± 5 | 1.2 ± 0.1 × 10⁷ | BHR-58 | ALGFQS | 710 ± 10 | 0.197 ± 0.009 | 3.6 ± 0.2 × 10⁶ |
| BHR-21 | VCRhQL | 683 ± 3 | 62 ± 7 | 1.1 ± 0.1 × 10⁷ | BHR-59 | ALGFNS | 1000 ± 10 | 0.128 ± 0.004 | 7.8 ± 0.3 × 10⁶ |
| BHR-22 | VCRhTL | 1450 ± 30 | 63 ± 6 | 2.3 ± 0.2 × 10⁷ | BHR-60 | AVGFNS | 610 ± 10 | 0.096 ± 0.005 | 6.4 ± 0.3 × 10⁶ |
| BHR-23 | iVRhSL | 580 ± 10 | 26 ± 3 | 2.2 ± 0.2 × 10⁷ | BHR-61 | ALRLQV | 2010 ± 60 | 0.15 ± 0.01 | 1.3 ± 0.1 × 10⁷ |
| BHR-24 | CLRhSL | 600 ± 6 | 24 ± 2 | 2.5 ± 0.2 × 10⁷ | BHR-62 | ACQLNs | 430 ± 10 | 0.14 ± 0.01 | 3.1 ± 0.2 × 10⁶ |
| BHR-25 | CARhNL | 610 ± 8 | 18 ± 2 | 3.4 ± 0.3 × 10⁷ | BHR-63 | VLGFQs | 930 ± 20 | 0.19 ± 0.01 | 4.9 ± 0.3 × 10⁶ |
| BHR-26 | iCQhis | 7400 ± 200 | 570 ± 40 | 1.3 ± 0.1 × 10⁷ | BHR-64 | AERLNL | 1640 ± 40 | 0.24 ± 0.01 | 6.8 ± 0.3 × 10⁶ |
| BHR-27 | CLGhTs | 3200 ± 100 | 310 ± 30 | 1.0 ± 0.1 × 10⁷ | BHR-65 | AERLQs | 2740 ± 30 | 0.262 ± 0.007 | 1.05 ± 0.03 × 10⁷ |
| BHR-28 | CVQhNL | 470 ± 10 | 12 ± 1 | 3.9 ± 0.4 × 10⁷ | BHR-66 | VVRLSL | 1990 ± 20 | 0.051 ± 0.002 | 3.9 ± 0.2 × 10⁷ |
| BHR-29 | CDQhQL | 1180 ± 30 | 76 ± 9 | 1.6 ± 0.2 × 10⁷ | BHR-67 | iCQFNs | 60 ± 1 | 0.014 ± 0.002 | 4.3 ± 0.6 × 10⁶ |
| BHR-30 | iCGhTs | 6000 ± 200 | 560 ± 40 | 1.1 ± 0.1 × 10⁷ | BHR-68 | CARLTL | 534 ± 5 | 0.028 ± 0.001 | 1.94 ± 0.7 × 10⁷ |
| BHR-31 | iCQhTL | 7400 ± 100 | 290 ± 20 | 2.5 ± 0.2 × 10⁷ | BHR-69 | iCQFis | 25.8 ± 0.5 | 0.01 ± 0.001 | 2.6 ± 0.3 × 10⁶ |
| BHR-32 | AVShQs | 7500 ± 100 | 0.308 ± 0.009 | 2.44 ± 0.08 × 10⁷ | BHR-70 | CVRLSL | 4370 ± 30 | 0.22 ± 0.004 | 1.99 ± 0.04 × 10⁷ |
| BHR-33 | VfRhTL | 640 ± 20 | 0.018 ± 0.002 | 3.5 ± 0.4 × 10⁷ | BHR-71 | AVQFNs | 229 ± 3 | 0.0159 ± 0.0009 | 1.44 ± 0.08 × 10⁷ |
| BHR-34 | iLGLNs | 500 ± 10 | 0.147 ± 0.008 | 3.4 ± 0.2 × 10⁶ | BHR-72 | ACGhNs | 11200 ± 400 | 0.84 ± 0.05 | 1.33 ± 0.09 × 10⁷ |
| BHR-35 | ACRhNs | 658 ± 8 | 0.020 ± 0.001 | 3.3 ± 0.1 × 10⁷ | BHR-73 | AEGhNs | 2810 ± 70 | 1.8 ± 0.06 | 1.56 ± 0.04 × 10⁶ |
| BHR-36 | AVQhQs | 9800 ± 100 | 0.37 ± 0.01 | 2.6 ± 0.1 × 10⁷ | BHR-74 | ACQFNs | 120 ± 2 | 0.006 ± 0.0007 | 2.0 ± 0.2 × 10⁷ |
| BHR-37 | iLGLTs | 670 ± 10 | 0.091 ± 0.005 | 7.3 ± 0.5 × 10⁶ | BHR-75 | AEQFQs | 423 ± 4 | 0.124 ± 0.004 | 3.4 ± .1 × 10⁶ |
| BHR-38 | ACQLiL | 331 ± 5 | 0.020 ± 0.001 | 1.6 ± 0.1 × 10⁷ | BHR-76 | AEQYQs | 302 ± 3 | 0.126 ± 0.003 | 2.40 ± 0.06 × 10⁶ |

^{a}Sequence given as single letter amino acid code for residues present at position 106/132/254/257/274/308.
nd = not determined.

The activity for OMVR (FIG. 23F) was measured via total hydrolysis reactions, which allowed the simultaneous measurement of k_{cat}/Kₘ values for both enantiomers and the assignment of stereochemical preference (Tables 19 and 20). Four of the variants exhibited more than 1000-fold improvements in catalytic efficiency with OMVR. The best variant for OMVR was BHR-7 with a k_{cat}/Kₘ of 4.4×10⁵ M⁻¹ s⁻¹, which is ˜6500-fold improved over wild-type PTE (Table 19). Unfortunately, the stereoselectivity for OMVR (FIG. 23F) was found to be for the less toxic S_{P}-enantiomer for all of the initial variants tested. Six of the variants were found to only hydrolyze the S_{P}-enantiomer, while the remaining variants exhibited between 2100- to 5-fold preferences (Table 20). The best variant for R_{P}-OMVR (FIG. 23F) was BHR-23 with a k_{cat}/Kₘ of 9.4×10² M⁻¹ s⁻¹ which is 138-fold improved over wild-type PTE (Table 19).

TABLE 19
Kinetic constants for PTE variants with chiral substrates.

| | | OMVR | | | | VX | | | | VR | | | |
|---|---|---|---|---|---|---|---|---|---|---|---|---|---|
| | | | | | R_{P}- | | | | R_{P}- | | | | R_{P}- |
| | | | | | Fold | | | | Fold | | | | Fold |
| | | k_{cat}/Kₘ₁ | k_{cat}/Kₘ₂ | Ratio | Im- | k_{cat}/Kₘ₁ | k_{cat}/Kₘ₂ | Ratio^{c} | Im- | k_{cat}/Kₘ₁ | k_{cat}/Kₘ₂ | Ratio^{c} | Im- |
| Variant | Sequence^{a} | (M⁻¹s⁻¹) | (M⁻¹s⁻¹) | S_{P}:R_{P} | proved | (M⁻¹s⁻¹) | (M⁻¹s⁻¹) | S_{P}:R_{P} | proved | (M⁻¹s⁻¹) | (M⁻¹s⁻¹) | S_{P}:R_{P} | proved |
| WT | ifhhis | 6.8 × 10¹ | <6.8 × 10⁰ | >10:1 | na | 8.4 × 10¹ | 8.4 × 10¹ | 1:1 | na | 1.1 × 10² | 4.3 × 10⁰ | 25:1* | na |
| BHR-4 | iCQhiL | 3.3 × 10⁴ | 5.8 × 10¹ | 560:1 | 9 | 2.3 × 10⁵ | 4.4 × 10⁴ | 1:5.2* | 2750 | 3.3 × 10⁴ | NO | >500:1 | na |
| BHR-7 | iCRhTL | 4.4 × 10⁵ | NO | >500:1 | na | 3.2 × 10⁴ | 3.2 × 10⁴ | 1:1 | 382 | 9.7 × 10⁴ | NO | >500:1 | na |
| BHR-19 | iCRhQL | 1.6 × 10⁵ | NO | >500:1 | na | 6.0 × 10⁴ | 6.0 × 10⁴ | 1:1 | 720 | 1.2 × 10⁵ | NO | >500:1* | na |
| BHR-23 | iVRhSL | 5.8 × 10⁴ | NO | >500:1 | na | 7.7 × 10⁵ | 4.2 × 10⁴ | 1:18* | 9167 | 3.0 × 10⁴ | NO | >500:1 | na |
| BHR-33 | VfRhTL | 6.2 × 10³ | NO | >500:1 | na | 7.8 × 10² | 7.8 × 10² | 1:1 | 9 | 1.3 × 10⁴ | NO | >500:1 | na |
| BHR-39 | ACQFQL | 2.2 × 10³ | 9.6 × 10² | 1:2.3 | 325 | 2.6 × 10⁴ | 2.9 × 10² | 1:89 | 310 | 8.2 × 10³ | 1.3 × 10³ | 6.5:1* | 293 |
| BHR-40 | ACRFQL | 4.6 × 10³ | 1.2 × 10³ | 4:1 | 169 | 1.2 × 10⁴ | 8.5 × 10² | 14:1 | 10 | 7.3 × 10³ | 4.3 × 10² | 17:1 | 100 |
| BHR-41 | ACRFSL | 8.4 × 10³ | 1.8 × 10³ | 4.6:1 | 268 | 1.4 × 10⁴ | 1.1 × 10³ | 12:1 | 13 | 1.0 × 10⁴ | 4.1 × 10² | 25:1 | 95 |
| BHR-42 | AERLTs | 6.2 × 10³ | 2.0 × 10² | 31:1 | 29 | 4.4 × 10² | 4.4 × 10² | 1:1 | 5 | 2.7 × 10⁴ | 3.8 × 10² | 72:1 | 88 |
| BHR-44 | ALQFTs | 2.2 × 10³ | NO | >1:500 | 331 | 6.4 × 10³ | 5.8 × 10² | 1:11 | 77 | 6.0 × 10³ | NO | >1:500* | 1393 |
| BHR-45 | ACQFNL | 1.8 × 10³ | 1.8 × 10³ | 1:1 | 268 | 1.3 × 10⁴ | 2.4 × 10² | 1:54* | 155 | 6.2 × 10³ | 6.7 × 10² | 1:9.3* | 1440 |
| BHR-53 | ACRLNL | 7.0 × 10⁴ | 2.2 × 10³ | 330:1 | 32 | 2.4 × 10⁴ | 3.0 × 10³ | 8:1 | 36 | 2.0 × 10⁵ | 3.6 × 10² | 550:1 | 84 |
| BHR-54 | ACQLTs | 2.0 × 10³ | 2.0 × 10³ | 1:1 | 301 | 5.0 × 10³ | 1.2 × 10³ | 4.1:1 | 60 | 2.2 × 10⁴ | 1.5 × 10³ | 17:1* | 342 |
| BHR-67 | iCQFNs | 5.4 × 10² | 5.4 × 10² | 1:1 | 80 | 9.2 × 10⁴ | 1.0 × 10⁴ | 1:8.9* | 1095 | 1.7 × 10³ | 1.7 × 10³ | 1:1 | 398 |
| BHR-69 | iCQFis | 3.2 × 10² | 3.2 × 10² | 1:1 | 46 | 1.4 × 10⁵ | 1.3 × 10⁴ | 1:11* | 1679 | 2.2 × 10³ | 2.2 × 10³ | 1:1 | 514 |
| BHR-70 | CVRLSL | 7.3 × 10⁴ | 1.4 × 10² | 520:1 | 21 | 1.8 × 10³ | 1.8 × 10² | 10:1 | 2 | 1.8 × 10⁴ | 2.2 × 10² | 81:1 | 51 |
| BHR-72 | ACGhNs | 2.0 × 10³ | 2.0 × 10³ | 1:1 | 290 | 9.9 × 10³ | 7.1 × 10² | 14:1 | 114 | 2.0 × 10⁴ | 8.0 × 10³ | 1:2.5* | 4558 |
| BHR-73 | AEGhNs | 9.8 × 10² | 9.8 × 10² | 1:1 | 144 | 2.4 × 10⁴ | 2.2 × 10³ | 1:11* | 292 | 4.0 × 10⁴ | 7.2 × 10³ | 1:5.5* | 9302 |
| BHR-74 | ACQFNs | 3.0 × 10³ | NO | >1:500 | 443 | 1.0 × 10⁴ | 1.6 × 10³ | 1:6.5* | 124 | 4.9 × 10³ | 1.1 × 10² | 1:44 | 1147 |
| BHR-75 | AEQFQs | 1.7 × 10³ | NO | >1:500 | 246 | 1.1 × 10⁴ | 7.0 × 10² | 1:16* | 131 | 1.3 × 10⁴ | 1.1 × 10² | 1:120* | 3093 |
| BHR-73- | AEGhNs- | 2.6 × 10³ | 2.6 × 10³ | 1:1 | 379 | 1.6 × 10⁴ | 1.9 × 10³ | 1:9 | 193 | 1.3 × 10⁴ | 3.0 × 10³ | 1:4.4 | 3047 |
| W | W | | | | | | | | | | | | |
| BHR-73- | AEGhNs- | 2.7 × 10³ | 2.7 × 10³ | 1:1 | 394 | 3.4 × 10⁴ | 3.1 × 10³ | 1:11 | 404 | 1.1 × 10⁴ | 2.9 × 10³ | 1:4 | 2558 |
| NW | NW | | | | | | | | | | | | |
| BHR-73- | AEGhNs- | 4.3 × 10³ | 4.3 × 10³ | 1:1 | 628 | 1.6 × 10⁴ | 2.0 × 10³ | 1:8* | 185 | 5.8 × 10⁴ | 1.1 × 10⁴ | 1:5* | 13465 |
| MNW | MNW | | | | | | | | | | | | |
| BHR-74- | ACQFNs- | 7.1 × 10³ | 1.2 × 10² | 1:59 | 1075 | 3.6 × 10⁴ | 4.2 × 10³ | 1:9 | 430 | 3.3 × 10³ | 1.3 × 10² | 1:26 | 767 |
| NW | NW | | | | | | | | | | | | |

^{a}Sequence given as single letter amino acid code for residues present at position 106/132/254/257/274/308.
For letters following the numerical designation
W = Y309W,
N = T173N,
M = C59M.
^{b}K₁ is for the faster phase and K₂ is for the slower phase from curve fits to 2-exponential.
^{c}Stereoselectivity was determined by LC/MS for variants marked with astrisk. For variants not explicitly tested, preference is assumed to be the same as OMVR.
nd = not determined.
NO = Not Observed.
Errors associated with kinetic measurements are reported in supplementary tables.

TABLE 20
Kinetic constants for purified PTE variants with OMVR.

| | | | | | | | R_{P}- |
|---|---|---|---|---|---|---|---|
| | | | | | | Fold | Fold |
| | | k_{cat}/Kₘ₁ | k_{cat}/Kₘ₂ | Ratio | Prefer- | Im- | Im- |
| Number | Sequence^{a} | (M⁻¹s⁻¹) | (M⁻¹s⁻¹) | S_{P}:R_{P} | ence | proved | proved |
| WT | ifhhis | 6.8 ± 0.1 × 10¹ | <6.8 × 10⁰ | >10:1 | S_{P} | na | na |
| BHR-1 | iCRhSs | 2.04 ± 0.01 × 10⁴ | 1.12 ± 0.01 × 10² | 160:1 | S_{P} | 300 | 16 |
| BHR-2 | iCRhTs | 1.32 ± 0.02 × 10⁴ | 8.6 ± 0.04 × 10¹ | 150:1 | S_{P} | 194 | 13 |
| BHR-3 | SCRhNL | 2.52 ± 0.04 × 10⁴ | 1.04 ± 0.01 × 10² | 240:1 | S_{P} | 371 | 15 |
| BHR-4 | iCQhiL | 3.26 ± 0.02 × 10⁴ | 5.8 ± 0.1 × 10¹ | 560:1 | S_{P} | 479 | 9 |
| BHR-5 | iCQhSL | 1.51 ± 0.01 × 10⁴ | 2.9. ± 0.02 × 10¹ | 510:1 | S_{P} | 222 | 4 |
| BHR-6 | sCQhNs | 1.26 ± 0.02 × 10³ | 1.5 ± 0.2 × 10² | 8.4:1 | S_{P} | 19 | 22 |
| BHR-7 | iCRhTL | 4.40 ± 0.02 × 10⁵ | NO | >500:1 | S_{P} | 6471 | na |
| BHR-8 | VCQhNL | 6.86 ± 0.02 × 10³ | 7.27 ± 0.07 × 10¹ | 95:1 | S_{P} | 101 | 11 |
| BHR-9 | CfRhQl | 8.74 ± 0.01 × 10³ | 1.06 ± 0.02 × 10¹ | 820:1 | S_{P} | 129 | 2 |
| BHR-10 | iChhNs | 8.6 ± 0.1 × 10² | 7.1 ± 0.2 × 10¹ | 13:1 | S_{P} | 13 | 10 |
| BHR-11 | CAQhQL | 6.53 ± 0.02 × 10⁴ | 3.10 ± 0.01 × 10¹ | 2100:1 | S_{P} | 960 | 5 |
| BHR-12 | ACQhTs | 9.6 ± 0.2 × 10² | 1.7 ± 0.2 × 10² | 5.5:1 | S_{P} | 14 | 25 |
| BHR-13 | iCGhNs | 8.2 ± 0.1 × 10³ | 3.7 ± 0.1 × 10² | 23:1 | S_{P} | 121 | 54 |
| BHR-14 | GCQhNL | 2.87 ± 0.04 × 10⁴ | 9.4 ± 0.1 × 10² | 31:1 | S_{P} | 422 | 138 |
| BHR-15 | AARhQL | 1.73 ± 0.06 × 10⁴ | 1.44 ± 0.02 × 10² | 120:1 | S_{P} | 254 | 21 |
| BHR-16 | VCRhNL | 1.07 ± 0.01 × 10⁵ | 5.99 ± 0.03 × 10¹ | 1800:1 | S_{P} | 1574 | 9 |
| BHR-17 | ACRhiL | 5.1 ± 0.1 × 10⁴ | 1.04 ± 0.07 × 10³ | 49:1 | S_{P} | 750 | 153 |
| BHR-18 | CLRhQL | 4.31 ± 0.01 × 10⁴ | 2.94 ± 0.02 × 10¹ | 1500:1 | S_{P} | 634 | 4 |
| BHR-19 | iCRhQL | 1.64 ± 0.02 × 10⁵ | NO | >500:1 | S_{P} | 2412 | na |
| BHR-20 | iCRhQs | 1.35 ± 0.02 × 10⁴ | 1.03 ± 0.01 × 10² | 130:1 | S_{P} | 199 | 15 |
| BHR-21 | VCRhQL | 6.74 ± 0.06 × 10⁴ | 6.23 ± 0.02 × 10¹ | 1100:1 | S_{P} | 991 | 9 |
| BHR-22 | VCRhTL | 2.95 ± 0.07 × 10⁴ | 9.40 ± 0.06 × 10² | 31:1 | S_{P} | 434 | 138 |
| BHR-23 | iVRhSL | 5.80 ± 0.04 × 10⁴ | NO | >500:1 | S_{P} | 853 | na |
| BHR-24 | CLRhSL | 3.26 ± 0.01 × 10⁴ | NO | >500:1 | S_{P} | 479 | na |
| BHR-25 | CARhNL | 5.10 ± 0.02 × 10⁴ | NO | >500:1 | S_{P} | 750 | na |
| BHR-26 | iCQhis | 1.77 ± 0.04 × 10³ | 3.8 ± 0.2 × 10² | 4.7:1 | S_{P} | 26 | 56 |
| BHR-27 | CLGhTs | 3.78 ± 0.03 × 10³ | 2.42 ± 0.01 × 10² | 16:1 | S_{P} | 56 | 36 |
| BHR-28 | CVQhNL | 4.80 ± 0.01 × 10⁴ | 2.47 ± 0.03 × 10¹ | 1900:1 | S_{P} | 706 | 4 |
| BHR-29 | CDQhQL | 7.25 ± 0.05 × 10⁴ | 6.76 ± 0.01 × 10¹ | 1100:1 | S_{P} | 1066 | 10 |
| BHR-30 | iCGhTs | 7.10 ± 0.01 × 10³ | 4.1 ± 0.1 × 10² | 17:1 | S_{P} | 104 | 60 |
| BHR-31 | iCQhTL | 1.25 ± 0.01 × 10⁴ | 5.48 ± 0.06 × 10¹ | 230:1 | S_{P} | | |
| BHR-32 | AVShQs | 2.77 ± 0.02 × 10² | 2.77 ± 0.02 × 10² | 1:1 | na | 4 | 41 |
| BHR-33 | VfRhTL | 6.17 ± 0.04 × 10³ | NO | >500:1 | S_{P} | 91 | na |
| BHR-34 | iLGLNs | 8.07 ± 0.02 × 10² | NO | >500:1 | S_{P} | 12 | na |
| BHR-35 | ACRhNs | 9.2 ± 0.6 × 10² | 3.7 ± 0.1 × 10² | 2.5:1 | S_{P} | 14 | 54 |
| BHR-36 | AVQhQs | 2.98 ± 0.01 × 10² | NO | >500:1 | S_{P} | 4 | na |
| BHR-37 | iLGLTs | 2.06 ± 0.01 × 10³ | 1.23 ± 0.01 × 10² | 16.7:1 | S_{P} | 30 | 18 |
| BHR-38 | ACQLiL | 2.25 ± 0.01 × 10³ | 1.04 ± 0.01 × 10³ | 1:2.2 | R_{P} | 33 | 331 |
| WT | ifhhis | 6.8 ± 0.1 × 10¹ | <6.8 × 10⁰ | >10:1 | S_{P} | na | na |
| BHR-39 | ACQFQL | 2.21 ± 0.05 × 10³ | 9.6 ± 0.3 × 10² | 1:2.3 | R_{P} | 33 | 325 |
| BHR-40 | ACRFQL | 4.6 ± 0.1 × 10³ | 1.15 ± 0.05 × 10³ | 4:1 | S_{P} | 68 | 169 |
| BHR-41 | ACRFSL | 8.4 ± 0.2 × 10³ | 1.82 ± 0.04 × 10³ | 4.6:1 | S_{P} | 124 | 268 |
| BHR-42 | AERLTs | 6.22 ± 0.08 × 10³ | 1.98 ± 0.01 × 10² | 31:1 | S_{P} | 91 | 29 |
| BHR-43 | AVQliNs | 3.46 ± 0.01 × 10² | 3.46 ± 0.01 × 10² | 1:1 | na | 5 | 51 |
| BHR-44 | ALQFTs | 2.25 ± 0.01 × 10³ | NO | >1:500 | R_{P} | 33 | 331 |
| BHR-45 | ACQFNL | 1.82 ± 0.05 × 10³ | 1.82 ± 0.05 × 10³ | 1:1 | na | 27 | 268 |
| BHR-46 | SERLTL | 5.53 ± 0.08 × 10⁴ | NO | >500:1 | S_{P} | 813 | na |
| BHR-47 | GEQLQs | 4.33 ± 0.01 × 10³ | NO | >500:1 | S_{P} | 64 | na |
| BHR-48 | LWQLTS | 8.34 ± 0.02 × 10³ | NO | >500:1 | S_{P} | 123 | na |
| BHR-49 | GCQhiL | 3.46 ± 0.09 × 10⁴ | 8.98 ± 0.07 × 10² | 39:1 | S_{P} | 509 | 132 |
| BHR-50 | iVQFNI | 1.80 ± 0.01 × 10² | 1.80 ± 0.01 × 10² | 1:1 | na | 3 | 26 |
| BHR-51 | iCRLNI | 1.48 ± 0.01 × 10⁴ | NO | >500:1 | S_{P} | 218 | na |
| BHR-52 | ACRhTs | 1.58 ± 0.01 × 10³ | 1.58 ± 0.01 × 10³ | 1:1 | na | 23 | 232 |
| BHR-53 | ACRLNL | 7.02 ± 0.08 × 10⁴ | 2.15 ± 0.01 × 10² | 330:1 | S_{P} | 1032 | 32 |
| BHR-54 | ACQLTs | 2.05 ± 0.01 × 10³ | 2.05 ± 0.01 × 10³ | 1:1 | na | 30 | 301 |
| BHR-55 | iLGFQs | 1.18 ± 0.01 × 10² | 1.18 ± 0.01 × 10² | 1:1 | na | 2 | 17 |
| BHR-56 | iLGFSs | 1.32 ± 0.01 × 102 | 1.32 ± 0.01 × 10² | 1:1 | na | 2 | 19 |
| BHR-57 | ACQHSs | 7.72 ± 0.03 × 102 | 7.72 ± 0.03 × 10² | 1:1 | na | 11 | 114 |
| BHR-58 | ALGFQS | 1.33 ± 0.01 × 10³ | 5.56 ± 0.06 × 10¹ | 1:24 | R_{P} | 20 | 196 |
| BHR-59 | ALGFNS | 1.26 ± 0.01 × 10³ | 6.99 ± 0.06 × 101 | 1:18 | R_{P} | 19 | 185 |
| BHR-60 | AVGFNS | 7.78 ± 0.07 × 10² | 7.29 ± 0.07 × 101 | 1:11 | R_{P} | 11 | 114 |
| BHR-61 | ALRLQV | 3.17 ± 0.02 × 10³ | 1.52 ± 0.00 × 10\hat{ } | 21:1 | S_{P} | 47 | 22 |
| BHR-62 | ACQLNs | 1.92 ± 0.01 × 10³ | 4.60 ± 0.02 × 10² | 4.2:1 | S_{P} | 28 | 68 |
| BHR-63 | VLGFQs | 3.44 ± 0.06 × 10² | 6.26 ± 0.06 × 10¹ | 1:5.5 | R_{P} | 5 | 51 |
| BHR-64 | AERLNL | 6.76 ± 0.04 × 10⁴ | 1.15 ± 0.01 × 10² | 590:1 | S_{P} | 994 | 17 |
| BHR-65 | AERLQs | 1.57 ± 0.01 × 10⁴ | 5.67 ± 0.02 × 10² | 28:1 | S_{P} | 231 | 83 |
| BHR-66 | WRLSL | 8.03 ± 0.05 × 10⁴ | NO | >500:1 | S_{P} | 1181 | na |
| BHR-67 | iCQFNs | 5.41 ± 0.01 × 10² | 5.41 ± 0.01 × 10² | 1:1 | na | 8 | 80 |
| BHR-68 | CARLTL | 3.44 ± 0.04 × 10⁴ | NO | >500:1 | S_{P} | 506 | na |
| BHR-69 | iCQFis | 3.15 ± 0.01 × 10² | 3.15 ± 0.01 × 10² | 1:1 | na | 5 | 46 |
| BHR-70 | CVRLSL | 7.31 ± 0.03 × 10⁴ | 1.40 ± 0.01 × 10² | 520:1 | S_{P} | 1075 | 21 |
| BHR-71 | AVQFNs | 1.53 ± 0.01 × 10³ | NO | >1:500 | R_{P} | 23 | 225 |
| BHR-72 | ACGhNs | 1.97 ± 0.01 × 10³ | 1.97 ± 0.01 × 10³ | 1:1 | na | 29 | 290 |
| BHR-73 | AEGhNs | 9.79 ± 0.04 × 10² | 9.79 ± 0.04 × 10² | 1:1 | na | 14 | 144 |
| BHR-74 | ACQFNs | 3.01 ± 0.02 × 10³ | NO | >1:500 | R_{P} | 44 | 443 |
| BHR-75 | AEQFQs | 1.67 ± 0.01 × 10³ | NO | >1:500 | R_{P} | 25 | 246 |
| BHR-76 | AEQYQs | 1.73 ± 0.01 × 10³ | NO | >1:500 | R_{P} | 25 | 254 |

^{a}Sequence given as single letter amino acid code for residues present at position 106/132/254/257/274/308.
nd = not determined.

TABLE 21
Kinetic constants for purified PTE variants with DEVX.

| | | | | | Fold | | | | | | Fold |
|---|---|---|---|---|---|---|---|---|---|---|---|
| | | k_{cat} | Kₘ | k_{cat}/Kₘ | im- | | | k_{cat} | Kₘ | k_{cat}/Kₘ | im- |
| Variant | Sequence^{a} | (s⁻¹) | (mM) | (M⁻¹s⁻¹) | proved | Number | Sequence^{a} | (s⁻¹) | (mM) | (M⁻¹s⁻¹) | proved |
| WT | ifhhis | 1.08 ± 0.02 | 0.87 ± 0.04 | 1.20 ± 0.06 × 10³ | na | WT | ifhhis | 1.08 ± 0.02 | 0.87 ± 0.04 | 1.20 ± 0.06 × 10³ | na |
| BHR-1 | iCRhSs | 48 ± 3 | 0.9 ± 0.1 | 5.3 ± 0.7 × 10⁴ | 44 | BHR-39 | ACQFQL | 75 ± 2 | 0.35 ± 0.02 | 2.2 ± 0.1 × 10⁵ | 180 |
| BHR-2 | iCRhTs | 62 ± 4 | 0.9 ± 0.1 | 6.9 ± 0.7 × 10⁴ | 58 | BHR-40 | ACRFQL | 99 ± 4 | 0.23 ± 0.02 | 4.2 ± 0.4 × 10⁵ | 350 |
| BHR-3 | SCRhNL | 54 ± 2 | 0.63 ± 0.05 | 8.6 ± 0.7 × 10⁴ | 72 | BHR-41 | ACRFSL | 105 ± 2 | 0.22 ± 0.01 | 4.8 ± 0.3 × 10⁵ | 400 |
| BHR-4 | iCQhiL | 78 ± 4 | 0.60 ± 0.06 | 1.3 ± 0.1 × 10⁵ | 110 | BHR-42 | AERLTs | 40 ± 5 | 3.0 ± 0.5 | 1.26 ± 0.01 × 10⁴ | 11 |
| BHR-5 | iCQhSL | 75 ± 4 | 0.63 ± 0.06 | 1.2 ± 0.1 × 10⁵ | 100 | BHR-43 | AVQhNs | nd | nd | 6.56 ± 0.05 × 10⁴ | 55 |
| BHR-6 | sCQhNs | 65 ± 3 | 0.77 ± 0.07 | 8.4 ± 0.8 × 10⁴ | 70 | BHR-44 | ALQFTs | 8 ± 2 | 2.2 ± 0.7 | 2.85 ± 0.06 × 10³ | 2 |
| BHR-7 | iCRhTL | 45 ± 1 | 0.29 ± 0.02 | 1.6 ± 0.1 × 10⁵ | 130 | BHR-45 | ACQFNL | 52.1 ± 0.8 | 0.25 ± 0.01 | 2.13 ± 0.08 × 10⁵ | 180 |
| BHR-8 | VCQhNL | 40 ± 3 | 1.1 ± 0.1 | 3.6 ± 0.4 × 10⁴ | 30 | BHR-46 | SERLTL | 33 ± 3 | 2.2 ± 10.3 | 1.54 ± 0.02 × 10⁴ | 13 |
| BHR-9 | CfRhQl | 7 ± 0.6 | 1.4 ± 0.2 | 5.0 ± 0.8 × 10³ | 4 | BHR-47 | GEQLQs | 14 ± 1 | 2.1 ± 0.2 | 6.7 ± 0.9 × 10³ | 6 |
| BHR-10 | iChhNs | 13 ± 1 | 0.73 ± 0.07 | 1.8 ± 0.2 × 10⁴ | 15 | BHR-48 | LWQLTS | 20 ± 2 | 4.0 ± 0.4 | 5.1 ± 0.7 × 10³ | 4 |
| BHR-11 | CAQhQL | 43 ± 3 | 0.8 ± 0.1 | 5.4 ± 0.8 × 10⁴ | 45 | BHR-49 | GCQhiL | 35 ± 1 | 0.40 ± 0.01 | 8.4 ± 0.5 × 10⁴ | 70 |
| BHR-12 | ACQhTs | 5.5 ± 0.4 | 1.0 ± 0.1 | 5.5 ± 0.7 × 10³ | 5 | BHR-50 | iVQFNI | 26 ± 1 | 0.77 ± 0.04 | 3.4 ± 0.2 × 10⁴ | 28 |
| BHR-13 | iCGhNs | 21 ± 1 | 0.76 ± 0.08 | 2.8 ± 0.3 × 10⁴ | 23 | BHR-51 | iCRLNI | 10 ± 2 | 1.5 ± 0.4 | 5.5 ± 0.1 × 10³ | 5 |
| BHR-14 | GCQhNL | 97 ± 7 | 1.5 ± 0.2 | 6.5 ± 0.8 × 10⁴ | 54 | BHR-52 | ACRhTs | 19 ± 2 | 0.9 ± 0.1 | 2.1 ± 0.3 × 10⁴ | 18 |
| BHR-15 | AARhQL | 75 ± 4 | 1.0 ± 0.1 | 7.5 ± 0.6 × 10⁴ | 63 | BHR-53 | ACRLNL | 69 ± 2 | 0.25 ± 0.01 | 2.7 ± 0.2 × 10⁵ | 230 |
| BHR-16 | VCRhNL | 32 ± 1 | 0.41 ± 0.05 | 8 ± 1 × 10⁴ | 64 | BHR-54 | ACQLTs | 0.208 ± 0.004 | 0.42 ± 0.01 | 4.9 ± 0.2 × 10² | 0 |
| BHR-17 | ACRhiL | 80 ± 4 | 0.43 ± 0.04 | 1.9 ± 0.2 × 10⁵ | 160 | BHR-55 | iLGFQs | 2.5 ± 0.3 | 5.0 ± 0.6 | 4.49 ± 0.03 × 10² | 0 |
| BHR-18 | CLRhQL | 28 ± 2 | 1.0 ± 0.1 | 2.8 ± 0.3 × 10⁴ | 23 | BHR-56 | iLGFSs | 16 ± 1 | 2.7 ± 0.3 | 5.48 ± 0.06 × 10³ | 5 |
| BHR-19 | iCRhQL | 65 ± 2 | 0.32 ± 0.02 | 2.0 ± 0.1 × 10⁵ | 170 | BHR-57 | ACQHSs | 10.4 ± 0.5 | 0.88 ± 0.06 | 1.2 ± 0.1 × 10⁴ | 10 |
| BHR-20 | iCRhQs | 60 ± 3 | 0.92 ± 0.07 | 6.5 ± 0.6 × 10⁴ | 54 | BHR-58 | ALGFQS | 14 ± 4 | 6 ± 2 | 2.17 ± 0.02 × 10³ | 2 |
| BHR-21 | VCRhQL | 39 ± 2 | 0.42 ± 0.04 | 9 ± 1 × 10⁴ | 78 | BHR-59 | ALGFNS | 5 ± 1 | 2.6 ± 0.6 | 2.10 ± 0.03 × 10³ | 2 |
| BHR-22 | VCRhTL | 46 ± 3 | 0.79 ± 0.09 | 5.8 ± 0.7 × 10⁴ | 48 | BHR-60 | AVGFNS | nd | nd | 2.62 ± 0.05 × 10³ | 2 |
| BHR-23 | iVRhSL | 19.3 ± 0.9 | 0.48 ± 0.05 | 4.0 ± 0.4 × 10⁴ | 33 | BHR-61 | ALRLQV | 15 ± 3 | 2.4 ± 0.6 | 5.9 ± 0.2 × 10³ | 5 |
| BHR-24 | CLRhSL | 18 ± 1 | 1.31 ± 0.1 | 1.4 ± 0.2 × 10⁴ | 12 | BHR-62 | ACQLNs | 13.4 ± 0.6 | 1.18 ± 0.07 | 1.14 ± 0.08 × 10⁴ | 10 |
| BHR-25 | CARhNL | 28 ± 2 | 0.87 ± 0.08 | 3.2 ± 0.4 × 10⁴ | 27 | BHR-63 | VLGFQs | 23 ± 8 | 5 ± 2 | 4.5 ± 0.1 × 10³ | 4 |
| BHR-26 | iCQhis | 40 ± 2 | 0.91 ± 0.08 | 4.4 ± 0.4 × 10⁴ | 37 | BHR-64 | AERLNL | 110 ± 10 | 1.5 ± 0.2 | 6.61 ± 0.06 × 10⁴ | 58 |
| BHR-27 | CLGhTs | nd | nd | 1.00 ± 0.03 × 10⁴ | 8 | BHR-65 | AERLQs | 24 ± 4 | 0.7 ± 0.1 | 3.39 ± 0.8 × 10⁴ | 28 |
| BHR-28 | CVQhNL | 31 ± 2 | 0.73 ± 0.06 | 4.2 ± 0.4 × 10⁴ | 35 | BHR-66 | VVRLSL | 25 ± 1 | 0.39 ± 0.03 | 6.4 ± 0.6 × 10⁴ | 53 |
| BHR-29 | CDQhQL | 31.3 ± 0.2 | 0.8 ± 0.1 | 3.9 ± 0.4 × 10⁴ | 33 | BHR-67 | iCQFNs | 28.6 ± 0.3 | 0.279 ± 0.007 | 1.03 ± 0.03 × 10⁵ | 86 |
| BHR-30 | iCGhTs | 28 ± 0.1 | 0.60 ± 0.06 | 4.7 ± 0.5 × 10⁴ | 39 | BHR-68 | CARLTL | 11 ± 0.5 | 1.11 ± 0.07 | 9.9 ± 0.8 × 10³ | 8 |
| BHR-31 | iCQhTL | 81 ± 4 | 0.57 ± 0.05 | 1.4 ± 0.1 × 10⁵ | 120 | BHR-69 | iCQFis | 10.9 ± 10.3 | 0.27 ± 0.02 | 4.0 ± 0.3 × 10⁴ | 33 |
| BHR-32 | AVShQs | 4.6 ± 0.4 | 3.0 ± 0.3 | 1.5 ± 0.2 × 10³ | 1 | BHR-70 | CVRLSL | 54 ± 2 | 1.1 ± 0.06 | 4.9 ± 0.3 × 10⁴ | 41 |
| BHR-33 | VfRhTL | 4.8 ± 0.3 | 1.6 ± 0.1 | 3.1 ± 0.3 × 10³ | 3 | BHR-71 | AVQFNs | 11 ± 1 | 2.6 ± 0.3 | 3.81 ± 0.04 × 10³ | 3 |
| BHR-34 | iLGLNs | 25 ± 15 | 11 ± 7 | 2.10 ± 0.02 × 10³ | 2 | BHR-72 | ACGhNs | 8 ± 0.5 | 1.17 ± 0.09 | 6.8 ± 0.7 × 10³ | 6 |
| BHR-35 | ACRhNs | 14.4 ± 0.8 | 1.6 ± 0.1 | 9.0 ± 0.8 × 10³ | 8 | BHR-73 | AEGhNs | 2.9 ± 0.4 | 2.5 ± 0.3 | 1.2 ± 0.2 × 10³ | 1 |
| BHR-36 | AVQhQs | 60 ± 20 | 18 ± 6 | 3.04 ± 0.01 × 10³ | 3 | BHR-74 | ACQFNs | 20 ± 2 | 0.9 ± 0.1 | 2.2 ± 0.3 × 10⁴ | 18 |
| BHR-37 | iLGLTs | 22 ± 6 | 6 ± 2 | 3.43 ± 0.04 × 10³ | 3 | BHR-75 | AEQFQs | 16 ± 5 | 4 ± 1 | 3.55 ± 0.03 × 10³ | 3 |
| BHR-38 | ACQLiL | 52 ± 1 | 0.34 ± 0.01 | 1.53 ± 0.07 × 10⁵ | 130 | BHR-76 | AEQVQs | 11 ± 4 | 8 ± 3 | 1.21 ± 0.01 × 10³ | 1 |

^{a}Sequence given as single letter amino acid code for residues present at position 106/132/254/257/274/308.
nd = not determined.

### Example 53

Selection, Purification and Characterization of Additional Variants. Given the severe stereoselectivity observed with the initial variants, an additional 39 variants (BHR-34-71) were selected based on broad spectrum activity and a demonstrated ability to hydrolyze R_{P}-OMVR (FIG. 23F). These variants were successfully purified and characterized with the screening analogs (Tables 20-24). An additional 6 variants demonstrated more than 100-fold improvements against DEVX (FIG. 23D), with the best variant being BHR-41 which has a k_{cat} of 105 s⁻¹, a Kₘ of 220 μM and k_{cat}/Kₘ of 4.8×10⁵M⁻¹s⁻¹, which is 400-fold improved over wild-type PTE (Table 17). An additional five variants were found to be more than 300-fold improved for hydrolysis of DMVX (FIG. 23E), with the best being BHR-45, which has an enzymatic efficiency improved over 450-fold to 8.7×10³M⁻¹s⁻¹. The majority of variants did not show substantial improvements with malathion (FIG. 23G), but four variants were more than 200-fold improved with the best being BHR-70 with a k_{cat}/Kₘ of 1.7×10⁴ M⁻¹ s⁻¹, which is 567-fold better than wild-type.

TABLE 22
Kinetic constants for purified PTE variants with DMVX.

| Number | Sequence^{a} | k_{cat} (s⁻¹) | Kₘ (mM) | k_{cat}/Kₘ (M⁻¹s⁻¹) | Fold improved |
|---|---|---|---|---|---|
| WT | ifhhis | 0.021 ± 0.004 | 1.1 ± 0.3 | 1.9 ± 0.7 × 10¹ | na |
| BHR-1 | iCRhSs | 0.73 ± 0.03 | 0.96 ± 0.01 | 7.6 ± 0.3 × 10² | 40 |
| BHR-2 | iCRhTs | 1.4 ± 0.1 | 1.5 ± 0.1 | 9.3 ± 0.9 × 10² | 49 |
| BHR-3 | SCRliNL | 0.97 ± 0.05 | 0.69 ± 0.06 | 1.4 ± 0.2 × 10³ | 74 |
| BHR-4 | iCQhiL | 3.9 ± 0.2 | 0.43 ± 0.04 | 9.1 ± 0.1 × 10³ | 480 |
| BHR-5 | iCQhSL | 3.2 ± 0.2 | 0.53 ± 0.05 | 6.0 ± 0.7 × 10³ | 320 |
| BHR-6 | sCQhNs | 2 ± 0.1 | 0.85 ± 0.07 | 2.4 ± 0.1 × 10³ | 130 |
| BHR-7 | iCRhTL | 9.1 ± 0.4 | 1.3 ± 0.1 | 7.0 ± 0.6 × 10³ | 370 |
| BHR-8 | VCQhNL | 0.93 ± 0.04 | 0.95 ± 0.05 | 9.8 ± 0.7 × 10² | 52 |
| BHR-9 | CfRhQl | 0.93 ± 0.07 | 1.3 ± 0.16 | 7 ± 1 × 10² | 37 |
| BHR-10 | iChhNs | 0.075 ± 0.004 | 0.48 ± 0.05 | 1.6 ± 0.2 × 10² | 8 |
| BHR-11 | CAQhQL | nd | nd | 5.0 ± 0.2 × 10² | 26 |
| BHR-12 | ACQhTs | 0.07 ± 0.004 | 1.4 ± 0.1 | 5.0 ± 0.5 × 10¹ | 3 |
| BHR-13 | iCGhNs | 0.25 ± 0.002 | 0.33 ± 0.05 | 8 ± 1 × 10² | 42 |
| BHR-14 | GCQhNL | 1.6 ± 0.1 | 1.5 ± 0.1 | 1.1 ± 0.1 × 10³ | 58 |
| BHR-15 | AARhQL | 1.6 ± 0.1 | 2.4 ± 0.3 | 6.7 ± 0.9 × 10² | 35 |
| BHR-16 | VCRhNL | 3.9 ± 0.2 | 1.2 ± 0.1 | 3.3 ± 0.3 × 10³ | 170 |
| BHR-17 | ACRhiL | 1.6 ± 0.06 | 1.2 ± 0.1 | 1.3 ± 0.1 × 10³ | 68 |
| BHR-18 | CLRhQL | 1.6 ± 0.1 | 2.5 ± 0.3 | 6.4 ± 0.9 × 10² | 34 |
| BHR-19 | iCRhQL | 10.5 ± 0.5 | 1.3 ± 0.1 | 8.1 ± 0.7 × 10³ | 430 |
| BHR-20 | iCRhQs | 1.9 ± 0.1 | 2.1 ± 0.2 | 9.0 ± 0.1 × 10² | 47 |
| BHR-21 | VCRhQL | 4.4 ± 0.2 | 1.2 ± 0.1 | 3.7 ± 0.4 × 10³ | 200 |
| BHR-22 | VCRhTL | 1.5 ± 0.1 | 1.6 ± 0.2 | 9 ± 1 × 10² | 49 |
| BHR-23 | iVRhSL | 2.9 ± 0.2 | 2.5 ± 0.3 | 1.2 ± 0.2 × 10³ | 63 |
| BHR-24 | CLRhSL | 1 ± 0.1 | 2.4 ± 0.2 | 4.2 ± 0.5 × 10² | 22 |
| BHR-25 | CARhNL | 2.2 ± 0.2 | 1.9 ± 0.2 | 1.2 ± 0.2 × 10³ | 63 |
| BHR-26 | iCQhis | 1 ± 0.04 | 0.52 ± 0.04 | 1.9 ± 0.2 × 10³ | 100 |
| BHR-27 | CLGhTs | nd | nd | 9.2 ± 0.2 × 10¹ | 5 |
| BHR-28 | CVQhNL | 2.3 ± 0.1 | 2.5 ± 0.2 | 9.2 ± 0.8 × 10² | 48 |
| BHR-29 | CDQhQL | nd | nd | 5.2 ± 0.2 × 10² | 27 |
| BHR-30 | iCGhTs | 1.7 ± 0.1 | 1.4 ± 0.1 | 1.2 ± 0.1 × 10³ | 63 |
| BHR-31 | iCQhTL | 5.9 ± 0.2 | 0.7 ± 0.05 | 8.4 ± 0.7 × 10³ | 440 |
| BHR-32 | AVShQs | 1 ± 0.25 | 6 ± 3 | 1.43 ± 0.02 × 10² | 8 |
| BHR-33 | VfRhTL | 0.12 ± 0.01 | 1.0 ± 0.1 | 1.2 ± 0.2 × 10² | 6 |
| BHR-34 | iLGLNs | nd | nd | 1.4 ± 0.1 × 10² | 7 |
| WT | ifhhis | 0.021 ± 0.004 | 1.1 ± 0.3 | 1.9 ± 0.7 × 10¹ | na |
| BHR-39 | ACQFQL | 5.9 ± 0.3 | 1.0 ± 0.1 | 5.9 ± 0.5 × 10³ | 310 |
| BHR-40 | ACRFQL | 6.3 ± 0.2 | 0.99 ± 0.04 | 6.3 ± 0.3 × 10³ | 330 |
| BHR-41 | ACRFSL | 4.7 ± 0.1 | 0.74 ± 0.03 | 6.3 ± 0.3 × 10³ | 330 |
| BHR-42 | AERLTs | nd | nd | 4.8 ± 0.1 × 10¹ | 3 |
| BHR-43 | AVQhNs | nd | nd | 1.20 ± 0.01 × 10² | 6 |
| BHR-44 | ALQFTs | nd | nd | 1.90 ± 0.02 × 10² | 10 |
| BHR-45 | ACQFKL | 6.2 ± 0.3 | 0.71 ± 0.06 | 8.7 ± 0.8 × 10³ | 460 |
| BHR-46 | SERLTL | nd | nd | 4.20 ± 0.05 × 10¹ | 2 |
| BHR-47 | GEQLQs | nd | nd | 4.10 ± 0.07 × 10¹ | 2 |
| BHR-48 | LWQLTS | nd | nd | 4.60 ± 0.05 × 10¹ | 2 |
| BHR-49 | GCQhiL | 0.76 ± 0.04 | 0.81 ± 0.06 | 9.50 ± 0.8 × 10² | 50 |
| BHR-50 | iVQFNI | 7 ± 1 | 6 ± 1 | 1.3 ± 0.3 × 10³ | 68 |
| BHR-51 | iCRLNI | 0.77 ± 0.06 | 2.6 ± 0.3 | 3.0 ± 0.4 × 10² | 16 |
| BHR-52 | ACRhTs | 0.44 ± 0.04 | 3.5 ± 0.4 | 1.3 ± 0.2 × 10² | 7 |
| BHR-53 | ACRLNL | 2.3 ± 0.2 | 2.0 ± 0.3 | 1.1 ± 0.2 × 10³ | 58 |
| BHR-54 | ACQLTs | 0.208 ± 0.004 | 0.44 ± 0.01 | 4.7 ± 0.1 × 10² | 25 |
| BHR-55 | iLGFQs | 2.5 ± 0.3 | 5.2 ± 0.6 | 4.8 ± 0.8 × 10² | 25 |
| BHR-56 | iLGFSs | nd | nd | 3.44 ± 0.02 × 10² | 18 |
| BHR-57 | ACQHSs | 0.09 ± 0.003 | 0.24 ± 0.02 | 3.8 ± 0.3 × 10² | 20 |
| BHR-58 | ALGFQS | 4 ± 2 | 30 ± 10 | 1.43 ± 0.01 × 10² | 8 |
| BHR-59 | ALGFNS | 1.1 ± 0.3 | 9 ± 3 | 1.10 ± 0.01 × 10² | 6 |
| BHR-60 | AVGFNS | nd | nd | 1.38 ± 0.01 × 10² | 7 |
| BHR-61 | ALRLQV | nd | nd | 1.25 ± 0.08 × 10¹ | 1 |
| BHR-62 | ACQLNs | 0.088 ± 0.005 | 0.33 ± 0.04 | 2.7 ± 0.4 × 10² | 14 |
| BHR-63 | VLGFQs | 2 ± 1 | 8 ± 4 | 2.66 ± 0.04 × 10² | 14 |
| BHR-64 | AERLNL | 0.09 ± 0.01 | 0.8 ± 0.2 | 1.1 ± 0.3 × 10² | 6 |
| BHR-65 | AERLQs | 0.11 ± 0.02 | 1.3 ± 0.3 | 8.4 ± 0.2 × 10¹ | 5 |
| BHR-66 | VVRLSL | 1.4 ± 0.2 | 1.0 ± 0.2 | 1.4 ± 0.3 × 10³ | 74 |
| BHR-67 | iCQFNs | 2.99 ± 0.03 | 0.523 ± 0.009 | 5.7 ± 0.1 × 10³ | 300 |
| BHR-68 | CARLTL | 0.5 ± 0.1 | 3.1 ± 0.7 | 1.6 ± 0.5 × 10² | 8 |
| BHR-69 | iCQFis | 0.85 ± 0.03 | 0.32 ± 0.02 | 2.7 ± 0.2 × 10³ | 140 |
| BHR-70 | CVRLSL | 0.4 ± 0.1 | 3 ± 1 | 1.45 ± 0.02 × 10² | 8 |
| BHR-71 | AVQFNs | 1.1 ± 0.2 | 2.2 ± 0.4 | 4.45 ± 0.03 × 10² | 26 |
| BHR-72 | ACGhNs | 0.1 ± 0.01 | 0.30 ± 0.05 | 3.3 ± 0.6 × 10² | 17 |

TABLE 23
Kinetic constants for purified PTE variants with malathion.

| | | k_{cat} | Kₘ | k_{cat}/Kₘ | Fold |
|---|---|---|---|---|---|
| Number | Sequence^{a} | (s⁻¹) | (mM) | (M⁻¹s⁻¹) | improved |
| WT | [symbol] | 0.047 ± 0.007 | 1.0 ± 0.2 | 3.0 ± 0.6 × 10¹ | na |
| BHR-1 | [symbol] | nd | nd | [symbol] | [symbol] |
| BHR-2 | [symbol] | nd | nd | [symbol] | [symbol] |
| BHR-3 | [symbol] | nd | nd | [symbol] | [symbol] |
| BHR-4 | [symbol] | nd | nd | 5.5 ± 0.1 × 10¹ | 2 |
| BHR-5 | [symbol] | nd | nd | [symbol] | 2 |
| BHR-6 | [symbol] | nd | nd | 7.0 ± 0.1 × 10⁰ | 0 |
| BHR-7 | [symbol] | nd | nd | [symbol] | [symbol] |
| BHR-8 | [symbol] | nd | nd | [symbol] | [symbol] |
| BHR-9 | [symbol] | nd | nd | [symbol] | [symbol] |
| BHR-10 | iChhNs | nd | nd | 1.10 ± 0.02 × 10⁰ | 0 |
| BHR-11 | CAQhQL | nd | nd | 2.7 ± 0.1 × 10³ | 90 |
| BHR-12 | ACQhTs | nd | nd | 4.8 ± 0.1 × 10¹ | 2 |
| BHR-13 | iCGhNs | nd | nd | 8.7 ± 0.1 × 10⁰ | 0 |
| BHR-14 | GCQhNL | nd | nd | 9.3 ± 0.2 × 10² | 31 |
| BHR-15 | AARhQL | nd | nd | 7.1 ± 0.2 × 10² | 24 |
| BHR-16 | VCRhNL | nd | nd | 1.40 ± 0.3 × 10³ | 47 |
| BHR-17 | ACRhiL | 1.20 ± 0.07 | 1.4 ± 0.1 | 8.6 ± 0.8 × 10² | 29 |
| BHR-18 | CLRhQL | 3.2 ± 0.2 | 1.8 ± 0.2 | 1.8 ± 0.2 × 10³ | 60 |
| BHR-19 | iCRhQL | 3.1 ± 0.2 | 1.9 ± 0.2 | 1.6 ± 0.2 × 10³ | 53 |
| BHR-20 | iCRhQs | nd | nd | 6.0 ± 0.2 × 10¹ | 2 |
| BHR-21 | VCRhQL | nd | nd | 1.00 ± 0.02 × 10³ | 33 |
| BHR-22 | VCRhTL | 1.4 ± 0.1 | 1.5 ± 0.2 | 9 ± 1 × 10² | 29 |
| BHR-23 | iVRhSL | 1.7 ± 0.2 | 1.7 ± 0.1 | 1.00 ± 0.08 × 10³ | 33 |
| BHR-24 | CLRhSL | 2.5 ± 0.2 | 3.6 ± 0.4 | 6.9 ± 0.9 × 10² | 23 |
| BHR-25 | CARhNL | 2.4 ± 0.2 | 1.9 ± 0.2 | 1.3 ± 0.2 × 10³ | 43 |
| BHR-26 | iCQhis | 0.010 ± 0.001 | 2.8 ± 0.3 | 3.6 ± 0.5 × 10⁰ | 0 |
| BHR-27 | CLGhTs | nd | nd | 4.2 ± 0.1 × 10⁰ | 0 |
| BHR-28 | CVQhNL | nd | nd | 1.30 ± 0.04 × 10³ | 43 |
| BHR-29 | CDQhQL | 1.10 ± 0.05 | 1.00 ± 0.07 | 1.10 ± 0.08 × 10³ | 37 |
| BHR-30 | iCGhTs | 0.010 ± 0.0004 | 1.10 ± 0.07 | 9.0 ± 0.7 × 10⁰ | 0 |
| BHR-31 | iCQhTL | 0.070 ± 0.004 | 0.87 ± 0.08 | 8.0 ± 0.9 × 10¹ | 3 |
| BHR-32 | AVShQs | 0.0014 ± 0.0003 | 0.1 ± 0.05 | 1.3 ± 0.7 × 10¹ | 0 |
| BHR-33 | VfRhTL | 7 ± 2 | 0.5 ± 0.1 | 1.5 ± 0.6 × 10⁴ | 500 |
| BHR-34 | iLGLNs | 0.009 ± 0.003 | 0.2 ± 0.1 | 2.6 ± 0.3 × 10¹ | 2 |
| BHR-35 | ACRhNs | 0.1 ± 0.02 | 0.5 ± 0.1 | 1.6 ± 0.6 × 10² | 5 |
| BHR-36 | AVQhQs | 0.0031 ± 0.0003 | 0.44 ± 0.06 | 7 ± 1 × 10⁰ | 0 |
| BHR-37 | iLGLTs | nd | nd | 1.75 ± 0.03 × 10¹ | 1 |
| BHR-38 | ACQLiL | 0.11 ± 0.03 | 0.6 ± 0.2 | 1.9 ± 0.7 × 10² | 6 |
| WT | ifhhis | 0.047 ± 0.007 | 1.0 ± 0.2 | [symbol] | na |
| BHR-39 | [symbol] | [symbol] | [symbol] | [symbol] | 7 |
| BHR-40 | ACRFQL | 1.2 ± 0.1 | 0.16 ± 0.02 | 8 ± 1 × 10³ | 270 |
| BHR-41 | [symbol] | [symbol] | [symbol] | [symbol] | 230 |
| BHR-42 | AERLTs | 0.0043 ± 0.0005 | 0.25 ± 0.05 | [symbol] | 6 |
| BHR-43 | [symbol] | [symbol] | [symbol] | [symbol] | 0 |
| BHR-44 | [symbol] | [symbol] | [symbol] | [symbol] | 1 |
| BHR-45 | [symbol] | [symbol] | [symbol] | [symbol] | 10 |
| BHR-46 | [symbol] | [symbol] | [symbol] | [symbol] | 11 |
| BHR-47 | [symbol] | [symbol] | [symbol] | 1.0 ± 0.2 × 10² | 3 |
| BHR-48 | LWQLTS | 0.076 ± 0.001 | 0.22 ± 0.01 | 3.42 ± 0.09 × 10² | 11 |
| BHR-49 | GCQhiL | 1.2 ± 0.04 | 0.22 ± 0.01 | 5.5 ± 0.3 × 10³ | 180 |
| BHR-50 | iVQFNI | 0.0066 ± 0.0006 | 0.45 ± 0.06 | 1.5 ± 0.2 × 10¹ | 1 |
| BHR-51 | iCRLNI | 0.23 ± 0.01 | 0.45 ± 0.04 | 5.0 ± 0.5 × 10² | 17 |
| BHR-52 | ACRhTs | 0.098 ± 0.002 | 0.25 ± 0.01 | 4.0 ± 0.2 × 10² | 13 |
| BHR-53 | ACRLNL | 1.39 ± 0.05 | 0.32 ± 0.02 | 4.3 ± 0.3 × 10³ | 140 |
| BHR-54 | ACQLTs | 0.006 ± 0.002 | 0.11 ± 0.06 | 3.1 ± 0.2 × 10¹ | 1 |
| BHR-55 | iLGFQs | 0.007 ± 0.002 | 0.2 ± 0.07 | 4 ± 2 × 10¹ | 1 |
| BHR-56 | iLGFSs | 0.008 ± 0.003 | 0.4 ± 0.2 | 2 ± 1 × 10¹ | 1 |
| BHR-57 | ACQHSs | 0.003 ± 0.0006 | 0.12 ± 0.05 | 3 ± 1 × 10¹ | 1 |
| BHR-58 | ALGFQS | nd | nd | 2.1 ± 0.1 × 10¹ | 1 |
| BHR-59 | ALGFNS | 0.0051 ± 0.0007 | 0.1 ± 0.03 | 5 ± 2 × 10¹ | 2 |
| BHR-60 | AVGFNS | nd | nd | 2.1 ± 0.1 × 10¹ | 1 |
| BHR-61 | ALRLQV | nd | nd | 8.7 ± 0.9 × 10¹ | 3 |
| BHR-62 | ACQLNs | 0.009 ± 0.001 | 0.13 ± 0.03 | 7 ± 2 × 10¹ | 2 |
| BHR-63 | VLGFQs | 0.013 ± 0.002 | 0.31 ± 0.06 | 4 ± 1 × 10¹ | 1 |
| BHR-64 | AERLNL | 0.08 ± 0.01 | 0.1 ± 0.03 | 8 ± 3 × 10² | 27 |
| BHR-65 | AERLQs | nd | nd | 8.9 ± 0.7 × 10⁰ | 0 |
| BHR-66 | VVRLSL | 1.8 ± 0.3 | 0.17 ± 0.03 | 1.1 ± 0.3 × 10⁴ | 370 |
| BHR-67 | iCQFNs | 0.0033 ± 0.0005 | 0.06 ± 0.02 | 6 ± 2 × 10¹ | 2 |
| BHR-68 | CARLTL | 0.52 ± 0.05 | 0.23 ± 0.03 | 2.3 ± 0.4 × 10³ | 77 |
| BHR-69 | iCQFis | 0.0014 ± 0.0001 | 0.04 ± 0.01 | 3.5 ± 0.9 × 10¹ | 1 |
| BHR-70 | CVRLSL | 3.3 ± 0.2 | 0.2 ± 0.02 | 1.7 ± 0.2 × 10⁴ | 570 |
| BHR-71 | AVQFNs | 0.003 ± 0.0002 | 0.37 ± 0.04 | 8 ± 1 × 10⁰ | 0 |
| BHR-72 | ACGhNs | 0.0043 ± 0.0003 | 0.013 ± 0.003 | 3.3 ± 0.8 × 10² | 11 |
| BHR-73 | AEGhNs | 0.0008 ± 0.00006 | 0.016 ± 0.004 | 5 ± 1 × 10¹ | 2 |
| BHR-74 | ACQFNs | 0.0039 ± 0.0002 | 0.001 ± 0.0003 | 4 ± 1 × 10³ | 130 |
| BHR-75 | AEQFQs | 0.0007 ± 0.00009 | 0.11 ± 0.03 | 6 ± 2 × 10⁰ | 0 |
| BHR-76 | AEQYQs | 0.0016 ± 0.0004 | 0.4 ± 0.1 | 4 ± 1 × 10⁰ | 0 |

^{a}Sequence given as single letter amino acid code for residues present at position 106/132/254/257/274/308.
nd = not determined. [symbol]
indicates data missing or illegible when filed

Against OMVR (FIG. 23F), three variants were identified as being more than 1000-fold improved in activity with the best being BHR-66 with a k_{cat}/Kₘ of 8.0×10⁴M⁻¹s⁻¹, which is 1180-fold improved (Table 20). The stereoselectivity of this set of variants was much improved, with 10 variants showing no preference and 12 variants being selective for the R_{P}-enantiomer. Four variants were found to exclusively hydrolyze the R_{P}-enantiomer. The two best variants with R_{P}-

TABLE 24
Kinetic constants for purified PTE variants with paraoxon.

| | | k_{cat} | Kₘ | k_{cat}/Kₘ | | | k_{cat} | Kₘ | k_{cat}/Kₘ |
|---|---|---|---|---|---|---|---|---|---|
| Number | Sequence^{a} | (s⁻¹) | (μM) | (M⁻¹s⁻¹) | Number | Sequence^{a} | (s⁻¹) | (mM) | (M⁻¹s⁻¹) |
| BHR-1 | iCRhSs | 410 ± 7 | 41 ± 4 | 1.0 ± 0.1 × 10⁷ | BHR-39 | ACQFQL | 419 ± 5 | 0.029 ± 0.001 | 1.45 ± 0.07 × 10⁷ |
| BHR-2 | iCRhTs | 415 ± 7 | 30 ± 3 | 1.4 ± 0.2 × 10⁷ | BHR-40 | ACRFQL | 890 ± 20 | 0.020 ± 0.001 | 4.4 ± 0.3 × 10⁷ |
| BHR-3 | SCRhNL | 770 ± 10 | 46 ± 4 | 1.7 ± 0.1 × 10⁷ | BHR-41 | ACRFSL | 800 ± 10 | 0.021 ± 0.001 | 3.8 ± 0.2 × 10⁷ |
| BHR-4 | iCQhiL | 5300 ± 200 | 190 ± 20 | 2.8 ± 0.3 × 10⁷ | BHR-42 | AERLTs | 2090 ± 20 | 0.194 ± 0.005 | 1.08 ± 0.03 × 10⁷ |
| BHR-5 | iCQhSL | 6000 ± 200 | 310 ± 30 | 1.9 ± 0.2 × 10⁷ | BHR-43 | AVQhNs | 9860 ± 80 | 0.352 ± 0.006 | 2.80 ± 00.6 × 10⁷ |
| BHR-6 | sCQhNs | 17000 ± 800 | 720 ± 70 | 2.4 ± 0.2 × 10⁷ | BHR-44 | ALQFTs | 249 ± 2 | 0.018 ± 0.001 | 1.40 ± 0.04 × 10⁷ |
| BHR-7 | iCRhTL | 230 ± 6 | 26 ± 3 | 9.0 ± 0.1 × 10⁶ | BHR-45 | ACQFNL | 318 ± 7 | 0.022 ± 0.002 | 1.4 ± 0.1 × 10⁷ |
| BHR-8 | VCQhNL | 7500 ± 200 | 390 ± 30 | 1.9 ± 0.2 × 10⁷ | BHR-46 | SERLTL | 2290 ± 20 | 0.227 ± 0.005 | 1.01 ± 0.03 × 10⁷ |
| BHR-9 | CfRhQI | 790 ± 20 | 24 ± 3 | 3.3 ± 0.3 × 10⁷ | BHR-47 | GEQLQs | 1260 ± 20 | 0.044 ± 0.002 | 2.9 ± 0.1 × 10⁷ |
| BHR-10 | iChhNs | 6500 ± 200 | 290 ± 20 | 2.2 ± 0.2 × 10⁷ | BHR-48 | LWQLTS | 706 ± 5 | 0.019 ± 0.001 | 3.9 ± 0.1 × 10⁷ |
| BHR-11 | CAQhQL | 690 ± 10 | 36 ± 3 | 1.9 ± 0.2 × 10⁷ | BHR-49 | GCQhiL | 1770 ± 50 | 0.061 ± 0.005 | 2.9 ± 0.2 × 10⁷ |
| BHR-12 | ACQhTs | 400 ± 6 | 30 ± 3 | 1.3 ± 0.1 × 10⁷ | BHR-50 | iVQFNI | 94 ± 1 | 0.0087 ± 0.0003 | 1.09 ± 0.04 × 10⁷ |
| BHR-13 | iCGhNs | 5100 ± 200 | 670 ± 60 | 7.6 ± 0.7 × 10⁷ | BHR-51 | iCRLNI | 384 ± 6 | 0.081 ± 0.004 | 4.8 ± 0.2 × 10⁶ |
| BHR-14 | GCQhNL | 1750 ± 30 | 61 ± 6 | 2.9 ± 0.3 × 10⁷ | BHR-52 | ACRhTs | 1100 ± 30 | 0.038 ± 0.004 | 2.9 ± 0.3 × 10⁷ |
| BHR-15 | AARhQL | 1400 ± 300 | 56 ± 6 | 2.5 ± 0.3 × 10⁷ | BHR-53 | ACRLNL | 3600 ± 300 | 0.13 ± 0.02 | 2.9 ± 0.5 × 10⁷ |
| BHR-16 | VCRhNL | 375 ± 6 | 26 ± 3 | 1.4 ± 0.1 × 10⁷ | BHR-54 | ACQLTs | 305 ± 6 | 0.032 ± 0.002 | 9.53 ± 0.06 × 10⁶ |
| BHR-17 | ACRhiL | 1070 ± 20 | 68 ± 7 | 1.6 ± 0.2 × 10⁷ | BHR-55 | iLGFQs | 352 ± 7 | 0.065 ± 0.005 | 5.4 ± 0.4 × 10⁶ |
| BHR-18 | CLRhQL | 553 ± 8 | 19 ± 2 | 2.9 ± 0.3 × 10⁷ | BHR-56 | iLGFSs | 370 ± 9 | 0.049 ± 0.004 | 7.6 ± 0.6 × 10⁶ |
| BHR-19 | iCRhQI | 392 ± 6 | 24 ± 2 | 1.6 ± 0.1 × 10⁷ | BHR-57 | ACQHSs | 15100 ± 400 | 0.64 ± 0.3 | 2.4 ± 0.1 × 10⁷ |
| BHR-20 | iCRhQs | 480 ± 8 | 41 ± 5 | 1.2 ± 0.1 × 10⁷ | BHR-58 | ALGFQS | 710 ± 10 | 0.197 ± 0.009 | 3.6 ± 0.2 × 10⁶ |
| BHR-21 | VCRhQL | 683 ± 3 | 62 ± 7 | 1.1 ± 0.1 × 10⁷ | BHR-59 | ALGFNS | 1000 ± 10 | 0.128 ± 0.004 | 7.8 ± 0.3 × 10⁶ |
| BHR-22 | VCRhTL | 1450 ± 30 | 63 ± 6 | 2.3 ± 0.2 × 10⁷ | BHR-60 | AVGFNS | 610 ± 10 | 0.096 ± 0.005 | 6.4 ± 0.3 × 10⁶ |
| BHR-23 | iVRhSL | 580 ± 10 | 26 ± 3 | 2.2 ± 0.2 × 10⁷ | BHR-61 | ALRLQV | 2010 ± 60 | 0.15 ± 0.01 | 1.3 ± 0.1 × 10⁷ |
| BHR-24 | CLRhSL | 600 ± 6 | 24 ± 2 | 2.5 ± 0.2 × 10⁷ | BHR-62 | ACQLNs | 430 ± 10 | 0.14 ± 0.01 | 3.1 ± 0.2 × 10⁶ |
| BHR-25 | CARhNL | 610 ± 8 | 18 ± 2 | 3.4 ± 0.3 × 10⁷ | BHR-63 | VLGFQs | 930 ± 20 | 0.19 ± 0.01 | 4.9 ± 0.3 × 10⁶ |
| BHR-26 | iCQhis | 7400 ± 200 | 570 ± 40 | 1.3 ± 0.1 × 10⁷ | BHR-64 | AERLNL | 1640 ± 40 | 0.24 ± 0.01 | 6.8 ± 0.3 × 10⁶ |
| BHR-27 | CLGhTs | 3200 ± 100 | 310 ± 30 | 1.0 ± 0.1 × 10⁷ | BHR-65 | AERLQs | 2740 ± 30 | 0.262 ± 0.007 | 1.05 ± 0.03 × 10⁷ |
| BHR-28 | CVQhNL | 470 ± 10 | 12 ± 1 | 3.9 ± 0.4 × 10⁷ | BHR-66 | VVRLSL | 1990 ± 20 | 0.051 ± 0.002 | 3.9 ± 0.2 × 10⁷ |
| BHR-29 | CDQhQL | 1180 ± 30 | 76 ± 9 | 1.6 ± 0.2 × 10⁷ | BHR-67 | iCQFNs | 60 ± 1 | 0.014 ± 0.002 | 4.3 ± 0.6 × 10⁶ |
| BHR-30 | iCGhTs | 6000 ± 200 | 560 ± 40 | 1.1 ± 0.1 × 10⁷ | BHR-68 | CARLTL | 534 ± 5 | 0.028 ± 0.001 | 1.94 ± 0.7 × 10⁷ |
| BHR-31 | iCQhTL | 7400 ± 100 | 290 ± 20 | 2.5 ± 0.2 × 10⁷ | BHR-69 | iCQFis | 25.8 ± 0.5 | 0.01 ± 0.001 | 2.6 ± 0.3 × 10⁶ |
| BHR-32 | AVShQs | 7500 ± 100 | 0.308 ± 0.009 | 2.44 ± 0.08 × 10⁷ | BHR-70 | CVRLSL | 4370 ± 30 | 0.22 ± 0.004 | 1.99 ± 0.04 × 10⁷ |
| BHR-33 | VfRhTL | 640 ± 20 | 0.018 ± 0.002 | 3.5 ± 0.4 × 10⁷ | BHR-71 | AVQFNs | 229 ± 3 | 0.0159 ± 0.0009 | 1.44 ± 0.08 × 10⁷ |
| BHR-34 | iLGLNs | 500 ± 10 | 0.147 ± 0.008 | 3.4 ± 0.2 × 10⁶ | BHR-72 | ACGhNs | 11200 ± 400 | 0.84 ± 0.05 | 1.33 ± 0.09 × 10⁷ |
| BHR-35 | ACRhNs | 658 ± 8 | 0.020 ± 0.001 | 3.3 ± 0.1 × 10⁷ | BHR-73 | AEGhNs | 2810 ± 70 | 1.8 ± 0.06 | 1.56 ± 0.04 × 10⁶ |
| BHR-36 | AVQhQs | 9800 ± 100 | 0.37 ± 0.01 | 2.6 ± 0.1 × 10⁷ | BHR-74 | ACQFNs | 120 ± 2 | 0.006 ± 0.0007 | 2.0 ± 0.2 × 10⁷ |
| BHR-37 | iLGLTs | 670 ± 10 | 0.091 ± 0.005 | 7.3 ± 0.5 × 10⁶ | BHR-75 | AEQFQs | 423 ± 4 | 0.124 ± 0.004 | 3.4 ± .1 × 10⁶ |
| BHR-38 | ACQLiL | 331 ± 5 | 0.020 ± 0.001 | 1.6 ± 0.1 × 10⁷ | BHR-76 | AEQYQs | 302 ± 3 | 0.126 ± 0.003 | 2.40 ± 0.06 × 10⁶ |

^{a}Sequence given as single letter amino acid code for residues present at position 106/132/254/257/274/308.
nd = not determined.

OMVR were BHR-39, which shows a 2-fold selectivity for the R_{P}-enantiomer and BHR-44, which is exclusive for hydrolysis of the R_{P}-enantiomer. Both variants have values of k_{cat}/Kₘ of ˜2.3×10³ M⁻¹ s⁻¹, which is 330-fold improved compared to wild-type PTE.

### Example 54

Quantitative Analysis of Screening Activity. Single amino acid substitutions of wild-type PTE were not constructed in this work. However, the large amount of data generated allowed the calculation of the effects of single mutations. Sequencing selected colonies from the screen identified 241 unique variants, which amounts to 28,920 pairs of sequences that differ by one to six amino acids. The activity for each variant identified in the screen was normalized and scaled according to eqs. 1-3. While these numbers represent the activity of each variant with each substrate, they do not reveal the magnitude compared to other variants. To obtain more directly comparable values, the raw activity was normalized by the average total activity measured for any variant in the screen according to eq. 5. The values of the resulting Aᵥₜⱼ allow the direct comparison of the activity of any variant with any substrate to any other. The quantitative effect of these mutations is then calculated by dividing the observed value of Aᵥₜⱼ for the first variant by the value for the second variant according to eq. 6, yielding an activity coefficient C₍ₖₗ₎ⱼ, which provides a quantitative measurement of the effect of the mutations. The subset of variants that only differ from each other by a single amino acid was used to calculate the activity coefficients for single amino acid substitutions according to eq. 6. The effects of substituting for a wild-type residue was calculated for each possible substitution according to eq. 8, or in the cases where a substitution was not directly observed in the single substitution set, it was calculated according to eq. 7. Because many of the variants show improvements for multiple substrates, the average activity coefficient for all substrates was also calculated according to eq. 9. The activity coefficients for mutation from the wild-type residues at each site are presented in Table 25.

TABLE 25
Activity coefficients for mutations from
the wild-type residue at each position.

| | C_{(wk)} | C_{(wk)} | C_{(wk)} | C_{(wk)} | C_{(wk)} | | |
|---|---|---|---|---|---|---|---|
| Position | _{DEVX} | _{OMVR} | _{Malathion} | _{DMVX} | _{Ethoprop} | n | C_{(wk)n} |
| 106 | | | | | | | |
| i→V | 1.25 | 1.30 | 0.57 | 1.05 | 0.32 | 13 | 1.0 |
| i→C | 1.34 | 1.20 | 0.66 | 1.28 | 0.34 | 6 | 1.1 |
| i→G | 1.86 | 0.49 | 0.08 | 21.27 | 0.87 | 1* | 1.1 |
| i→S | 1.14 | 1.94 | 1.48 | 4.18 | 0.32 | 1* | 1.9 |
| i→A | 1.60 | 1.16 | 2.57 | 3.47 | 0.15 | 13 | 2.0 |
| 132 | | | | | | | |
| f→C | 0.06 | 0.13 | 1.80 | 0.17 | 1.69 | 6 | 0.2 |
| f→V | 0.10 | 0.17 | 1.55 | 0.70 | NO | 1* | 0.3 |
| f→E | 0.16 | 0.11 | 52.76 | NO | NO | * | 0.4 |
| f→A | 0.34 | 2.36 | 0.17 | 0.11 | 0.08 | 1 | 0.4 |
| f→W | 1.31 | 0.43 | 0.03 | 3.02 | 9.58 | * | 0.5 |
| f→L | 0.17 | 0.12 | 12.02 | 0.25 | 0.12 | 3* | 0.5 |
| f→D | 0.34 | 2.41 | 0.39 | 0.19 | NO | * | 0.5 |
| 254 | | | | | | | |
| h→N | 0.02 | NO | 0.64 | 0.66 | NO | 0.2 | |
| h→Q | 0.67 | 0.71 | 0.12 | 0.12 | 0.48 | 7 | 0.3 |
| h→R | 0.82 | 0.30 | 0.04 | 0.81 | 1.32 | 7 | 0.3 |
| h→G | 1.00 | 0.51 | 0.37 | 0.17 | 0.07 | 3* | 0.4 |
| h→S | 27.77 | 7.48 | 0.78 | 0.23 | 2.81 | * | 2.5 |
| 257 | | | | | | | |
| h→F | 1.01 | 9.68 | 1.86 | 0.35 | 3.79 | 13 | 1.6 |
| h→C | 1.41 | 2.33 | 5.98 | 0.52 | 2.42 | 6 | 1.8 |
| h→L | 2.16 | 1.04 | 1.98 | 5.11 | 1.58 | 13 | 2.2 |
| h→W | 8.59 | 47.36 | 2.13 | 0.23 | 5.68 | 5 | 3.8 |
| 274 | | | | | | | |
| i→Q | 0.71 | 0.46 | 0.59 | 0.28 | 0.06 | 7 | 0.5 |
| i→S | 0.85 | 0.60 | 0.35 | 0.53 | 0.50 | 7 | 0.6 |
| i→T | 0.65 | 0.61 | 0.50 | 0.48 | 0.46 | 10 | 0.6 |
| i→N | 0.72 | 0.61 | 0.75 | 0.75 | 0.06 | 9 | 0.7 |
| 308 | | | | | | | |
| s→L | 0.88 | 0.27 | 0.22 | 0.29 | 5.39 | 15 | 0.3 |
| s→V | 5.53 | 1.31 | 0.78 | 0.23 | NO | * | 1.1 |

*Mutation not observed and mutations from wild-type with less than 5 occurrences were calculated from other observed mutations.
NO = not observed.

### Example 55

Construction and Characterization of Variants Based on Activity Coefficients. Efficient hydrolysis of S_{P}-VR (FIG. 23C) was a primary objective of this study. Evaluation of the activity coefficients with the chiral analog OMVR (FIG. 23F) suggested that some variants of potential interest had not been identified. At position 106, the wild-type residue isoleucine was most commonly seen, but this residue confers a stereochemical preference for the less desirable R_{P}-enantiomer. Interestingly, the only residue predicted to be better than isoleucine is glycine, which is known to alter the stereopreference of PTE.(58, 65) A set of variants was constructed by substituting glycine at position 106 in variants BHR-13, BHR-17, BHR-22, BHR-26 and BHR-30, all of which showed reasonable activity for R_{P}-OMVR (Table 26).

TABLE 26
Kinetic constants for I106G variants with OMVR

| Number | Sequence^{a} | S_{P} k_{cat}/Kₘ | R_{P} k_{cat}/Kₘ | | Ratio |
|---|---|---|---|---|---|
| BHR-13-G | GCGhNs | 3.59 ± 0.04 × 10³ | 5.68 ± 0.08 | × 10² | 6:1 |
| BHR-17-G | GCRhiL | 4.78 ± 0.03 × 10⁴ | 8.98 ± 0.07 | × 10² | 53:1 |
| BHR-22-G | GCRhTL | 2.47 ± 0.04 × 10⁴ | 8.29 ± 0.06 | × 10² | 30:1 |
| BHR-26-G | GCQhis | 4.23 ± 0.02 × 10² | 4.23 ± 0.02 | × 10² | 1:1 |
| BHR-30-G | GCGhTs | 2.44 ± 0.03 × 10³ | 3.2 ± 0.2 | × 10² | 8:1 |

^{a}Sequence given as single letter amino acid code for residues present at position 106/132/254/257/274/308.

The substitution of glycine for isoleucine in variants BHR-13, BHR-26 and BHR-30 resulted in a 2- to 3-fold decrease in activity for S_{P}-OMVR (Table 26). The substitution of glycine in BHR-13 gave a small increase in activity for the R_{P}-enantiomer. This resulted in less stereoselectivity, but still a 6-fold preference for the S_{P}-enantiomer. The substitution gave a slight increase in activity for the R_{P}-enantiomer in BHR-26, resulting in a loss of stereoselectivity for this variant, while the substitution in BHR-30 gave a decrease in activity for the R_{P}-enantiomer. Substitution of glycine for alanine or valine in BHR-17 and BHR-22, respectively, resulted in little change, although there was a slight loss of activity when alanine was replaced. More problematic was the finding that the stability of the I106G variants was compromised. It was observed that under typical assay conditions, these variants lost essentially all activity within 1 h.

While the results of substituting glycine at position 106 were disappointing, better results were achieved by substitution with alanine. Isoleucine was predicted to give better catalytic activity for racemic OMVR (FIG. 23F), but alanine was predicted to be the next best residue. The replacement of isoleucine with alanine in BHR-13 resulted in the variant BHR-72, which showed a 5-fold increase in activity for R_{P}-OMVR (FIG. 23F) and a complete loss of stereoselectivity (Table 19). With a k_{cat}/Kₘ of 2×10³ M⁻¹ s⁻¹, this variant is 290-fold improved over wild-type PTE.

The best activity coefficient for OMVR hydrolysis at position 132 was observed for glutamate. Substitution of glutamate for the cysteine at position 132 in BHR-72 resulted in BHR-73. The mutation reduced activity by approximately 2-fold, but neither variant showed stereochemical selectivity. Given this observation, a reverse substitution of glutamate for cysteine was done with BHR-75, which is specific for R_{P}-OMVR, resulting in variant BHR-74. The substitution of cysteine for glutamate resulted in approximately 2-fold improvement in activity for the R_{P}-enantiomer. With a k_{cat}/Kₘ of 3.0×10³ M⁻¹ s⁻¹, BHR-74 was the best enzyme observed for R_{P}-OMVR and is 443-fold improved over the wild-type PTE (Table 19).

### Example 56

Effects of DTNB on Enzymatic Activity. Variants were ultimately selected for their ability to catalyze the hydrolysis of specific compounds in the presence of DTNB. A significant number of the variants identified contain cysteine at either residue position 106 or 132. The possibility of DTNB reacting directly with either of these cysteine residues was addressed by incubating selected variants with DTNB under typical reaction conditions and following the release of 5-thio-2-nitrobenzoic acid due to the reaction with the protein. The two native cysteine residues in PTE were found to be unreactive toward DTNB over the course of typical enzymatic assays. Similar results were found with the variant BHR-70, which contains cysteine at residue position 106, suggesting that a cysteine at this position is inaccessible to DTNB. Variants that contained cysteine at residue position 132 were found to readily react with DTNB, releasing 1 equivalent of 5-thio-2-nitrobenzoic acid with t_{1/2} values between 30 and 60 s. To determine if the reaction of the variants containing cysteine at residue position 132 with DTNB substantially altered the kinetic properties of the variants, two of the most active variants (BHR-69 and BHR-74) were assayed with DEVX and OMVR in the absence of DTNB using a discontinuous assay. For both substrates and both variants tested, inclusion of DTNB resulted in somewhat higher enzymatic rates, but the observed rates were altered by only a factor of ˜2.

### Example 57

Characterization of Variants with VX and VR. Purified variants were tested for activity with VX (FIG. 23B) and VR (FIG. 23C) in total hydrolysis assays, which enabled the determination of stereochemical selectivity as well as the k_{cat}/Kₘ values for both enantiomers. Against VX, seven of the identified variants were improved more than 1000-fold over wild-type PTE (Tables 19 and 27). The best variant identified was BHR-23, which is 9200-fold improved relative to wild-type PTE, and with a k_{cat}/Kₘ of 8×10⁵ M⁻¹ s⁻¹, it is among the best variants ever described for hydrolysis of VX (FIG. 23B).(57,72) Also of interest are BHR-4 and BHR-69, which have k_{cat}/Kₘ values in excess of 10⁵ M⁻¹ s⁻¹. These variants are 2750- and 1700-fold improved, respectively, and have a demonstrated preference for the S_{P}-enantiomer of VX.

TABLE 27
Kinetic constants for purified PTE variants with VX.

| | | k_{cat}/Kₘ₁^{b} | k_{cat}/Kₘ₂^{b} | Ratio^{c} | Fold- | S_{P}-fold |
|---|---|---|---|---|---|---|
| Number | Sequence^{a} | (M⁻¹s⁻¹) | (M⁻¹s⁻¹) | R_{P}:S_{P} | improved | improved |
| wt | ifhhis | 8.4 × 10¹ | 8.4 × 10¹ | 1:1 | na | na |
| BHR-1 | iCRhSs | 8.86 ± 0.07 × 10³ | 8.86 ± 0.07 × 10³ | 1:1 | 110 | 110 |
| BHR-2 | iCRhTs | 1.09 ± 0.02 × 10⁴ | 2.2 ± 0.2 × 10³ | 5.0:1 | 130 | 27 |
| BHR-3 | SCRhNL | 4.16 ± 0.01 × 10³ | 4.16 ± 0.01 × 10³ | 1:1 | 50 | 50 |
| BHR-4 | iCQhiL | 2.31 ± 0.02 × 10⁵ | 4.43 ± 0.04 × 10⁴ | 1:5.2* | 2800 | 2800 |
| BHR-5 | iCQhSL | 1.71 ± 0.01 × 10⁵ | 3.18 ± 0.02 × 10⁴ | 1:5.4* | 2000 | 2000 |
| BHR-6 | sCQhNs | 3.79 ± 0.03 × 10⁴ | 5.25 ± 0.06 × 10³ | 7.2:1 | 450 | 63 |
| BHR-7 | iCRhTL | 3.21 ± 0.01 × 10⁴ | 3.21 ± 0.01 × 10⁴ | 1:1 | 380 | 380 |
| BHR-8 | VCQhNL | 3.68 ± 0.05 × 10⁴ | 4.1 ± 0.1 × 10³ | 9.0:1 | 440 | 49 |
| BHR-9 | CfRhQI | 2.30 ± 0.01 × 10³ | NO | >100:1 | 27 | Na |
| BHR-10 | iChhNs | 2.29 ± 0.01 × 10³ | NO | >100:1 | 27 | Na |
| BHR-11 | CAQhQL | 4.2 ± 0.5 × 10⁵ | 2.32 ± 0.03 × 10⁴ | 18:1 | 5000 | 280 |
| BHR-12 | ACQhTs | 1.13 ± 0.01 × 10³ | NO | >100:1 | 13 | Na |
| BHR-13 | iCGhNs | 1.26 ± 0.02 × 10⁵ | 2.53 ± 0.04 × 10³ | 1:50* | 1500 | 1500 |
| BHR-14 | GCQhNL | 4.92 ± 0.03 × 10³ | 4.92 ± 0.03 × 10³ | 1:1 | 59 | 59 |
| BHR-15 | AARhQL | 6.02 ± 0.02 × 10³ | 6.02 ± 0.02 × 10³ | 1:1 | 72 | 73 |
| BHR-16 | VCRhNL | 1.34 ± 0.01 × 10⁴ | 1.34 ± 0.01 × 10⁴ | 1:1 | 160 | 160 |
| BHR-17 | ACRhiL | 1.94 ± 0.01 × 10⁴ | 1.94 ± 0.01 × 10⁴ | 1:1 | 230 | 230 |
| BHR-18 | CLRhQL | 6.10 ± 0.01 × 10³ | 6.10 ± 0.01 × 10³ | 1:1 | 73 | 73 |
| BHR-19 | iCRhQl | 6.05 ± 0.02 × 10⁴ | 6.05 ± 0.02 × 10⁴ | 1:1 | 720 | 720 |
| BHR-20 | iCRhQs | 1.87 ± 0.01 × 10⁴ | 3.04 ± 0.01 × 10³ | 6.1:1 | 220 | 37 |
| BHR-21 | VCRhQL | 1.48 ± 0.01 × 10⁴ | 1.48 ± 0.01 × 10⁴ | 1:1 | 180 | 180 |
| BHR-22 | VCRhTL | 7.60 ± 0.06 × 10⁴ | 7.60 ± 0.06 × 10⁴ | 1:1 | 916 | 916 |
| BHR-23 | iVRhSL | 7.7 ± 0.3 × 10⁵ | 4.19 ± 0.08 × 10⁴ | 1:18* | 9200 | 9200 |
| BHR-24 | CLRhSL | 1.18 ± 0.02 × 10⁵ | 4.6 ± 0.1 × 10³ | 1:26* | 1400 | 1400 |
| BHR-25 | CARhNL | 5.99 ± 0.04 × 10⁴ | 5.99 ± 0.04 × 10⁴ | 1:1 | 720 | 720 |
| BHR-26 | iCQhis | 3.19 ± 0.02 × 10⁴ | 3.19 ± 0.02 × 10⁴ | 1:1 | 380 | 380 |
| BHR-27 | CLGhTs | 2.26 ± 0.02 × 10⁴ | 1.68 ± 0.03 × 10³ | 1:13* | 270 | 270 |
| BHR-28 | CVQhNL | 1.97 ± 0.01 × 10⁴ | 1.97 ± 0.01 × 10⁴ | 1:1 | 240 | 240 |
| BHR-29 | CDQhQL | 1.30 ± 0.01 × 10⁴ | 1.30 ± 0.01 × 10⁴ | 1:1 | 160 | 160 |
| BHR-30 | iCGhTs | 2.13 ± 0.02 × 10⁵ | 7.4 ± 0.1 × 10³ | 1:29* | 2500 | 2500 |
| BHR-31 | iCQhTL | 7.5 ± 0.1 × 10⁴ | NO | S | 890 | 890 |
| BHR-32 | AVShQs | 4.64 ± 0.01 × 10² | 4.64 ± 0.01 × 10² | 1:1 | 6 | 6 |
| BHR-33 | VfRhTL | 7.77 ± 0.03 × 10² | 7.77 ± 0.03 × 10² | 1:1 | 9 | 9 |
| BHR-34 | iLGLNs | 4.62 ± 0.03 × 10³ | 5.92 ± 0.06 × 10² | 7.8:1 | 55 | 7 |
| BHR-35 | ACRhNs | 2.99 ± 0.09 × 10³ | 4.96 ± 0.06 × 10² | 6:1 | 36 | 6 |
| BHR-36 | AVQhQs | 1.17 ± 0.04 × 10³ | 1.17 ± 0.04 × 10³ | 1:1 | 14 | 14 |
| BHR-37 | iLGLTs | 4.22 ± 0.04 × 10³ | 7.2 ± 0.1 × 10² | 5.9:1 | 50 | 9 |
| BHR-38 | ACQLiL | 1.63 ± 0.02 × 10⁴ | 3.3 ± 0.1 × 10² | 1:49* | 190 | 190 |
| WT | ifhhis | 8.4 × 10¹ | 8.4 × 10¹ | 1:1 | na | na |
| BHR-39 | ACQFQL | 2.57 ± 0.07 × 10⁴ | 2.9 ± 0.2 × 10² | 1:89 | 310 | 310 |
| BHR-40 | ACRFQL | 1.18 ± 0.01 × 10⁴ | 8.51 ± 0.06 × 10² | 14:1 | 140 | 10 |
| BHR-41 | ACRFSL | 1.36 ± 0.01 × 10⁴ | 1.10 ± 0.03 × 10³ | 12:1 | 160 | 13 |
| BHR-42 | AERLTs | 4.44 ± 0.02 × 10² | 4.44 ± 0.02 × 10² | 1:1 | 5 | 5 |
| BHR-43 | AVQhNs | 6.8 ± 0.1 × 10³ | 9.69 ± 0.02 × 10² | 7:1 | 82 | 82 |
| BHR-44 | ALQFTs | 6.4 ± 0.1 × 10³ | 5.76 ± 0.07 × 10² | 1:11 | 77 | 77 |
| BHR-45 | ACQFNL | 1.29 ± 0.01 × 10⁴ | 2.39 ± 0.05 × 10² | 1:54* | 160 | 160 |
| BHR-46 | SERLTL | 1.01 ± 0.01 × 10³ | 8.6 ± 0.1 × 10¹ | 12:1 | 12 | 1 |
| BHR-47 | GEQLQs | 1.19 ± 0.01 × 10³ | 2.49 ± 0.05 × 10² | 4.8:1 | 14 | 3 |
| BHR-48 | LWQLTS | 2.25 ± 0.01 × 10³ | 2.00 ± 0.01 × 10² | 11:1 | 27 | 2 |
| BHR-49 | GCQhiL | 6.9 ± 0.1 × 10³ | 1.53 ± 0.06 × 10³ | 4.5:1 | 82 | 18 |
| BHR-50 | iVQFNI | 5.39 ± 0.02 × 10⁴ | 5.77 ± 0.01 × 10³ | 9.3:1 | 640 | 70 |
| BHR-51 | iCRLNI | 7.72 ± 0.06 × 10³ | 3.16 ± 0.03 × 10² | 24:1 | 92 | 4 |
| BHR-52 | ACRhTs | 4.13 ± 0.04 × 10³ | 1.04 ± 0.01 × 10³ | 4:1 | 49 | 13 |
| BHR-53 | ACRLNL | 2.36 ± 0.01 × 10⁴ | 2.96 ± 0.02 × 10³ | 8:1 | 280 | 36 |
| BHR-54 | ACQLTs | 4.99 ± 0.05 × 10³ | 1.21 ± 0.02 × 10³ | 4.1:1 | 60 | 60 |
| BHR-55 | iLGFQs | 8.32 ± 0.07 × 10³ | 3.54 ± 0.01 × 10² | 1:24* | 100 | 100 |
| BHR-56 | iLGFSs | 7.36 ± 0.08 × 10³ | 3.31 ± 0.01 × 10² | 22:1 | 89 | 89 |
| BHR-57 | ACQHSs | 5.54 ± 0.09 × 10³ | 8.61 ± 0.06 × 10² | 6.4:1 | 67 | 67 |
| BHR-58 | ALGFQS | 2.19 ± 0.01 × 10³ | 2.07 ± 0.01 × 10² | 1:11 | 26 | 26 |
| BHR-59 | ALGFNS | 2.00 ± 0.01 × 10³ | 1.90 ± 0.01 × 10² | 1:11 | 24 | 24 |
| BHR-60 | AVGFNS | 3.15 ± 0.03 × 10³ | 3.41 ± 0.02 × 10² | 1:9.2 | 38 | 38 |
| BHR-61 | ALRLQV | 9.95 ± 0.06 × 10² | 1.82 ± 0.03 × 10² | 5.5:1 | 12 | 2 |
| BHR-62 | ACQLNs | 2.15 ± 0.02 × 10³ | 2.15 ± 0.02 × 10³ | 1:1 | 26 | 26 |
| BHR-63 | VLGFQs | 6.42 ± 0.01 × 10³ | 2.29 ± 0.01 × 10² | 1:28 | 77 | 77 |
| BHR-64 | AERLNL | 1.78 ± 0.01 × 10³ | 2.20 ± 0.02 × 10² | 8.1:1 | 21 | 3 |
| BHR-65 | AERLQs | 1.65 ± 0.01 × 10³ | 2.83 ± 0.03 × 10² | 5.8:1 | 20 | 3 |
| BHR-66 | VVRLSL | 1.10 ± 0.01 × 10⁴ | 9.69 ± 0.02 × 10² | 11:1 | 130 | 12 |
| BHR-67 | iCQFNs | 9.2 ± 0.2 × 10⁴ | 1.03 ± 0.01 × 10⁴ | 1:8.9* | 1100 | 1100 |
| BHR-68 | CARLTL | 8.8 ± 0.2 × 10³ | 4.07 ± 0.04 × 10² | 22:1 | 110 | 5 |
| BHR-69 | iCQFis | 1.41 ± 0.03 × 10⁵ | 1.28 ± 0.01 × 10⁴ | 1:11* | 1700 | 1700 |
| BHR-70 | CVRLSL | 1.83 ± 0.01 × 10³ | 1.83 ± 0.03 × 10² | 10:1 | 22 | 2 |
| BHR-71 | AVQFNs | 1.33 ± 0.01 × 10⁴ | 1.32 ± 0.01 × 10³ | 1:10 | 160 | 160 |
| BHR-72 | ACGhNs | 9.9 ± 0.2 × 10³ | 7.12 ± 0.04 × 10² | 1:14 | 110 | 110 |
| BHR-73 | AEGhNs | 2.45 ± 0.04 × 10⁴ | 2.15 ± 0.02 × 10³ | 1:11* | 290 | 290 |
| BHR-74 | ACQFNs | 1.03 ± 0.02 × 10⁴ | 1.58 ± 0.01 × 10³ | 1:6.5* | 120 | 120 |
| BHR-75 | AEQFQs | 1.10 ± 0.02 × 10⁴ | 7 ± 1 × 10² | 1:16* | 130 | 130 |
| BHR-76 | AEQYQs | 1.37 ± 0.01 × 10⁴ | 9.2 ± 0.1 × 10² | 1:15 | 170 | 170 |

^{a}Sequence given as single letter amino acid code for residues present at position 106/132/254/257/274/308.
^{b}K₁ is faster phase and K₂ for slower phase from curve fits to 2-exponential.
The stereopreference of variants marked with asterisk were tested by chiral LC/MS. For variants not explicitly tested, preference is assumed to be the same as OMVR.
nd = not determined.
NO = Not Observed

Against VR (FIG. 23C), multiple variants were identified with k_{cat}/Kₘ values in excess of 104 M⁻¹ s⁻¹, with the best being BHR-53 with a k_{cat}/Kₘ of 2×10⁵ M⁻¹ s⁻¹, which is 1809-fold improved over wild-type PTE and is the highest activity variant reported for the hydrolysis racemic of VR (Table 19).(72) Unfortunately, these variants strongly favor the less toxic R_{P}-enantiomer of VR. Against the more toxic S_{P}-enantiomer, multiple variants were also identified with more than a thousand-fold improvement, relative to wild-type PTE. Some, like BHR-39, maintain a preference for the R_{P}-enantiomer, but with a k_{cat}/Kₘ of 1.26×10³ M⁻¹ s⁻¹ for the S_{P}-enantiomer, it is improved 293-fold relative to wild-type PTE. Other variants, like BHR-75, have a strong preference for the S_{P}-enantiomer. With a k_{cat}/Kₘ for the hydrolysis of the S_{P}-enantiomer of VR of 1.3×10⁴ M⁻¹s⁻¹, BHR-75 is improved 3100-fold relative to the wild-type PTE. The best variant tested was BHR-73, which has a k_{cat}/Kₘ of 4×10⁴ and is improved 9300-fold for the S_{P}-enantiomer of VR (Table 19).

### Example 58

Inclusion of Additional Mutations. Previously described variants that are improved for hydrolysis of VR have included multiple mutations both in and away from the active site.(57, 71, 72) No attempt has been made to determine the effect of these mutations on the activity of the enzyme. Five of the more likely additional mutations were selected to determine what effect they might have on the activity of the best S_{P}-VR variants. The mutations C59M, K77A, T173N, Y309W and P342S were incorporated into BHR-73, singly and in combination. These variants were characterized with DEVX, DMVX, malathion, paraoxon, and OMVR (Tables 28-32). All of the single mutations in BHR-73 were found to diminish catalytic activity with DEVX and increase activity with DMVX, with the largest effect being a 2-fold increase in activity with the Y309W mutation (Table 17). All of the variants performed poorly with malathion, while the activity against paraoxon was slightly improved for all but the Y309W variant (Tables 30 and 32). Against OMVR, the mutations C59M and K77A reduced catalytic activity, while the remaining three mutations increased the activity. The two-place combinations of C59M, T173N, and Y209W were constructed. In the BHR-73 background, all combinations were improved for R_{P}-OMVR hydrolysis, although at somewhat less than additive levels.

TABLE 28
Kinetic constants for PTE variants with DEVX.

| | | | | k_{cat}/Kₘ | | Fold |
|---|---|---|---|---|---|---|
| Number | Sequence^{a} | k_{cat} (s⁻¹) | Kₘ (mM) | (M⁻¹ s⁻¹) | | improved |
| BHR-72 | ACGhNs | 8 ± 0.5 | 1.17 ± 0.09 | 6.8 ± 0.7 | × 10³ | 6 |
| BHR-73 | AEGhNs | 2.9 ± 0.4 | 2.5 ± 0.4 | 1.2 ± 0.2 | × 10³ | 1 |
| BHR-74 | ACQFNs | 20 ± 2 | 0.9 ± 0.1 | 2.2 ± 0.3 | × 10⁴ | 18 |
| BHR-75 | AEQFQs | 16 ± 5 | 4 ± 1 | 3.55 ± 0.03 | × 10³ | 3 |
| BHR-73-M | AEGhNs-M | 4.8 ± 0.5 | 2.9 ± 0.4 | 1.7 ± 0.3 | × 10³ | 1 |
| BHR-73-A | AEGhNs-A | 6.3 ± 0.5 | 3.2 ± 0.30 | 1.78 ± 0.01 | × 10³ | 1 |
| BHR-73-N | AEGhNs-N | 10 ± 3 | 7 ± 2 | 1.37 ± 0.01 | × 10³ | 1 |
| BHR-73-W | AEGhNs-W | 20 ± 10 | 20 ± 10 | 1.23 ± 0.01 | × 10³ | 1 |
| BHR-73-S | AEGhNs-S | 6.0 ± 0.7 | 4.0 ± 0.5 | 1.5 ± 0.3 | × 10³ | 1 |
| BHR-72-M | ACGhNs-M | 8.4 ± 0.4 | 1.44 ± 0.09 | 5.8 ± 0.5 | × 10³ | 5 |
| BHR-72-N | ACGhNs-N | 3.5 ± 0.2 | 1.7 ± 0.1 | 2.1 ± 0.2 | × 10³ | 2 |
| BHR-72-W | ACGhNs-W | 12.4 ± 0.9 | 0.76 ± 0.08 | 1.6 ± 0.2 | × 10⁴ | 14 |
| BHR-74-N | ACQFNS-N | 12.9 ± 0.9 | 1.4 ± 0.1 | 9.2 ± 0.9 | × 10³ | 8 |
| BHR-74-W | ACQFNS-W | 18.9 ± 0.5 | 0.41 ± 0.02 | 4.61 ± 0.02 | × 10⁴ | 38 |
| BHR-75-M | AEQFQs-M | nd ± nd | nd ± nd | 2.56 ± 0.02 | × 10³ | 2 |
| BHR-75-W | AEQFQs-W | 40 ± 20 | 11 ± 5 | 3.74 ± 0.03 | × 10³ | 3 |
| BHR-73-MN | AEGhNs-MN | 2.0 ± 0.3 | 1.2 ± 0.2 | 1.49 ± 0.01 | × 10³ | 1 |
| BHR-73-MW | AEGhNs-MW | 5.3 ± 0.3 | 2.7 ± 0.2 | 1.66 ± 0.02 | × 10³ | 1 |
| BHR-73-NW | AEGhNs-NW | 7 ± 1 | 4 ± 1 | 1.45 ± 0.02 | × 10³ | 1 |
| BHR-74-MW | ACQFNs-MW | 13.6 ± 0.5 | 0.41 ± 0.03 | 3.3 ± 0.3 | × 10⁴ | 28 |
| BHR-74-NW | ACQFNs-NW | 13.9 ± 0.4 | 0.31 ± 0.02 | 4.5 ± 0.3 | × 10⁴ | 38 |
| BHR-75-MN | AEQFQs-MN | 50 ± 10 | 13 ± 4 | 3.26 ± 0.02 | × 10³ | 3 |
| BHR-75-MW | AEQFQs-MW | nd ± nd | nd ± nd | 3.89 ± 0.02 | × 10³ | 3 |
| BHR-75-AW | AEQFQs-AW | nd ± nd | nd ± nd | 4.90 ± 0.03 | × 10³ | 4 |
| BHR-72-MNW | ACGhNs-MNW | 6.6 ± 0.8 | 1.4 ± 0.2 | 3.93 ± 0.08 | × 10³ | 3 |
| BHR-73-MNW | AEGhNs-MNW | 10 ± 1 | 4.6 ± 0.7 | 2.00 ± 0.02 | × 10³ | 2 |
| BHR-74-MNW | ACQFQs-MNW | 8.4 ± 0.4 | 0.56 ± 0.04 | 1.5 ± 0.1 | × 10⁴ | 13 |
| BHR-75-MNW | AEQFQs-MNW | 17 ± 4 | 2.7 ± 0.7 | 5.3 ± 0.1 | × 10³ | 4 |

^{a}Sequence given as single letter amino acid code for residues present at position 106/132/254/257/274/308.
Letters following hyphen are; M = C59M, A = K77A, N = T173N, W = Y309W, S = P342S.
nd = not determined.

TABLE 29
Kinetic constants for PTE variants with DMVX.

| | | | | k_{cat}/Kₘ | | Fold |
|---|---|---|---|---|---|---|
| Number | Sequence^{a} | k_{cat} (s⁻¹) | Kₘ (mM) | (M⁻¹ s⁻¹) | | improved |
| BHR-72 | ACGhNs | 0.10 ± 0.01 | 0.30 ± 0.05 | 3.3 ± 0.6 | × 10² | 17 |
| BHR-73 | AEGhNs | nd | nd | 7.24 ± 0.05 | × 10¹ | 4 |
| BHR-74 | ACQFNs | 1.02 ± 0.04 | 0.71 ± 0.05 | 1.4 ± 0.1 | × 10³ | 74 |
| BHR-75 | AEQFQs | nd | nd | 3.88 ± 0.02 | × 10² | 20 |
| BHR-73-M | AEGhNs-M | 0.4 ± 0.1 | 5 ± 1 | 8.57 ± 0.07 | × 10¹ | 5 |
| BHR-73-A | AEGhNs-A | 0.7 ± 0.1 | 5 ± 1 | 1.12 ± 0.01 | × 10² | 6 |
| BHR-73-N | AEGhNs-N | 0.9 ± 0.3 | 8 ± 3 | 1.05 ± 0.01 | × 10² | 6 |
| BHR-73-W | AEGhNs-W | 1.2 ± 0.3 | 7 ± 2 | 1.57 ± 0.02 | × 10² | 8 |
| BHR-73-S | AEGhNs-S | 0.54 ± 0.06 | 4.9 ± 0.6 | 1.1 ± 0.2 | × 10² | 6 |
| BHR-72-M | ACGhNs-M | 0.51 ± 0.03 | 2.1 ± 0.2 | 2.4 ± 0.3 | × 10² | 13 |
| BHR-72-N | ACGhNs-N | nd | nd | 9.1 ± 0.1 | × 10¹ | 5 |
| BHR-72-W | ACGhNs-W | 0.87 ± 0.08 | 1.0 ± 0.1 | 6.78 ± 0.08 | × 10² | 46 |
| BHR-74-N | ACQFNS-N | 0.51 ± 0.02 | 0.71 ± 0.04 | 7.2 ± 0.5 | × 10² | 38 |
| BHR-74-W | ACQFNS-W | 3.7 ± 0.2 | 0.94 ± 0.08 | 3.9 ± 0.4 | × 10³ | 210 |
| BHR-75-M | AEQFQs-M | nd | nd | 2.58 ± 0.02 | × 10² | 14 |
| BHR-75-W | AEQFQs-W | nd | nd | 3.39 ± 0.02 | × 10² | 18 |
| BHR-73-MN | AEGhNs-MN | 0.68 ± 0.06 | 7.1 ± 0.7 | 8.79 ± 0.05 | × 10¹ | 5 |
| BHR-73-MW | AEGhNs-MW | 0.51 ± 0.08 | 4.4 ± 0.7 | 1.03 ± 0.01 | × 10² | 5 |
| BHR-73-NW | AEGhNs-NW | 0.23 ± 0.03 | 1.8 ± 0.3 | 1.3 ± 0.3 | × 10² | 7 |
| BHR-74-MW | ACQFNs-MW | 1.33 ± 0.05 | 0.52 ± 0.04 | 2.6 ± 0.2 | × 10³ | 140 |
| BHR-74-NW | ACQFNs-NW | 2.3 ± 0.1 | 0.50 ± 0.04 | 4.6 ± 0.4 | × 10³ | 240 |
| BHR-75-MN | AEQFQs-MN | 2.3 ± 0.5 | 7 ± 2 | 3.04 ± 0.03 | × 10² | 16 |
| BHR-75-MW | AEQFQs-MW | nd | nd | 2.79 ± 0.04 | × 10² | 15 |
| BHR-75-AW | AEQFQs-AW | 3 ± 0.6 | 5 ± 1 | 5.74 ± 0.02 | × 10² | 30 |
| BHR-72-MNW | ACGhNs-MNW | 0.44 ± 0.03 | 1.6 ± 0.1 | 2.8 ± 0.3 | × 10² | 14 |
| BHR-73-MNW | AEGhNs-MNW | 0.44 ± 0.05 | 2.9 ± 0.4 | 1.37 ± 0.02 | × 10² | 7 |
| BHR-74-MNW | ACQFQs-MNW | 1.44 ± 0.06 | 1.02 ± 0.06 | 1.4 ± 0.1 | × 10³ | 74 |
| BHR-75-MNW | AEQFQs-MNW | 0.88 ± 0.08 | 1.7 ± 0.2 | 5.2 ± 0.8 | × 10² | 27 |

^{a}Sequence given as single letter amino acid code for residues present at position 106/132/254/257/274/308.
Letters following hyphen are; M = C59M, A = K77A, N = T173N, W = Y309W, S = P342S.
nd = not determined

TABLE 30
Kinetic constants for PTE variants with Malathion.

| | | | | k_{cat}/Kₘ | |
|---|---|---|---|---|---|
| Number | Sequence^{a} | k_{cat} (s⁻¹) | Kₘ (mM) | (M⁻¹ s⁻¹) | |
| BHR-72 | ACGhNs | 0.0043 ± 0.0003 | 0.013 ± 0.003 | 3.3 ± 0.8 | × 10² |
| BHR-73 | AEGhNs | 0.0008 ± 0.00006 | 0.016 ± 0.004 | 5 ± 1 | × 10¹ |
| BHR-74 | ACQFNs | 0.0039 ± 0.0002 | 0.001 ± 0.0003 | 4 ± 1 | × 10³ |
| BHR-75 | AEQFQs | 0.0007 ± 0.00009 | 0.11 ± 0.03 | 6 ± 2 | × 10⁰ |
| BHR-73-M | AEGhNs-M | 0.005 ± 0.002 | 1.4 ± 0.6 | 2.70 ± 0.07 | × 10⁰ |
| BHR-73-A | AEGhNs-A | 0.0022 ± 0.0005 | 0.11 ± 0.05 | 2 ± 1 | × 10¹ |
| BHR-73-N | AEGhNs-N | 0.0011 ± 0.0002 | 0.4 ± 0.2 | 3 ± 1 | × 10⁰ |
| BHR-73-W | AEGhNs-W | 0.0011 ± 0.0002 | 0.06 ± 0.02 | 1.8 ± 0.7 | × 10¹ |
| BHR-73-S | AEGhNs-S | 0.0016 ± 0.0004 | 0.11 ± 0.05 | 1.5 ± 0.8 | × 10¹ |
| BHR-72-M | ACGhNs-M | 0.012 ± 0.002 | 0.15 ± 0.06 | 8 ± 3 | × 10¹ |
| BHR-72-N | ACGhNs-N | 0.0072 ± 0.0007 | 0.09 ± 0.02 | 5.0 ± 0.6 | × 10¹ |
| BHR-72-W | ACGhNs-W | 0.02 ± 0.004 | 0.3 ± 0.1 | 7 ± 3 | × 10¹ |
| BHR-74-N | ACQFNS-N | 0.003 ± 0.001 | 1 ± 1 | 3 ± 3 | × 10⁰ |
| BHR-74-W | ACQFNS-W | 0.009 ± 0.002 | 0.7 ± 0.3 | 9.7 ± 0.4 | × 10⁰ |
| BHR-75-M | AEQFQs-M | 0.0004 ± 0.00007 | 0.07 ± 0.03 | 6 ± 3 | × 10⁰ |
| BHR-75-W | AEQFQs-W | 0.0006 ± 0.00008 | 0.28 ± 0.09 | 2.1 ± 0.7 | × 10⁰ |
| BHR-73-MN | AEGhNs-MN | 0.0006 ± 0.00007 | 0.18 ± 0.04 | 3.3 ± 0.8 | × 10⁰ |
| BHR-73-MW | AEGhNs-MW | 0.0016 ± 0.0004 | 0.5 ± 0.2 | 2.0 ± 0.1 | × 10⁰ |
| BHR-73-NW | AEGhNs-NW | nd | nd | 1.1 ± 0.2 | × 10⁰ |
| BHR-74-MW | ACQFNs-MW | 0.007 ± 0.001 | 0.4 ± 0.1 | 1.48 ± 0.03 | × 10¹ |
| BHR-74-NW | ACQFNs-NW | nd | nd | 1.35 ± 0.05 | × 10¹ |
| BHR-75-MN | AEQFQs-MN | nd | nd | 5.0 ± 0.5 | × 10¹ |
| BHR-75-MW | AEQFQs-MW | 0.0014 ± 0.0006 | 0.5 ± 0.3 | 1 ± 1 | × 10⁰ |
| BHR-75-AW | AEQFQs-AW | 0.0005 ± 0.00005 | 0.017 ± 0.006 | 2 ± l | × 10¹ |
| BHR-72-MNW | ACGhNs-MNW | nd | nd | 2.9 ± 0.1 | × 10¹ |
| BHR-73-MNW | AEGhNs-MNW | 0.0009 ± 0.0002 | 0.4 ± 0.1 | 2.3 ± 0.8 | × 10⁰ |
| BHR-74-MNW | ACQFQs-MNW | 0.025 ± 0.005 | 1.8 ± 0.4 | 1 ± 4 | × 10¹ |
| BHR-75-MNW | AEQFQs-MNW | 0.0001 ± 0.00001 | 0.03 ± 0.01 | 3 ± 1 | × 10⁰ |

^{a}Sequence given as single letter amino acid code for residues present at position 106/132/254/257/274/308.
Letters following hyphen are; M = C59M, A = K77A, N = T173N, W = Y309W, S = P342S.
nd = not determined

TABLE 31
Kinetic constants for PTE variants with paraoxon.

| | | | | k_{cat}/Kₘ | |
|---|---|---|---|---|---|
| Number | Sequence^{a} | k_{cat} (s⁻¹) | Kₘ (mM) | (M⁻¹ s⁻¹) | |
| BHR-72 | ACGhNs | 11200 ± 400 | 0.84 ± 0.05 | 1.33 ± 0.09 | × 10⁷ |
| BHR-73 | AEGhNs | 2810 ± 70 | 1.8 ± 0.06 | 1.56 ± 0.06 | × 10⁶ |
| BHR-74 | ACQFNs | 120 ± 2 | 0.006 ± 0.0007 | 2.0 ± 0.2 | × 10⁷ |
| BHR-75 | AEQFQs | 423 ± 4 | 0.124 ± 0.004 | 3.4 ± 0.1 | × 10⁶ |
| BHR-73-M | AEGhNs-M | 4300 ± 500 | 1.4 ± 0.2 | 3.1 ± 0.5 | × 10⁶ |
| BHR-73-A | AEGhNs-A | 8800 ± 300 | 2.9 ± 0.1 | 3.0 ± 0.1 | × 10⁶ |
| BHR-73-N | AEGhNs-N | 2900 ± 100 | 1.22 ± 0.008 | 2.38 ± 0.08 | × 10⁶ |
| BHR-73-W | AEGhNs-W | 2600 ± 200 | 3.1 ± 0.3 | 8 ± 1 | × 10⁵ |
| BHR-73-S | AEGhNs-S | 3500 ± 100 | 1.21 ± 0.06 | 2.9 ± 0.2 | × 10⁶ |
| BHR-72-M | ACGhNs-M | 3400 ± 300 | 1.6 ± 0.2 | 2.1 ± 0.3 | × 10⁶ |
| BHR-72-N | ACGhNs-N | 1430 ± 80 | 0.2 ± 0.03 | 7 ± 1 | × 10⁶ |
| BHR-72-W | ACGhNs-W | 1200 ± 60 | 0.1 ± 0.01 | 1.2 ± 0.1 | × 10⁷ |
| BHR-74-N | ACQFNS-N | 92 ± 3 | 0.011 ± 0.002 | 8.36 ± 0.01 | × 10⁶ |
| BHR-74-W | ACQFNS-W | 100 ± 2 | 0.01 ± 0.0007 | 1.00 ± 0.07 | × 10⁷ |
| BHR-75-M | AEQFQs-M | 627 ± 3 | 0.152 ± 0.002 | 4.13 ± 0.05 | × 10⁶ |
| BHR-75-W | AEQFQs-W | 264 ± 4 | 0.175 ± 0.008 | 1.51 ± 0.07 | × 10⁶ |
| BHR-73-MN | AEGhNs-MN | 1700 ± 200 | 1.2 ± 0.2 | 1.4 ± 0.3 | × 10⁶ |
| BHR-73-MW | AEGhNs-MW | 860 ± 60 | 1.3 ± 0.1 | 6.6 ± 0.7 | × 10⁵ |
| BHR-73-NW | AEGhNs-NW | 500 ± 100 | 3.2 ± 0.9 | 1.43 ± 0.02 | × 10⁵ |
| BHR-74-MW | ACQFNs-MW | 71 ± 2 | 0.016 ± 0.002 | 4.4 ± 0.6 | × 10⁶ |
| BHR-74-NW | ACQFNs-NW | 51.6 ± 0.7 | 0.0055 ± 0.0004 | 9.4 ± 0.7 | × 10⁶ |
| BHR-75-MN | AEQFQs-MN | 750 ± 10 | 0.29 ± 0.01 | 2.6 ± 0.1 | × 10⁶ |
| BHR-75-MW | AEQFQs-MW | 222 ± 6 | 0.26 ± 0.02 | 8.5 ± 0.7 | × 10⁵ |
| BHR-75-AW | AEQFQs-AW | 378 ± 1 | 0.202 ± 0.002 | 1.87 ± 0.01 | × 10⁶ |
| BHR-72-MNW | ACGhNs-MNW | 1900 ± 100 | 0.86 ± 0.07 | 2.2 ± 0.2 | × 10⁶ |
| BHR-73-MNW | AEGhNs-MNW | 390 ± 20 | 1.27 ± 0.09 | 3.1 ± 0.3 | × 10⁵ |
| BHR-74-MNW | ACQFQs-MNW | 72 ± 2 | 0.006 ± 0.001 | 1.2 ± 0.2 | × 10⁷ |
| BHR-75-MNW | AEQFQs-MNW | 212 ± 9 | 0.52 ± 0.04 | 4.1 ± 0.4 | × 10⁵ |

^{a}Sequence given as single letter amino acid code for residues present at position 106/132/254/257/274/308.
Letters following hyphen are; M = C59M, A = K77A, N = T173N, W = Y309W, S = P342S.

TABLE 32
Kinetic constants for PTE variants with OMVR.

| | | k_{cat}/Kₘ₁^{b} | k_{cat}/Kₘ₂^{b} | Ratio | | Fold | R_{P}-Fold |
|---|---|---|---|---|---|---|---|
| Number | Sequence^{a} | (M⁻¹s⁻¹) | (M⁻¹s⁻¹) | S_{P}:R_{P} | Preference | improved | improved |
| BHR-72 | ACGhNs | 1.97 ± 0.01 × 10³ | 1.97 ± 0.01 × 10³ | 1:1 | na | 29 | 290 |
| BHR-73 | AEGhNs | 9.79 ± 0.04 × 10² | 9.79 ± 0.04 × 10² | 1:1 | na | 14 | 140 |
| BHR-74 | ACQFNs | 3.01 ± 0.02 × 10³ | NO | >1:500 | R_{P} | 44 | 440 |
| BHR-75 | AEQFQs | 1.67 ± 0.09 × 10³ | NO | >1:500 | R_{P} | 25 | 250 |
| BHR-73-M | AEGhNs-M | 7.98 ± 0.02 × 10² | 7.98 ± 0.02 × 10² | 1:1 | na | 12 | 120 |
| BHR-73-A | AEGhNs-A | 8.15 ± 0.02 × 10² | 8.15 ± 0.02 × 10² | 1:1 | na | 12 | 120 |
| BHR-73-N | AEGhNs-N | 2.04 ± 0.006 × 10³ | 2.04 ± 0.006 × 10³ | 1:1 | na | 30 | 300 |
| BHR-73-W | AEGhNs-W | 2.58 ± 0.01 × 10³ | 2.58 ± 0.01 × 10³ | 1:1 | na | 38 | 380 |
| BHR-73-S | AEGhNs-S | 1.39 ± 0.004 × 10³ | 1.39 ± 0.004 × 10³ | 1:1 | na | 20 | 200 |
| BHR-72-M | ACGhNs-M | 1.60 ± 0.006 × 10³ | 1.60 ± 0.006 × 10³ | 1:1 | na | 24 | 240 |
| BHR-72-N | ACGhNs-N | 1.83 ± 0.006 × 10³ | 1.83 ± 0.006 × 10³ | 1:1 | na | 27 | 270 |
| BHR-72-W | ACGhNs-W | 3.38 ± 0.01 × 10³ | 3.38 ± 0.01 × 10³ | 1:1 | na | 50 | 500 |
| BHR-74-N | ACQFNS-N | 4.79 ± 0.02 × 10³ | NO | >1:500 | R_{P} | 70 | 700 |
| BHR-74-W | ACQFNS-W | 5.39 ± 0.02 × 10³ | 1.40 ± 0.003 × 10² | 1:46 | R_{P} | 79 | 790 |
| BHR-75-M | AEQFQs-M | 1.51 ± 0.005 × 10³ | NO | >1:500 | R_{P} | 17 | 170 |
| BHR-75-W | AEQFQs-W | 2.26 ± 0.01 × 10³ | 7.64 ± 0.03 × 10¹ | 1:30 | R_{P} | 33 | 330 |
| BHR-73-MN | AEGhNs-MN | 1.48 ± 0.002 × 10³ | 1.48 ± 0.002 × 10³ | 1:1 | na | 22 | 220 |
| BHR-73-MW | AEGhNs-MW | 1.48 ± 0.004 × 10³ | 1.48 ± 0.004 × 10³ | 1:1 | na | 22 | 220 |
| BHR-73-NW | AEGhNs-NW | 2.68 ± 0.007 × 10³ | 2.68 ± 0.007 × 10³ | 1:1 | na | 39 | 390 |
| BHR-74-MW | ACQFNs-MW | 3.82 ± 0.06 × 10³ | 1.01 ± 0.004 × 10² | 1:38 | R_{P} | 56 | 560 |
| BHR-74-NW | ACQFNs-NW | 7.31 ± 0.03 × 10³ | 1.24 ± 0.01 × 10² | 1:59 | R_{P} | 110 | 1100 |
| BHR-75-MN | AEQFQs-MN | 2.9 ± 0.007 × 10³ | 2.09 ± 0.02 × 10¹ | 1:140 | R_{P} | 43 | 430 |
| BHR-75-MW | AEQFQs-MW | 1.45 ± 0.006 × 10³ | 8.68 ± 0.07 × 10¹ | 1:17 | R_{P} | 21 | 210 |
| BHR-75-AW | AEQFQs-AW | 2.98 ± 0.02 × 10³ | 1.03 ± 0.004 × 10² | 1:29 | R_{P} | 44 | 440 |
| BHR-72-MNW | ACGhNs-MNW | 1.78 ± 0.006 × 10³ | 1.78 ± 0.006 × 10³ | 1:1 | na | 26 | 260 |
| BHR-73-MNW | AEGhNs-MNW | 4.27 ± 0.007 × 10³ | 4.27 ± 0.007 × 10³ | 1:1 | na | 63 | 630 |
| BHR-74-MNW | ACQFQs-MNW | 3.04 ± 0.02 × 10³ | 1.76 ± 0.008 × 10² | 1:17 | R_{P} | 45 | 450 |
| BHR-75-MNW | AEQFQs-MNW | 2.26 ± 0.02 × 10³ | 9.43 ± 0.04 × 10¹ | 1:29 | R_{P} | 33 | 330 |

^{a}Sequence given as single letter amino acid code for residues present at position 106/132/254/257/274/308.
Letters following hyphen are;
M = C59M,
A = K77A,
N = T173N,
W = Y309W,
S = P342S..
^{b}K₁ is faster phase and K₂ for slower phase from curve fits to 2-exponential.
NO = Not Observed

The mutations which were beneficial were also incorporated into the variants BHR-72, BHR-74, and BHR-75. The BHR-74 variant with the combination T173N/Y309W showed the best improvement for R_{P}-OMVR (FIG. 23F) with a k_{cat}/Kₘ of 7.3×10³ M⁻¹s⁻¹, which is 1075-fold improved (Table 19). The inclusion of all three mutations yielded less activity than the double or single mutants for all of the templates with the exception of BHR-73, which showed a substantial increase in activity to 4.3×10³ M⁻¹ s⁻¹, which is 628-fold improved relative to the wild-type PTE and shows no stereoselectivity for OMVR (FIG. 23F).

Against VX (FIG. 23B), these variants had up to about a 2-fold improvement, with the exception of the P342S mutation which showed severely diminished activity (Table 33). Similar to what was observed with OMVR (FIG. 23F), the activity of the double mutants tended to be better than the single mutants, but the triple mutant diminished the activity. All of the single mutations were found to be detrimental to catalytic activity against VR (FIG. 23C). While the combinations had less effect, the single mutations resulted in 3-fold or more reduction in activity (Table 34). While there is some suggestion that these variants might increase protein production and/or stability, these data suggest they are detrimental to catalytic activity against VR. However, the triple mutant BHR-73-MNW showed an increase in activity for S_{P}-VR with a k_{cat}/Kₘ of 5.8×10⁴ M⁻¹s⁻¹ that is 13,500-fold improved over wild-type PTE.

TABLE 33
Kinetic constants for PTE variants with VX.

| | | k_{cat}/Kₘ₁^{b} | | k_{cat}/Kₘ₂^{b} | | Ratio^{c} | S_{P}-Fold^{c} |
|---|---|---|---|---|---|---|---|
| Number | Sequence^{a} | (M⁻¹ s⁻¹) | | (M⁻¹ s⁻¹) | | R_{P}:S_{P} | improved |
| BHR-72 | ACGhNs | 9.9 ± 0.2 | × 10³ | 7.12 ± 0.04 | × 10² | 1:14 | 110 |
| BHR-73 | AEGhNs | 2.45 ± 0.04 | × 10⁴ | 2.15 ± 0.02 | × 10³ | 1:11* | 290 |
| BHR-74 | ACQFNs | 1.03 ± 0.02 | × 10⁴ | 1.58 ± 0.01 | × 10³ | 1:6.5* | 120 |
| BHR-75 | AEQFQs | 1.10 ± 0.02 | × 10⁴ | 7 ± 1 | × 10² | 1:16* | 130 |
| BHR-73-M | AEGhNs-M | 1.21 ± 0.02 | × 10⁴ | 1.53 ± 0.01 | × 10³ | 1:8 | 140 |
| BHR-73-A | AEGhNs-A | 1.94 ± 0.01 | × 10⁴ | 2.69 ± 0.01 | × 10³ | 1:7 | 230 |
| BHR-73-N | AEGhNs-N | 1.55 ± 0.03 | × 10⁴ | 3.24 ± 0.05 | × 10³ | 1:5 | 190 |
| BHR-73-W | AEGhNs-W | 1.62 ± 0.02 | × 10⁴ | 1.89 ± 0.01 | × 10³ | 1:9 | 190 |
| BHR-73-S | AEGhNs-S | 1.38 ± 0.02 | × 10³ | 1.38 ± 0.02 | × 10³ | 1:1 | 16 |
| BHR-72-M | ACGhNs-M | 6.35 ± 0.04 | × 10³ | 9.46 ± 0.1 | × 10² | 1:7 | 76 |
| BHR-72-N | ACGhNs-N | 1.05 ± 0.01 | × 10⁴ | 1.01 ± 0.01 | × 10³ | 1:10 | 130 |
| BHR-72-W | ACGhNs-W | 1.37 ± 0.01 | × 10⁴ | 7.07 ± 0.01 | × 10² | 1:19 | 160 |
| BHR-74-N | ACQFNS-N | 1.52 ± 0.01 | × 10⁴ | 2.11 ± 0.01 | × 10³ | 1:7 | 180 |
| BHR-74-W | ACQFNS-W | 2.62 ± 0.07 | × 10⁴ | 2.34 ± 0.03 | × 10³ | 1:11 | 310 |
| BHR-75-M | AEQFQs-M | 1.40 ± 0.01 | × 10⁴ | 1.84 ± 0.01 | × 10³ | 1:8 | 170 |
| BHR-75-W | AEQFQs-W | 2.12 ± 0.04 | × 10⁴ | 2.20 ± 0.02 | × 10³ | 1:10 | 250 |
| BHR-73-MN | AEGhNs-MN | 2.95 ± 0.07 | × 10⁴ | 3.61 ± 0.05 | × 10³ | 1:8 | 350 |
| BHR-73-MW | AEGhNs-MW | 1.58 ± 0.03 | × 10⁴ | 1.31 ± 0.01 | × 10³ | 1:12 | 190 |
| BHR-73-NW | AEGhNs-NW | 3.39 ± 0.2 | × 10⁴ | 3.14 ± 0.07 | × 10³ | 1:11 | 400 |
| BHR-74-MW | ACQFNs-MW | 3.63 ± 0.07 | × 10⁴ | 4.36 ± 0.02 | × 10³ | 1:8 | 430 |
| BHR-74-NW | ACQFNs-NW | 3.61 ± 0.08 | × 10⁴ | 4.16 ± 0.03 | × 10³ | 1:9 | 430 |
| BHR-75-MN | AEQFQs-MN | 1.68 ± 0.02 | × 10⁴ | 2.08 ± 0.01 | × 10³ | 1:8 | 200 |
| BHR-75-MW | AEQFQs-MW | 3.28 ± 0.04 | × 10⁴ | 3.24 ± 0.01 | × 10³ | 1:10 | 390 |
| BHR-75-AW | AEQFQs-AW | 3.41 ± 0.03 | × 10⁴ | 3.40 ± 0.02 | × 10³ | 1:10 | 400 |
| BHR-72-MNW | ACGhNs-MNW | 2.73 ± 0.01 | × 10⁴ | 1.74 ± 0.01 | × 10³ | 1:16 | 330 |
| BHR-73-MNW | AEGhNs-MNW | 1.55 ± 0.03 | × 10⁴ | 1.95 ± 0.02 | × 10³ | 1:8* | 190 |
| BHR-74-MNW | ACQFQs-MNW | 2.5 ± 0.1 | × 10⁴ | 3.63 ± 0.05 | × 10³ | 1:7 | 300 |
| BHR-75-MNW | AEQFQs-MNW | 1.78 ± 0.04 | × 10⁴ | 2.43 ± 0.02 | × 10³ | 1:7 | 210 |

^{a}Sequence given as single letter amino acid code for residues present at position 106/132/254/257/274/308.
Letters following hyphen are; M = C59M, A = K77A, N = T173N, W = Y309W, S = P342S.
^{b}K₁ is faster phase and K₂ for slower phase from curve fits to 2-exponential.
^{c}Stereopreference of variants marked with asterisk was tested by chiral LC/MS. Stereopreference when not explicitly determined is assumed to be the same as OMVR.

TABLE 34
Kinetic constants for PTE variants with VR.

| | | k_{cat}/Kₘ₁^{b} | k_{cat}/Kₘ₂^{b} | Ratio^{c} | Fold | S_{P}-Fold^{c} |
|---|---|---|---|---|---|---|
| Number | Sequence^{a} | (M⁻¹s⁻¹) | (M⁻¹s⁻¹) | S_{P}:R_{P} | improved | improved |
| BHR-72 | ACGhNs | 1.96 ± 0.03 × 10⁴ | 7.99 ± 0.02 × 10³ | 1:2.5* | 180 | 4600 |
| BHR-73 | AEGhNs | 4.00 ± 0.06 × 10⁴ | 7.25 ± 0.08 × 10³ | 1:5.5* | 360 | 9300 |
| BHR-74 | ACQFNs | 4.93 ± 0.09 × 10³ | 1.12 ± 0.01 × 10² | 44:1: | 45 | 1100 |
| BHR-75 | AEQFQs | 1.33 ± 0.007 × 10⁴ | 1.14 ± 0.06 × 10² | 1:120* | 120 | 3100 |
| BHR-73-M | AEGhNs-M | 6.7 ± 0.3 × 10³ | 1.25 ± 0.03 × 10³ | 1:6 | 61 | 1600 |
| BHR-73-A | AEGhNs-A | 8.32 ± 0.06 × 10³ | 1.51 ± 0.01 × 10³ | 1:6 | 76 | 1900 |
| BHR-73-N | AEGhNs-N | 1.17 ± 0.03 × 10⁴ | 2.20 ± 0.02 × 10³ | 1:5.3 | 110 | 2700 |
| BHR-73-W | AEGhNs-W | 1.31 ± 0.04 × 10⁴ | 2.99 ± 0.07 × 10³ | 1:4.4 | 120 | 3000 |
| BHR-73-S | AEGhNs-S | 7.5 ± 0.1 × 10³ | 1.46 ± 0.02 × 10³ | 1:5.1 | 68 | 1700 |
| BHR-72-M | ACGhNs-M | 3.3 ± 0.1 × 10³ | 1.3 ± 0.2 × 10³ | 1:12.7 | 30 | 770 |
| BHR-72-N | ACGhNs-N | 3.30 ± 0.02 × 10³ | 3.3 ± 0.02 × 10³ | 1:1 | 30 | 770 |
| BHR-72-W | ACGhNs-W | 6.0 ± 0.1 × 10³ | 1.5 ± 0.1 × 10³ | 1:4 | 55 | 1400 |
| BHR-74-N | ACQFNS-N | 3.12 ± 0.02 × 10³ | 7.42 ± 0.05 × 10¹ | 1:42 | 28 | 730 |
| BHR-74-W | ACQFNS-W | 3.15 ± 0.01 × 10³ | 1.17 ± 0.07 × 10² | 1:27 | 29 | 730 |
| BHR-75-M | AEQFQs-M | 1.37 ± 0.04 × 10³ | NO | >1:500 | 12 | 320 |
| BHR-75-W | AEQFQs-W | 3.22 ± 0.05 × 10³ | 1.19 ± 0.02 × 10² | 1:27 | 29 | 750 |
| BHR-73-MN | AEGhNs-MN | 9.7 ± 0.2 × 10³ | 1.36 ± 0.01 × 10³ | 1:7 | 88 | 2300 |
| BHR-73-MW | AEGhNs-MW | 7.5 ± 0.2 × 10³ | 1.24 ± 0.01 × 10³ | 1:6 | 68 | 1700 |
| BHR-73-NW | AEGhNs-NW | 1.1 ± 0.2 × 10⁴ | 2.9 ± 0.2 × 10³ | 1:4 | 100 | 260 |
| BHR-74-MW | ACQFNs-MW | 3.48 ± 0.04 × 10³ | 1.92 ± 0.08 × 10² | 1:18 | 32 | 810 |
| BHR-74-NW | ACQFNs-NW | 3.30 ± 0.02 × 10³ | 1.28 ± 0.08 × 10² | 1:26 | 30 | 770 |
| BHR-75-MN | AEQFQs-MN | 2.47 ± 0.03 × 10³ | 3.88 ± 0.06 × 10¹ | 1:64 | 22 | 570 |
| BHR-75-MW | AEQFQs-MW | 3.84 ± 0.03 × 10³ | 1.37 ± 0.01 × 10² | 1:28 | 35 | 890 |
| BHR-75-AW | AEQFQs-AW | 5.59 ± 0.06 × 10³ | 2.11 ± 0.03 × 10² | 1:27 | 51 | 1300 |
| BHR-72-MNW | ACGhNs-MNW | 5.50 ± 0.09 × 10³ | 1.44 ± 0.05 × 10³ | 1:4 | 50 | 1300 |
| BHR-73-MNW | AEGhNs-MNW | 5.79 ± 0.07 × 10⁴ | 1.13 ± 0.01 × 10⁴ | 1:5* | 530 | 13500 |
| BHR-74-MNW | ACQFQs-MNW | 3.11 ± 0.09 × 10³ | 2.22 ± 0.02 × 10² | 1:14 | 28 | 720 |
| BHR-75-MNW | AEQFQs-MNW | 4.17 ± 0.09 × 10³ | 2.08 ± 0.01 × 10² | 1:20 | 38 | 970 |

^{a}Sequence given as single letter amino acid code for residues present at position 106/132/254/257/274/308.
Letters following hyphen are;
M = C59M,
A = K77A,
N = T173N,
W = Y309W,
S = P342S..
^{b}K₁ is faster phase and K₂ for slower phase from curve fits to 2-exponential.
^{c}Stereopreference of variants marked with asterisk was tested for stereopreference by chiral LC/MS. Stereopreference when not explicitly determined is assumed to be the same as OMVR.

### Example 59

Sequence Analysis of Selected Variants. Typically, enzyme evolution experiments rely on either incomplete screening of large undefined libraries (the identity of variants being screened is unknown),(71, 76, 77) or small “smart” libraries where complete coverage can be achieved.(79, 82) In the former case, the assumption is made that each position modified is independent of all others and hence the complexity of the library is additive rather than exponential and very few variants need to be screened to identify all of the beneficial mutations. Consequently, no information on the effects of individual mutations is gained, and the presence of synergy will vastly diminish the usefulness of the library. In the small “smart” approach, the library size is drastically limited by experimental considerations. The “small smart” approach has been combined with a defined library approach (the identity of variants is known prior to screening) to achieve quantitative data on the effects of individual mutations, but if a significant number of variants are inactive no information can be gained from the library.(88)

The total unidentified library of 28,800 variants was screened against 5 different substrates yielding a vast data set, which provides ample opportunity to analyze the library as a whole. A total of 309 colonies were selected for DNA sequencing based on the activity observed in the screens. Of these, 309 colonies, 241 unique variants were identified. The library allowed six amino acids at positions 106 and 254, eight amino acids at position 132, five amino acids at positions 257 and 274, and four amino acids at position 308. Among the 241 variants identified, all of the possible substitutions were observed with a minimum of 3 replicates. The number of times each amino acid was found and the percentage of variants containing that mutation are presented in Table 35. While all amino acids were found, numerous substitutions were found to be under-represented (Poisson <0.05), while others were over-represented (Poisson >0.95) based on expected values for random selection. Some of these effects can be explained from previous information. For example, it was found that glycine at position 106 can lead to unstable variants. Other residues are harder to explain, for example, cysteine at position 132 was unexpectedly over-represented.

Because of the large amount of data collected, sequence analysis of the library has yielded valuable information that can be used to direct and simplify future efforts. For the current library there are a total of 15 two-site combinations possible. For the two-site combinations, minimal heat maps were constructed (FIG. 16). Overall, the trends follow the general observations when single substitutions are considered, although there are some combinations that are required, while others are forbidden. At positions 106-132 the Cys-Cys and Ala-Phe combinations are absent. In the case of the Cys-Cys combination, the residues 106 and 132 are in close proximity to one another in the tertiary structure of PTE, so disulfide bond formation may explain the absence of this combination. At positions 132-254, Phe-Gly, and Phe-Ser are absent. For positions 132-257 Ala-Cys is absent. In the 106-257 Cys-Cys is missing. In the 254-274 combinations, Ser-Ile is missing. The 254-257 combinations demonstrate that Cys at 257 requires Arg at 254 and Asn at 254 requires Trp at 257. Similarly, Ser at position 254 does not appear with either Ile at 274 or Phe at 132.

In addition to the information gained on allowed combinations of amino acids our data yields important sequence information on specificity for a given substrate. A subset of the variants selected for sequencing were selected based on activity with only a single substrate. The amino acid composition of these variants differed significantly from that of the general library (Table 36). The variants selected for malathion and DMVX stand out in particular. Phenylalanine at position 132 was found to be a strong marker for malathion activity, while the 254/257 combination of Arg-Trp was found in variants which are highly specific for malathion hydrolysis. The 254/257 combination of Gly-Trp was found to render highly specific activity for DMVX. Variants selected for R_{P}-OMVR hydrolysis represent a somewhat broader class of variants in that they were selected without consideration of other activity. The sequence composition of this set, however, differed significantly from the library as a whole. In particular, Ala or Gly at position 106 was much more likely in variants selected for R_{P}-OMVR hydrolysis. Similarly, glutamate at position 132 was a common mutation, as was glycine at position 254 and serine at position 308.

TABLE 36
Mutations found in substrate specific variants.

| Substrate | Mutations | Identifiers |
|---|---|---|
| Malathion | H254R/H257W | ⅗ are malathion specific; ⅖ are |
| | | malathion active with low DMVX activity. |
| | F132 | 74% of variants with Phe at 132 are |
| | | malathion specific. |
| DMVX | H254G/H257W | ⅘ are DMVX specific; ⅕ has DMVX |
| | | activity and low DEVX activity. |
| R_{P}-OMVR | I106A | Appears in 62% of R_{P}-OMVR active |
| | | variants. |
| | I106G | 2/4 occurrence were selected for R_{P}- |
| | | OMVR. |
| | F132E | 50% of F132E variants were selected |
| | | for R_{P}-OMVR. |
| | H254G | Occurs with twice the frequency as in the |
| | | library in general |
| | S308 | Found in 60% of variants selected for |
| | | R_{P}-OMVR |

### Example 60

Synergistic and Epistatic Effects. In typical enzymatic evolution experiments, synergistic effects are not taken into account. These effects are either assumed to be non-existent for large libraries with limited screening, or there is insufficient data to determine synergy in small libraries.(71, 76, 79, 81, 88) The large number of variants identified in this study provided an opportunity to examine the extent of synergy in the PTE system. The sequenced variants were sorted into pairs that only differed by one amino acid and then further separated by specific mutations. In the absence of any synergistic effects, a given substitution of one amino acid to another should have the same effect on activity regardless of the background sequence in which it appeared. The confidence level for significance in activity measurements was determined to be approximately a 2-fold change. In order to define synergy as conservatively as possible, the presence of synergy was defined as a given amino acid substitution resulting in both a greater than 2-fold increase in activity and a greater than 2-fold decrease in activity for a given substrate depending on the background in which the mutation took place. Even with this very limited definition of synergistic effects, between 50% and 80% of the variants contained a mutation that displayed a synergistic effect (Table 37). These effects were also found to be very selective for individual substrates and demonstrate the importance of being able to take synergistic effects into account in library design.

TABLE 37
Synergistic effects seen in
variants that differ by only one amino
acid.

| | % | | |
|---|---|---|---|
| Position | Variants | % Reactions | N^{a} |
| 106 | 71% | 18.2% | 52 |
| 132 | 80% | 10.5% | 39 |
| 254 | 67% | 27.5% | 55 |
| 257 | 63% | 24.4% | 59 |
| 274 | 70% | 36.4% | 112 |
| 308* | 50% | 20.0% | 17 |

*Data was obtained for only two variations at position 308.
^{a}N is number of variants that have the same change at a position in different backgrounds.

### Example 61

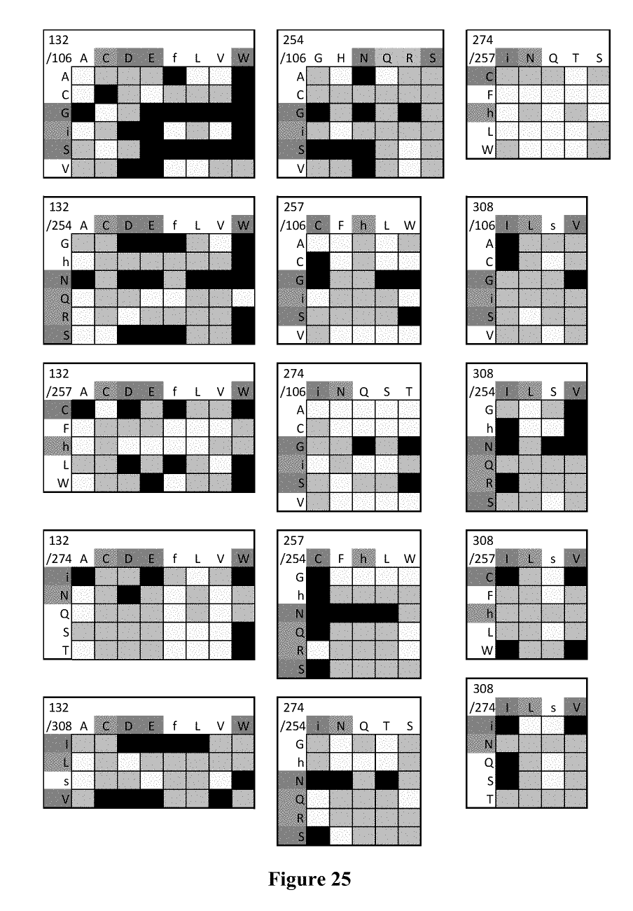

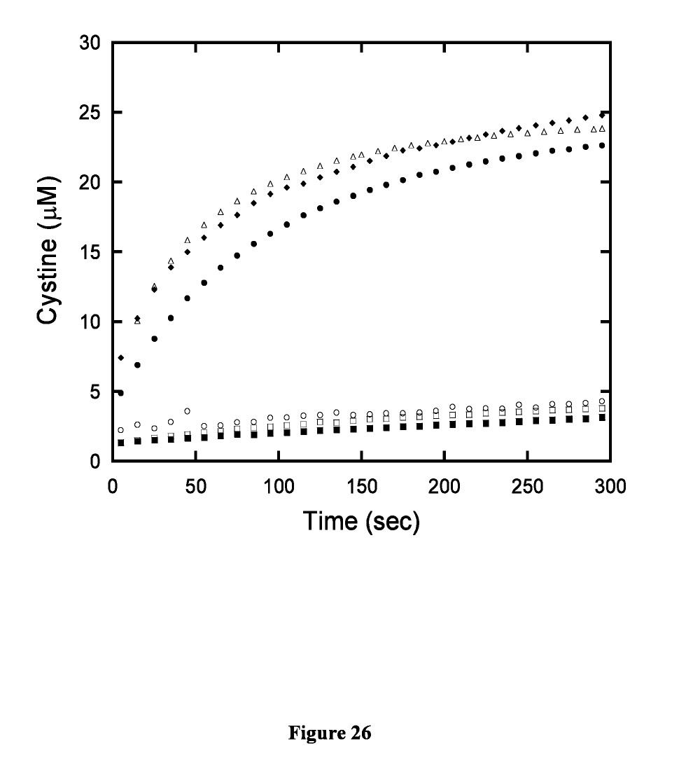

The Effects of Synergy on Library Variants. As expressed in eq. 10, if synergy were absent or minimal then the prediction of the best variants would be straightforward and the top variants for each substrate would be expected to have similar sequences. The variants identified here are presented as a sequence similarity network in FIG. 24 with the highest activities highlighted. A total of 35 of the variants identified are separated by two amino acid substitutions from all others. Among the top-25 variants for OMVR there are two related sets of sequences separated by two mutations and five of the 25 are separated from every other by two or more mutations. The case with malathion is even more extreme, with one closely related set and 8 individual mutants separated from every other by at least two amino acid changes. While it is clear that many of the highest activity variants for a given substrate closely cluster around particular sets of resides, more diverse variants with high activity are seen. This tendency is even more pronounced when considering the activity towards compounds that were not included in the original screen. With both S_{P}-VX (FIG. 23B) and S_{P}-VR (FIG. 23C) there is little obvious clustering of activity seen. These data clearly demonstrate that simplistic linear attempts to achieve improved variants are likely to miss the most improved variants due to the presence of synergistic and epistatic effects.

The synergistic and epistatic effects demonstrated in PTE also severely limit the ability to make straightforward predictions of activity based on the effects of single mutations. When the top-25 variants for DEVX were predicted using the activity coefficients for DEVX, only one of the variants was actually identified in the top-25 variants for DEVX. Six of the top-25 variants actually identified by screening contain alanine at position 106, which is predicted to be the second worst possible amino acid for this position. Similarly, the best variant identified for DMVX hydrolysis was BHR-4, which is 479-fold improved by 3 mutations from wild-type PTE. However, two of the mutations were required to even have detectable activity. BHR-45 was the second-best variant identified for DMVX and is improved 458-fold by 6 mutations from wild type PTE. An evolutionary pathway for this improvement can be predicted from the identified variants, but there is an apparent loss of activity on the addition of the fourth mutation.

### Example 62

Use of Analog Activity Profiles to Identify Enhanced Variants. While synergistic and epistatic effects are typically ignored in enzyme evolution studies, the data presented here demonstrates that with PTE, synergistic effects dominate the evolutionary landscape. The presence of these synergistic and epistatic effects, which are substrate specific effects, are further complicated by the frequent need to use analogs in high-throughput screening. While the use of analogs is often required to allow screening of large numbers of variants, ultimately the activity being screened for is not the activity desired. As the activity of the evolved variants gets higher the specificity will naturally increase as well, making analog data less and less applicable.(92) The problems of this effect can be overcome, as well as dramatically increasing the value of a library, by using an analog activity profile (AAP) approach presented here. In this approach, a library is screened for activity with a relevant set of analogs rather than a single analog. A limited set of library variants of potential interest is selected, purified, and characterized with the substrates of interest. The profile of activity with the analogs is then determined for variants showing substantial improvement against the target compound. Because the library has been screened with multiple substrates, each variant will have a unique activity profile. This profile can then be used to query the library for additional potential variants.

As a proof of concept, S_{P}-VX (FIG. 23B) and racemic VX were used as target compounds. The purified variants BHR-1 through BHR-75 were ranked for activity against S_{P}-VX and racemic VX. The variants ranked 2-6 for S_{P}-VX (FIG. 23B), and with the desired stereoselectivity for OMVR, were used to create activity profiles using the percent rank of each variant with each analog from the screen. Using the profiles for each variant the sequenced variants from the library were searched for variants displaying similar activity profiles. Using a stringency of ±20% activity to search the sequenced variants from the library, returned 16 of the 25 most active variants for S_{P}-VX. BHR-23, the best variant identified for S_{P}-VX, was correctly identified in the search. More significantly, all of the variants returned from the search were improved for S_{P}-VX. Similar results were obtained with searching based on maximal activity with VX and the variants ranked 2-6 used to create the activity profile. In this case, the stringency was set to ±30% and the most active variant, BHR-23, as well as 16 of the 25 most active variants, were identified. All variants identified were improved for VX hydrolysis with the least active variant found to be 38-fold improved.

### Example 63

Sequence Analysis of Improved Variants for V-agents. The top variants for S_{P}-VX (FIG. 23B) (BHR-4, BHR-23, and BHR-69) are widely divergent in sequence as can be seen in the sequence similarity network (FIG. 24). The best variant, BHR-23, contains a critical mutation F132V. Substitution to a cysteine, as seen in BHR-7, results in approximately 20-fold less activity. The second most active variant BHR-4 appears to have a critical mutation of S308L. When this mutation is removed in BHR-26 the activity falls approximately an order of magnitude.

Stereochemistry puts larger constraints on the activity against S_{P}-VR (FIG. 23C). Nearly all of the variants with substantial improvements against S_{P}-VR (FIG. 23C) have the mutation I106A and the serine at position 308 is conserved. As can be seen for the sequence similarity network, the three best variants identified in the screen for S_{P}-VR (FIG. 23C) are diverse in sequence (BHR-45, BHR-52, BHR-75), but do correlate to high activity with R_{P}-OMVR. Interestingly, the activity of these variants, as well as that of the more improved variants of BHR-73, display similar activity for both S_{P}-VX and S_{P}-VR, suggesting that these variants could serve as a single treatment for the toxicity of both V-agents.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations can be applied to the compositions and methods and in the steps or in the sequence of steps of the methods described herein without departing from the concept, spirit and scope of the disclosure. More specifically, it will be apparent that certain agents which are both chemically and physiologically related can be substituted for the agents described herein while the same or similar results would be achieved. All such similar substitutes and modifications apparent to those skilled in the art are deemed to be within the spirit, scope and concept of the disclosure as defined by the appended claims.

### REFERENCES

1. Maxwell, D. M.; Brecht, K. M.; Koplovitz, I.; Sweeney, R. E. Mol. Toxicol. 2006, 80, 756.
2. Leikin, J. B.; Thomas, R. G.; Walter, F. G.; Klein, R.; Meislin, H. W. Crit. Care. Med. 2002, 30, 2346.
3. Columbus, I.; Waysbort, D.; Marcovitch, I.; Yehezkel, L.; Mizrahi, D. M. Environ. Sci. Technol. 2012, 46, 3921.
4. Yang, Y. C. Chem. Ind. 1995, 9, 334.
5. Yang, Y. C. Accounts Chem. Res. 1999, 32, 109.
6. Saxina, A.; Sun, W.; Fedorko, J. M.; Koplovitz, I.; Doctor, B. P. Biochem. Pharmacol. 2011, 81, 164.
7. Ashani, Y.; Pistinner, S. Toxicol. Sci. 2004, 77, 358.
8. Saxena, A.; Tipparaju, P.; Luo, C.; Doctor, B. P. Process Biochem. 2010, 45, 1313.
9. Cheng, T.-C.; Liu, L.; Wang, B.; Wu, J.; DeFrank, J. J.; Anderson, D. M.; Rastogi, V. K.; Hamilton, A. B. J. Ind. Microbiol. Biotechnol. 1997, 18, 49.
10. Melzer, M.; Chen, J. C.-H.; Heidenreich, A; Gab, J; Koller, M; Kehe, K; Blum, M.-M. J. Am. Chem. Soc. 2009, 131, 17226
11. Kirby, S. D.; Norris, J. R.; Smith, J. R.; Bahnson, B. J.; Cerasoli, D. M. Chem. Biol. Interact. 2012, 203, 181.
12. Gupta, R. D.; Goldsmith, M.; Ashani, Y.; Simo, Y.; Mullokandov, G.; Bar, H.; Ben-David, M.; Leader, H.; Margalit, R.; Silman, I.; Sussman, J. L.; Tawfik, D. S. Nat. Chem. Biol. 2011, 7, 120.
13. Tsai, P.-C.; Fox, N.; Bigley, A. N.; Harvey, S. P.; Barondeau, D. P.; Raushel, F. M. Biochemistry 2012, 51, 6463.
14. Lai, K.; Grimsley, J. K.; Kuhlmann, B. D.; Scapozza, L.; Harvey, S. P.; DeFrank, J. J.; Kolakowski, J. E.; Wild, J. R. Chimia 1996, 50, 430.
15. Caldwell, S. R.; Newcomb, J. R.; Schlecht, K. A.; Raushel, F. M. Biochemistry 1991, 30, 7438.
16. Benschop, H. P.; De Jong, L. P. A. Accounts Chem. Res. 1988, 21, 368.
17. Ordentlich, A.; Barak, D.; Sod-Moriah, G.; Kaplan, D.; Mizrahi, D.; Segall, Y.; Kronman, C.; Karton, Y.; Lazar, A.; Marcus, D.; Velan, B.; Shafferman, A. Biochemistry 2004, 43, 11255.
18. Tsai, P. C.; Bigley, A, N.; Li, Y.; Ghanem, E.; Cadieux, C. L.; Kasten, S. A.; Reeves, T. E.; Cerasoli, D. M.; Raushel, F. M. Biochemistry 2010, 49, 7978.
19. Rastogi, V. K.; DeFrank, J. J.; Cheng, T.; Wild, J. R. Biochem. Bioph. Res. Co. 1997, 241, 294.
20. Hong, S.-B.; Raushel, F. M. Biochemistry 1996, 35, 10904.
21. Vanhooke, J. L.; Benning, M. M.; Raushel, F. M.; Holden, H. M. Biochemistry 1996, 35, 6020.
22. Chen-Goodspeed, M.; Sogorb, M. A.; Wu, F.; Hong, S.-B.; Raushel, F. M. Biochemistry 2001, 40, 1325.
23. Afriat-Jurnou, L; Jackson, C. J.; Tawfik, D. S. Biochemistry, 2012, 51, 6047.
24. Reeves, T. E.; Wales, M. E.; Grimsley, J. K.; Li, P.; Cerasoli, D. M.; Wild, J. R. Protein Eng. Des. Sel. 2008, 21, 405.
25. Schofield, D. A.; DiNovo, A. A. J. Appl. Microbiol. 2010, 109, 548
26. Horton, R. M.; Hunt, H. D.; Ho, S. N.; Pullen, J. K.; Pease, L. R. Gene 1989 77, 61
27. Goldsmith, M.; Kiss, C.; Bradbury, A. R. M.; Tawfik, D. S. Protein Eng. Del. Sel. 2007, 20, 315.
28. Cho, C. M.; Mulchandani, A.; Chen, W. Protein Eng. Del. Sel. 2006, 19, 99.
29. Roodveldt, C.; Tawfik, D. S. Protein Eng. Des. Sel. 2005, 18, 51.
30. Aubert, S. D.; Li, Y.; Raushel, F. M. Biochemistry 2004, 43, 5707.
31. Shaka, A. J.; Keeler, J.; Freeman, J. R. J. Magn. Reson. 1983, 53, 313.
32. Batley, M.; Redmond, J. W. J. Magn. Reson. 1982, 49, 172.
33. Benschop, H. P., and De Jong, L. P. A. (1988) Nerve agent stereoisomers: analysis, isolation and toxicology, Acc. Chem. Res. 21, 368-374.
34. Rosman, Y., Eisenkraft, A., Milk, N., Shiyovich, A., Ophir, N., Shrot, S., Kreiss, Y., and Kassirer, M. (2014) Lessons learned from the Syrian sarin attack: Evaluation of a clinical syndrome through social media, Ann. Intern. Med. 160, 644-648.
35. Leikin, J. B., Thomas, R. G., Walter, F. G., Klein, R., and Meislin, H. W. (2002) A review of nerve agent exposure for the critical care physician, Crit. Care Med. 30, 2346-2354.
36. Columbus, I., Waysbort, D., Marcovitch, I., Yehezkel, L., and Mizrahi, D. M. (2012) VX fate on common matrices: evaporation versus degradation, Environ. Sci. Technol. 46, 3921-3927.
37. Bigley, A. N., Xu, C., Henderson, T. J., Harvey, S. P., and Raushel, F. M. (2013) Enzymatic neutralization of the chemical warfare agent VX: evolution of phosphotriesterase for phosphorothiolate hydrolysis, J. Am. Chem. Soc. 135, 10426-10432.
38. Tsai, P. C., Fox, N., Bigley, A. N., Harvey, S. P., Barondeau, D. P., and Raushel, F. M. (2012) Enzymes for the homeland defense: Optimizing phosphotriesterase for the hydrolysis of organophosphate nerve agents, Biochemistry 51, 6463-6475.
39. Tsai, P. C., Bigley, A., Li, Y., Ghanem, E., Cadieux, C. L., Kasten, S. A., Reeves, T. E., Cerasoli, D. M., and Raushel, F. M. (2010) Stereoselective hydrolysis of organophosphate nerve agents by the bacterial phosphotriesterase, Biochemistry 49, 7978-7987.
40. Mee-Hie Cho, C., Mulchandani, A., and Chen, W. (2006) Functional analysis of organophosphorus hydrolase variants with high degradation activity towards organophosphate pesticides, Protein Eng., Des. Sel. 19, 99-105.
41. Roodveldt, C., and Tawfik, D. S. (2005) Directed evolution of phosphotriesterase from Pseudomonas diminuta for heterologous expression in Escherichia coli results in stabilization of the metal-free state, Protein Eng., Des. Sel. 18, 51-58.
42. Rastogi, V. K., DeFrank, J. J., Cheng, T. C., and Wild, J. R. (1997) Enzymatic hydrolysis of Russian-VX by organophosphorus hydrolase, Biochem. Biophys. Res. Commun. 241, 294-296.
43. Cherny, I., Greisen, P., Jr., Ashani, Y., Khare, S. D., Oberdorfer, G., Leader, H., Baker, D., and Tawfik, D. S. (2013) Engineering V-type nerve agents detoxifying enzymes using computationally focused libraries, ACS chem. Biol. 8, 2394-2403.
44. Amitai, G., Ashani, Y., Grunfeld, Y., Kalir, A., and Cohen, S. (1976) Synthesis and properties of 2-S—(N,N-dialkylamino)ethyl)thio-1,3,2-dioxaphosphorinane 2-oxide and of the corresponding quaternary derivatives as potential nontoxic antiglaucoma agents, J. Med. Chem. 19, 810-813.
45. Michaelis, L., and Menten, M. L. (1913) Die kinetik der invertinwirkung, Biochem. Z. 49, 333-369.
46. Otwinowski Z, M. W. (1997) Processing of X-ray diffraction data collected in oscillation mode, Methods Enzymol. 276A, 307-326.
47. Adams, P. D., Afonine, P. V., Bunkoczi, G., Chen, V. B., Davis, I. W., Echols, N., Headd, J. J., Hung, L.-W., Kapral, G. J., Grosse-Kunstleve, R. W., McCoy, A. J., Moriarty, N. W., Oeffner, R., Read, R. J., Richardson, D. C., Richardson, J. S., Terwilliger, T. C., and Zwart, P. H. (2010) PHENIX: a comprehensive Python-based system for macromolecular structure solution, Acta Crystallogr., Sect. D: Biol. Crystallogr. 66, 213-221.
48. Emsley, P., and Cowtan, K. (2004) Coot: model-building tools for molecular graphics, Acta Crystallogr., Sect. D: Biol. Crystallogr. 60, 2126-2132.
49. Trott, O., and Olson, A. J. (2010) AutoDock Vina: Improving the speed and accuracy of docking with a new scoring function, efficient optimization, and multithreading, J. Comput. Chem. 31, 455-461.
50. Chen-Goodspeed, M., Sogorb, M. A., Wu, F., Hong, S. B., and Raushel, F. M. (2001) Structural determinants of the substrate and stereochemical specificity of phosphotriesterase, Biochemistry 40, 1325-1331.
51. Caldwell, S. R., Newcomb, J. R., Schlecht, K. A., and Raushel, F. M. (1991) Limits of diffusion in the hydrolysis of substrates by the phosphotriesterase from Pseudomonas diminuta, Biochemistry 30, 7438-7444.
52. Hong, S. B., and Raushel, F. M. (1996) Metal-substrate interactions facilitate the catalytic activity of the bacterial phosphotriesterase, Biochemistry 35, 10904-10912.
53. Aubert, S. D., Li, Y., and Raushel, F. M. (2004) Mechanism for the hydrolysis of organophosphates by the bacterial phosphotriesterase, Biochemistry 43, 5707-5715.
54. Thoden, J. B., Phillips, G. N., Neal, T. M., Raushel, F. M., and Holden, H. M. (2001) Molecular Structure of Dihydroorotase: A paradigm for catalysis through the use of a binuclear metal center, Biochemistry 40, 6989-6997.
55. Caldwell, S. R., Raushel, F. M., Weiss, P. M., and Cleland, W. W. (1991) Transition-state structures for enzymatic and alkaline phosphotriester hydrolysis, Biochemistry 30, 7444-7450.
56. Vanhooke, J. L., Benning, M. M., Raushel, F. M., and Holden, H. M. (1996) Three-dimensional structure of the zinc-containing phosphotriesterase with the bound substrate analog diethyl 4-methylbenzylphosphonate, Biochemistry 35, 6020-6025.
57. Bigley, A. N., Mabanglo, M. F., Harvey, S. P., and Raushel, F. M. (2015) Variants of Phosphotriesterase for the Enhanced Detoxification of the Chemical Warfare Agent VR, Biochemistry 54, 5502-5512.
58. Tsai, P. C., Fox, N., Bigley, A. N., Harvey, S. P., Barondeau, D. P., and Raushel, F. M. (2012) Enzymes for the homeland defense: optimizing phosphotriesterase for the hydrolysis of organophosphate nerve agents, Biochemistry 51, 6463-6475.
59. Horne, I., Sutherland, T. D., Harcourt, R. L., Russell, R. J., and Oakeshott, J. G. (2002) Identification of an opd (organophosphate degradation) gene in an Agrobacterium isolate, Appl. Environ. Microbiol. 68, 3371-3376.
60. Nowlan, C., Li, Y., Hermann, J. C., Evans, T., Carpenter, J., Ghanem, E., Shoichet, B. K., and Raushel, F. M. (2006) Resolution of chiral phosphate, phosphonate, and phosphinate esters by an enantioselective enzyme library, J. Am. Chem. Soc. 128, 15892-15902.
61. Caldwell, S. R., Newcomb, J. R., Schlecht, K. A., and Raushel, F. M. (1991) Limits of diffusion in the hydrolysis of substrates by the phosphotriesterase from Pseudomonas diminuta, Biochemistry 30, 7438-7444.
62. Mee-Hie Cho, C., Mulchandani, A., and Chen, W. (2006) Functional analysis of organophosphorus hydrolase variants with high degradation activity towards organophosphate pesticides, PEDS 19, 99-105.
63. Schofield, D. A., and Dinovo, A. A. (2010) Generation of a mutagenized organophosphorus hydrolase for the biodegradation of the organophosphate pesticides malathion and demeton-S, J. Appl. Microbiol. 109, 548-557.
64. Scott, C. (2012) Landguard A900: An enzyme-based remdiant for the detoxification of organophosphate insecticides in animal dips, CSIRO Ecosystem Science, Canberra, Australia.
65. Tsai, P. C., Bigley, A., Li, Y., Ghanem, E., Cadieux, C. L., Kasten, S. A., Reeves, T. E., Cerasoli, D. M., and Raushel, F. M. (2010) Stereoselective hydrolysis of organophosphate nerve agents by the bacterial phosphotriesterase, Biochemistry 49, 7978-7987.
66. Benschop, H. P., and De Jong, L. P. A. (1988) Nerve agent stereoisomers: analysis, isolation and toxicology, Accout. Chem. Res. 21, 368-374.
67. Zhang, P., Liu, E. J., Tsao, C., Kasten, S. A., Boeri, M. V., Dao, T. L., DeBus, S. J., Cadieux, C. L., Baker, C. A., Otto, T. C., Cerasoli, D. M., Chen, Y., Jain, P., Sun, F., Li, W., Hung, H. C., Yuan, Z., Ma, J., Bigley, A. N., Raushel, F. M., and Jiang, S. (2019) Nanoscavenger provides long-term prophylactic protection against nerve agents in rodents, Sci. Transl. Med. 11.
68. Ordentlich, A., Barak, D., Sod-Moriah, G., Kaplan, D., Mizrahi, D., Segall, Y., Kronman, C., Karton, Y., Lazar, A., Marcus, D., Velan, B., and Shafferman, A. (2004) Stereoselectivity toward VX is determined by interactions with residues of the acyl pocket as well as of the peripheral anionic site of AChE, Biochemistry 43, 11255-11265.
69. Reeves, T. E., Wales, M. E., Grimsley, J. K., Li, P., Cerasoli, D. M., and Wild, J. R. (2008) Balancing the stability and the catalytic specificities of OP hydrolases with enhanced V-agent activities, PEDS 21, 405-412.
70. Bigley, A. N., Xu, C., Henderson, T. J., Harvey, S. P., and Raushel, F. M. (2013) Enzymatic neutralization of the chemical warfare agent VX: evolution of phosphotriesterase for phosphorothiolate hydrolysis, J. Am. Chem. Soc. 135, 10426-10432.
71. Cherny, I., Greisen, P., Jr., Ashani, Y., Khare, S. D., Oberdorfer, G., Leader, H., Baker, D., and Tawfik, D. S. (2013) Engineering V-type nerve agents detoxifying enzymes using computationally focused libraries, ACS Chem. Biol. 8, 2394-2403.
72. Goldsmith, M., Aggarwal, N., Ashani, Y., Jubran, H., Greisen, P. J., Ovchinnikov, S., Leader, H., Baker, D., Sussman, J. L., Goldenzweig, A., Fleishman, S. J., and Tawfik, D. S. (2017) Overcoming an optimization plateau in the directed evolution of highly efficient nerve agent bioscavengers, PEDS 30, 333-345.
73. Goldsmith, M., Eckstein, S., Ashani, Y., Greisen, P., Jr., Leader, H., Sussman, J. L., Aggarwal, N., Ovchinnikov, S., Tawfik, D. S., Baker, D., Thiermann, H., and Worek, F. (2016) Catalytic efficiencies of directly evolved phosphotriesterase variants with structurally different organophosphorus compounds in vitro, Arch. Toxicol. 90, 2711-2724.
74. Pettersen, E. F., Goddard, T. D., Huang, C. C., Couch, G. S., Greenblatt, D. M., Meng, E. C., and Ferrin, T. E. (2004) UCSF Chimera—a visualization system for exploratory research and analysis, J. Compd. Chem. 25, 1605-1612.
75. Vanhooke, J. L., Benning, M. M., Raushel, F. M., and Holden, H. M. (1996) Three-Dimensional Structure of the Zinc-Containing Phosphotriesterase with the Bound Substrate Analog Diethyl 4-Methylbenzylphosphonate, Biochemistry 35, 6020-6025.
76. Dong, Y., Yao, P., Cui, Y., Wu, Q., Zhu, D., Li, G., and Reetz, M. T. (2019) Manipulating the stereoselectivity of a thermostable alcohol dehydrogenase by directed evolution for efficient asymmetric synthesis of arylpropanols, Biol. Chem. 400, 313-321.
77. Jacquet, P., Hiblot, J., Daude, D., Bergonzi, C., Gotthard, G., Armstrong, N., Chabriere, E., and Elias, M. (2017) Rational engineering of a native hyperthermostable lactonase into a broad spectrum phosphotriesterase, Sci. Rep. 7, 16745.
78. Kaltenbach, M., Jackson, C. J., Campbell, E. C., Hollfelder, F., and Tokuriki, N. (2015) Reverse evolution leads to genotypic incompatibility despite functional and active site convergence, Elife 4, e06492.
79. Nobili, A., Gall, M. G., Pavlidis, I. V., Thompson, M. L., Schmidt, M., and Bornscheuer, U. T. (2013) Use of “small but smart’ libraries to enhance the enantioselectivity of an esterase from Bacillus stearothermophilus towards tetrahydrofuran-3-yl acetate, FEBS J. 280, 3084-3093.
80. Surmeli, Y., Ilgu, H., and Sanli-Mohamed, G. (2019) Improved activity of alpha-L-arabinofuranosidase from Geobacillus vulcani GS90 by directed evolution: Investigation on thermal and alkaline stability, Biotechnol. Appl. Biochem. 66, 101-107.
81. Yoshikuni, Y., Ferrin, T. E., and Keasling, J. D. (2006) Designed divergent evolution of enzyme function, Nature 440, 1078-1082.
82. Sun, Z., Wikmark, Y., Backvall, J. E., and Reetz, M. T. (2016) New Concepts for Increasing the Efficiency in Directed Evolution of Stereoselective Enzymes, Chemistry 22, 5046-5054.
83. Cheema, J., Faraldos, J. A., and O'Maille, P. E. (2017) REVIEW: Epistasis and dominance in the emergence of catalytic function as exemplified by the evolution of plant terpene synthases, Plant. Sci. 255, 29-38.
84. Harms, M. J., and Thornton, J. W. (2013) Evolutionary biochemistry: revealing the historical and physical causes of protein properties, Nat. Rev. Genet. 14, 559-571.
85. Sugrue, E., Scott, C., and Jackson, C. J. (2017) Constrained evolution of a bispecific enzyme: lessons for biocatalyst design, Org. Biomol. Chem. 15, 937-946.
86. Choi, S., Han, S., Lee, H., Chun, Y. J., and Kim, D. (2013) Evaluation of Luminescent P450 Analysis for Directed Evolution of Human CYP4A11, Biomol. Ther. (Seoul) 21, 487-492.
87. Christianson, D. W. (2006) Structural biology and chemistry of the terpenoid cyclases, Chem. Rev. 106, 3412-3442.
88. Moore, J. C., Rodriguez-Granillo, A., Crespo, A., Govindarajan, S., Welch, M., Hiraga, K., Lexa, K., Marshall, N., and Truppo, M. D. (2018) “Site and Mutation”-Specific Predictions Enable Minimal Directed Evolution Libraries, ACS Synth. Biol. 7, 1730-1741.
89. Ho, S. N., Hunt, H. D., Horton, R. M., Pullen, J. K., and Pease, L. R. (1989) Site-directed mutagenesis by overlap extension using the polymerase chain reaction, Gene 77, 51-59.
90. Bae, S. Y., and Winemiller, M. D. (2016) Separation of VX, RVX, and GB enantiomers using liquid chromatograph-time-of flight mass spectrometry, ECBC-TR-1341, (US Army Rearch, D., and Engineering Command Ed.), Edgewood Chemical Biological Center, Aberdeen Proving Grounds.
91. Shannon, P., Markiel, A., Ozier, O., Baliga, N. S., Wang, J. T., Ramage, D., Amin, N., Schwikowski, B., and Ideker, T. (2003) Cytoscape: a software environment for integrated models of biomolecular interaction networks, Genome. Res. 13, 2498-2504.
92. Cho, C. M., Mulchandani, A., and Chen, W. (2004) Altering the substrate specificity of organophosphorus hydrolase for enhanced hydrolysis of chlorpyrifos, Appl. Environ. Microbiol. 70, 4681-4685.

1. A synthetic amino acid sequence comprising mutations at one or more of positions 108, 132, 254, 257, 274, and 308 of VRN A80V/K185R/I274N) (SEQ ID NO: 26) and functioning by hydrolyzing an organophosphate nerve agent.

2. The synthetic amino acid sequence of claim 1 wherein the sequence is that of variant BHR-73-MNW, BHR-74, BHR-23, BHR-53, BHR-73, BHR-45, BHR-52, or BHR-75.

3. A synthetic DNA sequence encoding the synthetic amino acid sequence of claim 1.

4. A synthetic cDNA sequence comprising the coding sequence of the synthetic DNA sequence of claim 3.

5. A plasmid comprising the synthetic DNA sequence of claim 3.

6. A method of hydrolysis of an organophosphate nerve agent comprising contacting an organophosphate nerve agent with a synthetic DNA sequence encoding the synthetic amino acid sequence of claim 1.

7. The method of hydrolysis of claim 6, wherein the organophosphate is selected from the group consisting of paraoxon, S_{P}-VX, S_{P}-VR, DEVX, DMVX, R_{P}-OMVR, malathion, and ethoprophos.

8. A system for detoxifying an organophosphate nerve agent comprising contacting the synthetic amino acid sequence of claim 1 with an organophosphate nerve agent.

9. A kit for detoxifying an organophosphate nerve agent comprising the synthetic amino acid sequence of claim 1.

10. A method of producing variants of phosphotriesterase, wherein the variants are capable of detoxifying an organophosphate nerve agent, comprising the steps of:
obtaining a PTE gene;
inserting the PTE gene into a vector;
preparing a series of sequential mutational libraries wherein the PTE gene encodes a synthetic amino acid sequence of claim 1;
expressing the variant as a protein;
screening the variant for catalytic activity against one selected from the group consisting of paraoxon, S_{P}-VX, S_{P}-VR, DEVX, DMVX, R_{P}-OMVR, malathion, and ethoprophos to determine the hydrolytic activity; and
selecting the variant for use in hydrolysis of an organophosphate nerve agent based upon its hydrolytic activity.

11. The method of claim 10 wherein the variant synthetic amino acid sequence is at least 80% homogenous to the synthetic amino acid sequence of claim 1.

12. The method of hydrolysis of claim 10, wherein the organophosphate is VX.

13. The method of claim 12 wherein the hydrolysis is selective for the S_{P}-enantiomer of VX.

14. The method of hydrolysis of claim 10, wherein the organophosphate is VR.

15. The method of claim 14 wherein the hydrolysis is selective for the Sₚ-enantiomer of VR.


SEQUENCE LISTING (26 sequences)

SEQ ID NO: 1 - protein, 337 aa, Brevundimonas diminuta
MSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS

SEQ ID NO: 2 - protein, 337 aa, Brevundimonas diminuta
MSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWVDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDQIPFSAIGLEDNASASALLGIRSWQTRALLIKALIDQGYMKQILVSNDWLFGFSLYVTNIMDVMDRVNPDGMAFIPLRVIPFLREKGVPQETLAGITVTNPARFLSPTLRAS

SEQ ID NO: 3 - protein, 337 aa, Brevundimonas diminuta
MSIGTGDRINTVRGPITISEAGFTLTHEHICGSSAGFLRAWPEFFGSRKALAEKAVRGLRRARAAGVRTIVDVSTFDCGRDVSLLAEVSRAADVHIVAATGLWVDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDQIPFSAIGLEDNASASALLGIRSWQTRALLIKALIDQGYMKQILVSNDWLFGFSLYVTNIMDVMDRVNPDGMAFIPLRVIPFLREKGVPQETLAGITVTNPARFLSPTLRAS

SEQ ID NO: 4 - protein, 337 aa, Brevundimonas diminuta
MSIGTGDRINTVRGPITISEAGFTLTHEHICGSSAGFLRAWPEFFGSRKALVEKAVRGLRRARAAGVRTIVDVSTFDIGRDVSLLAEVSRAADVHIVAATGLWVDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDQIPFSAIGLEDNASASALLGNRSWQTRALLIKALIDQGYMKQILVSNDWLFGFSLYVTNIMDVMDRVNPDGMAFIPLRVIPFLREKGVPQETLAGITVTNPARFLSPTLRAS

SEQ ID NO: 5 - protein, 337 aa, Brevundimonas diminuta
  ref <301>: AUTHORS: Ping-Chuan Tsai, Nicholas Fox, Andrew N. Bigley, Steven P. Harvey, David P. Barondeau, and Frank M. Raushel
  ref <302>: TITLE: Enzymes for the Homeland Defense: Optimizing Phosphotriesterase for the Hydrolysis of Organophosphate Nerve Agents
  ref <303>: JOURNAL: Biochemistry
  ref <304>: VOLUME: 51
  ref <306>: PAGES: 6463-6475
  ref <307>: DATE: 2012-07-18
  ref <301>: AUTHORS: Andrew N. Bigley, Chengfu Xu, Terry J. Henderson, Steven P. Harvey, and Frank M. Raushel
  ref <302>: TITLE: Enzymatic Neutralization of the Chemical Warfare Agent VX: Evolution of Phosphotriesterase for Phosphorothiolate Hydrolysis
  ref <303>: JOURNAL: Journal of the American Chemical Society
  ref <304>: VOLUME: 135
  ref <306>: PAGES: 10426-10432
  ref <307>: DATE: 2013-06-21
MSIGTGDRINTVRGPITISEAGFTLTHEHICGSSAGFLRAWPEFFGSRKALAEKAVRGLRRARAAGVRTIVDVSTFDCGRDVSLLAEVSRAADVHIVAATGLWVDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDQIPYSAIGLEDNASASVLMGNRSWQTRALLIKALIDQGYMKQILVSNDWLFGFSLYVTNIMDVMDRVNPDGMAFIPLRVIPFLREKGVPQETLAGITVTNPARFLSPTLRAS

SEQ ID NO: 6 - protein, 337 aa, Brevundimonas diminuta
MSIGTGDRINTVRGPITISEAGFTLTHEHICGSSAGFLRAWPEFFGSRKALAEKAVRGLRRARAAGVRTIVDVSTFDGGRDVSLLAEVSRAADVHIVAATGLWVDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDQIPYSAIGLEDNASASVLMGNRSWQTRALLIKALIDQGYMKQILVSNDWLFGFSLYVTNIMDVMDRVNPDGMAFIPLRVIPFLREKGVPQETLAGITVTNPARFLSPTLRAS

SEQ ID NO: 7 - protein, 337 aa, Brevundimonas diminuata
MSIGTGDRINTVRGPITISEAGFTLTHEHICGSSAGFLRAWPEFFGSRKALVEKAVRGLRRARAAGVRTIVDVSTFDGGRDVSLLAEVSRAADVHIVAATGLWVDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDQIPFSAIGLEDNASASALLGNRSWQTRALLIKALIDQGYMKQILVSNDWLFGFSLYVTNIMDVMDRVNPDGMAFIPLRVIPFLREKGVPQETLAGITVTNPARFLSPTLRAS

SEQ ID NO: 8 - protein, 337 aa, Brevundimonas diminuata
MSIGTGDRINTVRGPITISEAGFTLTHEHICGSSAGFLRAWPEFFGSRKALVEKAVRGLRRARAAGVRTIVDVSTFDIGRDVSLLAEVSRAADVHIVAATGLWVDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDQIPFSAIGLEDNASASALLGNRSWQTRALLIKALIDQGYMKQILVSNDWLFGFSSYVTNIMDVMDRVNPDGMAFIPLRVIPFLREKGVPQETLAGITVTNPARFLSPTLRAS

SEQ ID NO: 9 - protein, 337 aa, Brevundimonas diminuata
MSIGTGDRINTVRGPITISEAGFTLTHEHICGSSAGFLRAWPEFFGSRKALVEKAVRGLRRARAAGVRTIVDVSTFDGGRDVSLLAEVSRAADVHIVAATGLWVDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDQIPFSAIGLEDNASASALLGNRSWQTRALLIKALIDQGYMKQILVSNDWLFGFSSYVTNIMDVMDRVNPDGMAFIPLRVIPFLREKGVPQETLAGITVTNPARFLSPTLRAS

SEQ ID NO: 10 - DNA, 33 bp, Artificial Sequence
  Other DNA
  misc_feature: B=G or C or T; S=G or C
  misc_feature: n=G or C or T; n=G or C
  misc_feature (17)..(18): n is a, c, g, or t
gtttccaccttcgacgnnggccgtgacgtttcc

SEQ ID NO: 11 - DNA, 33 bp, Artificial Sequence
  Other DNA
  misc_feature: S=G or C
  misc_feature: n=G or C
  misc_feature (17)..(17): n is a, c, g, or t
gtttccaccttcgactncggccgtgacgtttcc

SEQ ID NO: 12 - DNA, 33 bp, Artificial Sequence
  Other DNA
gtttccaccttcgacatcggccgtgacgtttcc

SEQ ID NO: 13 - DNA, 33 bp, Artificial Sequence
  Other DNA
  misc_feature: H=A or T or C; W=A or T
  misc_feature: n=A or T or C; n=A or T
  misc_feature (17)..(18): n is a, c, g, or t
gctaccggtctgtgggnngacccgccgctgagc

SEQ ID NO: 14 - DNA, 33 bp, Artificial Sequence
  Other DNA
  misc_feature: n=G or T; n =G or T
  misc_feature (17)..(18): n is a, c, g, or t
gctaccggtctgtggtnngacccgccgctgagc

SEQ ID NO: 15 - DNA, 36 bp, Artificial Sequence
  Other DNA
  misc_feature: n=A or G; n=A or C
  misc_feature (17)..(18): n is a, c, g, or t
ctgatcggtctggaccnnatcccgcactccgctatc

SEQ ID NO: 16 - DNA, 36 bp, Artificial Sequence
  Other DNA
  misc_feature: n=A or G
  misc_feature (17)..(17): n is a, c, g, or t
ctgatcggtctggacancatcccgcactccgctatc

SEQ ID NO: 17 - DNA, 36 bp, Artificial Sequence
  Other DNA
ctgatcggtctggacggcatcccgcactccgctatc

SEQ ID NO: 18 - DNA, 38 bp, Artificial Sequence
  Other DNA
  misc_feature: n=A or G; n=A or C; n=G or T; n=G or T
  misc_feature (15)..(16): n is a, c, g, or t
  misc_feature (24)..(25): n is a, c, g, or t
gatcggtctggaccnnatcccgtnntccgctatcggtc

SEQ ID NO: 19 - DNA, 38 bp, Artificial Sequence
  Other DNA
  misc_feature: n=A or G; n=G or T; n=G or T
  misc_feature (15)..(15): n is a, c, g, or t
  misc_feature (24)..(25): n is a, c, g, or t
gatcggtctggacancatcccgtnntccgctatcggtc

SEQ ID NO: 20 - DNA, 38 bp, Artificial Sequence
  Other DNA
  misc_feature: n=G or T; n=G or T
  misc_feature (24)..(25): n is a, c, g, or t
gatcggtctggacggcatcccgtnntccgctatcggtc

SEQ ID NO: 21 - DNA, 33 bp, Artificial Sequence
  Other DNA
  misc_feature: n=A or G or C or T
  misc_feature (16)..(16): n is a, c, g, or t
ccgctctgctgggtanccgttcctggcagaccc

SEQ ID NO: 22 - DNA, 33 bp, Artificial Sequence
  Other DNA
ccgctctgctgggtcagcgttcctggcagaccc

SEQ ID NO: 23 - DNA, 33 bp, Artificial Sequence
  Other DNA
  misc_feature: n=A or G
  misc_feature (16)..(16): n is a, c, g, or t
ctgttcggtttctcgntttacgttaccaacatc

SEQ ID NO: 24 - DNA, 33 bp, Artificial Sequence
  Other DNA
ctgttcggtttctcgagctacgttaccaacatc

SEQ ID NO: 25 - DNA, 33 bp, Artificial Sequence
  Other DNA
ctgttcggtttctcgctgtacgttaccaacatc

SEQ ID NO: 26 - protein, 337 aa, Brevundimonas diminuata
MSIGTGDRINTVRGPITISEAGFTLTHEHICGSSAGFLRAWPEFFGSRKALVEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDHIPHSAIGLEDNASASALLGNRSWQTRALLIKALIDQGYMKQILVSNDWLFGFSSYVTNIMDVMDRVNPDGMAFIPLRVIPFLREKGVPQETLAGITVTNPARFLSPTLRAS